(19) United States
(12) Patent Application Publication
(10) Pub. No.: US 2026/0226491 A1
(43) Pub. Date: Aug. 6, 2026

(54) MODIFIED TOBACCO PLANTS WITH ALTERED TRICHOME CHARACTERISTICS AND RELATED METHODS

(71) Applicant: University of Virginia Patent Foundation, Charlottesville, VA (US)

(72) Inventors: Michael Paul Timko, Charlottesville, VA (US); Hai Liu, Charlottesville, VA (US)

(73) Assignee: University of Virginia Patent Foundation, Charlottesville, VA (US)

(21) Appl. No.: 19/440,077

(22) Filed: Jan. 5, 2026

Related U.S. Application Data
(60) Provisional application No. 63/742,053, filed on Jan. 6, 2025.
(60) Provisional application No. 63/880,719, filed on Sep. 12, 2025.

(51) Int. Cl. C12N 15/82; C07K 14/415

(52) U.S. Cl. CPC C12N 15/8263; C07K 14/415

(57) ABSTRACT

The present disclosure provides compositions and methods related to improving trichome characteristics in tobacco plants. Plants comprising non-natural mutations in genes involved in trichome growth are provided, as are products and cured tobacco material made from said plants. Plants comprising RNAi constructs against genes involved in trichome growth are provided, as are products and cured tobacco material made from said plants. Methods of making and using the provided plants are also provided.

### CROSS-REFERENCE TO RELATED APPLICATIONS

This application claims the benefit of U.S. Provisional Application No. 63/742,053, Jan. 6, 2025, and U.S. Provisional Application No. 63/880,719, filed Sep. 12, 2025, both of which are incorporated by reference in their entireties herein.

### INCORPORATION OF SEQUENCE LISTING

A sequence listing contained in the file named “P35327US02_SL.xml” which is 1,392,960 bytes (measured in operating system MS-Windows®), produced on Jan. 2, 2026, containing a total number of 612 SEQ ID NOs, starting from SEQ ID NO: 1 to SEQ ID NO: 612, is filed electronically herewith and incorporated by reference in its entirety.

### FIELD

The present disclosure relates to tobacco plants comprising improved trichome characteristics and related compounds, including, but not limited to, cured tobacco material and products made from said tobacco plants. The present disclosure also relates to methods of improving trichome characteristics in tobacco plants.

A brief description of nucleic acid sequences and amino acid sequences referenced in this disclosure is provided in Table 1.

TABLE 1
Nucleic acid sequences and amino acid sequences.

| SEQ | | |
|---|---|---|
| ID | | |
| NO | Type | Description |
| 1 | Amino acid | NtHAP1a |
| 2 | Amino acid | NtHAP1b |
| 3 | Amino acid | NtHAP2 |
| 4 | Nucleic acid | NtHAP1a |
| | (coding) | |
| 5 | Nucleic acid | NtHAP1b |
| | (coding) | |
| 6 | Nucleic acid | NtHAP2 |
| | (coding) | |
| 7 | Nucleic acid | NtHAP1a |
| | (genomic) | |
| 8 | Nucleic acid | NtHAP1b |
| | (genomic) | |
| 9 | Nucleic acid | NtHAP2 |
| | (genomic) | |
| 10 | Nucleic acid | gRNA1 |
| | (RNA) | |
| 11 | Nucleic acid | gRNA2 |
| | (RNA) | |
| 12 | Nucleic acid | gRNA3 |
| | (RNA) | |
| 13 | Nucleic acid | gRNA4 |
| | (RNA) | |
| 14 | Nucleic acid | gRNA5 |
| | (RNA) | |
| 15 | Nucleic acid | gRNA6 |
| | (RNA) | |
| 16 | Nucleic acid | gRNA7 |
| | (RNA) | |
| 17 | Nucleic acid | gRNA8 |
| | (RNA) | |
| 18 | Nucleic Acid | HAP1a/b target |
| | | site for RNAi |
| 19 | Nucleic Acid | HAP2 target site |
| | | for RNAi |
| 20 | Nucleic Acid | EMS mutant |
| | | NtHapla |
| 21 | Nucleic Acid | EMS mutant |
| | | NtHapla |
| 22 | Nucleic Acid | EMS mutant |
| | | NtHapla |
| 23 | Nucleic Acid | EMS mutant |
| | | NtHapla |
| 24 | Nucleic Acid | EMS mutant |
| | | NtHap1b |
| 25 | Nucleic Acid | EMS mutant |
| | | NtHap1b |
| 26 | Nucleic Acid | EMS mutant |
| | | NtHap2 |
| 27 | Amino Acid | EMS mutant |
| | | NtHapla |
| 28 | Amino Acid | EMS mutant |
| | | NtHapla |
| 29 | Amino Acid | EMS mutant |
| | | NtHapla |
| 30 | Amino Acid | EMS mutant |
| | | NtHapla |
| 31 | Amino Acid | EMS mutant |
| | | NtHap1b |
| 32 | Amino Acid | EMS mutant |
| | | NtHap1b |
| 33 | Amino Acid | EMS mutant |
| | | NtHap2 |
| 34 | Nucleic Acid | NtHAP1a mutant |
| 35 | Nucleic Acid | NtHAP1a mutant |
| 36 | Nucleic Acid | NtHAP1b mutant |
| 37 | Nucleic Acid | NtHAP1a mutant |
| 38 | Nucleic Acid | NtHAP1b mutant |
| 39 | Nucleic Acid | NtHAP1b mutant |
| 40 | Nucleic Acid | NtHAP1a mutant |
| 41 | Nucleic Acid | NtHAP1b mutant |
| 42 | Nucleic Acid | NtHAP1a mutant |
| 43 | Nucleic Acid | NtHAP1b mutant |
| 44 | Nucleic Acid | NtHAP1a mutant |
| 45 | Nucleic Acid | NtHAP1b mutant |
| 46 | Nucleic Acid | NtHAP1a mutant |
| 47 | Nucleic Acid | NtHAP1a mutant |
| 48 | Nucleic Acid | NtHAP1b mutant |
| 49 | Nucleic Acid | NtHAP1a mutant |
| 50 | Nucleic Acid | NtHAP1a mutant |
| 51 | Nucleic Acid | NtHAP2 mutant |
| 52 | Nucleic Acid | NtHAP2 mutant |
| 53 | Nucleic Acid | NtHAP1a mutant |
| 54 | Nucleic Acid | NtHAP1b mutant |
| 55 | Nucleic Acid | NtHAP1b mutant |
| 56 | Nucleic Acid | NtHAP2 mutant |
| 57 | Nucleic Acid | NtHAP1a mutant |
| 58 | Nucleic Acid | NtHAP1a mutant |
| 59 | Nucleic Acid | NtHAP2 mutant |
| 60 | Nucleic Acid | NtHAP2 mutant |
| 61 | Nucleic Acid | NtHAP1a mutant |
| 62 | Nucleic Acid | NtHAP1a mutant |
| 63 | Nucleic Acid | NtHAP1a mutant |
| 64 | Nucleic Acid | NtHAP1a mutant |
| 65 | Nucleic Acid | NtHAP1a mutant |
| 66 | Nucleic Acid | NtHAP1a mutant |
| 67 | Nucleic Acid | NtHAP1a mutant |
| 68 | Nucleic Acid | NtHAP1a mutant |
| 69 | Nucleic Acid | NtHAP1a mutant |
| 70 | Nucleic Acid | NtHAP1a mutant |
| 71 | Nucleic Acid | NtHAP1a mutant |
| 72 | Nucleic Acid | NtHAP1a mutant |
| | | promoter |
| 73 | Nucleic Acid | NtHAP1b mutant |
| | | promoter |
| 74 | Nucleic Acid | NtHAP1b mutant |
| | | promoter |
| 75 | Nucleic Acid | NtHAP1a mutant |
| | | promoter |
| 76 | Nucleic Acid | NtHAP1b mutant |
| | | promoter |
| 77 | Nucleic Acid | NtHAP1a mutant |
| | | promoter |
| 78 | Nucleic Acid | NtHAP1b mutant |
| | | promoter |
| 79 | Nucleic Acid | NtHAP1a mutant |
| | | promoter |
| 80 | Nucleic Acid | NtHAP1a mutant |
| | | promoter |
| 81 | Nucleic Acid | NtHAP1b mutant |
| | | promoter |
| 82 | Nucleic Acid | NtHAP1b mutant |
| | | promoter |
| 83 | Nucleic Acid | NtHAP1a mutant |
| | | promoter |
| 84 | Nucleic Acid | NtHAP1b mutant |
| | | promoter |
| 85 | Nucleic Acid | NtHAP1b mutant |
| | | promoter |
| 86 | Nucleic Acid | NtHAP1a mutant |
| | | promoter |
| 87 | Nucleic Acid | NtHAP1a mutant |
| | | promoter |
| 88 | Nucleic Acid | NtHAP1b mutant |
| | | promoter |
| 89 | Nucleic Acid | NtHAP1b mutant |
| | | promoter |
| 90 | Nucleic Acid | NtHAP1b |
| | | deletion fragment |
| 91 | Nucleic Acid | NtHAP1b |
| | | deletion fragment |
| 92 | Nucleic Acid | NtHAP1b |
| | | deletion fragment |
| 93 | Nucleic Acid | NtHAP1a |
| | | deletion fragment |
| 94 | Nucleic Acid | NtHAP1a |
| | | deletion fragment |
| 95 | Nucleic Acid | NtHAP1a |
| | | deletion fragment |
| 96 | Nucleic Acid | NtHAP1a |
| | | insertion fragment |
| 97 | Amino Acid | NtHAP1a mutant |
| 98 | Amino Acid | NtHAP1a mutant |
| 99 | Amino Acid | NtHAP1b mutant |
| 100 | Amino Acid | NtHAP1a mutant |
| 101 | Amino Acid | NtHAP1b mutant |
| 102 | Amino Acid | NtHAP1b mutant |
| 103 | Amino Acid | NtHAP1a mutant |
| 104 | Amino Acid | NtHAP1b mutant |
| 105 | Amino Acid | NtHAP1a mutant |
| 106 | Amino Acid | NtHAP1b mutant |
| 107 | Amino Acid | NtHAP1a mutant |
| 108 | Amino Acid | NtHAP1b mutant |
| 109 | Amino Acid | NtHAP1a mutant |
| 110 | Amino Acid | NtHAP1a mutant |
| 111 | Amino Acid | NtHAP1b mutant |
| 112 | Amino Acid | NtHAP1a mutant |
| 113 | Amino Acid | NtHAP1a mutant |
| 114 | Amino Acid | NtHAP2 mutant |
| 115 | Amino Acid | NtHAP2 mutant |
| 116 | Amino Acid | NtHAP1a mutant |
| 117 | Amino Acid | NtHAP1b mutant |
| 118 | Amino Acid | NtHAP1b mutant |
| 119 | Amino Acid | NtHAP2 mutant |
| 120 | Amino Acid | NtHAP1a mutant |
| 121 | Amino Acid | NtHAP1a mutant |
| 122 | Amino Acid | NtHAP2 mutant |
| 123 | Amino Acid | NtHAP2 mutant |
| 124 | Amino Acid | NtHAP1a mutant |
| 125 | Amino Acid | NtHAP1a mutant |
| 126 | Amino Acid | NtHAP1a mutant |
| 127 | Amino Acid | NtHAP1a mutant |
| 128 | Amino Acid | NtHAP1a mutant |
| 129 | Amino Acid | NtHAP1a mutant |
| 130 | Amino Acid | NtHAP1a mutant |
| 131 | Amino Acid | NtHAP1a mutant |
| 132 | Amino Acid | NtHAP1a mutant |
| 133 | Amino Acid | NtHAP1a mutant |
| 134 | Amino Acid | NtHAP1a mutant |
| 135 | Nucleic Acid | NtHAP1a mutant |
| 136 | Nucleic Acid | NtHAP1a mutant |
| 137 | Nucleic Acid | NtHAP1a mutant |
| 138 | Nucleic Acid | NtHAP1b mutant |
| 139 | Nucleic Acid | NtHAP1b mutant |
| 140 | Nucleic Acid | NtHAP1a mutant |
| 141 | Nucleic Acid | NtHAP1a mutant |
| 142 | Nucleic Acid | NtHAP1a mutant |
| 143 | Nucleic Acid | NtHAP1b mutant |
| 144 | Nucleic Acid | NtHAP1a mutant |
| 145 | Nucleic Acid | NtHAP1a mutant |
| 146 | Nucleic Acid | NtHAP1b mutant |
| 147 | Nucleic Acid | NtHAP1b mutant |
| 148 | Nucleic Acid | NtHAP1a mutant |
| 149 | Nucleic Acid | NtHAP1a mutant |
| 150 | Nucleic Acid | NtHAP1b mutant |
| 151 | Nucleic Acid | NtHAP1b mutant |
| 152 | Nucleic Acid | NtHAP1a mutant |
| 153 | Nucleic Acid | NtHAP1a mutant |
| 154 | Nucleic Acid | NtHAP1b mutant |
| 155 | Nucleic Acid | NtHAP1b mutant |
| 156 | Nucleic Acid | NtHAP1a mutant |
| 157 | Nucleic Acid | NtHAP1a mutant |
| 158 | Nucleic Acid | NtHAP1b mutant |
| 159 | Nucleic Acid | NtHAP1b mutant |
| 160 | Nucleic Acid | NtHAP1a mutant |
| 161 | Nucleic Acid | NtHAP1a mutant |
| 162 | Nucleic Acid | NtHAP1b mutant |
| 163 | Nucleic Acid | NtHAP1b mutant |
| 164 | Nucleic Acid | NtHAP1a mutant |
| 165 | Nucleic Acid | NtHAP1b mutant |
| 166 | Nucleic Acid | NtHAP1a mutant |
| 167 | Nucleic Acid | NtHAP1a mutant |
| 168 | Nucleic Acid | NtHAP1b mutant |
| 169 | Nucleic Acid | NtHAP1a mutant |
| 170 | Nucleic Acid | NtHAP1a mutant |
| 171 | Nucleic Acid | NtHAP1b mutant |
| 172 | Nucleic Acid | NtHAP1b mutant |
| 173 | Nucleic Acid | NtHAP1a mutant |
| 174 | Nucleic Acid | NtHAP1a mutant |
| 175 | Nucleic Acid | NtHAP1b mutant |
| 176 | Nucleic Acid | NtHAP1b mutant |
| 177 | Nucleic Acid | NtHAP1a mutant |
| 178 | Nucleic Acid | NtHAP1a mutant |
| 179 | Nucleic Acid | NtHAP1b mutant |
| 180 | Nucleic Acid | NtHAP1b mutant |
| 181 | Nucleic Acid | NtHAP1b mutant |
| 182 | Nucleic Acid | NtHAP2 mutant |
| 183 | Nucleic Acid | NtHAP2 mutant |
| 184 | Nucleic Acid | NtHAP1a mutant |
| 185 | Nucleic Acid | NtHAP1b mutant |
| 186 | Nucleic Acid | NtHAP1b mutant |
| 187 | Nucleic Acid | NtHAP2 mutant |
| 188 | Nucleic Acid | NtHAP2 mutant |
| 189 | Nucleic Acid | NtHAP2 mutant |
| 190 | Nucleic Acid | NtHAP2 mutant |
| 191 | Nucleic Acid | NtHAP1b mutant |
| 192 | Nucleic Acid | NtHAP2 mutant |
| 193 | Nucleic Acid | NtHAP2 mutant |
| 194 | Nucleic Acid | NtHAP1b mutant |
| 195 | Nucleic Acid | NtHAP1b mutant |
| 196 | Nucleic Acid | NtHAP2 mutant |
| 197 | Nucleic Acid | NtHAP2 mutant |
| 198 | Nucleic Acid | NtHAP1a mutant |
| 199 | Nucleic Acid | NtHAP1b mutant |
| 200 | Nucleic Acid | NtHAP2 mutant |
| 201 | Nucleic Acid | NtHAP2 mutant |
| 202 | Nucleic Acid | NtHAP1a mutant |
| 203 | Nucleic Acid | NtHAP1b mutant |
| 204 | Nucleic Acid | NtHAP2 mutant |
| 205 | Nucleic Acid | NtHAP1b mutant |
| 206 | Nucleic Acid | NtHAP2 mutant |
| 207 | Nucleic Acid | NtHAP1a mutant |
| 208 | Nucleic Acid | NtHAP2 mutant |
| 209 | Nucleic Acid | NtHAP1a mutant |
| 210 | Nucleic Acid | NtHAP1b mutant |
| 211 | Nucleic Acid | NtHAP2 mutant |
| 212 | Nucleic Acid | NtHAP2 mutant |
| 213 | Nucleic Acid | NtHAP2 mutant |
| 214 | Nucleic Acid | NtHAP2 mutant |
| 215 | Nucleic Acid | NtHAP1a mutant |
| 216 | Nucleic Acid | NtHAP1a mutant |
| 217 | Nucleic Acid | NtHAP2 mutant |
| 218 | Nucleic Acid | NtHAP1b mutant |
| 219 | Nucleic Acid | NtHAP2 mutant |
| 220 | Nucleic Acid | NtHAP2 mutant |
| 221 | Nucleic Acid | NtHAP1a mutant |
| 222 | Nucleic Acid | NtHAP1a mutant |
| 223 | Nucleic Acid | NtHAP1a mutant |
| 224 | Nucleic Acid | NtHAP1a mutant |
| 225 | Nucleic Acid | NtHAP1a mutant |
| 226 | Nucleic Acid | NtHAP1a mutant |
| 227 | Nucleic Acid | NtHAP1a mutant |
| 228 | Nucleic Acid | NtHAP1a mutant |
| 229 | Nucleic Acid | NtHAP1a mutant |
| 230 | Nucleic Acid | NtHAP1a mutant |
| 231 | Nucleic Acid | NtHAP1a mutant |
| 232 | Nucleic Acid | NtHAP1a mutant |
| 233 | Nucleic Acid | NtHAP1a mutant |
| 234 | Nucleic Acid | NtHAP1a mutant |
| 235 | Nucleic Acid | NtHAP1a mutant |
| 236 | Nucleic Acid | NtHAP1a mutant |
| 237 | Nucleic Acid | NtHAP1a mutant |
| 238 | Nucleic Acid | NtHAP1a mutant |
| 239 | Nucleic Acid | NtHAP1a mutant |
| 240 | Nucleic Acid | NtHAP1a mutant |
| 241 | Nucleic Acid | NtHAP1a mutant |
| 242 | Nucleic Acid | NtHAP1a mutant |
| 243 | Nucleic Acid | NtHAP1a mutant |
| 244 | Nucleic Acid | NtHAP1a mutant |
| 245 | Nucleic Acid | NtHAP1a mutant |
| 246 | Nucleic Acid | NtHAP1a mutant |
| 247 | Nucleic Acid | NtHAP1a mutant |
| 248 | Nucleic Acid | NtHAP1a mutant |
| 249 | Nucleic Acid | NtHAP1a mutant |
| 250 | Nucleic Acid | NtHAP1a mutant |
| 251 | Nucleic Acid | NtHAP1a mutant |
| 252 | Nucleic Acid | NtHAP1a mutant |
| 253 | Nucleic Acid | NtHAP1a mutant |
| 254 | Nucleic Acid | NtHAP1a mutant |
| 255 | Nucleic Acid | NtHAP1a mutant |
| 256 | Nucleic Acid | NtHAP1a mutant |
| 257 | Nucleic Acid | NtHAP1a mutant |
| 258 | Nucleic Acid | NtHAP1a mutant |
| 259 | Nucleic Acid | NtHAP1a mutant |
| 260 | Nucleic Acid | NtHAP1a mutant |
| 261 | Nucleic Acid | NtHAP1a mutant |
| 262 | Nucleic Acid | NtHAP1a mutant |
| 263 | Nucleic Acid | NtHAP1a mutant |
| 264 | Nucleic Acid | NtHAP1a mutant |
| 265 | Nucleic Acid | NtHAP1a mutant |
| 266 | Nucleic Acid | NtHAP1a mutant |
| 267 | Nucleic Acid | NtHAP1a mutant |
| 268 | Nucleic Acid | NtHAP1a mutant |
| 269 | Nucleic Acid | NtHAP1a mutant |
| 270 | Nucleic Acid | NtHAP1b mutant |
| 271 | Nucleic Acid | NtHAP1b mutant |
| 272 | Nucleic Acid | NtHAP1b mutant |
| 273 | Nucleic Acid | NtHAP1b mutant |
| 274 | Nucleic Acid | NtHAP1b mutant |
| 275 | Nucleic Acid | NtHAP1b mutant |
| 276 | Nucleic Acid | NtHAP1b mutant |
| 277 | Nucleic Acid | NtHAP1b mutant |
| 278 | Nucleic Acid | NtHAP2 mutant |
| 279 | Nucleic Acid | NtHAP2 mutant |
| 280 | Nucleic Acid | NtHAP2 mutant |
| 281 | Nucleic Acid | NtHAP2 mutant |
| 282 | Nucleic Acid | NtHAP2 mutant |
| 283 | Nucleic Acid | NtHAP2 mutant |
| 284 | Nucleic Acid | NtHAP2 mutant |
| 285 | Nucleic Acid | NtHAP1b |
| | | promoter |
| 286 | Nucleic Acid | NtHAP1a |
| | | promoter |
| 287 | Nucleic Acid | NtHAP1a |
| | | promoter |
| 288 | Nucleic Acid | NtHAP1b |
| | | promoter |
| 289 | Nucleic Acid | NtHAP1b |
| | | promoter |
| 290 | Nucleic Acid | NtHAP1a |
| | | promoter |
| 291 | Nucleic Acid | NtHAP1a |
| | | promoter |
| 292 | Nucleic Acid | NtHAP1b |
| | | promoter |
| 293 | Nucleic Acid | NtHAP1b |
| | | promoter |
| 294 | Nucleic Acid | NtHAP1a |
| | | promoter |
| 295 | Nucleic Acid | NtHAP1a |
| | | promoter |
| 296 | Nucleic Acid | NtHAP1b |
| | | promoter |
| 297 | Nucleic Acid | NtHAP1a |
| | | promoter |
| 298 | Nucleic Acid | NtHAP1a |
| | | promoter |
| 299 | Nucleic Acid | NtHAP1a |
| | | promoter |
| 300 | Nucleic Acid | NtHAP1b |
| | | promoter |
| 301 | Nucleic Acid | NtHAP1a |
| | | promoter |
| 302 | Nucleic Acid | NtHAP1b |
| | | promoter |
| 303 | Nucleic Acid | NtHAP1a |
| | | promoter |
| 304 | Nucleic Acid | NtHAP1a |
| | | promoter |
| 305 | Nucleic Acid | NtHAP1b |
| | | promoter |
| 306 | Nucleic Acid | NtHAP1b |
| | | promoter |
| 307 | Nucleic Acid | NtHAP1a |
| | | promoter |
| 308 | Nucleic Acid | NtHAP1a |
| | | promoter |
| 309 | Nucleic Acid | NtHAP1a |
| | | promoter |
| 310 | Nucleic Acid | NtHAP1a |
| | | promoter |
| 311 | Nucleic Acid | NtHAP1b |
| | | promoter |
| 312 | Nucleic Acid | NtHAP1b |
| | | promoter |
| 313 | Nucleic Acid | NtHAP1a |
| | | promoter |
| 314 | Nucleic Acid | NtHAP1b |
| | | promoter |
| 315 | Nucleic Acid | NtHAP1b |
| | | promoter |
| 316 | Nucleic Acid | NtHAP1a |
| | | promoter |
| 317 | Nucleic Acid | NtHAP1a |
| | | promoter |
| 318 | Nucleic Acid | NtHAP1a |
| | | promoter |
| 319 | Nucleic Acid | NtHAP1b |
| | | promoter |
| 320 | Nucleic Acid | NtHAP1b |
| | | promoter |
| 321 | Nucleic Acid | NtHAP2 |
| | | promoter |
| 322 | Nucleic Acid | NtHAP2 |
| | | promoter |
| 323 | Nucleic Acid | NtHAP1b |
| | | promoter |
| 324 | Nucleic Acid | NtHAP1b |
| | | promoter |
| 325 | Nucleic Acid | NtHAP1b |
| | | promoter |
| 326 | Nucleic Acid | NtHAP1a |
| | | promoter |
| 327 | Nucleic Acid | NtHAP1a |
| | | promoter |
| 328 | Nucleic Acid | NtHAP1b |
| | | promoter |
| 329 | Nucleic Acid | NtHAP1a |
| | | promoter |
| 330 | Nucleic Acid | NtHAP1b |
| | | promoter |
| 331 | Nucleic Acid | NtHAP1a |
| | | promoter |
| 332 | Nucleic Acid | NtHAP1b |
| | | promoter |
| 333 | Nucleic Acid | NtHAP1b |
| | | promoter |
| 334 | Nucleic Acid | NtHAP1b |
| | | promoter |
| 335 | Nucleic Acid | NtHAP1b |
| | | promoter |
| 336 | Nucleic Acid | NtHAP1a |
| | | promoter |
| 337 | Nucleic Acid | NtHAP1b |
| | | promoter |
| 338 | Nucleic Acid | NtHAP1b |
| | | promoter |
| 339 | Nucleic Acid | NtHAP1a |
| | | promoter |
| 340 | Nucleic Acid | NtHAP1b |
| | | promoter |
| 341 | Nucleic Acid | NtHAP1a |
| | | promoter |
| 342 | Nucleic Acid | NtHAP1b |
| | | promoter |
| 343 | Nucleic Acid | NtHAP1a |
| | | promoter |
| 344 | Nucleic Acid | NtHAP1b |
| | | promoter |
| 345 | Nucleic Acid | NtHAP1a |
| | | promoter |
| 346 | Nucleic Acid | NtHAP1a |
| | | promoter |
| 347 | Nucleic Acid | NtHAP1b |
| | | promoter |
| 348 | Nucleic Acid | NtHAP1b |
| | | promoter |
| 349 | Nucleic Acid | NtHAP1a |
| | | promoter |
| 350 | Nucleic Acid | NtHAP1a |
| | | promoter |
| 351 | Nucleic Acid | NtHAP1b |
| | | promoter |
| 352 | Nucleic Acid | NtHAP1a |
| | | deletion fragment |
| 353 | Nucleic Acid | NtHAP1b |
| | | deletion fragment |
| 354 | Nucleic Acid | NtHAP1b |
| | | deletion fragment |
| 355 | Nucleic Acid | NtHAP1a |
| | | deletion fragment |
| 356 | Nucleic Acid | NtHAP1a |
| | | deletion fragment |
| 357 | Nucleic Acid | NtHAP1a |
| | | deletion fragment |
| 358 | Nucleic Acid | NtHAP1b |
| | | deletion fragment |
| 359 | Nucleic Acid | NtHAP1a |
| | | deletion fragment |
| 360 | Nucleic Acid | NtHAP1a |
| | | deletion fragment |
| 361 | Nucleic Acid | NtHAP1a |
| | | deletion fragment |
| 363 | Nucleic Acid | NtHAP1b |
| | | deletion fragment |
| 363 | Nucleic Acid | NtHAP1a |
| | | deletion fragment |
| 364 | Nucleic Acid | NtHAP1a |
| | | deletion fragment |
| 365 | Nucleic Acid | NtHAP1b |
| | | deletion fragment |
| 366 | Nucleic Acid | NtHAP1a |
| | | deletion fragment |
| 367 | Nucleic Acid | NtHAP2 |
| | | deletion fragment |
| 368 | Nucleic Acid | NtHAP1a |
| | | deletion fragment |
| 369 | Nucleic Acid | NtHAP1b |
| | | deletion fragment |
| 370 | Nucleic Acid | NtHAP1a |
| | | deletion fragment |
| 371 | Nucleic Acid | NtHAP1b |
| | | deletion fragment |
| 372 | Nucleic Acid | NtHAP1a |
| | | deletion fragment |
| 373 | Nucleic Acid | NtHAP1b |
| | | deletion fragment |
| 374 | Nucleic Acid | NtHAP1a |
| | | deletion fragment |
| 375 | Nucleic Acid | NtHAP1a |
| | | deletion fragment |
| 376 | Nucleic Acid | NtHAP1a |
| | | deletion fragment |
| 377 | Nucleic Acid | NtHAP1a |
| | | deletion fragment |
| 378 | Nucleic Acid | NtHAP1a |
| | | deletion fragment |
| 379 | Nucleic Acid | NtHAP1a |
| | | deletion fragment |
| 380 | Nucleic Acid | NtHAP1a |
| | | deletion fragment |
| 381 | Nucleic Acid | NtHAP1a |
| | | deletion fragment |
| 382 | Nucleic Acid | NtHAP1a |
| | | deletion fragment |
| 383 | Nucleic Acid | NtHAP1a |
| | | deletion fragment |
| 384 | Nucleic Acid | NtHAP1b |
| | | deletion fragment |
| 385 | Amino Acid | NtHAP1a mutant |
| 386 | Amino Acid | NtHAP1a mutant |
| 387 | Amino Acid | NtHAP1a mutant |
| 388 | Amino Acid | NtHAP1b mutant |
| 389 | Amino Acid | NtHAP1b mutant |
| 390 | Amino Acid | NtHAP1a mutant |
| 391 | Amino Acid | NtHAP1a mutant |
| 392 | Amino Acid | NtHAP1a mutant |
| 393 | Amino Acid | NtHAP1b mutant |
| 394 | Amino Acid | NtHAP1a mutant |
| 395 | Amino Acid | NtHAP1a mutant |
| 396 | Amino Acid | NtHAP1b mutant |
| 397 | Amino Acid | NtHAP1b mutant |
| 398 | Amino Acid | NtHAP1a mutant |
| 399 | Amino Acid | NtHAP1a mutant |
| 400 | Amino Acid | NtHAP1b mutant |
| 401 | Amino Acid | NtHAP1b mutant |
| 402 | Amino Acid | NtHAP1a mutant |
| 403 | Amino Acid | NtHAP1a mutant |
| 404 | Amino Acid | NtHAP1b mutant |
| 405 | Amino Acid | NtHAP1b mutant |
| 406 | Amino Acid | NtHAP1a mutant |
| 407 | Amino Acid | NtHAP1a mutant |
| 408 | Amino Acid | NtHAP1b mutant |
| 409 | Amino Acid | NtHAP1b mutant |
| 410 | Amino Acid | NtHAP1a mutant |
| 411 | Amino Acid | NtHAP1a mutant |
| 412 | Amino Acid | NtHAP1b mutant |
| 413 | Amino Acid | NtHAP1b mutant |
| 414 | Amino Acid | NtHAP1a mutant |
| 415 | Amino Acid | NtHAP1b mutant |
| 416 | Amino Acid | NtHAP1a mutant |
| 417 | Amino Acid | NtHAP1a mutant |
| 418 | Amino Acid | NtHAP1b mutant |
| 419 | Amino Acid | NtHAP1a mutant |
| 420 | Amino Acid | NtHAP1a mutant |
| 421 | Amino Acid | NtHAP1b mutant |
| 422 | Amino Acid | NtHAP1b mutant |
| 423 | Amino Acid | NtHAP1a mutant |
| 424 | Amino Acid | NtHAP1a mutant |
| 425 | Amino Acid | NtHAP1b mutant |
| 426 | Amino Acid | NtHAP1b mutant |
| 427 | Amino Acid | NtHAP1a mutant |
| 428 | Amino Acid | NtHAP1a mutant |
| 429 | Amino Acid | NtHAP1b mutant |
| 430 | Amino Acid | NtHAP1b mutant |
| 431 | Amino Acid | NtHAP1b mutant |
| 432 | Amino Acid | NtHAP2 mutant |
| 433 | Amino Acid | NtHAP2 mutant |
| 434 | Amino Acid | NtHAP1a mutant |
| 435 | Amino Acid | NtHAP1b mutant |
| 436 | Amino Acid | NtHAP1b mutant |
| 437 | Amino Acid | NtHAP2 mutant |
| 438 | Amino Acid | NtHAP2 mutant |
| 439 | Amino Acid | NtHAP2 mutant |
| 440 | Amino Acid | NtHAP2 mutant |
| 441 | Amino Acid | NtHAP1b mutant |
| 442 | Amino Acid | NtHAP2 mutant |
| 443 | Amino Acid | NtHAP2 mutant |
| 444 | Amino Acid | NtHAP1b mutant |
| 445 | Amino Acid | NtHAP1b mutant |
| 446 | Amino Acid | NtHAP2 mutant |
| 447 | Amino Acid | NtHAP2 mutant |
| 448 | Amino Acid | NtHAP1a mutant |
| 449 | Amino Acid | NtHAP1b mutant |
| 450 | Amino Acid | NtHAP2 mutant |
| 451 | Amino Acid | NtHAP2 mutant |
| 452 | Amino Acid | NtHAP1a mutant |
| 453 | Amino Acid | NtHAP1b mutant |
| 454 | Amino Acid | NtHAP2 mutant |
| 455 | Amino Acid | NtHAP1b mutant |
| 456 | Amino Acid | NtHAP2 mutant |
| 457 | Amino Acid | NtHAP1a mutant |
| 458 | Amino Acid | NtHAP2 mutant |
| 459 | Amino Acid | NtHAP1a mutant |
| 460 | Amino Acid | NtHAP1b mutant |
| 461 | Amino Acid | NtHAP2 mutant |
| 462 | Amino Acid | NtHAP2 mutant |
| 463 | Amino Acid | NtHAP2 mutant |
| 464 | Amino Acid | NtHAP2 mutant |
| 465 | Amino Acid | NtHAP1a mutant |
| 466 | Amino Acid | NtHAP1a mutant |
| 467 | Amino Acid | NtHAP2 mutant |
| 468 | Amino Acid | NtHAP1b mutant |
| 469 | Amino Acid | NtHAP2 mutant |
| 470 | Amino Acid | NtHAP2 mutant |
| 471 | Amino Acid | NtHAP1a mutant |
| 472 | Amino Acid | NtHAP1b mutant |
| 473 | Amino Acid | NtHAP1a mutant |
| 474 | Amino Acid | NtHAP1a mutant |
| 475 | Amino Acid | NtHAP1a mutant |
| 476 | Amino Acid | NtHAP1a mutant |
| 477 | Amino Acid | NtHAP1a mutant |
| 478 | Amino Acid | NtHAP1a mutant |
| 479 | Amino Acid | NtHAP1a mutant |
| 480 | Amino Acid | NtHAP1a mutant |
| 481 | Amino Acid | NtHAP1a mutant |
| 482 | Amino Acid | NtHAP1a mutant |
| 483 | Amino Acid | NtHAP1a mutant |
| 484 | Amino Acid | NtHAP1a mutant |
| 485 | Amino Acid | NtHAP1a mutant |
| 486 | Amino Acid | NtHAP1a mutant |
| 487 | Amino Acid | NtHAP1a mutant |
| 488 | Amino Acid | NtHAP1a mutant |
| 489 | Amino Acid | NtHAP1a mutant |
| 490 | Amino Acid | NtHAP1a mutant |
| 491 | Amino Acid | NtHAP1a mutant |
| 492 | Amino Acid | NtHAP1a mutant |
| 493 | Amino Acid | NtHAP1a mutant |
| 494 | Amino Acid | NtHAP1a mutant |
| 495 | Amino Acid | NtHAP1a mutant |
| 496 | Amino Acid | NtHAP1a mutant |
| 497 | Amino Acid | NtHAP1a mutant |
| 498 | Amino Acid | NtHAP1a mutant |
| 499 | Amino Acid | NtHAP1a mutant |
| 500 | Amino Acid | NtHAP1a mutant |
| 501 | Amino Acid | NtHAP1a mutant |
| 502 | Amino Acid | NtHAP1a mutant |
| 503 | Amino Acid | NtHAP1a mutant |
| 504 | Amino Acid | NtHAP1a mutant |
| 505 | Amino Acid | NtHAP1a mutant |
| 506 | Amino Acid | NtHAP1a mutant |
| 507 | Amino Acid | NtHAP1a mutant |
| 508 | Amino Acid | NtHAP1a mutant |
| 509 | Amino Acid | NtHAP1a mutant |
| 510 | Amino Acid | NtHAP1a mutant |
| 511 | Amino Acid | NtHAP1a mutant |
| 512 | Amino Acid | NtHAP1a mutant |
| 513 | Amino Acid | NtHAP1a mutant |
| 514 | Amino Acid | NtHAP1a mutant |
| 515 | Amino Acid | NtHAP1a mutant |
| 516 | Amino Acid | NtHAP1a mutant |
| 517 | Amino Acid | NtHAP1a mutant |
| 518 | Amino Acid | NtHAP1a mutant |
| 519 | Amino Acid | NtHAP1a mutant |
| 520 | Amino Acid | NtHAP1b mutant |
| 521 | Amino Acid | NtHAP1b mutant |
| 522 | Amino Acid | NtHAP1b mutant |
| 523 | Amino Acid | NtHAP1b mutant |
| 524 | Amino Acid | NtHAP1b mutant |
| 525 | Amino Acid | NtHAP1b mutant |
| 526 | Amino Acid | NtHAP1b mutant |
| 527 | Amino Acid | NtHAP2 mutant |
| 528 | Amino Acid | NtHAP2 mutant |
| 529 | Amino Acid | NtHAP2 mutant |
| 530 | Amino Acid | NtHAP2 mutant |
| 531 | Amino Acid | NtHAP2 mutant |
| 532 | Amino Acid | NtHAP2 mutant |
| 533 | Amino Acid | NtHAP2 mutant |
| 534 | Nucleic Acid | NtHAP2 deletion |
| | | fragment |
| 535 | Nucleic Acid | NtHAP2 deletion |
| | | fragment |
| 536 | Nucleic Acid | NtHAP1a |
| | | deletion fragment |
| 537 | Nucleic Acid | NtHAP3 |
| | (genomic) | |
| 538 | Amino Acid | NtHAP3 |
| 539 | Amino Acid | Nicotiana |
| | | attenuata |
| | | XP_019264549.1 |
| 540 | Amino Acid | Nicotiana |
| | | attenuata |
| | | XP_019226481.1 |
| 541 | Amino Acid | Nicotiana |
| | | benthamiana |
| | | UUF87240.1 |
| 542 | Amino Acid | Nicotiana |
| | | benthamiana |
| | | UUF87241.1 |
| 543 | Amino Acid | Nicotiana |
| | | sylvestris |
| | | XP_009790622.1 |
| 544 | Amino Acid | Nicotiana |
| | | sylvestris |
| | | XP_009795312.1 |
| 545 | Amino Acid | Nicotiana |
| | | tomentosiformis |
| | | XP_009592261.1 |
| 546 | Amino Acid | Nicotiana |
| | | tomentosiformis |
| | | XP_009628751.1 |
| 547 | Amino Acid | Anisodus |
| | | acutangulus |
| | | KAJ8534341.1 |
| 548 | Amino Acid | Anisodus |
| | | acutangulus |
| | | KAJ8570293.1 |
| 549 | Amino Acid | Anisodus |
| | | acutangulus |
| | | KAJ8560981.1 |
| 550 | Amino Acid | Anisodus |
| | | tanguticus |
| | | KAK4378121.1 |
| 551 | Amino Acid | Anisodus |
| | | tanguticus |
| | | KAK4346119.1 |
| 552 | Amino Acid | Anisodus |
| | | tanguticus |
| | | KAK4373912.1 |
| 553 | Amino Acid | Capsicum annuum |
| | | XP_016544016.1 |
| 554 | Amino Acid | Capsicum annuum |
| | | PHT63471.1 |
| 555 | Amino Acid | Capsicum annuum |
| | | KAF3665665.1 |
| 556 | Amino Acid | Capsicum annuum |
| | | XP_016565843.1 |
| 557 | Amino Acid | Capsicum |
| | | baccatum |
| | | PHT37175.1 |
| 558 | Amino Acid | Capsicum |
| | | baccatum |
| | | PHT38681.1 |
| 559 | Amino Acid | Capsicum |
| | | chinense |
| | | PHU21889.1 |
| 560 | Amino Acid | Datura |
| | | stramonium |
| | | MCD7472448.1 |
| 561 | Amino Acid | Datura |
| | | stramonium |
| | | MCD7465925.1 |
| 562 | Amino Acid | Lycium barbarum |
| | | XP_060177647.1 |
| 563 | Amino Acid | Lycium barbarum |
| | | XP_060209129.1 |
| 564 | Amino Acid | Lycium |
| | | ferocissimum |
| | | XP_059295899.1 |
| 565 | Amino Acid | Lycium |
| | | ferocissimum |
| | | XP_059277299.1 |
| 566 | Amino Acid | Solanum |
| | | bulbocastanum |
| | | KAK6779121.1 |
| 567 | Amino Acid | Solanum |
| | | bulbocastanum |
| | | KAK6780870.1 |
| 568 | Amino Acid | Solanum chilense |
| | | TMW90306.1 |
| 569 | Amino Acid | Solanum chilense |
| | | TMW85142.1 |
| 570 | Amino Acid | Solanum |
| | | commersonii |
| | | KAG5593582.1 |
| 571 | Amino Acid | Solanum |
| | | commersonii |
| | | KAG5587825.1 |
| 572 | Amino Acid | Solanum |
| | | dulcamara |
| | | XP_055806821.1 |
| 573 | Amino Acid | Solanum |
| | | dulcamara |
| | | XP_055813521.1 |
| 574 | Amino Acid | Solanum |
| | | lycopersicum |
| | | XP_004247560.1 |
| 575 | Amino Acid | Solanum pennellii |
| | | XP_015055664.1 |
| 576 | Amino Acid | Solanum pennellii |
| | | XP_015088639.1 |
| 577 | Amino Acid | Solanum |
| | | pinnatisectum |
| | | KAK4725616.1 |
| 578 | Amino Acid | Solanum |
| | | pinnatisectum |
| | | KAK4723224.1 |
| 579 | Amino Acid | Solanum |
| | | pinnatisectum |
| | | KAK4732357.1 |
| 580 | Amino Acid | Solanum |
| | | stenotomum |
| | | XP_049402688.1 |
| 581 | Amino Acid | Solanum |
| | | stenotomum |
| | | XP_049407092.1 |
| 582 | Amino Acid | Solanum |
| | | tuberosum |
| | | XP_006364759.1 |
| 583 | Amino Acid | Solanum |
| | | tuberosum |
| | | KAH0723346.1 |
| 584 | Amino Acid | Solanum |
| | | tuberosum |
| | | KAH0640254.1 |
| 585 | Amino Acid | Solanum |
| | | tuberosum |
| | | XP_006360793.1 |
| 586 | Amino Acid | Solanum |
| | | tuberosum |
| | | KAH0635004.1 |
| 587 | Amino Acid | Solanum |
| | | verrucosum |
| | | XP_049369784.1 |
| 588 | Amino Acid | Solanum |
| | | verrucosum |
| | | XP_049374436.1 |
| 589 | Amino Acid | Solanum |
| | | lycopersicum |
| | | XP_004249103.1 |
| | | (SIHAP) |
| 590 | Nucleic Acid | Forward primer |
| | | sequence used for |
| | | amplifying |
| | | NtHAP1a DNA |
| | | region harboring |
| | | gRNA1 target |
| 591 | Nucleic Acid | Reverse primer |
| | | sequence used for |
| | | amplifying |
| | | NtHAP1a DNA |
| | | region harboring |
| | | gRNA 1 target |
| 592 | Nucleic Acid | Forward primer |
| | | sequence used for |
| | | amplifying |
| | | NtHAP1b DNA |
| | | region harboring |
| | | gRNA1 target |
| 593 | Nucleic Acid | Reverse primer |
| | | sequence used for |
| | | amplifying |
| | | NtHAP1b DNA |
| | | region harboring |
| | | gRNA 1 target |
| 594 | Nucleic Acid | Forward primer |
| | | sequence used for |
| | | amplifying |
| | | NtHAPI DNA |
| | | region harboring |
| | | gRNA2 target |
| 595 | Nucleic Acid | Reverse primer |
| | | sequence used for |
| | | amplifying |
| | | NtHAPI DNA |
| | | region harboring |
| | | gRNA2 target |
| 596 | Nucleic Acid | Forward primer |
| | | sequence used for |
| | | amplifying |
| | | NtHAP1a DNA |
| | | region harboring |
| | | gRNA3 target |
| 597 | Nucleic Acid | Reverse primer |
| | | sequence used for |
| | | amplifying |
| | | NtHAP1a DNA |
| | | region harboring |
| | | gRNA3 target |
| 598 | Nucleic Acid | Forward primer |
| | | sequence used for |
| | | amplifying |
| | | NtHAPI DNA |
| | | region harboring |
| | | gRNA4 or gRNA6 |
| | | target |
| 599 | Nucleic Acid | Reverse primer |
| | | sequence used for |
| | | amplifying |
| | | NtHAPI DNA |
| | | region harboring |
| | | gRNA4 or gRNA6 |
| | | target |
| 600 | Nucleic Acid | Forward primer |
| | | sequence used for |
| | | amplifying |
| | | NtHAP1b DNA |
| | | region harboring |
| | | gRNA5 target |
| 601 | Nucleic Acid | Reverse primer |
| | | sequence used for |
| | | amplifying |
| | | NtHAP1b DNA |
| | | region harboring |
| | | gRNA5 target |
| 602 | Nucleic Acid | Forward primer |
| | | sequence used for |
| | | amplifying |
| | | NtHAP2 DNA |
| | | region harboring |
| | | gRNA7 target |
| 603 | Nucleic Acid | Reverse primer |
| | | sequence used for |
| | | amplifying |
| | | NtHAP2 DNA |
| | | region harboring |
| | | gRNA7 target |
| 604 | Nucleic Acid | Forward primer |
| | | sequence used for |
| | | amplifying |
| | | NtHAP2 DNA |
| | | region harboring |
| | | gRNA8 target |
| 605 | Nucleic Acid | Reverse primer |
| | | sequence used for |
| | | amplifying |
| | | NtHAP2 DNA |
| | | region harboring |
| | | gRNA8 target |
| 606 | Nucleic Acid | Forward primer |
| | | sequence used for |
| | | RT-PCR or qPCR |
| | | on NtHAP1a |
| | | transcript |
| | | on NtHAP1a |
| | | transcript |
| 608 | Nucleic Acid | Forward primer |
| | | sequence used for |
| | | RT-PCR or qPCR |
| | | on NtHAP1b |
| | | transcript |
| 609 | Nucleic Acid | Reverse primer |
| | | sequence used for |
| | | RT-PCR or qPCR |
| | | on NtHAP1b |
| | | transcript |
| 610 | Nucleic Acid | Forward primer |
| | | sequence used for |
| | | RT-PCR or qPCR |
| | | on NtHAP2 |
| | | transcript |
| 611 | Nucleic Acid | Reverse primer |
| | | sequence used for |
| | | RT-PCR or qPCR |
| | | on NtHAP2 |
| | | transcript |
| 612 | Nucleic Acid | NtHAP 3 coding |
| | | sequence |

### BACKGROUND

Trichomes are epidermal outgrowths in plants. Their presence on stem, leaf, and floral tissues provides protection for plants against various biotic and abiotic stresses. Some trichomes, such as glandular trichomes, are the site of metabolic compound synthesis and storage. Other trichomes, such as non-glandular trichomes, can protect plants from predators (e.g., by providing a physical barrier) or ultraviolet light. Trichomes can be found on adaxial (upper) and abaxial (lower) leaf surfaces, albeit at different densities in some plant species.

Glandular trichomes are known to play a role in the biosynthesis, storage, and secretion of specialized or secondary metabolites such as terpenoids, phenylpropanoids, flavonoids, methyl ketones, and acylsugars.

The HAIRPLUS gene was recently identified as playing a role in glandular trichome formation in tomato (Solanum lycopersicum). See Fonseca et al., Hortic. Res., 9:uhab015 (2022). In this disclosure, four HAIRPLUS homologues are identified in tobacco, which are modulated to improve trichome characteristics in tobacco plants.

### SUMMARY

In an aspect, this disclosure provides a modified tobacco plant, or part thereof, comprising a non-natural mutation in an endogenous nucleic acid molecule, wherein the endogenous nucleic acid molecule encodes a HAIRPLUS1a (NtHAP1a) protein comprising the amino acid sequence of SEQ ID NO: 1, and wherein expression or activity of the NtHAP1a protein is reduced in the modified tobacco plant as compared to a control tobacco plant lacking the non-natural mutation when grown under comparable conditions.

In an aspect, this disclosure provides a modified tobacco plant, or part thereof, comprising a non-natural mutation in an endogenous nucleic acid molecule, wherein the endogenous nucleic acid molecule encodes a HAIRPLUS1b (NtHAP1b) protein comprising the amino acid sequence of SEQ ID NO: 2, and wherein expression or activity of the NtHAP1b protein is reduced in the modified tobacco plant as compared to a control tobacco plant lacking the non-natural mutation when grown under comparable conditions.

In an aspect, this disclosure provides a modified tobacco plant, or part thereof, comprising a non-natural mutation in an endogenous nucleic acid molecule, wherein the endogenous nucleic acid molecule encodes a HAIRPLUS2 (NtHAP2) protein comprising the amino acid sequence of SEQ ID NO: 3, and wherein expression or activity of the NtHAP2 protein is reduced in the modified tobacco plant as compared to a control tobacco plant lacking the non-natural mutation when grown under comparable conditions.

In an aspect, this disclosure provides a modified tobacco plant, or part thereof, comprising a non-natural mutation in an endogenous nucleic acid molecule, wherein the endogenous nucleic acid molecule encodes a HAIRPLUS3 (NtHAP3) protein comprising the amino acid sequence of SEQ ID NO: 538, and wherein expression or activity of the NtHAP2 protein is reduced in the modified tobacco plant as compared to a control tobacco plant lacking the non-natural mutation when grown under comparable conditions.

In an aspect, this disclosure provides a modified tobacco plant, or part thereof, comprising a recombinant nucleic acid construct comprising a heterologous promoter operably linked to a polynucleotide that encodes a non-coding RNA molecule, where the non-coding RNA molecule suppresses the transcription or translation of at least one RNA molecule encoding a protein comprising an amino acid sequence at least 90% identical or similar to an amino acid sequence selected from the group consisting of SEQ ID NOs: 1, 2, 3, and 538, and where the transcription or translation is as compared to a control tobacco plant lacking the recombinant nucleic acid construct when grown under comparable conditions.

In an aspect, this disclosure provides a modified tobacco plant, or part thereof, comprising a non-natural mutation in a nucleic acid molecule encoding a HAIRPLUS1a (NtHAP1a) protein, where the nucleic acid molecule comprises a nucleic acid sequence selected from the group consisting of SEQ ID NOs: 20 to 23.

In an aspect, this disclosure provides a modified tobacco plant, or part thereof, comprising a mutated HAIRPLUS1a (NtHAP1a) protein, where the mutated NtHAP1a protein comprises an amino acid sequence selected from the group consisting of SEQ ID NOs: 27 to 30.

In an aspect, this disclosure provides a modified tobacco plant, or part thereof, comprising a non-natural mutation in a nucleic acid molecule encoding a HAIRPLUS1b (NtHAP1b) protein, where the nucleic acid molecule comprises a nucleic acid sequence selected from the group consisting of SEQ ID NOs: 24 and 25.

In an aspect, this disclosure provides a modified tobacco plant, or part thereof, comprising a mutated HAIRPLUS1b (NtHAP1b) protein, where the mutated NtHAP1b protein comprises an amino acid sequence selected from the group consisting of SEQ ID NOs: 31 and 32.

In an aspect, this disclosure provides a modified tobacco plant, or part thereof, comprising a non-natural mutation in a nucleic acid molecule encoding a HAIRPLUS2 (NtHAP2) protein, where the nucleic acid molecule comprises the nucleic acid sequence of SEQ ID NO: 26.

In an aspect, this disclosure provides a modified tobacco plant, or part thereof, comprising a mutated HAIRPLUS2 (NtHAP2) protein, where the mutated NtHAP2 protein comprises the amino acid sequence of SEQ ID NO: 33.

In an aspect, this disclosure provides a seed obtained from any modified tobacco plant provided herein.

In an aspect, this disclosure provides cured tobacco material from any modified tobacco plant, or part thereof, provided herein. In an aspect, this disclosure provides a tobacco product comprising cured tobacco material from any modified tobacco plant, or part thereof, provided herein. In an aspect, this disclosure provides a tobacco blend comprising cured tobacco material from any modified tobacco plant, or part thereof, provided herein. In an aspect, this disclosure provides fermented tobacco comprising tobacco material from any modified tobacco plant, or part thereof, provided herein. In an aspect, this disclosure provides a reconstituted tobacco comprising tobacco material from any modified tobacco plant, or part thereof, provided herein.

In an aspect, this disclosure provides a method of producing a modified tobacco plant, the method comprising: (a) inducing at least one non-natural mutation in at least one endogenous nucleic acid molecule encoding an amino acid sequence selected from the group consisting of SEQ ID NOs: 1, 2, 3, and 538 in at least one tobacco cell; and (b) regenerating a modified tobacco plant from the at least one tobacco cell of step (a), where the tobacco plant comprises the at least one non-natural mutation, and where the modified tobacco plant exhibits reduced expression or activity of the at least one endogenous nucleic acid molecule as compared to a control tobacco plant lacking the at least one non-natural mutation when grown under comparable conditions.

In an aspect, this disclosure provides a method of producing a modified tobacco plant, the method comprising: (a) introducing a recombinant nucleic acid construct to at least one tobacco cell, where the recombinant nucleic acid construct comprises a heterologous promoter operably linked to a polynucleotide that encodes a non-coding RNA molecule; and (b) regenerating a modified tobacco plant from the at least one tobacco cell of step (a), where the tobacco plant comprises the recombinant nucleic acid construct, and where the non-coding RNA molecule suppresses the transcription or translation of at least one RNA molecule encoding an amino acid sequence at least 90% identical or similar to an amino acid sequence selected from the group consisting of SEQ ID NOs: 1, 2, 3, and 538 as compared to a control tobacco plant lacking the recombinant nucleic acid construct when grown under comparable conditions.

In an aspect, this disclosure provides a method for producing a tobacco plant, the method comprising: (a) crossing a modified tobacco plant with a second tobacco plant to produce at least one progeny tobacco seed, where the modified tobacco plant comprises at least one non-natural mutation in at least one endogenous nucleic acid molecule encoding an amino acid sequence selected from the group consisting of SEQ ID NOs: 1, 2, 3, and 538; and (b) selecting at least one progeny tobacco seed, or a tobacco plant germinated therefrom, comprising the at least one non-natural mutation.

In an aspect, this disclosure provides a method for producing a tobacco plant, the method comprising: (a) crossing a first tobacco plant with a second tobacco plant to produce at least one progeny tobacco seed, where the first tobacco plant comprises a recombinant nucleic acid construct comprising a heterologous promoter operably linked to a polynucleotide that encodes a non-coding RNA molecule that binds to and suppresses the transcription or translation of at least one RNA molecule encoding an amino acid sequence at least 90% identical or similar to an amino acid sequence selected from the group consisting of SEQ ID NOs: 1, 2, 3, and 538; and (b) selecting at least one progeny tobacco seed, or a tobacco plant germinated therefrom, comprising the recombinant nucleic acid construct.

In an aspect, this disclosure provides a method of producing a tobacco product, the method comprising: (a) curing tobacco material to produce cured tobacco material, where the tobacco material is from a modified tobacco plant comprising a non-natural mutation in an endogenous nucleic acid molecule encoding a HAIRPLUS1a (NtHAP1a) protein, where the endogenous NtHAP1a protein comprises the amino acid sequence of SEQ ID NO: 1, and wherein expression or activity of the NtHAP1a protein is reduced in the modified tobacco plant as compared to a control tobacco plant lacking the non-natural mutation when grown under comparable conditions; and (b) producing a tobacco product comprising the cured tobacco material from step (a).

In an aspect, this disclosure provides a method of producing a tobacco product, the method comprising: (a) curing tobacco material to produce cured tobacco material, where the tobacco material is from a modified tobacco plant comprising a non-natural mutation in an endogenous nucleic acid molecule encoding a HAIRPLUS1b (NtHAP1b) protein, where the endogenous NtHAP1a protein comprises the amino acid sequence of SEQ ID NO: 2, and wherein expression or activity of the NtHAP1b protein is reduced in the modified tobacco plant as compared to a control tobacco plant lacking the non-natural mutation when grown under comparable conditions; and (b) producing a tobacco product comprising the cured tobacco material from step (a).

In an aspect, this disclosure provides a method of producing a tobacco product, the method comprising: (a) curing tobacco material to produce cured tobacco material, where the tobacco material is from a modified tobacco plant comprising a non-natural mutation in an endogenous nucleic acid molecule encoding a HAIRPLUS2 (NtHAP2) protein, where the endogenous NtHAP2 protein comprises the amino acid sequence of SEQ ID NO: 3, and wherein expression or activity of the NtHAP2 protein is reduced in the modified tobacco plant as compared to a control tobacco plant lacking the non-natural mutation when grown under comparable conditions; (b) producing a tobacco product comprising the cured tobacco material from step (a).

In an aspect, this disclosure provides a method of producing a tobacco product, the method comprising: (a) curing tobacco material to produce cured tobacco material, where the tobacco material is from a modified tobacco plant comprising a non-natural mutation in an endogenous nucleic acid molecule encoding a HAIRPLUS3 (NtHAP3) protein, where the endogenous NtHAP3 protein comprises the amino acid sequence of SEQ ID NO: 538, and wherein expression or activity of the NtHAP3 protein is reduced in the modified tobacco plant as compared to a control tobacco plant lacking the non-natural mutation when grown under comparable conditions; (b) producing a tobacco product comprising the cured tobacco material from step (a).

### BRIEF DESCRIPTION OF THE DRAWINGS

FIG. 1 depicts the structure of NtHAP1a, NtHAP1b, NtHAP2, and NtHAP3 genes, including the approximate position of RNAi target sequences. UTR refers to untranslated region, ATG represents the start codon position, and STOP represents the position of the stop codon.

FIG. 2 depicts the approximate location of guide RNA (gRNA) binding sites used to edit the NtHAP genes in tobacco. UTR refers to untranslated region, ATG represents the start codon position, and STOP represents the position of the stop codon.

FIG. 3 depicts trichomes observed on an Izmir tobacco leaf. Representative trichome types are circled: tall glandular trichomes are shown as I; short glandular trichomes are shown as II; non-glandular trichomes are shown as III; and immature tall or short glandular trichomes are shown as IV.

FIG. 4 depicts an Izmir wild type (WT) tobacco plant and a modified tobacco plant from the line IG1278-112 comprising non-natural mutations in the NtHAP1a and NtHAP2 genes. Increased trichome density is observed in the modified tobacco plant.

FIG. 5 depicts an Izmir wild type (WT) tobacco plant and a modified tobacco plant from the line IG1278-112 comprising non-natural mutations in the NtHAP1a and NtHAP2 genes. Increased trichome density is observed in the modified tobacco plant.

FIG. 6 depicts an Izmir wild type (WT) tobacco plant and a modified tobacco plant from the line IG56-75 comprising a non-natural mutation in the NtHAP1b gene. The modified tobacco plant exhibits an increased trichome density as compared to the wild type tobacco plant.

FIG. 7 depicts an Izmir wild type (WT) tobacco plant a modified tobacco plant from the line IG12-09 comprising non-natural mutations in the NtHAP1a and NtHAP1b genes. Increased trichome density is observed in the modified tobacco plant.

FIG. 8 depicts an Izmir wild type (WT) tobacco plant and a modified tobacco plant from the line IG34-15 comprising a non-natural mutation in the NtHAP1a gene. The modified tobacco plant exhibits an increased trichome density as compared to the wild type tobacco plant.

FIG. 9 depicts an Izmir wild type (WT) tobacco plant and a modified tobacco plant from the line IG56-75 comprising a non-natural mutation in the NtHAP1b gene. The modified tobacco plant exhibits an increased trichome density as compared to the wild type tobacco plant.

FIG. 10 depicts images of trichome density in Izmir wild type (WT) tobacco, NtHAP1a mutant (line IG34-15) tobacco, NtHAP1a/NtHAP1b mutant (line IG12-09) tobacco, and NtHAP1b mutant (line IG56-75) tobacco.

FIG. 11 depicts trichomes on Izmir wild type (WT), NtHAP1a/NtHAP1b mutant (line IG12-09), NtHAP1b mutant (line IG56-75), and NtHAP1a/NtHAP2 mutant (line IG1278-112) tobacco leaves after flash freezing with liquid nitrogen, which aids visualization of trichomes.

FIG. 12 and FIG. 13 each depict representative images of trichomes on tobacco leaves at different growth stages from Izmir wild type (WT), NtHAP1b mutant (line IG56-75), NtHAP1a/NtHAP1b mutant (line IG12-09), and NHAP1a/NtHAP2 mutant (line IG1278-112) tobacco leaves and quantitative measurements of trichome density for each line. All HAP mutant lines exhibit increased trichome density as compared to WT.

FIG. 14 depicts relative expression levels of NtHAP1 (a and b) and NtHAP2 in wildtype (WT) and two RNAi lines suppressing the expression of NtHAP1a, NtHAP1b, and NtHAP2 in a K326 tobacco background. Error bars represent one standard error of the mean.

FIG. 15, FIG. 16, FIG. 17, and FIG. 18 each depict trichomes on K326 wild type (WT) tobacco and a K326 tobacco line expressing SEQ ID NOs: 18 and 19 (RNAi line 1), which are designed to collectively suppress the expression of NtHAP1a, NtHAP1b, and NtHAP2. The RNAi line exhibits increased trichome number and density as compared to the WT plant.

FIG. 19 and FIG. 20 depict K326 tobacco leaves at different growth stages in wild type (WT) and a tobacco line expressing SEQ ID NOs: 18 and 19 (RNAi line 1). FIG. 19 depicts a slight decrease in trichome density in the RNAi line in young seedlings with partially expanded leaves, but FIG. 20 depicts a roughly three-fold increase in trichome density in the RNAi line as compared to WT in in fully expanded leaves.

FIG. 21 and FIG. 22 depict trichomes on K326 wild type (WT) tobacco and two independent K326 tobacco lines expressing SEQ ID NOs: 18 and 19 (RNAi lines 2 and 3), which are designed to collectively suppress the expression of NtHAP1a, NtHAP1b, and NtHAP2. The RNAi lines exhibit increased trichome number and density as compared to the WT plant. FIG. 21 shows whole leaves, and FIG. 22 shows trichomes on the adaxial surface of leaves.

FIG. 23 and FIG. 24 depict K326 tobacco leaves at different growth stages in wild type (WT) and two independent tobacco line expressing SEQ ID NOs: 18 and 19 (RNAi lines 2 and 3). FIG. 23 depicts a slight decrease in trichome density in RNAi line 3 and a slight increase in trichome density in RNAi line 2 in young seedlings with partially expanded leaves, but FIG. 24 depicts a roughly three-fold increase in trichome density in both RNAi lines as compared to WT in fully expanded leaves.

FIG. 25 depicts the average trichome length in wildtype (WT) and two RNAi lines suppressing the expression of NtHAP1a, NtHAP1b, and NtHAP2 in a K326 tobacco background. ** refers to a p-value of <0.01 and *** refers to a p-value of <0.001 using a Student's t-test.

FIG. 26 depicts inflorescence phenotypes of Izmir wildtype (WT) and RNAi line plants of the same age (top), and whole plant phenotypes of K326 WT and RNAi line plants of the same age (bottom). The RNAi lines contain RNAi constructs designed to suppress the expression of NtHAP1a, NtHAP1b, and NtHAP2.

FIG. 27 depicts relative amounts of total terpenoid pathway compounds in Izmir wild type (WT), an NtHAP1a/NtHAP1b mutant (line IG12-09), an NtHAP1a mutant (line IG34-15), an NtHAP1b mutant (line IG56-75), an NtHAP1a/NtHAP2 mutant (line IG1278-112), and two RNAi lines (RNAi line 2 and RNAi line 3) expressing SEQ ID NOs: 18 and 19.

FIG. 28 depicts relative amounts of specific terpenoids in the tobacco lines depicted in FIG. 27.

FIG. 29 depicts relative amounts of total terpenoid pathway compounds in K326 wild type (WT) tobacco and two K326 RNAi tobacco lines (RNAi line 1 and RNAi line 4) expressing SEQ ID NOs: 18 and 19.

FIG. 30 depicts the phylogenetic relationship between NtHAP1a (SEQ ID NO: 1), NtHAP1b (SEQ ID NO: 2), NtHAP2 (SEQ ID NO: 3), and NtHAP3 (SEQ ID NO: 538) amino acid sequences with related amino acid sequences from other Nicotiana species. Nicotiana benthamiana UUF87241.1 refers to SEQ ID NO: 542; Nicotiana sylvestris XP_009795312.1 refers to SEQ ID NO: 544; Nicotiana tomentosiformis XP_009628751.1 refers to SEQ ID NO: 546; Nicotiana attenuata XP_019226481.1 refers to SEQ ID NO: 540; Nicotiana tomentosiformis XP_009592261.1 refers to SEQ ID NO: 545; Nicotiana attenuata XP_019264549.1 refers to SEQ ID NO: 539; Nicotiana benthamiana UUF87240.1 refers to SEQ ID NO: 541; and Nicotiana sylvestris XP_009790622.1 refers to SEQ ID NO: 543.

FIG. 31 depicts the phylogenetic relationship between Nicotiana HAP amino acid sequences and HAP amino acid sequences from other Solanaceae species. Nicotiana benthamiana UUF87241.1 refers to SEQ ID NO: 542; Nicotiana sylvestris XP_009795312.1 refers to SEQ ID NO: 544; Nicotiana tomentosiformis XP_009628751.1 refers to SEQ ID NO: 546; Nicotiana attenuata XP_019226481.1 refers to SEQ ID NO: 540; Nicotiana tomentosiformis XP_009592261.1 refers to SEQ ID NO: 545; Nicotiana attenuata XP_019264549.1 refers to SEQ ID NO: 539; Nicotiana benthamiana UUF87240.1 refers to SEQ ID NO: 541; Nicotiana sylvestris XP_009790622.1 refers to SEQ ID NO: 543; NtHAP1a refers to SEQ ID NO: 1; NtHAP1b refers to SEQ ID NO: 2; NtHAP2 refers to SEQ ID NO: 3; NtHAP3 refers to SEQ ID NO: 538; Anisodus acutangulus KAJ8534341.1 refers to SEQ ID NO: 547; Anisodus acutangulus KAJ8570293.1 refers to SEQ ID NO: 548; Anisodus acutangulus KAJ8560981.1 refers to SEQ ID NO: 549; Anisodus tanguticus KAK4378121.1 refers to SEQ ID NO: 550; Anisodus tanguticus KAK4346119.1 refers to SEQ ID NO: 551; Anisodus tanguticus KAK4373912.1 refers to SEQ ID NO: 552; Capsicum annuum XP_016544016.1 refers to SEQ ID NO: 553; Capsicum annuum PHT63471.1 refers to SEQ ID NO: 554; Capsicum annuum KAF3665665.1 refers to SEQ ID NO: 555; Capsicum annuum XP_016565843.1 refers to SEQ ID NO: 556; Capsicum baccatum PHT37175.1 refers to SEQ ID NO: 557; Capsicum baccatum PHT38681.1 refers to SEQ ID NO: 558; Capsicum chinense PHU21889.1 refers to SEQ ID NO: 559; Datura stramonium MCD7472448.1 refers to SEQ ID NO: 560; Datura stramonium MCD7465925.1 refers to SEQ ID NO: 561; Lycium barbarum XP_060177647.1 refers to SEQ ID NO: 562; Lycium barbarum XP_060209129.1 refers to SEQ ID NO: 563; Lycium ferocissimum XP_059295899.1 refers to SEQ ID NO: 564; Lycium ferocissimum XP_059277299.1 refers to SEQ ID NO: 565; Solanum bulbocastanum KAK6779121.1 refers to SEQ ID NO: 566; Solanum bulbocastanum KAK6780870.1 refers to SEQ ID NO: 567; Solanum chilense TMW90306.1 refers to SEQ ID NO: 568; Solanum chilense TMW85142.1 refers to SEQ ID NO: 569; Solanum commersonii KAG5593582.1 refers to SEQ ID NO: 570; Solanum commersonii KAG5587825.1 refers to SEQ ID NO: 571; Solanum dulcamara XP_055806821.1 refers to SEQ ID NO: 572; Solanum dulcamara XP_055813521.1 refers to SEQ ID NO: 573; Solanum lycopersicum XP_004247560.1 refers to SEQ ID NO: 574; Solanum pennellii XP_015055664.1 refers to SEQ ID NO: 575; Solanum pennellii XP_015088639.1 refers to SEQ ID NO: 576; Solanum pinnatisectum KAK4725616.1 refers to SEQ ID NO: 577; Solanum pinnatisectum KAK4723224.1 refers to SEQ ID NO: 578; Solanum pinnatisectum KAK4732357.1 refers to SEQ ID NO: 579; Solanum stenotomum XP_049402688.1 refers to SEQ ID NO: 580; Solanum stenotomum XP_049407092.1 refers to SEQ ID NO: 581; Solanum tuberosum XP_006364759.1 refers to SEQ ID NO: 582; Solanum tuberosum KAH0723346.1 refers to SEQ ID NO: 583; Solanum tuberosum KAH0640254.1 refers to SEQ ID NO: 584; Solanum tuberosum XP_006360793.1 refers to SEQ ID NO: 585; Solanum tuberosum KAH0635004.1 refers to SEQ ID NO: 586; Solanum verrucosum XP_049369784.1 refers to SEQ ID NO: 587; Solanum verrucosum XP_049374436.1 refers to SEQ ID NO: 588; and Solanum lycopersicum XP_004249103.1 (SIHAP) refers to SEQ ID NO: 589.

FIG. 32 depicts neophytadiene levels in wildtype (WT) and RNAi lines in both Izmir and K326 tobacco backgrounds. The RNAi lines contain constructs designed to suppress the expression of NtHAP1a, NtHAP1b, and NtHAP2. ** refers to a p-value of <0.01 and *** refers to a p-value of <0.001 using a Student's t-test.

FIG. 33 depicts cis-abienol, 4,8,13-duvatriene-1,3-diol, and thunbergol levels in wildtype (WT) and RNAi lines in both Izmir and K326 tobacco backgrounds. The RNAi lines contain constructs designed to suppress the expression of NtHAP1a, NtHAP1b, and NtHAP2. NS refers to not significant, ** refers to a p-value of <0.01, and *** refers to a p-value of <0.001 using a Student's t-test.

### DETAILED DESCRIPTION

Unless defined otherwise, all technical and scientific terms used have the same meaning as commonly understood by one of ordinary skill in the art to which this disclosure belongs. Where a term is provided in the singular, the inventors also contemplate aspects of the disclosure described by the plural of that term. Where there are discrepancies in terms and definitions used in references that are incorporated by reference, the terms used in this application shall have the definitions given herein. Other technical terms used have their ordinary meaning in the art in which they are used, as exemplified by various art-specific dictionaries, for example, “The American Heritage® Science Dictionary” (Editors of the American Heritage Dictionaries, 2011, Houghton Mifflin Harcourt, Boston and New York), the “McGraw-Hill Dictionary of Scientific and Technical Terms” (6th edition, 2002, McGraw-Hill, New York), or the “Oxford Dictionary of Biology” (6th edition, 2008, Oxford University Press, Oxford and New York).

Any references cited herein, including, e.g., all patents, published patent applications, and non-patent publications, are incorporated herein by reference in their entireties.

When a grouping of alternatives is presented, any and all combinations of the members that make up that grouping of alternatives is specifically envisioned. For example, if an item is selected from a group consisting of A, B, C, and D, the inventors specifically envision each alternative individually (e.g., A alone, B alone, etc.), as well as combinations such as A, B, and D; A and C; B and C; etc. The term “and/or” when used in a list of two or more items means any one of the listed items by itself or in combination with any one or more of the other listed items. For example, the expression “A and/or B” is intended to mean either or both of A and B—e.g., A alone, B alone, or A and B in combination. The expression “A, B and/or C” is intended to mean A alone, B alone, C alone, A and B in combination, A and C in combination, B and C in combination, or A, B, and C in combination.

When a range of numbers is provided herein, the range is understood to inclusive of the edges of the range as well as any number between the defined edges of the range. For example, “between 1 and 10” includes any number between 1 and 10, as well as the number 1 and the number 10.

As used herein, the singular forms “a,” “an,” and “the” include plural references unless the context clearly dictates otherwise. For example, the term “a compound” or “at least one compound” may include a plurality of compounds, including mixtures thereof.

Any modified tobacco plant, or part thereof, is specifically envisioned for use with any method provided herein. Any nucleic acid sequence, amino acid sequence, or other composition provided herein is specifically envisioned for use with any method or tobacco plant provided herein. Any modified tobacco plant, or part thereof, is specifically envisioned for use in any tobacco product or any cured tobacco material provided herein.

This disclosure provides a seed obtained from any modified tobacco plant provided herein. This disclosure provides a cell of any modified tobacco plant provided herein.

Trichomes, in general, are hair-like epidermal outgrowths covering most aerial plant tissues. Trichomes tend to be multicellular, but unicellular trichomes are known as well. Multiple types of trichomes can be found on an individual plant, and trichomes vary in shape, size, and cellular organization. An individual trichome can be classified as a glandular trichome or a non-glandular trichome.

Glandular trichomes are characterized by the presence of a head made of cells that can secrete or store large quantities of specialized metabolites (e.g., without being limiting, terpenoids, phenylpropanoids, flavonoids, methyl ketones, acylsugars). Within the group of glandular trichomes, a trichome can be further characterized as being peltate or capitate. A capitate glandular trichome typically possesses a stalk with a length that is more than twice the height of the head, and the number of cells in the trichome is highly variable. A peltate trichome is a short-stalked trichome with a large head made of between four and eighteen cells arranged in one or two concentric circles.

Non-glandular trichomes typically provide physical protection for plants against biotic and abiotic stresses. For example, they can form a physical barrier against low humidity, high light intensity (e.g., ultraviolet light), high or low temperatures, and feeding and/or egg-laying by insects or arachnids (e.g., mites).

In an aspect, a trichome is a glandular trichome. In an aspect, a glandular trichome is a capitate glandular trichome. In an aspect, a glandular trichome is a peltate glandular trichome. In an aspect, a glandular trichome is selected from the group consisting of a capitate glandular trichome and a peltate glandular trichome. In an aspect, a trichome is a non-glandular trichome.

In some instances, it may be desirable to increase the total number of trichomes on a plant or plant part. It may also be desirable to increase the density of trichomes on a plant or plant part. Without being limited by any scientific theory, a plant comprising an increased number of trichomes can produce an increased amount of one or more desired compounds as compared to a control. See, for example, U.S. Patent Application Publication Nos. 2022/0243214 A1; 2022/0243215 A1; and 2022/0243216 A1.

In tomato (Solanum lycopersicum), the HAIRPLUS (SIHAP) gene (SEQ ID NO: 589) has been shown to play a role in regulating trichome density. See Fonseca et al., Hortic. Res., 9:uhab015 (2022). As is described in the non-limiting Examples below, this disclosure provides four tobacco homologues of HAP that are referred to herein as HAIRPLUS1a (NtHAP1a), HAIRPLUS1b (NtHAP1b), HAIRPLUS2 (NtHAP2), and HAIRPLUS3 (NtHAP3) respectively. When “HAIRPLUS” is used herein without a number appended to the end of the word in reference to tobacco, it is intended to encompass NtHAP1a, NtHAP1b, NtHAP2, NtHAP3, and/or HAIRPLUS genes from Nicotiana species other than N. tabacum as appropriate for the circumstances of its use.

The amino acid sequence of NtHAP1a is provided as SEQ ID NO: 1. The amino acid sequence of NtHAP1b is provided as SEQ ID NO: 2. The amino acid sequence of NtHAP2 is provided as SEQ ID NO: 3. The amino acid sequence of NtHAP3 is provided as SEQ ID NO: 538. The amino acid sequences of additional Nicotiana HAIRPLUS proteins are provided as SEQ ID NOs: 539 to 546. The coding nucleic acid sequence of NtHAP1a is provided as SEQ ID NO: 4. The coding nucleic acid sequence of NtHAP1b is provided as SEQ ID NO: 5. The coding nucleic acid sequence of NtHAP2 is provided as SEQ ID NO: 6. The coding nucleic acid sequence of NtHAP3 is provided as SEQ ID NO: 612. The genomic nucleic acid sequence of NtHAP1a is provided as SEQ ID NO: 7. The genomic nucleic acid sequence of NtHAP1b is provided as SEQ ID NO: 8. The genomic nucleic acid sequence of NtHAP2 is provided as SEQ ID NO: 9. The genomic nucleic acid sequence of NtHAP3 is provided as SEQ ID NO: 537.

The terms “coding nucleic acid sequence” and “coding sequence” are used interchangeably herein. The terms “genomic nucleic acid sequence” and “genomic sequence” are used interchangeably herein.

In an aspect, an endogenous nucleic acid molecule is a genomic sequence comprising SEQ ID NO: 7. In an aspect, an endogenous nucleic acid molecule is a genomic sequence comprising SEQ ID NO: 8. In an aspect, an endogenous nucleic acid molecule is a genomic sequence comprising SEQ ID NO: 9. In an aspect, an endogenous nucleic acid molecule is a genomic sequence comprising SEQ ID NO: 537. As used herein, a “genomic sequence” refers to a sequence present in the genome of a cell. A genomic sequence can include, but is not limited to, a promoter, a 5′-untranslated region (UTR), an exon, an intron, a 3′ UTR, and a terminator.

In an aspect, an endogenous nucleic acid molecule is a coding sequence comprising SEQ ID NO: 4. In an aspect, an endogenous nucleic acid molecule is a coding sequence comprising SEQ ID NO: 5. In an aspect, an endogenous nucleic acid molecule is a coding sequence comprising SEQ ID NO: 6. In an aspect, an endogenous nucleic acid molecule is a coding sequence comprising SEQ ID NO: 612. As used herein, a “coding sequence” refers to the part of a sequence that codes for a protein. Coding sequences do not typically contain intronic sequences, and can comprise a spliced subset of a corresponding genomic sequence.

As used herein, an “endogenous” nucleic acid molecule or amino acid molecule refers to a molecule that originates within a tobacco plant and does not contain any non-natural modifications (e.g., an endogenous nucleic acid molecule or amino acid molecule lacks a non-natural mutation). Endogenous nucleic acid sequences do not include heterologous sequences inserted into a genome via deliberate human intervention. An endogenous nucleic acid molecule or amino acid molecule can also be referred to as a “wild type” (WT) or control version of a given nucleic acid molecule or amino acid molecule. For example, SEQ ID NOs: 1 to 3 and 538 and 538 represent wild type versions of NtHAP1a, NtHAP1b, NtHAP2, and NtHAP3 amino acid sequences, respectively (also referred to as wild type amino acid molecules); SEQ ID NOs: 4 to 6 and 612 represent wild type versions of NtHAP1a, NtHAP1b, NtHAP2, and NtHAP3 coding nucleic acid sequences, respectively (also referred to as wild type nucleic acid molecules); and SEQ ID NOs: 7 to 9 and 537 represent wild type versions of NtHAP1a, NtHAP1b, NtHAP2, and NtHAP3 genomic nucleic acid sequences, respectively (also referred to as wild type nucleic acid molecules). Further, SEQ ID NOs: 539 to 546 represent wild type versions of additional Nicotiana HAIRPLUS amino acid sequences, and SEQ ID NOs: 547 to 588 represent wild type versions of Solanaceae HAIRPLUS amino acid sequences.

In an aspect, a plant provided herein is a modified plant. In an aspect, a seed provided herein is a modified seed. In an aspect, a plant part provided herein is a modified plant part. As used herein, “modified,” in the context of a plant, seed, or plant part, refers to a plant, seed, or plant part, comprising a genetic alteration introduced for certain purposes and beyond natural polymorphisms. Without being limiting, a modified plant, seed, or plant part comprises a recombinant nucleic acid molecule. As used herein, a “recombinant nucleic acid construct” refers to a nucleic acid molecule formed by laboratory methods of genetic recombination, such as, without being limiting, molecular cloning. In another aspect, a modified plant, seed, or plant part comprises a genetic modification. In an aspect, a modified plant, seed, or plant part is a transgenic plant, seed, or plant part.

In an aspect, a plant is a tobacco plant. In an aspect, a plant is a Nicotiana plant. In an aspect, a tobacco plant is a Nicotiana tabacum plant.

In an aspect, a Nicotiana plant, seed, or plant part is selected from the group consisting of Nicotiana tabacum, Nicotiana amplexicaulis PI 271989; Nicotiana benthamiana PI 555478; Nicotiana bigelovii PI 555485; Nicotiana debneyi; Nicotiana excelsior PI 224063; Nicotiana glutinosa PI 555507; Nicotiana goodspeedii PI 241012; Nicotiana gossei PI 230953; Nicotiana hesperis PI 271991; Nicotiana knightiana PI 555527; Nicotiana maritima PI 555535; Nicotiana megalosiphon PI 555536; Nicotiana nudicaulis PI 555540; Nicotiana paniculata PI 555545; Nicotiana plumbaginifolia PI 555548; Nicotiana repanda PI 555552; Nicotiana rustica; Nicotiana suaveolens PI 230960; Nicotiana sylvestris PI 555569; Nicotiana tomentosa PI 266379; Nicotiana tomentosiformis; and Nicotiana trigonophylla PI 555572.

In an aspect, a seed is a tobacco seed. In an aspect, a seed is a Nicotiana seed. In an aspect, a tobacco seed is a Nicotiana tabacum seed.

In an aspect, a plant part is a tobacco plant part. In an aspect, a plant part is a Nicotiana plant part. In an aspect, a tobacco plant part is a Nicotiana tabacum plant part.

In an aspect, a plant part provided includes, but is not limited to, a leaf, a stem, a root, a trichome, a seed, a flower, pollen, an anther, an ovule, a pedicel, a fruit, a meristem, a cotyledon, a hypocotyl, a pod, an embryo, endosperm, an explant, a callus, a tissue culture, a shoot, a cell, and a protoplast. In an aspect, a plant part does not include a seed. In an aspect, this disclosure provides plant cells, tissues, and organs that are not reproductive material and do not mediate the natural reproduction of the plant. In another aspect, this disclosure also provides plant cells, tissues, and organs that are reproductive material and mediate the natural reproduction of the plant. In an aspect, this disclosure provides plant cells, tissues, and organs that cannot maintain themselves via photosynthesis. In another aspect, this disclosure provides somatic plant cells. Somatic cells, contrary to germline cells, do not mediate plant reproduction.

Cells, tissues and organs can be from seed, fruit, leaf, cotyledon, hypocotyl, meristem, embryos, endosperm, root, shoot, stem, trichome, pod, flower, inflorescence, stalk, pedicel, style, stigma, receptacle, petal, sepal, pollen, anther, filament, ovary, ovule, pericarp, phloem, vascular tissue. In another aspect, this disclosure provides a plant chloroplast. In a further aspect, this disclosure provides epidermal cells, stomata cell, leaf or root hairs, a storage root, or a tuber. In another aspect, this disclosure provides a tobacco protoplast.

Skilled artisans understand that tobacco plants naturally reproduce via seeds, not via asexual reproduction or vegetative propagation. In an aspect, this disclosure provides plant endosperm.

In an aspect, this disclosure provides a modified tobacco plant, or part thereof, comprising a non-natural mutation in an endogenous nucleic acid molecule encoding a HAIRPLUS1a (NtHAP1a) protein, where the endogenous NtHAP1a protein comprises the amino acid sequence of SEQ ID NO: 1, and where expression or activity of the NtHAP1a protein is reduced in the modified tobacco plant as compared to a control tobacco plant lacking the non-natural mutation when grown under comparable conditions. In an aspect, an endogenous nucleic acid molecule is a coding sequence comprising SEQ ID NO: 4. In an aspect, an endogenous nucleic acid molecule is a genomic sequence comprising SEQ ID NO: 7. In an aspect, this disclosure provides a modified tobacco plant, or part thereof, comprising a non-natural mutation in an endogenous nucleic acid molecule encoding a HAIRPLUS1a (NtHAP1a) gene, where the endogenous nucleic acid molecule comprises the nucleic acid sequence of SEQ ID NO: 4 or 7, and where expression or activity of the NtHAP1a gene is reduced in the modified tobacco plant as compared to a control tobacco plant lacking the non-natural mutation when grown under comparable conditions.

In an aspect, this disclosure provides a modified tobacco plant, or part thereof, comprising a non-natural mutation in an endogenous nucleic acid molecule encoding a HAIRPLUS1b (NtHAP1b) protein, where the endogenous NtHAP1b protein comprises the amino acid sequence of SEQ ID NO: 2, and where expression or activity of the NtHAP1b protein is reduced in the modified tobacco plant as compared to a control tobacco plant lacking the non-natural mutation when grown under comparable conditions. In an aspect, an endogenous nucleic acid molecule is a coding sequence comprising SEQ ID NO: 5. In an aspect, an endogenous nucleic acid molecule is a genomic sequence comprising SEQ ID NO: 8. In an aspect, this disclosure provides a modified tobacco plant, or part thereof, comprising a non-natural mutation in an endogenous nucleic acid molecule encoding a HAIRPLUS1b (NtHAP1b) gene, where the endogenous nucleic acid molecule comprises the nucleic acid sequence of SEQ ID NO: 5 or 8, and where expression or activity of the NtHAP1b gene is reduced in the modified tobacco plant as compared to a control tobacco plant lacking the non-natural mutation when grown under comparable conditions.

In an aspect, this disclosure provides a modified tobacco plant, or part thereof, comprising a non-natural mutation in an endogenous nucleic acid molecule encoding a HAIRPLUS2 (NtHAP2) protein, where the endogenous NtHAP2 protein comprises the amino acid sequence of SEQ ID NO: 3, and where expression or activity of the NtHAP2 protein is reduced in the modified tobacco plant as compared to a control tobacco plant lacking the non-natural mutation when grown under comparable conditions. In an aspect, an endogenous nucleic acid molecule is a coding sequence comprising SEQ ID NO: 6. In an aspect, an endogenous nucleic acid molecule is a genomic sequence comprising SEQ ID NO: 9. In an aspect, this disclosure provides a modified tobacco plant, or part thereof, comprising a non-natural mutation in an endogenous nucleic acid molecule encoding a HAIRPLUS2 (NtHAP2) gene, where the endogenous nucleic acid molecule comprises the nucleic acid sequence of SEQ ID NO: 6 or 9, and where expression or activity of the NtHAP2 gene is reduced in the modified tobacco plant as compared to a control tobacco plant lacking the non-natural mutation when grown under comparable conditions.

In an aspect, this disclosure provides a modified tobacco plant, or part thereof, comprising a non-natural mutation in an endogenous nucleic acid molecule encoding a HAIRPLUS3 (NtHAP3) protein, where the endogenous NtHAP3 protein comprises the amino acid sequence of SEQ ID NO: 538, and where expression or activity of the NtHAP3 protein is reduced in the modified tobacco plant as compared to a control tobacco plant lacking the non-natural mutation when grown under comparable conditions. In an aspect, an endogenous nucleic acid molecule is a coding sequence comprising SEQ ID NO: 612. In an aspect, an endogenous nucleic acid molecule is a genomic sequence comprising SEQ ID NO: 537. In an aspect, this disclosure provides a modified tobacco plant, or part thereof, comprising a non-natural mutation in an endogenous nucleic acid molecule encoding a HAIRPLUS3 (NtHAP3) gene, where the endogenous nucleic acid molecule comprises the nucleic acid sequence of SEQ ID NO: 537 or 612, and where expression or activity of the NtHAP3 gene is reduced in the modified tobacco plant as compared to a control tobacco plant lacking the non-natural mutation when grown under comparable conditions.

In an aspect, a modified tobacco plant, or part thereof, comprises a first mutation in an endogenous nucleic acid molecule encoding an NtHAP1a protein and a second mutation in an endogenous nucleic acid molecule encoding an NtHAP1b protein. In an aspect, a modified tobacco plant, or part thereof, comprises a first mutation in an endogenous nucleic acid molecule encoding an NtHAP1a protein and a second mutation in an endogenous nucleic acid molecule encoding an NtHAP2 protein. In an aspect, a modified tobacco plant, or part thereof, comprises a first mutation in an endogenous nucleic acid molecule encoding an NtHAP1a protein and a second mutation in an endogenous nucleic acid molecule encoding an NtHAP3 protein. In an aspect, a modified tobacco plant, or part thereof, comprises a first mutation in an endogenous nucleic acid molecule encoding an NtHAP1b protein and a second mutation in an endogenous nucleic acid molecule encoding an NtHAP2 protein. In an aspect, a modified tobacco plant, or part thereof, comprises a first mutation in an endogenous nucleic acid molecule encoding an NtHAP1b protein and a second mutation in an endogenous nucleic acid molecule encoding an NtHAP3 protein. In an aspect, a modified tobacco plant, or part thereof, comprises a first mutation in an endogenous nucleic acid molecule encoding an NtHAP2 protein and a second mutation in an endogenous nucleic acid molecule encoding an NtHAP3 protein. In an aspect, a modified tobacco plant, or part thereof, comprises a first mutation in an endogenous nucleic acid molecule encoding an NtHAP1a protein, a second mutation in an endogenous nucleic acid molecule encoding an NtHAP1b protein, and a third mutation in an endogenous nucleic acid molecule encoding an NtHAP2 protein. In an aspect, a modified tobacco plant, or part thereof, comprises a first mutation in an endogenous nucleic acid molecule encoding an NtHAP1a protein, a second mutation in an endogenous nucleic acid molecule encoding an NtHAP1b protein, and a third mutation in an endogenous nucleic acid molecule encoding an NtHAP3 protein. In an aspect, a modified tobacco plant, or part thereof, comprises a first mutation in an endogenous nucleic acid molecule encoding an NtHAP1a protein, a second mutation in an endogenous nucleic acid molecule encoding an NtHAP2 protein, and a third mutation in an endogenous nucleic acid molecule encoding an NtHAP3 protein. In an aspect, a modified tobacco plant, or part thereof, comprises a first mutation in an endogenous nucleic acid molecule encoding an NtHAP1b protein, a second mutation in an endogenous nucleic acid molecule encoding an NtHAP2 protein, and a third mutation in an endogenous nucleic acid molecule encoding an NtHAP3 protein. In an aspect, a modified tobacco plant, or part thereof, comprises a first mutation in an endogenous nucleic acid molecule encoding an NtHAP1a protein, a second mutation in an endogenous nucleic acid molecule encoding an NtHAP1b protein, a third mutation in an endogenous nucleic acid molecule encoding an NtHAP2 protein, and a fourth mutation in an endogenous nucleic acid molecule encoding an NtHAP3 protein.

In an aspect, a modified tobacco plant, or part thereof, comprises (a) a first non-natural mutation in an endogenous nucleic acid molecule encoding a HAIRPLUS1a (NtHAP1a) protein, where the endogenous NtHAP1a protein comprises the amino acid sequence of SEQ ID NO: 1, and where expression or activity of the NtHAP1a protein is reduced in the modified tobacco plant as compared to a control tobacco plant lacking the first non-natural mutation when grown under comparable conditions; and (b) a second non-natural mutation in an endogenous nucleic acid molecule encoding a HAIRPLUS1b (NtHAP1b) protein, where the endogenous NtHAP1b protein comprises the amino acid sequence of SEQ ID NO: 2, and where expression or activity of the NtHAP1b protein is reduced in the modified tobacco plant as compared to a control tobacco plant lacking the second non-natural mutation when grown under comparable conditions. In an aspect, a modified tobacco plant, or part thereof, comprises (a) a first non-natural mutation in an endogenous nucleic acid molecule encoding a HAIRPLUS1a (NtHAP1a) gene, where the endogenous nucleic acid molecule comprises the nucleic acid sequence of SEQ ID NO: 4 or 7, and where expression or activity of the NtHAP1a gene is reduced in the modified tobacco plant as compared to a control tobacco plant lacking the first non-natural mutation when grown under comparable conditions; and (b) a second non-natural mutation in an endogenous nucleic acid molecule encoding a HAIRPLUS1b (NtHAP1b) gene, where the endogenous nucleic acid molecule comprises the nucleic acid sequence of SEQ ID NO: 5 or 8, and where expression or activity of the NtHAP1b gene is reduced in the modified tobacco plant as compared to a control tobacco plant lacking the second non-natural mutation when grown under comparable conditions.

In an aspect, a modified tobacco plant, or part thereof, comprises (a) a first non-natural mutation in an endogenous nucleic acid molecule encoding a HAIRPLUS1a (NtHAP1a) protein, where the endogenous NtHAP1a protein comprises the amino acid sequence of SEQ ID NO: 1, and where expression or activity of the NtHAP1a protein is reduced in the modified tobacco plant as compared to a control tobacco plant lacking the first non-natural mutation when grown under comparable conditions; and (b) a second non-natural mutation in an endogenous nucleic acid molecule encoding a HAIRPLUS2 (NtHAP2) protein, where the endogenous NtHAP2 protein comprises the amino acid sequence of SEQ ID NO: 3, and where expression or activity of the NtHAP2 protein is reduced in the modified tobacco plant as compared to a control tobacco plant lacking the second non-natural mutation when grown under comparable conditions. In an aspect, a modified tobacco plant, or part thereof, comprises (a) a first non-natural mutation in an endogenous nucleic acid molecule encoding a HAIRPLUS1a (NtHAP1a) gene, where the endogenous nucleic acid molecule comprises the nucleic acid sequence of SEQ ID NO: 4 or 7, and where expression or activity of the NtHAP1a gene is reduced in the modified tobacco plant as compared to a control tobacco plant lacking the first non-natural mutation when grown under comparable conditions; and (b) a second non-natural mutation in an endogenous nucleic acid molecule encoding a HAIRPLUS2 (NtHAP2) gene, where the endogenous nucleic acid molecule comprises the nucleic acid sequence of SEQ ID NO: 6 or 9, and where expression or activity of the NtHAP2 gene is reduced in the modified tobacco plant as compared to a control tobacco plant lacking the second non-natural mutation when grown under comparable conditions.

In an aspect, a modified tobacco plant, or part thereof, comprises (a) a first non-natural mutation in an endogenous nucleic acid molecule encoding a HAIRPLUS1a (NtHAP1a) protein, where the endogenous NtHAP1a protein comprises the amino acid sequence of SEQ ID NO: 1, and where expression or activity of the NtHAP1a protein is reduced in the modified tobacco plant as compared to a control tobacco plant lacking the first non-natural mutation when grown under comparable conditions; and (b) a second non-natural mutation in an endogenous nucleic acid molecule encoding a HAIRPLUS3 (NtHAP3) protein, where the endogenous NtHAP3 protein comprises the amino acid sequence of SEQ ID NO: 538, and where expression or activity of the NtHAP3 protein is reduced in the modified tobacco plant as compared to a control tobacco plant lacking the second non-natural mutation when grown under comparable conditions. In an aspect, a modified tobacco plant, or part thereof, comprises (a) a first non-natural mutation in an endogenous nucleic acid molecule encoding a HAIRPLUS1a (NtHAP1a) gene, where the endogenous nucleic acid molecule comprises the nucleic acid sequence of SEQ ID NO: 4 or 7, and where expression or activity of the NtHAP1a gene is reduced in the modified tobacco plant as compared to a control tobacco plant lacking the first non-natural mutation when grown under comparable conditions; and (b) a second non-natural mutation in an endogenous nucleic acid molecule encoding a HAIRPLUS3 (NtHAP3) gene, where the endogenous nucleic acid molecule comprises the nucleic acid sequence of SEQ ID NO: 537 or 612, and where expression or activity of the NtHAP3 gene is reduced in the modified tobacco plant as compared to a control tobacco plant lacking the second non-natural mutation when grown under comparable conditions.

In an aspect, a modified tobacco plant, or part thereof, comprises (a) a first non-natural mutation in an endogenous nucleic acid molecule encoding a HAIRPLUS1b (NtHAP1b) protein, where the endogenous NtHAP1b protein comprises the amino acid sequence of SEQ ID NO: 2, and where expression or activity of the NtHAP1b protein is reduced in the modified tobacco plant as compared to a control tobacco plant lacking the first non-natural mutation when grown under comparable conditions; and (b) a second non-natural mutation in an endogenous nucleic acid molecule encoding a HAIRPLUS2 (NtHAP2) protein, where the endogenous NtHAP2 protein comprises the amino acid sequence of SEQ ID NO: 3, and where expression or activity of the NtHAP2 protein is reduced in the modified tobacco plant as compared to a control tobacco plant lacking the second non-natural mutation when grown under comparable conditions. In an aspect, a modified tobacco plant, or part thereof, comprises (a) a first non-natural mutation in an endogenous nucleic acid molecule encoding a HAIRPLUS1b (NtHAP1b) gene, where the endogenous nucleic acid molecule comprises the nucleic acid sequence of SEQ ID NO: 5 or 8, and where expression or activity of the NtHAP1b gene is reduced in the modified tobacco plant as compared to a control tobacco plant lacking the first non-natural mutation when grown under comparable conditions; and (b) a second non-natural mutation in an endogenous nucleic acid molecule encoding a HAIRPLUS2 (NtHAP2) gene, where the endogenous nucleic acid molecule comprises the nucleic acid sequence of SEQ ID NO: 6 or 9, and where expression or activity of the NtHAP2 gene is reduced in the modified tobacco plant as compared to a control tobacco plant lacking the second non-natural mutation when grown under comparable conditions.

In an aspect, a modified tobacco plant, or part thereof, comprises (a) a first non-natural mutation in an endogenous nucleic acid molecule encoding a HAIRPLUS1b (NtHAP1b) protein, where the endogenous NtHAP1b protein comprises the amino acid sequence of SEQ ID NO: 2, and where expression or activity of the NtHAP1b protein is reduced in the modified tobacco plant as compared to a control tobacco plant lacking the first non-natural mutation when grown under comparable conditions; and (b) a second non-natural mutation in an endogenous nucleic acid molecule encoding a HAIRPLUS3 (NtHAP3) protein, where the endogenous NtHAP3 protein comprises the amino acid sequence of SEQ ID NO: 538, and where expression or activity of the NtHAP3 protein is reduced in the modified tobacco plant as compared to a control tobacco plant lacking the second non-natural mutation when grown under comparable conditions. In an aspect, a modified tobacco plant, or part thereof, comprises (a) a first non-natural mutation in an endogenous nucleic acid molecule encoding a HAIRPLUS1b (NtHAP1b) gene, where the endogenous nucleic acid molecule comprises the nucleic acid sequence of SEQ ID NO: 5 or 8, and where expression or activity of the NtHAP1b gene is reduced in the modified tobacco plant as compared to a control tobacco plant lacking the first non-natural mutation when grown under comparable conditions; and (b) a second non-natural mutation in an endogenous nucleic acid molecule encoding a HAIRPLUS3 (NtHAP3) gene, where the endogenous nucleic acid molecule comprises the nucleic acid sequence of SEQ ID NO: 537 or 612, and where expression or activity of the NtHAP3 gene is reduced in the modified tobacco plant as compared to a control tobacco plant lacking the second non-natural mutation when grown under comparable conditions.

In an aspect, a modified tobacco plant, or part thereof, comprises (a) a first non-natural mutation in an endogenous nucleic acid molecule encoding a HAIRPLUS2 (NtHAP2) protein, where the endogenous NtHAP2 protein comprises the amino acid sequence of SEQ ID NO: 3, and where expression or activity of the NtHAP2 protein is reduced in the modified tobacco plant as compared to a control tobacco plant lacking the first non-natural mutation when grown under comparable conditions; and (b) a second non-natural mutation in an endogenous nucleic acid molecule encoding a HAIRPLUS3 (NtHAP3) protein, where the endogenous NtHAP3 protein comprises the amino acid sequence of SEQ ID NO: 538, and where expression or activity of the NtHAP3 protein is reduced in the modified tobacco plant as compared to a control tobacco plant lacking the second non-natural mutation when grown under comparable conditions. In an aspect, a modified tobacco plant, or part thereof, comprises (a) a first non-natural mutation in an endogenous nucleic acid molecule encoding a HAIRPLUS2 (NtHAP2) gene, where the endogenous nucleic acid molecule comprises the nucleic acid sequence of SEQ ID NO: 6 or 9, and where expression or activity of the NtHAP2 gene is reduced in the modified tobacco plant as compared to a control tobacco plant lacking the first non-natural mutation when grown under comparable conditions; and (b) a second non-natural mutation in an endogenous nucleic acid molecule encoding a HAIRPLUS3 (NtHAP3) gene, where the endogenous nucleic acid molecule comprises the nucleic acid sequence of SEQ ID NO: 537 or 612, and where expression or activity of the NtHAP3 gene is reduced in the modified tobacco plant as compared to a control tobacco plant lacking the second non-natural mutation when grown under comparable conditions.

In an aspect, a modified tobacco plant, or part thereof, comprises (a) a first non-natural mutation in an endogenous nucleic acid molecule encoding a HAIRPLUS1a (NtHAP1a) protein, where the endogenous NtHAP1a protein comprises the amino acid sequence of SEQ ID NO: 1, and where expression or activity of the NtHAP1a protein is reduced in the modified tobacco plant as compared to a control tobacco plant lacking the first non-natural mutation when grown under comparable conditions; (b) a second non-natural mutation in an endogenous nucleic acid molecule encoding a HAIRPLUS1b (NtHAP1b) protein, where the endogenous NtHAP1b protein comprises the amino acid sequence of SEQ ID NO: 2, and where expression or activity of the NtHAP1b protein is reduced in the modified tobacco plant as compared to a control tobacco plant lacking the second non-natural mutation when grown under comparable conditions; and (c) a third non-natural mutation in an endogenous nucleic acid molecule encoding a HAIRPLUS2 (NtHAP2) protein, where the endogenous NtHAP2 protein comprises the amino acid sequence of SEQ ID NO: 3, and where expression or activity of the NtHAP2 protein is reduced in the modified tobacco plant as compared to a control tobacco plant lacking the third non-natural mutation when grown under comparable conditions. In an aspect, a modified tobacco plant, or part thereof, comprises (a) a first non-natural mutation in an endogenous nucleic acid molecule encoding a HAIRPLUS1a (NtHAP1a) gene, where the endogenous nucleic acid molecule comprises the nucleic acid sequence of SEQ ID NO: 4 or 7, and where expression or activity of the NtHAP1a gene is reduced in the modified tobacco plant as compared to a control tobacco plant lacking the first non-natural mutation when grown under comparable conditions; (b) a second non-natural mutation in an endogenous nucleic acid molecule encoding a HAIRPLUS1b (NtHAP1b) gene, where the endogenous nucleic acid molecule comprises the nucleic acid sequence of SEQ ID NO: 5 or 8, and where expression or activity of the NtHAP1b gene is reduced in the modified tobacco plant as compared to a control tobacco plant lacking the second non-natural mutation when grown under comparable conditions; and (c) a third non-natural mutation in an endogenous nucleic acid molecule encoding a HAIRPLUS2 (NtHAP2) gene, where the endogenous nucleic acid molecule comprises the nucleic acid sequence of SEQ ID NO: 6 or 9, and where expression or activity of the NtHAP2 gene is reduced in the modified tobacco plant as compared to a control tobacco plant lacking the third non-natural mutation when grown under comparable conditions.

In an aspect, a modified tobacco plant, or part thereof, comprises (a) a first non-natural mutation in an endogenous nucleic acid molecule encoding a HAIRPLUS1a (NtHAP1a) protein, where the endogenous NtHAP1a protein comprises the amino acid sequence of SEQ ID NO: 1, and where expression or activity of the NtHAP1a protein is reduced in the modified tobacco plant as compared to a control tobacco plant lacking the first non-natural mutation when grown under comparable conditions; (b) a second non-natural mutation in an endogenous nucleic acid molecule encoding a HAIRPLUS1b (NtHAP1b) protein, where the endogenous NtHAP1b protein comprises the amino acid sequence of SEQ ID NO: 2, and where expression or activity of the NtHAP1b protein is reduced in the modified tobacco plant as compared to a control tobacco plant lacking the second non-natural mutation when grown under comparable conditions; and (c) a third non-natural mutation in an endogenous nucleic acid molecule encoding a HAIRPLUS3 (NtHAP3) protein, where the endogenous NtHAP3 protein comprises the amino acid sequence of SEQ ID NO: 538, and where expression or activity of the NtHAP3 protein is reduced in the modified tobacco plant as compared to a control tobacco plant lacking the third non-natural mutation when grown under comparable conditions. In an aspect, a modified tobacco plant, or part thereof, comprises (a) a first non-natural mutation in an endogenous nucleic acid molecule encoding a HAIRPLUS1a (NtHAP1a) gene, where the endogenous nucleic acid molecule comprises the nucleic acid sequence of SEQ ID NO: 4 or 7, and where expression or activity of the NtHAP1a gene is reduced in the modified tobacco plant as compared to a control tobacco plant lacking the first non-natural mutation when grown under comparable conditions; (b) a second non-natural mutation in an endogenous nucleic acid molecule encoding a HAIRPLUS1b (NtHAP1b) gene, where the endogenous nucleic acid molecule comprises the nucleic acid sequence of SEQ ID NO: 5 or 8, and where expression or activity of the NtHAP1b gene is reduced in the modified tobacco plant as compared to a control tobacco plant lacking the second non-natural mutation when grown under comparable conditions; and (c) a third non-natural mutation in an endogenous nucleic acid molecule encoding a HAIRPLUS3 (NtHAP3) gene, where the endogenous nucleic acid molecule comprises the nucleic acid sequence of SEQ ID NO: 537 or 612, and where expression or activity of the NtHAP3 gene is reduced in the modified tobacco plant as compared to a control tobacco plant lacking the third non-natural mutation when grown under comparable conditions.

In an aspect, a modified tobacco plant, or part thereof, comprises (a) a first non-natural mutation in an endogenous nucleic acid molecule encoding a HAIRPLUS1a (NtHAP1a) protein, where the endogenous NtHAP1a protein comprises the amino acid sequence of SEQ ID NO: 1, and where expression or activity of the NtHAP1a protein is reduced in the modified tobacco plant as compared to a control tobacco plant lacking the first non-natural mutation when grown under comparable conditions; (b) a second non-natural mutation in an endogenous nucleic acid molecule encoding a HAIRPLUS2 (NtHAP2) protein, where the endogenous NtHAP2 protein comprises the amino acid sequence of SEQ ID NO: 3, and where expression or activity of the NtHAP2 protein is reduced in the modified tobacco plant as compared to a control tobacco plant lacking the second non-natural mutation when grown under comparable conditions; and (c) a third non-natural mutation in an endogenous nucleic acid molecule encoding a HAIRPLUS3 (NtHAP3) protein, where the endogenous NtHAP3 protein comprises the amino acid sequence of SEQ ID NO: 538, and where expression or activity of the NtHAP3 protein is reduced in the modified tobacco plant as compared to a control tobacco plant lacking the third non-natural mutation when grown under comparable conditions. In an aspect, a modified tobacco plant, or part thereof, comprises (a) a first non-natural mutation in an endogenous nucleic acid molecule encoding a HAIRPLUS1a (NtHAP1a) gene, where the endogenous nucleic acid molecule comprises the nucleic acid sequence of SEQ ID NO: 4 or 7, and where expression or activity of the NtHAP1a gene is reduced in the modified tobacco plant as compared to a control tobacco plant lacking the first non-natural mutation when grown under comparable conditions; (b) a second non-natural mutation in an endogenous nucleic acid molecule encoding a HAIRPLUS2 (NtHAP2) gene, where the endogenous nucleic acid molecule comprises the nucleic acid sequence of SEQ ID NO: 6 or 9, and where expression or activity of the NtHAP2 gene is reduced in the modified tobacco plant as compared to a control tobacco plant lacking the second non-natural mutation when grown under comparable conditions; and (c) a third non-natural mutation in an endogenous nucleic acid molecule encoding a HAIRPLUS3 (NtHAP3) gene, where the endogenous nucleic acid molecule comprises the nucleic acid sequence of SEQ ID NO: 537 or 612, and where expression or activity of the NtHAP3 gene is reduced in the modified tobacco plant as compared to a control tobacco plant lacking the third non-natural mutation when grown under comparable conditions.

In an aspect, a modified tobacco plant, or part thereof, comprises (a) a first non-natural mutation in an endogenous nucleic acid molecule encoding a HAIRPLUS1b (NtHAP1b) protein, where the endogenous NtHAP1b protein comprises the amino acid sequence of SEQ ID NO: 2, and where expression or activity of the NtHAP1b protein is reduced in the modified tobacco plant as compared to a control tobacco plant lacking the first non-natural mutation when grown under comparable conditions; (b) a second non-natural mutation in an endogenous nucleic acid molecule encoding a HAIRPLUS2 (NtHAP2) protein, where the endogenous NtHAP2 protein comprises the amino acid sequence of SEQ ID NO: 3, and where expression or activity of the NtHAP2 protein is reduced in the modified tobacco plant as compared to a control tobacco plant lacking the second non-natural mutation when grown under comparable conditions; and (c) a third non-natural mutation in an endogenous nucleic acid molecule encoding a HAIRPLUS3 (NtHAP3) protein, where the endogenous NtHAP3 protein comprises the amino acid sequence of SEQ ID NO: 538, and where expression or activity of the NtHAP3 protein is reduced in the modified tobacco plant as compared to a control tobacco plant lacking the third non-natural mutation when grown under comparable conditions. In an aspect, a modified tobacco plant, or part thereof, comprises (a) a first non-natural mutation in an endogenous nucleic acid molecule encoding a HAIRPLUS1b (NtHAP1b) gene, where the endogenous nucleic acid molecule comprises the nucleic acid sequence of SEQ ID NO: 5 or 8, and where expression or activity of the NtHAP1b gene is reduced in the modified tobacco plant as compared to a control tobacco plant lacking the first non-natural mutation when grown under comparable conditions; (b) a second non-natural mutation in an endogenous nucleic acid molecule encoding a HAIRPLUS2 (NtHAP2) gene, where the endogenous nucleic acid molecule comprises the nucleic acid sequence of SEQ ID NO: 6 or 9, and where expression or activity of the NtHAP2 gene is reduced in the modified tobacco plant as compared to a control tobacco plant lacking the second non-natural mutation when grown under comparable conditions; and (c) a third non-natural mutation in an endogenous nucleic acid molecule encoding a HAIRPLUS3 (NtHAP3) gene, where the endogenous nucleic acid molecule comprises the nucleic acid sequence of SEQ ID NO: 537 or 612, and where expression or activity of the NtHAP3 gene is reduced in the modified tobacco plant as compared to a control tobacco plant lacking the third non-natural mutation when grown under comparable conditions.

In an aspect, a modified tobacco plant, or part thereof, comprises (a) a first non-natural mutation in an endogenous nucleic acid molecule encoding a HAIRPLUS1a (NtHAP1a) protein, where the endogenous NtHAP1a protein comprises the amino acid sequence of SEQ ID NO: 1, and where expression or activity of the NtHAP1a protein is reduced in the modified tobacco plant as compared to a control tobacco plant lacking the first non-natural mutation when grown under comparable conditions; (b) a second non-natural mutation in an endogenous nucleic acid molecule encoding a HAIRPLUS1b (NtHAP1b) protein, where the endogenous NtHAP1b protein comprises the amino acid sequence of SEQ ID NO: 2, and where expression or activity of the NtHAP1b protein is reduced in the modified tobacco plant as compared to a control tobacco plant lacking the second non-natural mutation when grown under comparable conditions; (c) a third non-natural mutation in an endogenous nucleic acid molecule encoding a HAIRPLUS2 (NtHAP2) protein, where the endogenous NtHAP2 protein comprises the amino acid sequence of SEQ ID NO: 3, and where expression or activity of the NtHAP2 protein is reduced in the modified tobacco plant as compared to a control tobacco plant lacking the third non-natural mutation when grown under comparable conditions; and (d) a fourth non-natural mutation in an endogenous nucleic acid molecule encoding a HAIRPLUS3 (NtHAP3) protein, where the endogenous NtHAP3 protein comprises the amino acid sequence of SEQ ID NO: 538, and where expression or activity of the NtHAP3 protein is reduced in the modified tobacco plant as compared to a control tobacco plant lacking the fourth non-natural mutation when grown under comparable conditions. In an aspect, a modified tobacco plant, or part thereof, comprises (a) a first non-natural mutation in an endogenous nucleic acid molecule encoding a HAIRPLUS1a (NtHAP1a) gene, where the endogenous nucleic acid molecule comprises the nucleic acid sequence of SEQ ID NO: 4 or 7, and where expression or activity of the NtHAP1a gene is reduced in the modified tobacco plant as compared to a control tobacco plant lacking the first non-natural mutation when grown under comparable conditions; (b) a second non-natural mutation in an endogenous nucleic acid molecule encoding a HAIRPLUS1b (NtHAP1b) gene, where the endogenous nucleic acid molecule comprises the nucleic acid sequence of SEQ ID NO: 5 or 8, and where expression or activity of the NtHAP1b gene is reduced in the modified tobacco plant as compared to a control tobacco plant lacking the second non-natural mutation when grown under comparable conditions; (c) a third non-natural mutation in an endogenous nucleic acid molecule encoding a HAIRPLUS2 (NtHAP2) gene, where the endogenous nucleic acid molecule comprises the nucleic acid sequence of SEQ ID NO: 6 or 9, and where expression or activity of the NtHAP2 gene is reduced in the modified tobacco plant as compared to a control tobacco plant lacking the third non-natural mutation when grown under comparable conditions; and (d) a fourth non-natural mutation in an endogenous nucleic acid molecule encoding a HAIRPLUS3 (NtHAP3) gene, where the endogenous nucleic acid molecule comprises the nucleic acid sequence of SEQ ID NO: 537 or 612, and where expression or activity of the NtHAP3 gene is reduced in the modified tobacco plant as compared to a control tobacco plant lacking the fourth non-natural mutation when grown under comparable conditions.

In an aspect, this disclosure provides a modified tobacco plant, or part thereof, comprising a non-natural mutation in a nucleic acid molecule encoding a HAIRPLUS1a protein, where the nucleic acid molecule comprises a nucleic acid sequence selected from the group consisting of SEQ ID NOs: 20 to 23. In an aspect, this disclosure provides a modified tobacco plant, or part thereof, comprising a mutated HAIRPLUS1a protein, where the mutated NtHAP1a protein comprises an amino acid sequence selected from the group consisting of SEQ ID NOs: 27 to 30. In an aspect, expression or activity of the NtHAP1a protein is reduced in a modified tobacco plant comprising any one of SEQ ID NOs: 20 to 23 and 27 to 30 as compared to a control tobacco plant when grown under comparable conditions.

In an aspect, this disclosure provides a modified tobacco plant, or part thereof, comprising a non-natural mutation in a nucleic acid molecule encoding a HAIRPLUS1b protein, where the nucleic acid molecule comprises a nucleic acid sequence selected from the group consisting of SEQ ID NOs: 24 and 25. In an aspect, this disclosure provides a modified tobacco plant, or part thereof, comprising a mutated HAIRPLUS1b (NtHAP1b) protein, where the mutated NtHAP1b protein comprises an amino acid sequence selected from the group consisting of SEQ ID NOs: 31 and 32. In an aspect, expression or activity of the NtHAP1b protein is reduced in a modified tobacco plant comprising any one of SEQ ID NOs: 24, 25, 31, and 32 as compared to a control tobacco plant when grown under comparable conditions.

In an aspect, this disclosure provides a modified tobacco plant, or part thereof, comprising a non-natural mutation in a nucleic acid molecule encoding a HAIRPLUS2 protein, where the nucleic acid molecule comprises the nucleic acid sequence of SEQ ID NO: 26. In an aspect, this disclosure provides a modified tobacco plant, or part thereof, comprising a mutated HAIRPLUS2 (NtHAP2) protein, where the mutated NtHAP2 protein comprises the amino acid sequence of SEQ ID NO: 33. In an aspect, expression or activity of the NtHAP2 protein is reduced in a modified tobacco plant comprising SEQ ID NO: 26 or SEQ ID NO: 32 as compared to a control tobacco plant when grown under comparable conditions.

The expression (e.g., accumulation) or activity of endogenous and/or modified versions of NtHAP1a, NtHAP1b, NtHAP2, and/or NtHAP3 can be measured using any suitable method known in the art, such as, without being limiting, protein sequencing, RNA sequencing, qRT-PCR, RNA gel blots, ELISA, Western blots, measuring fluorescence intensity, measuring bioluminescence intensity, and specific protein-activity assays. Gene expression or activity encompasses either or both transcription and translation of the target gene. Non-limiting examples of measuring expression include quantitative reverse transcriptase polymerase chain reaction (qRT-PCR), RNA blot (e.g., a Northern blot), RNA sequencing. Differences in expression can be described as an absolute quantification or a relative quantification. See, for example, Livak and Schmittgen, Methods, 25:402-408 (2001). If an endogenous nucleic acid sequence encodes a protein, changes in expression can be inferred by examining the accumulation of the encoded protein. Non-limiting examples of measuring protein accumulation include Western blots and enzyme-linked immunosorbent assays (ELISAs).

In some aspects, a visible plant phenotype (e.g., without being limiting, increased trichome number, increased trichome density, increased trichome size) in a modified plant as compared to a control plant is evidence of reduced expression or activity of a mutated or suppressed gene or genes (e.g., NtHAP1a, NtHAP1b, NtHAP2, NtHAP3).

In an aspect, expression or activity of an NtHAP1a protein comprising a non-natural modification is reduced by at least 1% as compared to expression of a wild type version of the NtHAP1a protein in a control tobacco plant when grown under comparable conditions. In an aspect, expression or activity of an NtHAP1a protein comprising a non-natural modification is reduced by at least 5% as compared to expression of a wild type version of the NtHAP1a protein in a control tobacco plant when grown under comparable conditions. In an aspect, expression or activity of an NtHAP1a protein comprising a non-natural modification is reduced by at least 10% as compared to expression of a wild type version of the NtHAP1a protein in a control tobacco plant when grown under comparable conditions. In an aspect, expression or activity of an NtHAP1a protein comprising a non-natural modification is reduced by at least 20% as compared to expression of a wild type version of the NtHAP1a protein in a control tobacco plant when grown under comparable conditions. In an aspect, expression or activity of an NtHAP1a protein comprising a non-natural modification is reduced by at least 30% as compared to expression of a wild type version of the NtHAP1a protein in a control tobacco plant when grown under comparable conditions. In an aspect, expression or activity of an NtHAP1a protein comprising a non-natural modification is reduced by at least 40% as compared to expression of a wild type version of the NtHAP1a protein in a control tobacco plant when grown under comparable conditions. In an aspect, expression or activity of an NtHAP1a protein comprising a non-natural modification is reduced by at least 50% as compared to expression of a wild type version of the NtHAP1a protein in a control tobacco plant when grown under comparable conditions. In an aspect, expression or activity of an NtHAP1a protein comprising a non-natural modification is reduced by at least 60% as compared to expression of a wild type version of the NtHAP1a protein in a control tobacco plant when grown under comparable conditions. In an aspect, expression or activity of an NtHAP1a protein comprising a non-natural modification is reduced by at least 70% as compared to expression of a wild type version of the NtHAP1a protein in a control tobacco plant when grown under comparable conditions. In an aspect, expression or activity of an NtHAP1a protein comprising a non-natural modification is reduced by at least 80% as compared to expression of a wild type version of the NtHAP1a protein in a control tobacco plant when grown under comparable conditions. In an aspect, expression or activity of an NtHAP1a protein comprising a non-natural modification is reduced by at least 90% as compared to expression of a wild type version of the NtHAP1a protein in a control tobacco plant when grown under comparable conditions. In an aspect, expression or activity of an NtHAP1a protein comprising a non-natural modification is reduced by at least 95% as compared to expression of a wild type version of the NtHAP1a protein in a control tobacco plant when grown under comparable conditions. In an aspect, expression or activity of an NtHAP1a protein comprising a non-natural modification is reduced by at least 99% as compared to expression of a wild type version of the NtHAP1a protein in a control tobacco plant when grown under comparable conditions. In an aspect, expression or activity of an NtHAP1a protein comprising a non-natural modification is reduced by 100% as compared to expression of a wild type version of the NtHAP1a protein in a control tobacco plant when grown under comparable conditions.

In an aspect, expression of a NtHAP1a gene comprising a non-natural mutation is reduced by at least 1% as compared to expression of an wild type version of the NtHAP1a gene in a control tobacco plant when grown under comparable conditions. In an aspect, expression of a NtHAP1a gene comprising a non-natural mutation is reduced by at least 5% as compared to expression of an wild type version of the NtHAP1a gene in a control tobacco plant when grown under comparable conditions. In an aspect, expression of a NtHAP1a gene comprising a non-natural mutation is reduced by at least 10% as compared to expression of an wild type version of the NtHAP1a gene in a control tobacco plant when grown under comparable conditions. In an aspect, expression of a NtHAP1a gene comprising a non-natural mutation is reduced by at least 20% as compared to expression of an wild type version of the NtHAP1a gene in a control tobacco plant when grown under comparable conditions. In an aspect, expression of a NtHAP1a gene comprising a non-natural mutation is reduced by at least 30% as compared to expression of an wild type version of the NtHAP1a gene in a control tobacco plant when grown under comparable conditions. In an aspect, expression of a NtHAP1a gene comprising a non-natural mutation is reduced by at least 40% as compared to expression of an wild type version of the NtHAP1a gene in a control tobacco plant when grown under comparable conditions. In an aspect, expression of a NtHAP1a gene comprising a non-natural mutation is reduced by at least 50% as compared to expression of an wild type version of the NtHAP1a gene in a control tobacco plant when grown under comparable conditions. In an aspect, expression of a NtHAP1a gene comprising a non-natural mutation is reduced by at least 60% as compared to expression of an wild type version of the NtHAP1a gene in a control tobacco plant when grown under comparable conditions. In an aspect, expression of a NtHAP1a gene comprising a non-natural mutation is reduced by at least 70% as compared to expression of an wild type version of the NtHAP1a gene in a control tobacco plant when grown under comparable conditions. In an aspect, expression of a NtHAP1a gene comprising a non-natural mutation is reduced by at least 80% as compared to expression of an wild type version of the NtHAP1a gene in a control tobacco plant when grown under comparable conditions. In an aspect, expression of a NtHAP1a gene comprising a non-natural mutation is reduced by at least 90% as compared to expression of an wild type version of the NtHAP1a gene in a control tobacco plant when grown under comparable conditions. In an aspect, expression of a NtHAP1a gene comprising a non-natural mutation is reduced by at least 95% as compared to expression of an wild type version of the NtHAP1a gene in a control tobacco plant when grown under comparable conditions. In an aspect, expression of a NtHAP1a gene comprising a non-natural mutation is reduced by at least 99% as compared to expression of an wild type version of the NtHAP1a gene in a control tobacco plant when grown under comparable conditions. In an aspect, expression of a NtHAP1a gene comprising a non-natural mutation is reduced by 100% as compared to expression of an wild type version of the NtHAP1a gene in a control tobacco plant when grown under comparable conditions.

In an aspect, expression or activity of an NtHAP1b protein comprising a non-natural modification is reduced by at least 1% as compared to expression of a wild type version of the NtHAP1b protein in a control tobacco plant when grown under comparable conditions. In an aspect, expression or activity of an NtHAP1b protein comprising a non-natural modification is reduced by at least 5% as compared to expression of a wild type version of the NtHAP1b protein in a control tobacco plant when grown under comparable conditions. In an aspect, expression or activity of an NtHAP1b protein comprising a non-natural modification is reduced by at least 10% as compared to expression of a wild type version of the NtHAP1b protein in a control tobacco plant when grown under comparable conditions. In an aspect, expression or activity of an NtHAP1b protein comprising a non-natural modification is reduced by at least 20% as compared to expression of a wild type version of the NtHAP1b protein in a control tobacco plant when grown under comparable conditions. In an aspect, expression or activity of an NtHAP1b protein comprising a non-natural modification is reduced by at least 30% as compared to expression of a wild type version of the NtHAP1b protein in a control tobacco plant when grown under comparable conditions. In an aspect, expression or activity of an NtHAP1b protein comprising a non-natural modification is reduced by at least 40% as compared to expression of a wild type version of the NtHAP1b protein in a control tobacco plant when grown under comparable conditions. In an aspect, expression or activity of an NtHAP1b protein comprising a non-natural modification is reduced by at least 50% as compared to expression of a wild type version of the NtHAP1b protein in a control tobacco plant when grown under comparable conditions. In an aspect, expression or activity of an NtHAP1b protein comprising a non-natural modification is reduced by at least 60% as compared to expression of a wild type version of the NtHAP1b protein in a control tobacco plant when grown under comparable conditions. In an aspect, expression or activity of an NtHAP1b protein comprising a non-natural modification is reduced by at least 70% as compared to expression of a wild type version of the NtHAP1b protein in a control tobacco plant when grown under comparable conditions. In an aspect, expression or activity of an NtHAP1b protein comprising a non-natural modification is reduced by at least 80% as compared to expression of a wild type version of the NtHAP1b protein in a control tobacco plant when grown under comparable conditions. In an aspect, expression or activity of an NtHAP1b protein comprising a non-natural modification is reduced by at least 90% as compared to expression of a wild type version of the NtHAP1b protein in a control tobacco plant when grown under comparable conditions. In an aspect, expression or activity of an NtHAP1b protein comprising a non-natural modification is reduced by at least 95% as compared to expression of a wild type version of the NtHAP1b protein in a control tobacco plant when grown under comparable conditions. In an aspect, expression or activity of an NtHAP1b protein comprising a non-natural modification is reduced by at least 99% as compared to expression of a wild type version of the NtHAP1b protein in a control tobacco plant when grown under comparable conditions. In an aspect, expression or activity of an NtHAP1b protein comprising a non-natural modification is reduced by 100% as compared to expression of a wild type version of the NtHAP1b protein in a control tobacco plant when grown under comparable conditions.

In an aspect, expression of a NtHAP1b gene comprising a non-natural mutation is reduced by at least 1% as compared to expression of an wild type version of the NtHAP1b gene in a control tobacco plant when grown under comparable conditions. In an aspect, expression of a NtHAP1b gene comprising a non-natural mutation is reduced by at least 5% as compared to expression of an wild type version of the NtHAP1b gene in a control tobacco plant when grown under comparable conditions. In an aspect, expression of a NtHAP1b gene comprising a non-natural mutation is reduced by at least 10% as compared to expression of an wild type version of the NtHAP1b gene in a control tobacco plant when grown under comparable conditions. In an aspect, expression of a NtHAP1b gene comprising a non-natural mutation is reduced by at least 20% as compared to expression of an wild type version of the NtHAP1b gene in a control tobacco plant when grown under comparable conditions. In an aspect, expression of a NtHAP1b gene comprising a non-natural mutation is reduced by at least 30% as compared to expression of an wild type version of the NtHAP1b gene in a control tobacco plant when grown under comparable conditions. In an aspect, expression of a NtHAP1b gene comprising a non-natural mutation is reduced by at least 40% as compared to expression of an wild type version of the NtHAP1b gene in a control tobacco plant when grown under comparable conditions. In an aspect, expression of a NtHAP1b gene comprising a non-natural mutation is reduced by at least 50% as compared to expression of an wild type version of the NtHAP1b gene in a control tobacco plant when grown under comparable conditions. In an aspect, expression of a NtHAP1b gene comprising a non-natural mutation is reduced by at least 60% as compared to expression of an wild type version of the NtHAP1b gene in a control tobacco plant when grown under comparable conditions. In an aspect, expression of a NtHAP1b gene comprising a non-natural mutation is reduced by at least 70% as compared to expression of an wild type version of the NtHAP1b gene in a control tobacco plant when grown under comparable conditions. In an aspect, expression of a NtHAP1b gene comprising a non-natural mutation is reduced by at least 80% as compared to expression of an wild type version of the NtHAP1b gene in a control tobacco plant when grown under comparable conditions. In an aspect, expression of a NtHAP1b gene comprising a non-natural mutation is reduced by at least 90% as compared to expression of an wild type version of the NtHAP1b gene in a control tobacco plant when grown under comparable conditions. In an aspect, expression of a NtHAP1b gene comprising a non-natural mutation is reduced by at least 95% as compared to expression of an wild type version of the NtHAP1b gene in a control tobacco plant when grown under comparable conditions. In an aspect, expression of a NtHAP1b gene comprising a non-natural mutation is reduced by at least 99% as compared to expression of an wild type version of the NtHAP1b gene in a control tobacco plant when grown under comparable conditions. In an aspect, expression of a NtHAP1b gene comprising a non-natural mutation is reduced by 100% as compared to expression of an wild type version of the NtHAP1b gene in a control tobacco plant when grown under comparable conditions.

In an aspect, expression or activity of an NtHAP2 protein comprising a non-natural modification is reduced by at least 1% as compared to expression of a wild type version of the NtHAP2 protein in a control tobacco plant when grown under comparable conditions. In an aspect, expression or activity of an NtHAP2 protein comprising a non-natural modification is reduced by at least 5% as compared to expression of a wild type version of the NtHAP2 protein in a control tobacco plant when grown under comparable conditions. In an aspect, expression or activity of an NtHAP2 protein comprising a non-natural modification is reduced by at least 10% as compared to expression of a wild type version of the NtHAP2 protein in a control tobacco plant when grown under comparable conditions. In an aspect, expression or activity of an NtHAP2 protein comprising a non-natural modification is reduced by at least 20% as compared to expression of a wild type version of the NtHAP2 protein in a control tobacco plant when grown under comparable conditions. In an aspect, expression or activity of an NtHAP2 protein comprising a non-natural modification is reduced by at least 30% as compared to expression of a wild type version of the NtHAP2 protein in a control tobacco plant when grown under comparable conditions. In an aspect, expression or activity of an NtHAP2 protein comprising a non-natural modification is reduced by at least 40% as compared to expression of a wild type version of the NtHAP2 protein in a control tobacco plant when grown under comparable conditions. In an aspect, expression or activity of an NtHAP2 protein comprising a non-natural modification is reduced by at least 50% as compared to expression of a wild type version of the NtHAP2 protein in a control tobacco plant when grown under comparable conditions. In an aspect, expression or activity of an NtHAP2 protein comprising a non-natural modification is reduced by at least 60% as compared to expression of a wild type version of the NtHAP2 protein in a control tobacco plant when grown under comparable conditions. In an aspect, expression or activity of an NtHAP2 protein comprising a non-natural modification is reduced by at least 70% as compared to expression of a wild type version of the NtHAP2 protein in a control tobacco plant when grown under comparable conditions. In an aspect, expression or activity of an NtHAP2 protein comprising a non-natural modification is reduced by at least 80% as compared to expression of a wild type version of the NtHAP2 protein in a control tobacco plant when grown under comparable conditions. In an aspect, expression or activity of an NtHAP2 protein comprising a non-natural modification is reduced by at least 90% as compared to expression of a wild type version of the NtHAP2 protein in a control tobacco plant when grown under comparable conditions. In an aspect, expression or activity of an NtHAP2 protein comprising a non-natural modification is reduced by at least 95% as compared to expression of a wild type version of the NtHAP2 protein in a control tobacco plant when grown under comparable conditions. In an aspect, expression or activity of an NtHAP2 protein comprising a non-natural modification is reduced by at least 99% as compared to expression of a wild type version of the NtHAP2 protein in a control tobacco plant when grown under comparable conditions. In an aspect, expression or activity of an NtHAP2 protein comprising a non-natural modification is reduced by 100% as compared to expression of a wild type version of the NtHAP2 protein in a control tobacco plant when grown under comparable conditions.

In an aspect, expression of a NtHAP2 gene comprising a non-natural mutation is reduced by at least 1% as compared to expression of an wild type version of the NtHAP2 gene in a control tobacco plant when grown under comparable conditions. In an aspect, expression of a NtHAP2 gene comprising a non-natural mutation is reduced by at least 5% as compared to expression of an wild type version of the NtHAP2 gene in a control tobacco plant when grown under comparable conditions. In an aspect, expression of a NtHAP2 gene comprising a non-natural mutation is reduced by at least 10% as compared to expression of an wild type version of the NtHAP2 gene in a control tobacco plant when grown under comparable conditions. In an aspect, expression of a NtHAP2 gene comprising a non-natural mutation is reduced by at least 20% as compared to expression of an wild type version of the NtHAP2 gene in a control tobacco plant when grown under comparable conditions. In an aspect, expression of a NtHAP2 gene comprising a non-natural mutation is reduced by at least 30% as compared to expression of an wild type version of the NtHAP2 gene in a control tobacco plant when grown under comparable conditions. In an aspect, expression of a NtHAP2 gene comprising a non-natural mutation is reduced by at least 40% as compared to expression of an wild type version of the NtHAP2 gene in a control tobacco plant when grown under comparable conditions. In an aspect, expression of a NtHAP2 gene comprising a non-natural mutation is reduced by at least 50% as compared to expression of an wild type version of the NtHAP2 gene in a control tobacco plant when grown under comparable conditions. In an aspect, expression of a NtHAP2 gene comprising a non-natural mutation is reduced by at least 60% as compared to expression of an wild type version of the NtHAP2 gene in a control tobacco plant when grown under comparable conditions. In an aspect, expression of a NtHAP2 gene comprising a non-natural mutation is reduced by at least 70% as compared to expression of an wild type version of the NtHAP2 gene in a control tobacco plant when grown under comparable conditions. In an aspect, expression of a NtHAP2 gene comprising a non-natural mutation is reduced by at least 80% as compared to expression of an wild type version of the NtHAP2 gene in a control tobacco plant when grown under comparable conditions. In an aspect, expression of a NtHAP2 gene comprising a non-natural mutation is reduced by at least 90% as compared to expression of an wild type version of the NtHAP2 gene in a control tobacco plant when grown under comparable conditions. In an aspect, expression of a NtHAP2 gene comprising a non-natural mutation is reduced by at least 95% as compared to expression of an wild type version of the NtHAP2 gene in a control tobacco plant when grown under comparable conditions. In an aspect, expression of a NtHAP2 gene comprising a non-natural mutation is reduced by at least 99% as compared to expression of an wild type version of the NtHAP2 gene in a control tobacco plant when grown under comparable conditions. In an aspect, expression of a NtHAP2 gene comprising a non-natural mutation is reduced by 100% as compared to expression of an wild type version of the NtHAP2 gene in a control tobacco plant when grown under comparable conditions.

In an aspect, expression or activity of an NtHAP3 protein comprising a non-natural modification is reduced by at least 1% as compared to expression of a wild type version of the NtHAP3 protein in a control tobacco plant when grown under comparable conditions. In an aspect, expression or activity of an NtHAP3 protein comprising a non-natural modification is reduced by at least 5% as compared to expression of a wild type version of the NtHAP3 protein in a control tobacco plant when grown under comparable conditions. In an aspect, expression or activity of an NtHAP3 protein comprising a non-natural modification is reduced by at least 10% as compared to expression of a wild type version of the NtHAP3 protein in a control tobacco plant when grown under comparable conditions. In an aspect, expression or activity of an NtHAP3 protein comprising a non-natural modification is reduced by at least 20% as compared to expression of a wild type version of the NtHAP3 protein in a control tobacco plant when grown under comparable conditions. In an aspect, expression or activity of an NtHAP3 protein comprising a non-natural modification is reduced by at least 30% as compared to expression of a wild type version of the NtHAP3 protein in a control tobacco plant when grown under comparable conditions. In an aspect, expression or activity of an NtHAP3 protein comprising a non-natural modification is reduced by at least 40% as compared to expression of a wild type version of the NtHAP3 protein in a control tobacco plant when grown under comparable conditions. In an aspect, expression or activity of an NtHAP3 protein comprising a non-natural modification is reduced by at least 50% as compared to expression of a wild type version of the NtHAP3 protein in a control tobacco plant when grown under comparable conditions. In an aspect, expression or activity of an NtHAP3 protein comprising a non-natural modification is reduced by at least 60% as compared to expression of a wild type version of the NtHAP3 protein in a control tobacco plant when grown under comparable conditions. In an aspect, expression or activity of an NtHAP3 protein comprising a non-natural modification is reduced by at least 70% as compared to expression of a wild type version of the NtHAP3 protein in a control tobacco plant when grown under comparable conditions. In an aspect, expression or activity of an NtHAP3 protein comprising a non-natural modification is reduced by at least 80% as compared to expression of a wild type version of the NtHAP3 protein in a control tobacco plant when grown under comparable conditions. In an aspect, expression or activity of an NtHAP3 protein comprising a non-natural modification is reduced by at least 90% as compared to expression of a wild type version of the NtHAP3 protein in a control tobacco plant when grown under comparable conditions. In an aspect, expression or activity of an NtHAP3 protein comprising a non-natural modification is reduced by at least 95% as compared to expression of a wild type version of the NtHAP3 protein in a control tobacco plant when grown under comparable conditions. In an aspect, expression or activity of an NtHAP3 protein comprising a non-natural modification is reduced by at least 99% as compared to expression of a wild type version of the NtHAP3 protein in a control tobacco plant when grown under comparable conditions. In an aspect, expression or activity of an NtHAP3 protein comprising a non-natural modification is reduced by 100% as compared to expression of a wild type version of the NtHAP3 protein in a control tobacco plant when grown under comparable conditions.

In an aspect, expression of a NtHAP3 gene comprising a non-natural mutation is reduced by at least 1% as compared to expression of an wild type version of the NtHAP3 gene in a control tobacco plant when grown under comparable conditions. In an aspect, expression of a NtHAP3 gene comprising a non-natural mutation is reduced by at least 5% as compared to expression of an wild type version of the NtHAP3 gene in a control tobacco plant when grown under comparable conditions. In an aspect, expression of a NtHAP3 gene comprising a non-natural mutation is reduced by at least 10% as compared to expression of an wild type version of the NtHAP3 gene in a control tobacco plant when grown under comparable conditions. In an aspect, expression of a NtHAP3 gene comprising a non-natural mutation is reduced by at least 20% as compared to expression of an wild type version of the NtHAP3 gene in a control tobacco plant when grown under comparable conditions. In an aspect, expression of a NtHAP3 gene comprising a non-natural mutation is reduced by at least 30% as compared to expression of an wild type version of the NtHAP3 gene in a control tobacco plant when grown under comparable conditions. In an aspect, expression of a NtHAP3 gene comprising a non-natural mutation is reduced by at least 40% as compared to expression of an wild type version of the NtHAP3 gene in a control tobacco plant when grown under comparable conditions. In an aspect, expression of a NtHAP3 gene comprising a non-natural mutation is reduced by at least 50% as compared to expression of an wild type version of the NtHAP3 gene in a control tobacco plant when grown under comparable conditions. In an aspect, expression of a NtHAP3 gene comprising a non-natural mutation is reduced by at least 60% as compared to expression of an wild type version of the NtHAP3 gene in a control tobacco plant when grown under comparable conditions. In an aspect, expression of a NtHAP3 gene comprising a non-natural mutation is reduced by at least 70% as compared to expression of an wild type version of the NtHAP3 gene in a control tobacco plant when grown under comparable conditions. In an aspect, expression of a NtHAP3 gene comprising a non-natural mutation is reduced by at least 80% as compared to expression of an wild type version of the NtHAP3 gene in a control tobacco plant when grown under comparable conditions. In an aspect, expression of a NtHAP3 gene comprising a non-natural mutation is reduced by at least 90% as compared to expression of an wild type version of the NtHAP3 gene in a control tobacco plant when grown under comparable conditions. In an aspect, expression of a NtHAP3 gene comprising a non-natural mutation is reduced by at least 95% as compared to expression of an wild type version of the NtHAP3 gene in a control tobacco plant when grown under comparable conditions. In an aspect, expression of a NtHAP3 gene comprising a non-natural mutation is reduced by at least 99% as compared to expression of an wild type version of the NtHAP3 gene in a control tobacco plant when grown under comparable conditions. In an aspect, expression of a NtHAP3 gene comprising a non-natural mutation is reduced by 100% as compared to expression of an wild type version of the NtHAP3 gene in a control tobacco plant when grown under comparable conditions.

In an aspect, a reduction in expression comprises a statistically significant reduction as compared to expression in the same tissue of a control plant grown under comparable conditions. One of ordinary skill in the art would recognize that any level of reduction is envisioned, so long as the level of reduction has been determined to be statistically significant using an accepted statistical hypothesis test. As a non-limiting example, a Student's t-test is one statistical hypothesis test that can be used to determine if a reduction in expression between a modified plant and a control plant is statistically significant. As used herein, “statistically significant” refers to a p-value of less than or equal to 0.05 when using an appropriate statistical test.

As used herein, a “mutation” refers to an inheritable genetic modification introduced into a gene to alter the expression or activity of a product encoded by the gene. Such a modification can be in any sequence region of a gene, for example, in a promoter, 5′-untranslated region (UTR), exon, intron, 3′-UTR, or terminator region. In an aspect, a mutation reduces, inhibits, or eliminates the expression or activity of a gene product. In another aspect, a mutation increases, elevates, strengthens, or augments the expression or activity of a gene product.

As used herein, when referring to a protein, “activity” refers to the ability to carry out an enzymatic function. Without being limited by any scientific theory, NtHAP1a, NtHAP1b, NtHAP2, and/or NtHAP3 may be involved in processes related to histone tail modifications or methylated DNA binding.

In an aspect, a mutation is a “non-natural” or “non-naturally occurring” mutation. As used herein, a “non-natural” or “non-naturally occurring” mutation refers to a non-spontaneous mutation generated via human intervention, and does not correspond to a spontaneous mutation generated without human intervention. Non-limiting examples of human intervention include mutagenesis (e.g., chemical mutagenesis, ionizing radiation mutagenesis) and targeted genetic modifications (e.g., CRISPR-based methods, TALEN-based methods, zinc finger-based methods). Non-natural mutations and non-naturally occurring mutations do not include spontaneous mutations that arise naturally (e.g., via aberrant DNA replication in a germ line of a plant).

In an aspect, a modified tobacco plant is heterozygous for a non-natural mutation. In an aspect, a modified tobacco plant is homozygous for a non-natural mutation. In an aspect, a modified tobacco seed is heterozygous for a non-natural mutation. In an aspect, a modified tobacco seed is homozygous for a non-natural mutation. In an aspect, a modified tobacco plant part is heterozygous for a non-natural mutation. In an aspect, a modified tobacco plant part is homozygous for a non-natural mutation.

It will be appreciated that, when identifying a mutation, the reference DNA sequence should be from the same variety of tobacco. For example, if a modified tobacco plant comprising a mutation is from the variety TN90, then the endogenous reference sequence must be the endogenous TN90 sequence, not a homologous sequence from a different tobacco variety (e.g., K326). Similarly, if a modified tobacco cell comprising a mutation is a TN90 cell, then the endogenous reference sequence must be the endogenous TN90 sequence, not a homologous sequence from a tobacco cell from a different tobacco variety (e.g., K326).

As used herein, a “control plant” refers to a plant of identical, or nearly identical, genetic makeup as the modified plant being compared, except for the non-natural mutation provided herein that was introduced to the modified plant. As a non-limiting example, a modified TN90 tobacco plant comprises a non-natural mutation in at least one of NtHAP1a, NtHAP1b, NtHAP2, or NtHAP3, while the control plant is an unmodified TN90 plant.

Unless specified otherwise, all comparisons to control plants require similar growth conditions or comparable growth conditions for the two plants being compared. As used herein, “grown under comparable conditions” or “comparable growth conditions” refer to similar environmental conditions and/or agronomic practices for growing and making meaningful comparisons between two or more plant genotypes so that neither environmental conditions nor agronomic practices would contribute to or explain any difference observed between the two or more plant genotypes. Environmental conditions include, for example, light, temperature, soil moisture, humidity, and nutrition (e.g., nitrogen and phosphorus). Agronomic practices include, for example, seeding, clipping, undercutting, transplanting, topping, and suckering. See Chapters 4B and 4C of Tobacco, Production, Chemistry and Technology, Davis & Nielsen, eds., Blackwell Publishing, Oxford (1999), pp 70-103.

In an aspect, a non-natural mutation is a null mutation. As used herein, a “null mutation” refers to a mutation that confers a complete loss-of-function for a protein encoded by a gene comprising the mutation. A null mutation can cause lack of mRNA transcript production, a lack of protein function, or both.

Mutations in coding regions of genes (e.g., exonic mutations) can result in a truncated protein or polypeptide when a mutated messenger RNA (mRNA) is translated into a protein or polypeptide. In an aspect, this disclosure provides a mutation that results in the truncation of a protein or polypeptide. As used herein, a “truncated” protein or polypeptide comprises at least one fewer amino acid as compared to an endogenous control protein or polypeptide. For example, if endogenous Protein A comprises 100 amino acids, a truncated version of Protein A can comprise between 1 and 99 amino acids. In an aspect, a non-natural mutation results in a truncation of a polypeptide.

Without being limited by any scientific theory, one way to cause a protein or polypeptide truncation is by the introduction of a premature stop codon in an mRNA transcript of an endogenous gene. In an aspect, this disclosure provides a mutation that results in a premature stop codon in an mRNA transcript of an endogenous gene. As used herein, a “stop codon” refers to a nucleotide triplet within an mRNA transcript that signals a termination of protein translation. A “premature stop codon” refers to a stop codon positioned earlier (e.g., on the 5′-side) than the normal stop codon position in an endogenous mRNA transcript. Without being limiting, several stop codons are known in the art, including “UAG,” “UAA,” “UGA,” “TAG,” “TAA,” and “TGA.”

In an aspect, a non-natural mutation comprises a premature stop codon in a coding region of gene encoding a protein as compared to the corresponding endogenous nucleic acid molecule. In an aspect, a non-natural mutation comprises a premature stop codon as compared to the endogenous nucleic acid molecule. In an aspect, a nucleic acid molecule comprising a non-natural mutation comprises a premature stop codon as compared to an endogenous nucleic acid molecule lacking the non-natural mutation.

In an aspect, a non-natural mutation results in a truncated NtHAP1a protein as compared to SEQ ID NO: 1. In an aspect, a non-natural mutation results in a truncated NtHAP1b protein as compared to SEQ ID NO: 2. In an aspect, a non-natural mutation results in a truncated NtHAP2 protein as compared to SEQ ID NO: 3. In an aspect, a non-natural mutation results in a truncated NtHAP3 protein as compared to SEQ ID NO: 538.

In an aspect, a truncated protein comprises a null mutation.

In an aspect, a non-natural mutation comprises one or more mutation types selected from the group consisting of a nonsense mutation, a missense mutation, a frameshift mutation, a splice-site mutation, and any combinations thereof. As used herein, a “nonsense mutation” refers to a mutation to a nucleic acid sequence that introduces a premature stop codon to an amino acid sequence by the nucleic acid sequence. As used herein, a “missense mutation” refers to a mutation to a nucleic acid sequence that causes a substitution within the amino acid sequence encoded by the nucleic acid sequence. As used herein, a “frameshift mutation” refers to an insertion or deletion to a nucleic acid sequence that shifts the frame for translating the nucleic acid sequence to an amino acid sequence. A “splice-site mutation” refers to a mutation in a nucleic acid sequence that causes an intron to be retained for protein translation, or, alternatively, for an exon to be excluded from protein translation. Splice-site mutations can cause nonsense, missense, or frameshift mutations.

A non-natural mutation provided herein can be positioned in any part of an endogenous gene. In an aspect, a non-natural mutation provided herein is positioned within an exon of an endogenous gene. In an aspect, a non-natural mutation provided herein is positioned within a 5′-UTR of an endogenous gene. In an aspect, a non-natural mutation provided herein is positioned within a 3′-UTR of an endogenous gene. In an aspect, a non-natural mutation provided herein is positioned within a promoter of an endogenous gene. In an aspect, a non-natural mutation provided herein is positioned within a terminator of an endogenous gene. In an aspect, a non-natural mutation provided herein comprises a mutation in a sequence region selected from the group consisting of a promoter, a 5′-UTR, a 3′-UTR, an exon, an intron, and a terminator.

In an aspect, a promoter comprises a nucleic acid sequence at least 85% identical to a nucleic acid sequence selected from the group consisting of SEQ ID NOs: 72 to 89 and 285 to 351. In an aspect, a promoter comprises a nucleic acid sequence at least 90% identical to a nucleic acid sequence selected from the group consisting of SEQ ID NOs: 72 to 89 and 285 to 351. In an aspect, a promoter comprises a nucleic acid sequence at least 92.5% identical to a nucleic acid sequence selected from the group consisting of SEQ ID NOs: 72 to 89 and 285 to 351. In an aspect, a promoter comprises a nucleic acid sequence at least 95% identical to a nucleic acid sequence selected from the group consisting of SEQ ID NOs: 72 to 89 and 285 to 351. In an aspect, a promoter comprises a nucleic acid sequence at least 96% identical to a nucleic acid sequence selected from the group consisting of SEQ ID NOs: 72 to 89 and 285 to 351. In an aspect, a promoter comprises a nucleic acid sequence at least 97% identical to a nucleic acid sequence selected from the group consisting of SEQ ID NOs: 72 to 89 and 285 to 351. In an aspect, a promoter comprises a nucleic acid sequence at least 98% identical to a nucleic acid sequence selected from the group consisting of SEQ ID NOs: 72 to 89 and 285 to 351. In an aspect, a promoter comprises a nucleic acid sequence at least 99% identical to a nucleic acid sequence selected from the group consisting of SEQ ID NOs: 72 to 89 and 285 to 351. In an aspect, a promoter comprises a nucleic acid sequence 100% identical to a nucleic acid sequence selected from the group consisting of SEQ ID NOs: 72 to 89 and 285 to 351.

The screening and selection of mutagenized tobacco plants can be through any methodologies known to those having ordinary skill in the art. Examples of screening and selection methodologies include, but are not limited to, Southern analysis, PCR amplification for detection of a polynucleotide, Northern blots, RNase protection, primer-extension, RT-PCR amplification for detecting RNA transcripts, Sanger sequencing, Next Generation sequencing technologies (e.g., Illumina, PacBio, Ion Torrent, Oxford Nanopore) enzymatic assays for detecting enzyme or ribozyme activity of polypeptides and polynucleotides, and protein gel electrophoresis, Western blots, immunoprecipitation, and enzyme-linked immunoassays to detect polypeptides. Other techniques such as in situ hybridization, enzyme staining, and immunostaining also can be used to detect the presence or expression of polypeptides and/or polynucleotides. Methods for performing all of the referenced techniques are known in the art.

Several types of mutations are known in the art. In an aspect, a mutation comprises an insertion. In an aspect, a non-natural mutation comprises an insertion. An “insertion” refers to the addition of one or more nucleotides or amino acids to a given polynucleotide or amino acid sequence, respectively, as compared to wild type reference polynucleotide or amino acid sequence. Insertions increase the length of a given sequence.

In an aspect, a non-natural mutation comprises an insertion of at least 1 nucleotide in a nucleic acid molecule as compared to a wild type nucleic acid molecule. In an aspect, a non-natural mutation comprises an insertion of at least 2 nucleotides in a nucleic acid molecule as compared to a wild type nucleic acid molecule. In an aspect, a non-natural mutation comprises an insertion of at least 3 nucleotides in a nucleic acid molecule as compared to a wild type nucleic acid molecule. In an aspect, a non-natural mutation comprises an insertion of at least 4 nucleotides in a nucleic acid molecule as compared to a wild type nucleic acid molecule. In an aspect, a non-natural mutation comprises an insertion of at least 5 nucleotides in a nucleic acid molecule as compared to a wild type nucleic acid molecule. In an aspect, a non-natural mutation comprises an insertion of at least 6 nucleotides in a nucleic acid molecule as compared to a wild type nucleic acid molecule. In an aspect, a non-natural mutation comprises an insertion of at least 7 nucleotides in a nucleic acid molecule as compared to a wild type nucleic acid molecule. In an aspect, a non-natural mutation comprises an insertion of at least 8 nucleotides in a nucleic acid molecule as compared to a wild type nucleic acid molecule. In an aspect, a non-natural mutation comprises an insertion of at least 9 nucleotides in a nucleic acid molecule as compared to a wild type nucleic acid molecule. In an aspect, a non-natural mutation comprises an insertion of at least 10 nucleotides in a nucleic acid molecule as compared to a wild type nucleic acid molecule. In an aspect, a non-natural mutation comprises an insertion of at least 15 nucleotides in a nucleic acid molecule as compared to a wild type nucleic acid molecule. In an aspect, a non-natural mutation comprises an insertion of at least 20 nucleotides in a nucleic acid molecule as compared to a wild type nucleic acid molecule. In an aspect, a non-natural mutation comprises an insertion of at least 30 nucleotides in a nucleic acid molecule as compared to a wild type nucleic acid molecule. In an aspect, a non-natural mutation comprises an insertion of at least 40 nucleotides in a nucleic acid molecule as compared to a wild type nucleic acid molecule. In an aspect, a non-natural mutation comprises an insertion of at least 50 nucleotides in a nucleic acid molecule as compared to a wild type nucleic acid molecule. In an aspect, a non-natural mutation comprises an insertion of at least 75 nucleotides in a nucleic acid molecule as compared to a wild type nucleic acid molecule. In an aspect, a non-natural mutation comprises an insertion of at least 100 nucleotides in a nucleic acid molecule as compared to a wild type nucleic acid molecule. In an aspect, a non-natural mutation comprises an insertion of at least 250 nucleotides in a nucleic acid molecule as compared to a wild type nucleic acid molecule. In an aspect, a non-natural mutation comprises an insertion of at least 500 nucleotides in a nucleic acid molecule as compared to a wild type nucleic acid molecule. In an aspect, a non-natural mutation comprises an insertion of at least 1000 nucleotides in a nucleic acid molecule as compared to a wild type nucleic acid molecule.

In an aspect, a non-natural mutation comprises an insertion of between 1 nucleotide and 1000 nucleotides in a nucleic acid molecule as compared to a wild type nucleic acid molecule. In an aspect, a non-natural mutation comprises an insertion of between 1 nucleotide and 750 nucleotides in a nucleic acid molecule as compared to a wild type nucleic acid molecule. In an aspect, a non-natural mutation comprises an insertion of between 1 nucleotide and 500 nucleotides in a nucleic acid molecule as compared to a wild type nucleic acid molecule. In an aspect, a non-natural mutation comprises an insertion of between 1 nucleotide and 250 nucleotides in a nucleic acid molecule as compared to a wild type nucleic acid molecule. In an aspect, a non-natural mutation comprises an insertion of between 1 nucleotide and 100 nucleotides in a nucleic acid molecule as compared to a wild type nucleic acid molecule. In an aspect, a non-natural mutation comprises an insertion of between 1 nucleotide and 75 nucleotides in a nucleic acid molecule as compared to a wild type nucleic acid molecule. In an aspect, a non-natural mutation comprises an insertion of between 1 nucleotide and 50 nucleotides in a nucleic acid molecule as compared to a wild type nucleic acid molecule. In an aspect, a non-natural mutation comprises an insertion of between 1 nucleotide and 25 nucleotides in a nucleic acid molecule as compared to a wild type nucleic acid molecule. In an aspect, a non-natural mutation comprises an insertion of between 1 nucleotide and 10 nucleotides in a nucleic acid molecule as compared to a wild type nucleic acid molecule. In an aspect, a non-natural mutation comprises an insertion of between 1 nucleotide and 5 nucleotides in a nucleic acid molecule as compared to a wild type nucleic acid molecule.

In an aspect, a non-natural mutation comprises an insertion of at least 1 amino acid residue in an amino acid sequence as compared to a wild type amino acid sequence. In an aspect, a non-natural mutation comprises an insertion of at least 2 amino acid residues in an amino acid sequence as compared to a wild type amino acid sequence. In an aspect, a non-natural mutation comprises an insertion of at least 3 amino acid residues in an amino acid sequence as compared to a wild type amino acid sequence. In an aspect, a non-natural mutation comprises an insertion of at least 4 amino acid residues in an amino acid sequence as compared to a wild type amino acid sequence. In an aspect, a non-natural mutation comprises an insertion of at least 5 amino acid residues in an amino acid sequence as compared to a wild type amino acid sequence. In an aspect, a non-natural mutation comprises an insertion of at least 6 amino acid residues in an amino acid sequence as compared to a wild type amino acid sequence. In an aspect, a non-natural mutation comprises an insertion of at least 7 amino acid residues in an amino acid sequence as compared to a wild type amino acid sequence. In an aspect, a non-natural mutation comprises an insertion of at least 8 amino acid residues in an amino acid sequence as compared to a wild type amino acid sequence. In an aspect, a non-natural mutation comprises an insertion of at least 9 amino acid residues in an amino acid sequence as compared to a wild type amino acid sequence. In an aspect, a non-natural mutation comprises an insertion of at least 10 amino acid residues in an amino acid sequence as compared to a wild type amino acid sequence. In an aspect, a non-natural mutation comprises an insertion of at least 15 amino acid residues in an amino acid sequence as compared to a wild type amino acid sequence. In an aspect, a non-natural mutation comprises an insertion of at least 20 amino acid residues in an amino acid sequence as compared to a wild type amino acid sequence. In an aspect, a non-natural mutation comprises an insertion of at least 30 amino acid residues in an amino acid sequence as compared to a wild type amino acid sequence. In an aspect, a non-natural mutation comprises an insertion of at least 40 amino acid residues in an amino acid sequence as compared to a wild type amino acid sequence. In an aspect, a non-natural mutation comprises an insertion of at least 50 amino acid residues in an amino acid sequence as compared to a wild type amino acid sequence. In an aspect, a non-natural mutation comprises an insertion of at least 75 amino acid residues in an amino acid sequence as compared to a wild type amino acid sequence. In an aspect, a non-natural mutation comprises an insertion of at least 100 amino acid residues in an amino acid sequence as compared to a wild type amino acid sequence. In an aspect, a non-natural mutation comprises an insertion of at least 250 amino acid residues in an amino acid sequence as compared to a wild type amino acid sequence. In an aspect, a non-natural mutation comprises an insertion of at least 500 amino acid residues in an amino acid sequence as compared to a wild type amino acid sequence. In an aspect, a non-natural mutation comprises an insertion of at least 1000 amino acid residues in an amino acid sequence as compared to a wild type amino acid sequence.

In an aspect, a non-natural mutation comprises an insertion of between 1 amino acid residue and 1000 amino acid residues in an amino acid sequence as compared to a wild type amino acid sequence. In an aspect, a non-natural mutation comprises an insertion of between 1 amino acid residue and 750 amino acid residues in an amino acid sequence as compared to a wild type amino acid sequence. In an aspect, a non-natural mutation comprises an insertion of between 1 amino acid residue and 500 amino acid residues in an amino acid sequence as compared to a wild type amino acid sequence. In an aspect, a non-natural mutation comprises an insertion of between 1 amino acid residue and 250 amino acid residues in an amino acid sequence as compared to a wild type amino acid sequence. In an aspect, a non-natural mutation comprises an insertion of between 1 amino acid residue and 100 amino acid residues in an amino acid sequence as compared to a wild type amino acid sequence. In an aspect, a non-natural mutation comprises an insertion of between 1 amino acid residue and 75 amino acid residues in an amino acid sequence as compared to a wild type amino acid sequence. In an aspect, a non-natural mutation comprises an insertion of between 1 amino acid residue and 50 amino acid residues in an amino acid sequence as compared to a wild type amino acid sequence. In an aspect, a non-natural mutation comprises an insertion of between 1 amino acid residue and 25 amino acid residues in an amino acid sequence as compared to a wild type amino acid sequence. In an aspect, a non-natural mutation comprises an insertion of between 1 amino acid residue and 10 amino acid residues in an amino acid sequence as compared to a wild type amino acid sequence. In an aspect, a non-natural mutation comprises an insertion of between 1 amino acid residue and 5 amino acid residues in an amino acid sequence as compared to a wild type amino acid sequence.

In an aspect, a non-natural mutation comprises a deletion. A “deletion” refers to the removal of one or more nucleotides or amino acids from a given polynucleotide or amino acid sequence, respectively, as compared to wild type reference polynucleotide or amino acid sequence. Deletions reduce the length of a given sequence.

In an aspect, a non-natural mutation comprises a deletion of at least 1 nucleotide in a nucleic acid molecule as compared to a wild type nucleic acid molecule. In an aspect, a non-natural mutation comprises a deletion of at least 2 nucleotides in a nucleic acid molecule as compared to a wild type nucleic acid molecule. In an aspect, a non-natural mutation comprises a deletion of at least 3 nucleotides in a nucleic acid molecule as compared to a wild type nucleic acid molecule. In an aspect, a non-natural mutation comprises a deletion of at least 4 nucleotides in a nucleic acid molecule as compared to a wild type nucleic acid molecule. In an aspect, a non-natural mutation comprises a deletion of at least 5 nucleotides in a nucleic acid molecule as compared to a wild type nucleic acid molecule. In an aspect, a non-natural mutation comprises a deletion of at least 6 nucleotides in a nucleic acid molecule as compared to a wild type nucleic acid molecule. In an aspect, a non-natural mutation comprises a deletion of at least 7 nucleotides in a nucleic acid molecule as compared to a wild type nucleic acid molecule. In an aspect, a non-natural mutation comprises a deletion of at least 8 nucleotides in a nucleic acid molecule as compared to a wild type nucleic acid molecule. In an aspect, a non-natural mutation comprises a deletion of at least 9 nucleotides in a nucleic acid molecule as compared to a wild type nucleic acid molecule. In an aspect, a non-natural mutation comprises a deletion of at least 10 nucleotides in a nucleic acid molecule as compared to a wild type nucleic acid molecule. In an aspect, a non-natural mutation comprises a deletion of at least 15 nucleotides in a nucleic acid molecule as compared to a wild type nucleic acid molecule. In an aspect, a non-natural mutation comprises a deletion of at least 20 nucleotides in a nucleic acid molecule as compared to a wild type nucleic acid molecule. In an aspect, a non-natural mutation comprises a deletion of at least 30 nucleotides in a nucleic acid molecule as compared to a wild type nucleic acid molecule. In an aspect, a non-natural mutation comprises a deletion of at least 40 nucleotides in a nucleic acid molecule as compared to a wild type nucleic acid molecule. In an aspect, a non-natural mutation comprises a deletion of at least 50 nucleotides in a nucleic acid molecule as compared to a wild type nucleic acid molecule. In an aspect, a non-natural mutation comprises a deletion of at least 75 nucleotides in a nucleic acid molecule as compared to a wild type nucleic acid molecule. In an aspect, a non-natural mutation comprises a deletion of at least 100 nucleotides in a nucleic acid molecule as compared to a wild type nucleic acid molecule. In an aspect, a non-natural mutation comprises a deletion of at least 250 nucleotides in a nucleic acid molecule as compared to a wild type nucleic acid molecule. In an aspect, a non-natural mutation comprises a deletion of at least 500 nucleotides in a nucleic acid molecule as compared to a wild type nucleic acid molecule. In an aspect, a non-natural mutation comprises a deletion of at least 1000 nucleotides in a nucleic acid molecule as compared to a wild type nucleic acid molecule. In an aspect, a non-natural mutation comprises a deletion of at least 2500 nucleotides in a nucleic acid molecule as compared to a wild type nucleic acid molecule. In an aspect, a non-natural mutation comprises a deletion of at least 3000 nucleotides in a nucleic acid molecule as compared to a wild type nucleic acid molecule.

In an aspect, a non-natural mutation comprises a deletion of between 1 nucleotide and 4000 nucleotides in a nucleic acid molecule as compared to a wild type nucleic acid molecule. In an aspect, a non-natural mutation comprises a deletion of between 1 nucleotide and 3000 nucleotides in a nucleic acid molecule as compared to a wild type nucleic acid molecule. In an aspect, a non-natural mutation comprises a deletion of between 1 nucleotide and 2000 nucleotides in a nucleic acid molecule as compared to a wild type nucleic acid molecule. In an aspect, a non-natural mutation comprises a deletion of between 1 nucleotide and 1000 nucleotides in a nucleic acid molecule as compared to a wild type nucleic acid molecule. In an aspect, a non-natural mutation comprises a deletion of between 1 nucleotide and 750 nucleotides in a nucleic acid molecule as compared to a wild type nucleic acid molecule. In an aspect, a non-natural mutation comprises a deletion of between 1 nucleotide and 500 nucleotides in a nucleic acid molecule as compared to a wild type nucleic acid molecule. In an aspect, a non-natural mutation comprises a deletion of between 1 nucleotide and 250 nucleotides in a nucleic acid molecule as compared to a wild type nucleic acid molecule. In an aspect, a non-natural mutation comprises a deletion of between 1 nucleotide and 100 nucleotides in a nucleic acid molecule as compared to a wild type nucleic acid molecule. In an aspect, a non-natural mutation comprises a deletion of between 1 nucleotide and 75 nucleotides in a nucleic acid molecule as compared to a wild type nucleic acid molecule. In an aspect, a non-natural mutation comprises a deletion of between 1 nucleotide and 50 nucleotides in a nucleic acid molecule as compared to a wild type nucleic acid molecule. In an aspect, a non-natural mutation comprises a deletion of between 1 nucleotide and 25 nucleotides in a nucleic acid molecule as compared to a wild type nucleic acid molecule. In an aspect, a non-natural mutation comprises a deletion of between 1 nucleotide and 10 nucleotides in a nucleic acid molecule as compared to a wild type nucleic acid molecule. In an aspect, a non-natural mutation comprises a deletion of between 1 nucleotide and 5 nucleotides in a nucleic acid molecule as compared to a wild type nucleic acid molecule.

In an aspect, a non-natural mutation comprises a deletion of at least 1 amino acid residue in an amino acid sequence as compared to a wild type amino acid sequence. In an aspect, a non-natural mutation comprises a deletion of at least 2 amino acid residues in an amino acid sequence as compared to a wild type amino acid sequence. In an aspect, a non-natural mutation comprises a deletion of at least 3 amino acid residues in an amino acid sequence as compared to a wild type amino acid sequence. In an aspect, a non-natural mutation comprises a deletion of at least 4 amino acid residues in an amino acid sequence as compared to a wild type amino acid sequence. In an aspect, a non-natural mutation comprises a deletion of at least 5 amino acid residues in an amino acid sequence as compared to a wild type amino acid sequence. In an aspect, a non-natural mutation comprises a deletion of at least 6 amino acid residues in an amino acid sequence as compared to a wild type amino acid sequence. In an aspect, a non-natural mutation comprises a deletion of at least 7 amino acid residues in an amino acid sequence as compared to a wild type amino acid sequence. In an aspect, a non-natural mutation comprises a deletion of at least 8 amino acid residues in an amino acid sequence as compared to a wild type amino acid sequence. In an aspect, a non-natural mutation comprises a deletion of at least 9 amino acid residues in an amino acid sequence as compared to a wild type amino acid sequence. In an aspect, a non-natural mutation comprises a deletion of at least 10 amino acid residues in an amino acid sequence as compared to a wild type amino acid sequence. In an aspect, a non-natural mutation comprises a deletion of at least 15 amino acid residues in an amino acid sequence as compared to a wild type amino acid sequence. In an aspect, a non-natural mutation comprises a deletion of at least 20 amino acid residues in an amino acid sequence as compared to a wild type amino acid sequence. In an aspect, a non-natural mutation comprises a deletion of at least 30 amino acid residues in an amino acid sequence as compared to a wild type amino acid sequence. In an aspect, a non-natural mutation comprises a deletion of at least 40 amino acid residues in an amino acid sequence as compared to a wild type amino acid sequence. In an aspect, a non-natural mutation comprises a deletion of at least 50 amino acid residues in an amino acid sequence as compared to a wild type amino acid sequence. In an aspect, a non-natural mutation comprises a deletion of at least 75 amino acid residues in an amino acid sequence as compared to a wild type amino acid sequence. In an aspect, a non-natural mutation comprises a deletion of at least 100 amino acid residues in an amino acid sequence as compared to a wild type amino acid sequence. In an aspect, a non-natural mutation comprises a deletion of at least 250 amino acid residues in an amino acid sequence as compared to a wild type amino acid sequence. In an aspect, a non-natural mutation comprises a deletion of at least 500 amino acid residues in an amino acid sequence as compared to a wild type amino acid sequence. In an aspect, a non-natural mutation comprises a deletion of at least 600 amino acid residues in an amino acid sequence as compared to a wild type amino acid sequence. In an aspect, a non-natural mutation comprises a deletion of at least 700 amino acid residues in an amino acid sequence as compared to a wild type amino acid sequence.

In an aspect, a non-natural mutation comprises a deletion of between 1 amino acid residue and 710 amino acid residues in an amino acid sequence as compared to a wild type amino acid sequence. In an aspect, a non-natural mutation comprises a deletion of between 1 amino acid residue and 700 amino acid residues in an amino acid sequence as compared to a wild type amino acid sequence. In an aspect, a non-natural mutation comprises a deletion of between 1 amino acid residue and 600 amino acid residues in an amino acid sequence as compared to a wild type amino acid sequence. In an aspect, a non-natural mutation comprises a deletion of between 1 amino acid residue and 500 amino acid residues in an amino acid sequence as compared to a wild type amino acid sequence. In an aspect, a non-natural mutation comprises a deletion of between 1 amino acid residue and 250 amino acid residues in an amino acid sequence as compared to a wild type amino acid sequence. In an aspect, a non-natural mutation comprises a deletion of between 1 amino acid residue and 100 amino acid residues in an amino acid sequence as compared to a wild type amino acid sequence. In an aspect, a non-natural mutation comprises a deletion of between 1 amino acid residue and 75 amino acid residues in an amino acid sequence as compared to a wild type amino acid sequence. In an aspect, a non-natural mutation comprises a deletion of between 1 amino acid residue and 50 amino acid residues in an amino acid sequence as compared to a wild type amino acid sequence. In an aspect, a non-natural mutation comprises a deletion of between 1 amino acid residue and 25 amino acid residues in an amino acid sequence as compared to a wild type amino acid sequence. In an aspect, a non-natural mutation comprises a deletion of between 1 amino acid residue and 10 amino acid residues in an amino acid sequence as compared to a wild type amino acid sequence. In an aspect, a non-natural mutation comprises a deletion of between 1 amino acid residue and 5 amino acid residues in an amino acid sequence as compared to a wild type amino acid sequence.

In an aspect, a non-natural mutation comprises a substitution. A “substitution” refers to the replacement of one or more nucleotides or amino acids to a given polynucleotide or amino acid sequence, respectively, as compared to a wild type reference polynucleotide or amino acid sequence. Substitutions do not change the length of a given sequence.

In an aspect, a non-natural mutation comprises at least 1 nucleotide substitution in a nucleic acid molecule as compared to a wild type nucleic acid sequence. In an aspect, a non-natural mutation comprises at least 2 nucleotide substitutions in a nucleic acid molecule as compared to a wild type nucleic acid sequence. In an aspect, a non-natural mutation comprises at least 3 nucleotide substitutions in a nucleic acid molecule as compared to a wild type nucleic acid sequence. In an aspect, a non-natural mutation comprises at least 4 nucleotide substitutions in a nucleic acid molecule as compared to a wild type nucleic acid sequence. In an aspect, a non-natural mutation comprises at least 5 nucleotide substitutions in a nucleic acid molecule as compared to a wild type nucleic acid sequence. In an aspect, a non-natural mutation comprises at least 6 nucleotide substitutions in a nucleic acid molecule as compared to a wild type nucleic acid sequence. In an aspect, a non-natural mutation comprises at least 7 nucleotide substitutions in a nucleic acid molecule as compared to a wild type nucleic acid sequence. In an aspect, a non-natural mutation comprises at least 8 nucleotide substitutions in a nucleic acid molecule as compared to a wild type nucleic acid sequence. In an aspect, a non-natural mutation comprises at least 9 nucleotide substitutions in a nucleic acid molecule as compared to a wild type nucleic acid sequence. In an aspect, a non-natural mutation comprises at least 10 nucleotide substitutions in a nucleic acid molecule as compared to a wild type nucleic acid sequence. In an aspect, a non-natural mutation comprises at least 20 nucleotide substitutions in a nucleic acid molecule as compared to a wild type nucleic acid sequence. In an aspect, a non-natural mutation comprises at least 50 nucleotide substitutions in a nucleic acid molecule as compared to a wild type nucleic acid sequence.

In an aspect, a non-natural mutation comprises between 1 nucleotide substitution and 100 nucleotide substitutions in a nucleic acid sequence as compared to a wild type nucleic acid sequence. In an aspect, a non-natural mutation comprises between 1 nucleotide substitution and 50 nucleotide substitutions in a nucleic acid sequence as compared to a wild type nucleic acid sequence. In an aspect, a non-natural mutation comprises between 1 nucleotide substitution and 25 nucleotide substitutions in a nucleic acid sequence as compared to a wild type nucleic acid sequence. In an aspect, a non-natural mutation comprises between 1 nucleotide substitution and 15 nucleotide substitutions in a nucleic acid sequence as compared to a wild type nucleic acid sequence. In an aspect, a non-natural mutation comprises between 1 nucleotide substitution and 10 nucleotide substitutions in a nucleic acid sequence as compared to a wild type nucleic acid sequence. In an aspect, a non-natural mutation comprises between 1 nucleotide substitution and 5 nucleotide substitutions in an acid sequence as compared to a wild type nucleic acid sequence.

In an aspect, a non-natural mutation comprises at least 1 amino acid residue substitution in an amino acid sequence as compared to a wild type amino acid sequence. In an aspect, a non-natural mutation comprises at least 2 amino acid residue substitutions in an amino acid sequence as compared to a wild type amino acid sequence. In an aspect, a non-natural mutation comprises at least 3 amino acid residue substitutions in an amino acid sequence as compared to a wild type amino acid sequence. In an aspect, a non-natural mutation comprises at least 4 amino acid residue substitutions in an amino acid sequence as compared to a wild type amino acid sequence. In an aspect, a non-natural mutation comprises at least 5 amino acid residue substitutions in an amino acid sequence as compared to a wild type amino acid sequence. In an aspect, a non-natural mutation comprises at least 6 amino acid residue substitutions in an amino acid sequence as compared to a wild type amino acid sequence. In an aspect, a non-natural mutation comprises at least 7 amino acid residue substitutions in an amino acid sequence as compared to a wild type amino acid sequence. In an aspect, a non-natural mutation comprises at least 8 amino acid residue substitutions in an amino acid sequence as compared to a wild type amino acid sequence. In an aspect, a non-natural mutation comprises at least 9 amino acid residue substitutions in an amino acid sequence as compared to a wild type amino acid sequence. In an aspect, a non-natural mutation comprises at least 10 amino acid residue substitutions in an amino acid sequence as compared to a wild type amino acid sequence. In an aspect, a non-natural mutation comprises at least 20 amino acid residue substitutions in an amino acid sequence as compared to a wild type amino acid sequence. In an aspect, a non-natural mutation comprises at least 50 amino acid residue substitutions in an amino acid sequence as compared to a wild type amino acid sequence.

In an aspect, a non-natural mutation comprises between 1 amino acid residue substitution and 100 amino acid residue substitutions in an amino acid sequence as compared to a wild type amino acid sequence. In an aspect, a non-natural mutation comprises between 1 amino acid residue substitution and 50 amino acid residue substitutions in an amino acid sequence as compared to a wild type amino acid sequence. In an aspect, a non-natural mutation comprises between 1 amino acid residue substitution and 25 amino acid residue substitutions in an amino acid sequence as compared to a wild type amino acid sequence. In an aspect, a non-natural mutation comprises between 1 amino acid residue substitution and 15 amino acid residue substitutions in an amino acid sequence as compared to a wild type amino acid sequence. In an aspect, a non-natural mutation comprises between 1 amino acid residue substitution and 10 amino acid residue substitutions in an amino acid sequence as compared to a wild type amino acid sequence. In an aspect, a non-natural mutation comprises between 1 amino acid residue substitution and 5 amino acid residue substitutions in an amino acid sequence as compared to a wild type amino acid sequence.

In an aspect, a non-natural mutation comprises an inversion. An “inversion” refers to when a segment of a polynucleotide or amino acid sequence is reversed end-to-end. An inversion does not change the length of a given sequence.

In an aspect, a non-natural mutation comprises an inversion of at least 2 nucleotides in a nucleic acid molecule as compared to a wild type nucleic acid molecule sequence. In an aspect, a non-natural mutation comprises an inversion of at least 5 nucleotides in a nucleic acid molecule as compared to a wild type nucleic acid molecule sequence. In an aspect, a non-natural mutation comprises an inversion of at least 10 nucleotides in a nucleic acid molecule as compared to a wild type nucleic acid molecule sequence. In an aspect, a non-natural mutation comprises an inversion of at least 25 nucleotides in a nucleic acid molecule as compared to a wild type nucleic acid molecule sequence. In an aspect, a non-natural mutation comprises an inversion of at least 50 nucleotides in a nucleic acid molecule as compared to a wild type nucleic acid molecule sequence. In an aspect, a non-natural mutation comprises an inversion of at least 100 nucleotides in a nucleic acid molecule as compared to a wild type nucleic acid molecule sequence.

In an aspect, a non-natural mutation comprises an inversion of between 2 nucleotides and 1000 nucleotides in a nucleic acid molecule as compared to a wild type nucleic acid molecule sequence. In an aspect, a non-natural mutation comprises an inversion of between 2 nucleotides and 500 nucleotides in a nucleic acid molecule as compared to a wild type nucleic acid molecule sequence. In an aspect, a non-natural mutation comprises an inversion of between 2 nucleotides and 250 nucleotides in a nucleic acid molecule as compared to a wild type nucleic acid molecule sequence. In an aspect, a non-natural mutation comprises an inversion of between 2 nucleotides and 100 nucleotides in a nucleic acid molecule as compared to a wild type nucleic acid molecule sequence. In an aspect, a non-natural mutation comprises an inversion of between 2 nucleotides and 50 nucleotides in a nucleic acid molecule as compared to a wild type nucleic acid molecule sequence. In an aspect, a non-natural mutation comprises an inversion of between 2 nucleotides and 25 nucleotides in a nucleic acid molecule as compared to a wild type nucleic acid molecule sequence. In an aspect, a non-natural mutation comprises an inversion of between 2 nucleotides and 10 nucleotides in a nucleic acid molecule as compared to a wild type nucleic acid molecule sequence.

In an aspect, a non-natural mutation comprises an inversion of at least 2 amino acid residues in an amino acid sequence as compared to a wild type amino acid sequence. In an aspect, a non-natural mutation comprises an inversion of at least 5 amino acid residues in an amino acid sequence as compared to a wild type amino acid sequence. In an aspect, a non-natural mutation comprises an inversion of at least 10 amino acid residues in an amino acid sequence as compared to a wild type amino acid sequence. In an aspect, a non-natural mutation comprises an inversion of at least 25 amino acid residues in an amino acid sequence as compared to a wild type amino acid sequence. In an aspect, a non-natural mutation comprises an inversion of at least 50 amino acid residues in an amino acid sequence as compared to a wild type amino acid sequence. In an aspect, a non-natural mutation comprises an inversion of at least 100 amino acid residues in an amino acid sequence as compared to a wild type amino acid sequence.

In an aspect, a non-natural mutation comprises an inversion of between 2 amino acid residues and 1000 amino acid residues in an amino acid sequence as compared to a wild type amino acid sequence. In an aspect, a non-natural mutation comprises an inversion of between 2 amino acid residues and 500 amino acid residues in an amino acid sequence as compared to a wild type amino acid sequence. In an aspect, a non-natural mutation comprises an inversion of between 2 amino acid residues and 250 amino acid residues in an amino acid sequence as compared to a wild type amino acid sequence. In an aspect, a non-natural mutation comprises an inversion of between 2 amino acid residues and 100 amino acid residues in an amino acid sequence as compared to a wild type amino acid sequence. In an aspect, a non-natural mutation comprises an inversion of between 2 amino acid residues and 50 amino acid residues in an amino acid sequence as compared to a wild type amino acid sequence. In an aspect, a non-natural mutation comprises an inversion of between 2 amino acid residues and 25 amino acid residues in an amino acid sequence as compared to a wild type amino acid sequence. In an aspect, a non-natural mutation comprises an inversion of between 2 amino acid residues and 10 amino acid residues in an amino acid sequence as compared to a wild type amino acid sequence.

In an aspect, a non-natural mutation comprises a duplication. A “duplication” refers to when a segment of a polynucleotide or amino acid sequence is repeated. The repeated segment can immediately follow the original segment, or it can be separated from the original segment by one or more nucleotides or amino acid residues. When a duplication consists of at least n nucleotides, it will be appreciated that this refers to n consecutive nucleotides being duplicated as a block (e.g., it does not refer to an individual nucleotide being duplicated x times). For example, a duplication of the nucleic acid sequence 5′-ATGC-3′ would result in the nucleotide sequence 5′-ATGCATGC-3′.

In an aspect, a non-natural mutation comprises a duplication of at least 2 nucleotides. In an aspect, a non-natural mutation comprises a duplication of at least 5 nucleotides. In an aspect, a non-natural mutation comprises a duplication of at least 10 nucleotides. In an aspect, a non-natural mutation comprises a duplication of at least 25 nucleotides. In an aspect, a non-natural mutation comprises a duplication of at least 50 nucleotides. In an aspect, a non-natural mutation comprises a duplication of at least 75 nucleotides. In an aspect, a non-natural mutation comprises a duplication of at least 100 nucleotides. In an aspect, a non-natural mutation comprises a duplication of at least 250 nucleotides. In an aspect, a non-natural mutation comprises a duplication of at least 500 nucleotides. In an aspect, a non-natural mutation comprises a duplication of at least 750 nucleotides. In an aspect, a non-natural mutation comprises a duplication of at least 1000 nucleotides.

In an aspect, a non-natural mutation comprises a duplication of between 2 nucleotides and 2500 nucleotides. In an aspect, a non-natural mutation comprises a duplication of between 2 nucleotides and 1500 nucleotides. In an aspect, a non-natural mutation comprises a duplication of between 2 nucleotides and 1000 nucleotides. In an aspect, a non-natural mutation comprises a duplication of between 2 nucleotides and 500 nucleotides. In an aspect, a non-natural mutation comprises a duplication of between 2 nucleotides and 250 nucleotides. In an aspect, a non-natural mutation comprises a duplication of between 2 nucleotides and 100 nucleotides. In an aspect, a non-natural mutation comprises a duplication of between 2 nucleotides and 50 nucleotides. In an aspect, a non-natural mutation comprises a duplication of between 2 nucleotides and 25 nucleotides.

In an aspect, a non-natural mutation comprises a duplication of at least 2 amino acid residues. In an aspect, a non-natural mutation comprises a duplication of at least 5 amino acid residues. In an aspect, a non-natural mutation comprises a duplication of at least 10 amino acid residues. In an aspect, a non-natural mutation comprises a duplication of at least 25 amino acid residues. In an aspect, a non-natural mutation comprises a duplication of at least 50 amino acid residues. In an aspect, a non-natural mutation comprises a duplication of at least 75 amino acid residues. In an aspect, a non-natural mutation comprises a duplication of at least 100 amino acid residues.

In an aspect, a non-natural mutation comprises a duplication of between 2 amino acid residues and 500 amino acid residues. In an aspect, a non-natural mutation comprises a duplication of between 2 amino acid residues and 250 amino acid residues. In an aspect, a non-natural mutation comprises a duplication of between 2 amino acid residues and 100 amino acid residues. In an aspect, a non-natural mutation comprises a duplication of between 2 amino acid residues and 75 amino acid residues. In an aspect, a non-natural mutation comprises a duplication of between 2 amino acid residues and 50 amino acid residues. In an aspect, a non-natural mutation comprises a duplication of between 2 amino acid residues and 25 amino acid residues. In an aspect, a non-natural mutation comprises a duplication of between 2 amino acid residues and 10 amino acid residues.

In an aspect, a mutation provided herein comprises a mutation selected from the group consisting of an insertion, a deletion, a substitution, a duplication, and an inversion.

Unless otherwise noted, when comparing trichome number, trichome length, and/or trichome density between a leaf from a modified tobacco plant and a leaf from a control tobacco plant, it shall be understood that the comparison must be performed between leaves of the same age/growth stage and the same physical size area of the leaf (e.g., a 1 cm² section on each leaf). Any age or growth stage of leaves can be compared, so long as they are the same growth stage. For example, a V5 leaf of a modified tobacco plant should be compared to a V5 leaf from a control tobacco plant. However, unless otherwise noted, trichome density, trichome length, and/or trichome number is measured using fully expanded adult leaves.

As a point of clarification, it would be improper to compare trichome number, trichome length, and/or trichome density between a newly emerged leaf with a mature, senescing leaf for the purposes of this disclosure. It would also be improper, for example, to compare trichome number, trichome length, and/or trichome density between a 1 cm² section of a modified tobacco plant leaf to a 3 cm² section of a control tobacco plant leaf.

In an aspect, a modified tobacco plant comprising a non-natural mutation in an endogenous nucleic acid molecule encoding a HAIRPLUS1a protein comprises an increased density of trichomes on at least one leaf as compared to a control tobacco plant lacking the non-natural mutation when grown under comparable conditions. In an aspect, a modified tobacco plant comprising a non-natural mutation in an endogenous nucleic acid molecule encoding a HAIRPLUS1b protein comprises an increased density of trichomes on at least one leaf as compared to a control tobacco plant lacking the non-natural mutation when grown under comparable conditions. In an aspect, a modified tobacco plant comprising a non-natural mutation in an endogenous nucleic acid molecule encoding a HAIRPLUS2 protein comprises an increased density of trichomes on at least one leaf as compared to a control tobacco plant lacking the non-natural mutation when grown under comparable conditions. In an aspect, a modified tobacco plant comprising a non-natural mutation in an endogenous nucleic acid molecule encoding a HAIRPLUS3 protein comprises an increased density of trichomes on at least one leaf as compared to a control tobacco plant lacking the non-natural mutation when grown under comparable conditions.

In an aspect, a modified tobacco plant comprising a first non-natural mutation in an endogenous nucleic acid molecule encoding a HAIRPLUS1a protein and a second non-natural mutation in an endogenous nucleic acid molecule encoding a HAIRPLUS1b protein comprises an increased density of trichomes on at least one leaf as compared to a control tobacco plant lacking the non-natural mutations when grown under comparable conditions. In an aspect, a modified tobacco plant comprising a first non-natural mutation in an endogenous nucleic acid molecule encoding a HAIRPLUS1a protein and a second non-natural mutation in an endogenous nucleic acid molecule encoding a HAIRPLUS2 protein comprises an increased density of trichomes on at least one leaf as compared to a control tobacco plant lacking the non-natural mutations when grown under comparable conditions. In an aspect, a modified tobacco plant comprising a first non-natural mutation in an endogenous nucleic acid molecule encoding a HAIRPLUS1b protein and a second non-natural mutation in an endogenous nucleic acid molecule encoding a HAIRPLUS2 protein comprises an increased density of trichomes on at least one leaf as compared to a control tobacco plant lacking the non-natural mutations when grown under comparable conditions. In an aspect, a modified tobacco plant comprising a first non-natural mutation in an endogenous nucleic acid molecule encoding a HAIRPLUS1a protein and a second non-natural mutation in an endogenous nucleic acid molecule encoding a HAIRPLUS3 protein comprises an increased density of trichomes on at least one leaf as compared to a control tobacco plant lacking the non-natural mutations when grown under comparable conditions. In an aspect, a modified tobacco plant comprising a first non-natural mutation in an endogenous nucleic acid molecule encoding a HAIRPLUS1b protein and a second non-natural mutation in an endogenous nucleic acid molecule encoding a HAIRPLUS3 protein comprises an increased density of trichomes on at least one leaf as compared to a control tobacco plant lacking the non-natural mutations when grown under comparable conditions. In an aspect, a modified tobacco plant comprising a first non-natural mutation in an endogenous nucleic acid molecule encoding a HAIRPLUS2 protein and a second non-natural mutation in an endogenous nucleic acid molecule encoding a HAIRPLUS3 protein comprises an increased density of trichomes on at least one leaf as compared to a control tobacco plant lacking the non-natural mutations when grown under comparable conditions.

In an aspect, a modified tobacco plant comprising a first non-natural mutation in an endogenous nucleic acid molecule encoding a HAIRPLUS1a protein, a second non-natural mutation in an endogenous nucleic acid molecule encoding a HAIRPLUS1b protein, and a third non-natural mutation in an endogenous nucleic acid molecule encoding a HAIRPLUS2 protein comprises an increased density of trichomes on at least one leaf as compared to a control tobacco plant lacking the non-natural mutations when grown under comparable conditions. In an aspect, a modified tobacco plant comprising a first non-natural mutation in an endogenous nucleic acid molecule encoding a HAIRPLUS1a protein, a second non-natural mutation in an endogenous nucleic acid molecule encoding a HAIRPLUS1b protein, and a third non-natural mutation in an endogenous nucleic acid molecule encoding a HAIRPLUS3 protein comprises an increased density of trichomes on at least one leaf as compared to a control tobacco plant lacking the non-natural mutations when grown under comparable conditions. In an aspect, a modified tobacco plant comprising a first non-natural mutation in an endogenous nucleic acid molecule encoding a HAIRPLUS1a protein, a second non-natural mutation in an endogenous nucleic acid molecule encoding a HAIRPLUS2 protein, and a third non-natural mutation in an endogenous nucleic acid molecule encoding a HAIRPLUS3 protein comprises an increased density of trichomes on at least one leaf as compared to a control tobacco plant lacking the non-natural mutations when grown under comparable conditions. In an aspect, a modified tobacco plant comprising a first non-natural mutation in an endogenous nucleic acid molecule encoding a HAIRPLUS1b protein, a second non-natural mutation in an endogenous nucleic acid molecule encoding a HAIRPLUS2 protein, and a third non-natural mutation in an endogenous nucleic acid molecule encoding a HAIRPLUS3 protein comprises an increased density of trichomes on at least one leaf as compared to a control tobacco plant lacking the non-natural mutations when grown under comparable conditions.

In an aspect, a modified tobacco plant comprising a first non-natural mutation in an endogenous nucleic acid molecule encoding a HAIRPLUS1a protein, a second non-natural mutation in an endogenous nucleic acid molecule encoding a HAIRPLUS1b protein, a third non-natural mutation in an endogenous nucleic acid molecule encoding a HAIRPLUS2 protein comprises an increased density of trichomes on at least one leaf as compared to a control tobacco plant lacking the non-natural mutations when grown under comparable conditions, and a fourth non-natural mutation in an endogenous nucleic acid molecule encoding a HAIRPLUS3 protein comprises an increased density of trichomes on at least one leaf as compared to a control tobacco plant lacking the first, second, third, and fourth non-natural mutations when grown under comparable conditions.

In an aspect, a modified tobacco plant comprising a non-natural mutation in an endogenous nucleic acid molecule comprising a HAIRPLUS1a coding sequence or genomic sequence comprises an increased density of trichomes on at least one leaf as compared to a control tobacco plant lacking the non-natural mutation when grown under comparable conditions. In an aspect, a modified tobacco plant comprising a non-natural mutation in an endogenous nucleic acid molecule comprising a HAIRPLUS1b coding sequence or genomic sequence comprises an increased density of trichomes on at least one leaf as compared to a control tobacco plant lacking the non-natural mutation when grown under comparable conditions. In an aspect, a modified tobacco plant comprising a non-natural mutation in an endogenous nucleic acid molecule comprising a HAIRPLUS2 coding sequence or genomic sequence comprises an increased density of trichomes on at least one leaf as compared to a control tobacco plant lacking the non-natural mutation when grown under comparable conditions. In an aspect, a modified tobacco plant comprising a non-natural mutation in an endogenous nucleic acid molecule comprising a HAIRPLUS3 coding sequence or genomic sequence comprises an increased density of trichomes on at least one leaf as compared to a control tobacco plant lacking the non-natural mutation when grown under comparable conditions.

In an aspect, a modified tobacco plant comprising a first non-natural mutation in an endogenous nucleic acid molecule comprising a HAIRPLUS1a coding sequence or genomic sequence and a second non-natural mutation in an endogenous nucleic acid molecule comprising a HAIRPLUS1b coding sequence or genomic sequence comprises an increased density of trichomes on at least one leaf as compared to a control tobacco plant lacking the non-natural mutations when grown under comparable conditions. In an aspect, a modified tobacco plant comprising a first non-natural mutation in an endogenous nucleic acid molecule comprising a HAIRPLUS1a coding sequence or genomic sequence and a second non-natural mutation in an endogenous nucleic acid molecule comprising a HAIRPLUS2 coding sequence or genomic sequence comprises an increased density of trichomes on at least one leaf as compared to a control tobacco plant lacking the non-natural mutations when grown under comparable conditions. In an aspect, a modified tobacco plant comprising a first non-natural mutation in an endogenous nucleic acid molecule comprising a HAIRPLUS1b coding sequence or genomic sequence and a second non-natural mutation in an endogenous nucleic acid molecule comprising a HAIRPLUS2 coding sequence or genomic sequence comprises an increased density of trichomes on at least one leaf as compared to a control tobacco plant lacking the non-natural mutations when grown under comparable conditions. In an aspect, a modified tobacco plant comprising a first non-natural mutation in an endogenous nucleic acid molecule comprising a HAIRPLUS1a coding sequence or genomic sequence and a second non-natural mutation in an endogenous nucleic acid molecule comprising a HAIRPLUS3 coding sequence or genomic sequence comprises an increased density of trichomes on at least one leaf as compared to a control tobacco plant lacking the non-natural mutations when grown under comparable conditions. In an aspect, a modified tobacco plant comprising a first non-natural mutation in an endogenous nucleic acid molecule comprising a HAIRPLUS1b coding sequence or genomic sequence and a second non-natural mutation in an endogenous nucleic acid molecule comprising a HAIRPLUS3 coding sequence or genomic sequence comprises an increased density of trichomes on at least one leaf as compared to a control tobacco plant lacking the non-natural mutations when grown under comparable conditions. In an aspect, a modified tobacco plant comprising a first non-natural mutation in an endogenous nucleic acid molecule comprising a HAIRPLUS2 coding sequence or genomic sequence and a second non-natural mutation in an endogenous nucleic acid molecule comprising a HAIRPLUS3 coding sequence or genomic sequence comprises an increased density of trichomes on at least one leaf as compared to a control tobacco plant lacking the non-natural mutations when grown under comparable conditions.

In an aspect, a modified tobacco plant comprising a first non-natural mutation in an endogenous nucleic acid molecule comprising a HAIRPLUS1a coding sequence or genomic sequence, a second non-natural mutation in an endogenous nucleic acid molecule comprising a HAIRPLUS1b coding sequence or genomic sequence, and a third non-natural mutation in an endogenous nucleic acid molecule comprising a HAIRPLUS2 coding sequence or genomic sequence comprises an increased density of trichomes on at least one leaf as compared to a control tobacco plant lacking the non-natural mutations when grown under comparable conditions. In an aspect, a modified tobacco plant comprising a first non-natural mutation in an endogenous nucleic acid molecule comprising a HAIRPLUS1a coding sequence or genomic sequence, a second non-natural mutation in an endogenous nucleic acid molecule comprising a HAIRPLUS1b coding sequence or genomic sequence, and a third non-natural mutation in an endogenous nucleic acid molecule comprising a HAIRPLUS3 coding sequence or genomic sequence comprises an increased density of trichomes on at least one leaf as compared to a control tobacco plant lacking the non-natural mutations when grown under comparable conditions. In an aspect, a modified tobacco plant comprising a first non-natural mutation in an endogenous nucleic acid molecule comprising a HAIRPLUS1a coding sequence or genomic sequence, a second non-natural mutation in an endogenous nucleic acid molecule comprising a HAIRPLUS2 coding sequence or genomic sequence, and a third non-natural mutation in an endogenous nucleic acid molecule comprising a HAIRPLUS3 coding sequence or genomic sequence comprises an increased density of trichomes on at least one leaf as compared to a control tobacco plant lacking the non-natural mutations when grown under comparable conditions. In an aspect, a modified tobacco plant comprising a first non-natural mutation in an endogenous nucleic acid molecule comprising a HAIRPLUS1b coding sequence or genomic sequence, a second non-natural mutation in an endogenous nucleic acid molecule comprising a HAIRPLUS2 coding sequence or genomic sequence, and a third non-natural mutation in an endogenous nucleic acid molecule comprising a HAIRPLUS3 coding sequence or genomic sequence comprises an increased density of trichomes on at least one leaf as compared to a control tobacco plant lacking the non-natural mutations when grown under comparable conditions.

In an aspect, a modified tobacco plant comprising a first non-natural mutation in an endogenous nucleic acid molecule comprising a HAIRPLUS1a coding sequence or genomic sequence, a second non-natural mutation in an endogenous nucleic acid molecule comprising a HAIRPLUS1b coding sequence or genomic sequence, a third non-natural mutation in an endogenous nucleic acid molecule comprising a HAIRPLUS2 coding sequence or genomic sequence, and a fourth non-natural mutation in an endogenous nucleic acid molecule comprising a HAIRPLUS3 coding sequence or genomic sequence comprises an increased density of trichomes on at least one leaf as compared to a control tobacco plant lacking the non-natural mutations when grown under comparable conditions.

In an aspect, at least two leaves of a modified tobacco plant comprise an increased density of trichomes as compared to a control tobacco plant when grown under comparable conditions. In an aspect, at least three leaves of a modified tobacco plant comprise an increased density of trichomes as compared to a control tobacco plant when grown under comparable conditions. In an aspect, at least four leaves of a modified tobacco plant comprise an increased density of trichomes as compared to a control tobacco plant when grown under comparable conditions. In an aspect, at least five leaves of a modified tobacco plant comprise an increased density of trichomes as compared to a control tobacco plant when grown under comparable conditions. In an aspect, at least six leaves of a modified tobacco plant comprise an increased density of trichomes as compared to a control tobacco plant when grown under comparable conditions. In an aspect, at least seven leaves of a modified tobacco plant comprise an increased density of trichomes as compared to a control tobacco plant when grown under comparable conditions. In an aspect, at least eight leaves of a modified tobacco plant comprise an increased density of trichomes as compared to a control tobacco plant when grown under comparable conditions. In an aspect, at least nine leaves of a modified tobacco plant comprise an increased density of trichomes as compared to a control tobacco plant when grown under comparable conditions. In an aspect, at least 10 leaves of a modified tobacco plant comprise an increased density of trichomes as compared to a control tobacco plant when grown under comparable conditions.

In an aspect, at least 10% of the leaves of a modified tobacco plant comprise an increased density of trichomes as compared to a control tobacco plant when grown under comparable conditions. In an aspect, at least 20% of the leaves of a modified tobacco plant comprise an increased density of trichomes as compared to a control tobacco plant when grown under comparable conditions. In an aspect, at least 30% of the leaves of a modified tobacco plant comprise an increased density of trichomes as compared to a control tobacco plant when grown under comparable conditions. In an aspect, at least 40% of the leaves of a modified tobacco plant comprise an increased density of trichomes as compared to a control tobacco plant when grown under comparable conditions. In an aspect, at least 50% of the leaves of a modified tobacco plant comprise an increased density of trichomes as compared to a control tobacco plant when grown under comparable conditions. In an aspect, at least 60% of the leaves of a modified tobacco plant comprise an increased density of trichomes as compared to a control tobacco plant when grown under comparable conditions. In an aspect, at least 70% of the leaves of a modified tobacco plant comprise an increased density of trichomes as compared to a control tobacco plant when grown under comparable conditions. In an aspect, at least 75% of the leaves of a modified tobacco plant comprise an increased density of trichomes as compared to a control tobacco plant when grown under comparable conditions. In an aspect, at least 80% of the leaves of a modified tobacco plant comprise an increased density of trichomes as compared to a control tobacco plant when grown under comparable conditions. In an aspect, at least 85% of the leaves of a modified tobacco plant comprise an increased density of trichomes as compared to a control tobacco plant when grown under comparable conditions. In an aspect, at least 90% of the leaves of a modified tobacco plant comprise an increased density of trichomes as compared to a control tobacco plant when grown under comparable conditions. In an aspect, at least 95% of the leaves of a modified tobacco plant comprise an increased density of trichomes as compared to a control tobacco plant when grown under comparable conditions. In an aspect, at least 97.5% of the leaves of a modified tobacco plant comprise an increased density of trichomes as compared to a control tobacco plant when grown under comparable conditions. In an aspect, 100% of the leaves of a modified tobacco plant comprise an increased density of trichomes as compared to a control tobacco plant when grown under comparable conditions.

In an aspect, a modified tobacco plant comprises an increased density of trichomes on the adaxial surface of one or more leaves as compared to a control tobacco plant when grown under comparable conditions. In an aspect, a modified tobacco plant comprises an increased density of trichomes on the abaxial surface of one or more leaves as compared to a control tobacco plant when grown under comparable conditions. In an aspect, a modified tobacco plant comprises an increased density of trichomes on the adaxial surface and the abaxial surface of one or more leaves as compared to a control tobacco plant when grown under comparable conditions.

In an aspect, a modified tobacco plant comprises an increased density of glandular trichomes on one or more leaves as compared to a control tobacco plant when grown under comparable conditions.

Density of trichomes can be measured by counting the number of trichomes present in a unit of area. Determination of trichome density can be performed using tools including, but not limited to, a stereomicroscope, an image capture device (e.g., a camera), and computer software. See, for example, Mirnezami et al., Appl Plant Sci., 8(7):e11375 (2020). Similarly, the length of trichomes can be measured using tools including, but not limited to, a stereomicroscope, an image capture device (e.g., a camera), and computer software (e.g., ImageJ).

In an aspect, a leaf of a modified tobacco plant comprises at least 70 trichomes per square centimeter. In an aspect, a leaf of a modified tobacco plant comprises at least 75 trichomes per square centimeter. In an aspect, a leaf of a modified tobacco plant comprises at least 80 trichomes per square centimeter. In an aspect, a leaf of a modified tobacco plant comprises at least 85 trichomes per square centimeter. In an aspect, a leaf of a modified tobacco plant comprises at least 90 trichomes per square centimeter. In an aspect, a leaf of a modified tobacco plant comprises at least 95 trichomes per square centimeter. In an aspect, a leaf of a modified tobacco plant comprises at least 100 trichomes per square centimeter. In an aspect, a leaf of a modified tobacco plant comprises at least 105 trichomes per square centimeter. In an aspect, a leaf of a modified tobacco plant comprises at least 110 trichomes per square centimeter. In an aspect, a leaf of a modified tobacco plant comprises at least 115 trichomes per square centimeter. In an aspect, a leaf of a modified tobacco plant comprises at least 120 trichomes per square centimeter. In an aspect, a leaf of a modified tobacco plant comprises at least 125 trichomes per square centimeter. In an aspect, a leaf of a modified tobacco plant comprises at least 130 trichomes per square centimeter. In an aspect, a leaf of a modified tobacco plant comprises at least 140 trichomes per square centimeter. In an aspect, a leaf of a modified tobacco plant comprises at least 150 trichomes per square centimeter. In an aspect, a leaf of a modified tobacco plant comprises at least 250 trichomes per square centimeter. In an aspect, a leaf of a modified tobacco plant comprises at least 350 trichomes per square centimeter. In an aspect, a leaf of a modified tobacco plant comprises at least 400 trichomes per square centimeter. In an aspect, a leaf of a modified tobacco plant comprises at least 500 trichomes per square centimeter. In an aspect, a leaf of a modified tobacco plant comprises at least 600 trichomes per square centimeter. In an aspect, a leaf of a modified tobacco plant comprises at least 700 trichomes per square centimeter. In an aspect, a leaf of a modified tobacco plant comprises at least 800 trichomes per square centimeter. In an aspect, a leaf of a modified tobacco plant comprises at least 900 trichomes per square centimeter. In an aspect, a leaf of a modified tobacco plant comprises at least 1000 trichomes per square centimeter. In an aspect, a leaf of a modified tobacco plant comprises at least 1100 trichomes per square centimeter. In an aspect, a leaf of a modified tobacco plant comprises at least 1200 trichomes per square centimeter. In an aspect, a leaf of a modified tobacco plant comprises at least 1250 trichomes per square centimeter. In an aspect, a leaf of a modified tobacco plant comprises at least 1300 trichomes per square centimeter. In an aspect, a leaf of a modified tobacco plant comprises at least 1400 trichomes per square centimeter. In an aspect, a leaf of a modified tobacco plant comprises at least 1500 trichomes per square centimeter. In an aspect, a leaf of a modified tobacco plant comprises at least 1750 trichomes per square centimeter. In an aspect, a leaf of a modified tobacco plant comprises at least 2000 trichomes per square centimeter. In an aspect, a leaf of a modified tobacco plant comprises at least 2250 trichomes per square centimeter. In an aspect, a leaf of a modified tobacco plant comprises at least 2500 trichomes per square centimeter. In an aspect, a leaf of a modified tobacco plant comprises at least 2750 trichomes per square centimeter. In an aspect, a leaf of a modified tobacco plant comprises at least 3000 trichomes per square centimeter. In an aspect, a leaf of a modified tobacco plant comprises at least 4000 trichomes per square centimeter.

In an aspect, a leaf of a modified tobacco plant comprises between 60 and 200 trichomes per square centimeter. In an aspect, a leaf of a modified tobacco plant comprises between 60 and 190 trichomes per square centimeter. In an aspect, a leaf of a modified tobacco plant comprises between 60 and 180 trichomes per square centimeter. In an aspect, a leaf of a modified tobacco plant comprises between 60 and 170 trichomes per square centimeter. In an aspect, a leaf of a modified tobacco plant comprises between 60 and 160 trichomes per square centimeter. In an aspect, a leaf of a modified tobacco plant comprises between 60 and 150 trichomes per square centimeter. In an aspect, a leaf of a modified tobacco plant comprises between 60 and 140 trichomes per square centimeter. In an aspect, a leaf of a modified tobacco plant comprises between 60 and 130 trichomes per square centimeter. In an aspect, a leaf of a modified tobacco plant comprises between 60 and 120 trichomes per square centimeter. In an aspect, a leaf of a modified tobacco plant comprises between 60 and 110 trichomes per square centimeter. In an aspect, a leaf of a modified tobacco plant comprises between 60 and 100 trichomes per square centimeter. In an aspect, a leaf of a modified tobacco plant comprises between 60 and 90 trichomes per square centimeter. In an aspect, a leaf of a modified tobacco plant comprises between 60 and 80 trichomes per square centimeter. In an aspect, a leaf of a modified tobacco plant comprises between 60 and 70 trichomes per square centimeter.

In an aspect, a leaf of a modified tobacco plant comprises between 80 and 130 trichomes per square centimeter. In an aspect, a leaf of a modified tobacco plant comprises between 80 and 120 trichomes per square centimeter. In an aspect, a leaf of a modified tobacco plant comprises between 80 and 100 trichomes per square centimeter. In an aspect, a leaf of a modified tobacco plant comprises between 75 and 140 trichomes per square centimeter. In an aspect, a leaf of a modified tobacco plant comprises between 75 and 130 trichomes per square centimeter. In an aspect, a leaf of a modified tobacco plant comprises between 75 and 125 trichomes per square centimeter.

In an aspect, a leaf of a modified tobacco plant comprises between 400 and 4000 trichomes per square centimeter. In an aspect, a leaf of a modified tobacco plant comprises between 400 and 3500 trichomes per square centimeter. In an aspect, a leaf of a modified tobacco plant comprises between 400 and 3000 trichomes per square centimeter. In an aspect, a leaf of a modified tobacco plant comprises between 400 and 2500 trichomes per square centimeter. In an aspect, a leaf of a modified tobacco plant comprises between 400 and 2000 trichomes per square centimeter. In an aspect, a leaf of a modified tobacco plant comprises between 400 and 1500 trichomes per square centimeter. In an aspect, a leaf of a modified tobacco plant comprises between 400 and 1250 trichomes per square centimeter. In an aspect, a leaf of a modified tobacco plant comprises between 400 and 1000 trichomes per square centimeter. In an aspect, a leaf of a modified tobacco plant comprises between 400 and 900 trichomes per square centimeter. In an aspect, a leaf of a modified tobacco plant comprises between 400 and 800 trichomes per square centimeter. In an aspect, a leaf of a modified tobacco plant comprises between 400 and 700 trichomes per square centimeter. In an aspect, a leaf of a modified tobacco plant comprises between 400 and 600 trichomes per square centimeter. In an aspect, a leaf of a modified tobacco plant comprises between 400 and 500 trichomes per square centimeter.

In an aspect, a leaf of a modified tobacco plant comprises between 500 and 4000 trichomes per square centimeter. In an aspect, a leaf of a modified tobacco plant comprises between 500 and 3500 trichomes per square centimeter. In an aspect, a leaf of a modified tobacco plant comprises between 500 and 3000 trichomes per square centimeter. In an aspect, a leaf of a modified tobacco plant comprises between 500 and 2500 trichomes per square centimeter. In an aspect, a leaf of a modified tobacco plant comprises between 500 and 2000 trichomes per square centimeter. In an aspect, a leaf of a modified tobacco plant comprises between 500 and 1500 trichomes per square centimeter. In an aspect, a leaf of a modified tobacco plant comprises between 500 and 1250 trichomes per square centimeter. In an aspect, a leaf of a modified tobacco plant comprises between 500 and 1000 trichomes per square centimeter. In an aspect, a leaf of a modified tobacco plant comprises between 500 and 900 trichomes per square centimeter. In an aspect, a leaf of a modified tobacco plant comprises between 500 and 800 trichomes per square centimeter. In an aspect, a leaf of a modified tobacco plant comprises between 500 and 700 trichomes per square centimeter. In an aspect, a leaf of a modified tobacco plant comprises between 500 and 600 trichomes per square centimeter.

In an aspect, a leaf of a modified tobacco plant comprises between 600 and 4000 trichomes per square centimeter. In an aspect, a leaf of a modified tobacco plant comprises between 600 and 3500 trichomes per square centimeter. In an aspect, a leaf of a modified tobacco plant comprises between 600 and 3000 trichomes per square centimeter. In an aspect, a leaf of a modified tobacco plant comprises between 600 and 2500 trichomes per square centimeter. In an aspect, a leaf of a modified tobacco plant comprises between 600 and 2000 trichomes per square centimeter. In an aspect, a leaf of a modified tobacco plant comprises between 600 and 1500 trichomes per square centimeter. In an aspect, a leaf of a modified tobacco plant comprises between 600 and 1250 trichomes per square centimeter. In an aspect, a leaf of a modified tobacco plant comprises between 600 and 1000 trichomes per square centimeter. In an aspect, a leaf of a modified tobacco plant comprises between 600 and 900 trichomes per square centimeter. In an aspect, a leaf of a modified tobacco plant comprises between 600 and 800 trichomes per square centimeter. In an aspect, a leaf of a modified tobacco plant comprises between 600 and 700 trichomes per square centimeter.

In an aspect, a leaf of a modified tobacco plant comprises between 700 and 4000 trichomes per square centimeter. In an aspect, a leaf of a modified tobacco plant comprises between 700 and 3500 trichomes per square centimeter. In an aspect, a leaf of a modified tobacco plant comprises between 700 and 3000 trichomes per square centimeter. In an aspect, a leaf of a modified tobacco plant comprises between 700 and 2500 trichomes per square centimeter. In an aspect, a leaf of a modified tobacco plant comprises between 700 and 2000 trichomes per square centimeter. In an aspect, a leaf of a modified tobacco plant comprises between 700 and 1500 trichomes per square centimeter. In an aspect, a leaf of a modified tobacco plant comprises between 700 and 1250 trichomes per square centimeter. In an aspect, a leaf of a modified tobacco plant comprises between 700 and 1000 trichomes per square centimeter. In an aspect, a leaf of a modified tobacco plant comprises between 700 and 900 trichomes per square centimeter. In an aspect, a leaf of a modified tobacco plant comprises between 700 and 800 trichomes per square centimeter.

In an aspect, a leaf of a modified tobacco plant comprises between 800 and 4000 trichomes per square centimeter. In an aspect, a leaf of a modified tobacco plant comprises between 800 and 3500 trichomes per square centimeter. In an aspect, a leaf of a modified tobacco plant comprises between 800 and 3000 trichomes per square centimeter. In an aspect, a leaf of a modified tobacco plant comprises between 800 and 2500 trichomes per square centimeter. In an aspect, a leaf of a modified tobacco plant comprises between 800 and 2000 trichomes per square centimeter. In an aspect, a leaf of a modified tobacco plant comprises between 800 and 1500 trichomes per square centimeter. In an aspect, a leaf of a modified tobacco plant comprises between 800 and 1250 trichomes per square centimeter. In an aspect, a leaf of a modified tobacco plant comprises between 800 and 1000 trichomes per square centimeter. In an aspect, a leaf of a modified tobacco plant comprises between 800 and 900 trichomes per square centimeter.

In an aspect, a leaf of a modified tobacco plant comprises between 900 and 4000 trichomes per square centimeter. In an aspect, a leaf of a modified tobacco plant comprises between 900 and 3500 trichomes per square centimeter. In an aspect, a leaf of a modified tobacco plant comprises between 900 and 3000 trichomes per square centimeter. In an aspect, a leaf of a modified tobacco plant comprises between 900 and 2500 trichomes per square centimeter. In an aspect, a leaf of a modified tobacco plant comprises between 900 and 2000 trichomes per square centimeter. In an aspect, a leaf of a modified tobacco plant comprises between 900 and 1500 trichomes per square centimeter. In an aspect, a leaf of a modified tobacco plant comprises between 900 and 1250 trichomes per square centimeter. In an aspect, a leaf of a modified tobacco plant comprises between 900 and 1000 trichomes per square centimeter.

In an aspect, a leaf of a modified tobacco plant comprises between 1000 and 4000 trichomes per square centimeter. In an aspect, a leaf of a modified tobacco plant comprises between 1000 and 3500 trichomes per square centimeter. In an aspect, a leaf of a modified tobacco plant comprises between 1000 and 3000 trichomes per square centimeter. In an aspect, a leaf of a modified tobacco plant comprises between 1000 and 2500 trichomes per square centimeter. In an aspect, a leaf of a modified tobacco plant comprises between 1000 and 2000 trichomes per square centimeter. In an aspect, a leaf of a modified tobacco plant comprises between 1000 and 1500 trichomes per square centimeter. In an aspect, a leaf of a modified tobacco plant comprises between 1000 and 1250 trichomes per square centimeter.

In an aspect, a leaf of a modified tobacco plant comprises at least 1% more trichomes per square centimeter as compared to a leaf from a control tobacco plant when grown under comparable conditions. In an aspect, a leaf of a modified tobacco plant comprises at least 5% more trichomes per square centimeter as compared to a leaf from a control tobacco plant when grown under comparable conditions. In an aspect, a leaf of a modified tobacco plant comprises at least 10% more trichomes per square centimeter as compared to a leaf from a control tobacco plant when grown under comparable conditions. In an aspect, a leaf of a modified tobacco plant comprises at least 15% more trichomes per square centimeter as compared to a leaf from a control tobacco plant when grown under comparable conditions. In an aspect, a leaf of a modified tobacco plant comprises at least 20% more trichomes per square centimeter as compared to a leaf from a control tobacco plant when grown under comparable conditions. In an aspect, a leaf of a modified tobacco plant comprises at least 25% more trichomes per square centimeter as compared to a leaf from a control tobacco plant when grown under comparable conditions. In an aspect, a leaf of a modified tobacco plant comprises at least 30% more trichomes per square centimeter as compared to a leaf from a control tobacco plant when grown under comparable conditions. In an aspect, a leaf of a modified tobacco plant comprises at least 40% more trichomes per square centimeter as compared to a leaf from a control tobacco plant when grown under comparable conditions. In an aspect, a leaf of a modified tobacco plant comprises at least 50% more trichomes per square centimeter as compared to a leaf from a control tobacco plant when grown under comparable conditions. In an aspect, a leaf of a modified tobacco plant comprises at least 60% more trichomes per square centimeter as compared to a leaf from a control tobacco plant when grown under comparable conditions. In an aspect, a leaf of a modified tobacco plant comprises at least 70% more trichomes per square centimeter as compared to a leaf from a control tobacco plant when grown under comparable conditions. In an aspect, a leaf of a modified tobacco plant comprises at least 80% more trichomes per square centimeter as compared to a leaf from a control tobacco plant when grown under comparable conditions. In an aspect, a leaf of a modified tobacco plant comprises at least 90% more trichomes per square centimeter as compared to a leaf from a control tobacco plant when grown under comparable conditions. In an aspect, a leaf of a modified tobacco plant comprises at least 100% more trichomes per square centimeter as compared to a leaf from a control tobacco plant when grown under comparable conditions. In an aspect, a leaf of a modified tobacco plant comprises at least 110% more trichomes per square centimeter as compared to a leaf from a control tobacco plant when grown under comparable conditions. In an aspect, a leaf of a modified tobacco plant comprises at least 125% more trichomes per square centimeter as compared to a leaf from a control tobacco plant when grown under comparable conditions. In an aspect, a leaf of a modified tobacco plant comprises at least 150% more trichomes per square centimeter as compared to a leaf from a control tobacco plant when grown under comparable conditions. In an aspect, a leaf of a modified tobacco plant comprises at least 175% more trichomes per square centimeter as compared to a leaf from a control tobacco plant when grown under comparable conditions. In an aspect, a leaf of a modified tobacco plant comprises at least 200% more trichomes per square centimeter as compared to a leaf from a control tobacco plant when grown under comparable conditions. In an aspect, a leaf of a modified tobacco plant comprises at least 250% more trichomes per square centimeter as compared to a leaf from a control tobacco plant when grown under comparable conditions. In an aspect, a leaf of a modified tobacco plant comprises at least 300% more trichomes per square centimeter as compared to a leaf from a control tobacco plant when grown under comparable conditions. In an aspect, a leaf of a modified tobacco plant comprises at least 400% more trichomes per square centimeter as compared to a leaf from a control tobacco plant when grown under comparable conditions. In an aspect, a leaf of a modified tobacco plant comprises at least 500% more trichomes per square centimeter as compared to a leaf from a control tobacco plant when grown under comparable conditions.

The number of trichomes on a leaf can be measured by counting the total number of trichomes present on an entire leaf, including all surfaces of its petiole and blade (lamina), unless otherwise noted. Determination of the number of trichomes on a leaf can be performed using tools including, but not limited to, a stereomicroscope, an image capture device (e.g., a camera), and computer software. See, for example, Mirnezami et al., Appl Plant Sci., 8(7):e11375 (2020).

In an aspect, a modified tobacco plant comprising a non-natural mutation in an endogenous nucleic acid molecule encoding a HAIRPLUS1a protein comprises an increased number of trichomes on at least one leaf as compared to a control tobacco plant lacking the non-natural mutation when grown under comparable conditions. In an aspect, a modified tobacco plant comprising a non-natural mutation in an endogenous nucleic acid molecule encoding a HAIRPLUS1b protein comprises an increased number of trichomes on at least one leaf as compared to a control tobacco plant lacking the non-natural mutation when grown under comparable conditions. In an aspect, a modified tobacco plant comprising a non-natural mutation in an endogenous nucleic acid molecule encoding a HAIRPLUS2 protein comprises an increased number of trichomes on at least one leaf as compared to a control tobacco plant lacking the non-natural mutation when grown under comparable conditions. In an aspect, a modified tobacco plant comprising a non-natural mutation in an endogenous nucleic acid molecule encoding a HAIRPLUS3 protein comprises an increased number of trichomes on at least one leaf as compared to a control tobacco plant lacking the non-natural mutation when grown under comparable conditions.

In an aspect, a modified tobacco plant comprising a first non-natural mutation in an endogenous nucleic acid molecule encoding a HAIRPLUS1a protein and a second non-natural mutation in an endogenous nucleic acid molecule encoding a HAIRPLUS1b protein comprises an increased number of trichomes on at least one leaf as compared to a control tobacco plant lacking the non-natural mutations when grown under comparable conditions. In an aspect, a modified tobacco plant comprising a first non-natural mutation in an endogenous nucleic acid molecule encoding a HAIRPLUS1a protein and a second non-natural mutation in an endogenous nucleic acid molecule encoding a HAIRPLUS2 protein comprises an increased number of trichomes on at least one leaf as compared to a control tobacco plant lacking the non-natural mutations when grown under comparable conditions. In an aspect, a modified tobacco plant comprising a first non-natural mutation in an endogenous nucleic acid molecule encoding a HAIRPLUS1b protein and a second non-natural mutation in an endogenous nucleic acid molecule encoding a HAIRPLUS2 protein comprises an increased number of trichomes on at least one leaf as compared to a control tobacco plant lacking the non-natural mutations when grown under comparable conditions.

In an aspect, a modified tobacco plant comprising a first non-natural mutation in an endogenous nucleic acid molecule encoding a HAIRPLUS1a protein, a second non-natural mutation in an endogenous nucleic acid molecule encoding a HAIRPLUS1b protein, and a third non-natural mutation in an endogenous nucleic acid molecule encoding a HAIRPLUS2 protein comprises an increased number of trichomes on at least one leaf as compared to a control tobacco plant lacking the non-natural mutations when grown under comparable conditions. In an aspect, a modified tobacco plant comprising a first non-natural mutation in an endogenous nucleic acid molecule encoding a HAIRPLUS1a protein, a second non-natural mutation in an endogenous nucleic acid molecule encoding a HAIRPLUS1b protein, and a third non-natural mutation in an endogenous nucleic acid molecule encoding a HAIRPLUS3 protein comprises an increased number of trichomes on at least one leaf as compared to a control tobacco plant lacking the non-natural mutations when grown under comparable conditions. In an aspect, a modified tobacco plant comprising a first non-natural mutation in an endogenous nucleic acid molecule encoding a HAIRPLUS1a protein, a second non-natural mutation in an endogenous nucleic acid molecule encoding a HAIRPLUS2 protein, and a third non-natural mutation in an endogenous nucleic acid molecule encoding a HAIRPLUS3 protein comprises an increased number of trichomes on at least one leaf as compared to a control tobacco plant lacking the non-natural mutations when grown under comparable conditions. In an aspect, a modified tobacco plant comprising a first non-natural mutation in an endogenous nucleic acid molecule encoding a HAIRPLUS1b protein, a second non-natural mutation in an endogenous nucleic acid molecule encoding a HAIRPLUS2 protein, and a third non-natural mutation in an endogenous nucleic acid molecule encoding a HAIRPLUS3 protein comprises an increased number of trichomes on at least one leaf as compared to a control tobacco plant lacking the non-natural mutations when grown under comparable conditions.

In an aspect, a modified tobacco plant comprising a first non-natural mutation in an endogenous nucleic acid molecule encoding a HAIRPLUS1a protein, a second non-natural mutation in an endogenous nucleic acid molecule encoding a HAIRPLUS1b protein, a third non-natural mutation in an endogenous nucleic acid molecule encoding a HAIRPLUS2 protein, and a fourth non-natural mutation in an endogenous nucleic acid molecule encoding a HAIRPLUS3 protein comprises an increased number of trichomes on at least one leaf as compared to a control tobacco plant lacking the non-natural mutations when grown under comparable conditions.

In an aspect, a modified tobacco plant comprising a non-natural mutation in an endogenous nucleic acid molecule comprising a HAIRPLUS1a coding sequence or genomic sequence comprises an increased number of trichomes on at least one leaf as compared to a control tobacco plant lacking the non-natural mutation when grown under comparable conditions. In an aspect, a modified tobacco plant comprising a non-natural mutation in an endogenous nucleic acid molecule comprising a HAIRPLUS1b coding sequence or genomic sequence comprises an increased number of trichomes on at least one leaf as compared to a leaf of the control tobacco plant lacking the non-natural mutation when grown under comparable conditions. In an aspect, a modified tobacco plant comprising a non-natural mutation in an endogenous nucleic acid molecule comprising a HAIRPLUS2 coding sequence or genomic sequence comprises an increased number of trichomes on at least one leaf as compared to a control tobacco plant lacking the non-natural mutation when grown under comparable conditions. In an aspect, a modified tobacco plant comprising a non-natural mutation in an endogenous nucleic acid molecule comprising a HAIRPLUS3 coding sequence or genomic sequence comprises an increased number of trichomes on at least one leaf as compared to a control tobacco plant lacking the non-natural mutation when grown under comparable conditions.

In an aspect, a modified tobacco plant comprising a first non-natural mutation in an endogenous nucleic acid molecule comprising a HAIRPLUS1a coding sequence or genomic sequence and a second non-natural mutation in an endogenous nucleic acid molecule comprising a HAIRPLUS1b coding sequence or genomic sequence comprises an increased number of trichomes on at least one leaf as compared to a control tobacco plant lacking the non-natural mutations when grown under comparable conditions. In an aspect, a modified tobacco plant comprising a first non-natural mutation in an endogenous nucleic acid molecule comprising a HAIRPLUS1a coding sequence or genomic sequence and a second non-natural mutation in an endogenous nucleic acid molecule comprising a HAIRPLUS2 coding sequence or genomic sequence comprises an increased number of trichomes on at least one leaf as compared to a control tobacco plant lacking the non-natural mutations when grown under comparable conditions. In an aspect, a modified tobacco plant comprising a first non-natural mutation in an endogenous nucleic acid molecule comprising a HAIRPLUS1b coding sequence or genomic sequence and a second non-natural mutation in an endogenous nucleic acid molecule comprising a HAIRPLUS2 coding sequence or genomic sequence comprises an increased number of trichomes on at least one leaf as compared to a control tobacco plant lacking the non-natural mutations when grown under comparable conditions. In an aspect, a modified tobacco plant comprising a first non-natural mutation in an endogenous nucleic acid molecule comprising a HAIRPLUS1a coding sequence or genomic sequence and a second non-natural mutation in an endogenous nucleic acid molecule comprising a HAIRPLUS3 coding sequence or genomic sequence comprises an increased number of trichomes on at least one leaf as compared to a control tobacco plant lacking the non-natural mutations when grown under comparable conditions. In an aspect, a modified tobacco plant comprising a first non-natural mutation in an endogenous nucleic acid molecule comprising a HAIRPLUS1b coding sequence or genomic sequence and a second non-natural mutation in an endogenous nucleic acid molecule comprising a HAIRPLUS3 coding sequence or genomic sequence comprises an increased number of trichomes on at least one leaf as compared to a control tobacco plant lacking the non-natural mutations when grown under comparable conditions. In an aspect, a modified tobacco plant comprising a first non-natural mutation in an endogenous nucleic acid molecule comprising a HAIRPLUS2 coding sequence or genomic sequence and a second non-natural mutation in an endogenous nucleic acid molecule comprising a HAIRPLUS3 coding sequence or genomic sequence comprises an increased number of trichomes on at least one leaf as compared to a control tobacco plant lacking the non-natural mutations when grown under comparable conditions.

In an aspect, a modified tobacco plant comprising a first non-natural mutation in an endogenous nucleic acid molecule comprising a HAIRPLUS1a coding sequence or genomic sequence, a second non-natural mutation in an endogenous nucleic acid molecule comprising a HAIRPLUS1b coding sequence or genomic sequence, and a third non-natural mutation in an endogenous nucleic acid molecule comprising a HAIRPLUS2 coding sequence or genomic sequence comprises an increased number of trichomes on at least one leaf as compared to a control tobacco plant lacking the non-natural mutations when grown under comparable conditions. In an aspect, a modified tobacco plant comprising a first non-natural mutation in an endogenous nucleic acid molecule comprising a HAIRPLUS1a coding sequence or genomic sequence, a second non-natural mutation in an endogenous nucleic acid molecule comprising a HAIRPLUS1b coding sequence or genomic sequence, and a third non-natural mutation in an endogenous nucleic acid molecule comprising a HAIRPLUS3 coding sequence or genomic sequence comprises an increased number of trichomes on at least one leaf as compared to a control tobacco plant lacking the non-natural mutations when grown under comparable conditions. In an aspect, a modified tobacco plant comprising a first non-natural mutation in an endogenous nucleic acid molecule comprising a HAIRPLUS1a coding sequence or genomic sequence, a second non-natural mutation in an endogenous nucleic acid molecule comprising a HAIRPLUS2 coding sequence or genomic sequence, and a third non-natural mutation in an endogenous nucleic acid molecule comprising a HAIRPLUS3 coding sequence or genomic sequence comprises an increased number of trichomes on at least one leaf as compared to a control tobacco plant lacking the non-natural mutations when grown under comparable conditions. In an aspect, a modified tobacco plant comprising a first non-natural mutation in an endogenous nucleic acid molecule comprising a HAIRPLUS1b coding sequence or genomic sequence, a second non-natural mutation in an endogenous nucleic acid molecule comprising a HAIRPLUS2 coding sequence or genomic sequence, and a third non-natural mutation in an endogenous nucleic acid molecule comprising a HAIRPLUS3 coding sequence or genomic sequence comprises an increased number of trichomes on at least one leaf as compared to a control tobacco plant lacking the non-natural mutations when grown under comparable conditions.

In an aspect, a modified tobacco plant comprising a first non-natural mutation in an endogenous nucleic acid molecule comprising a HAIRPLUS1a coding sequence or genomic sequence, a second non-natural mutation in an endogenous nucleic acid molecule comprising a HAIRPLUS1b coding sequence or genomic sequence, a third non-natural mutation in an endogenous nucleic acid molecule comprising a HAIRPLUS2 coding sequence or genomic sequence, and a fourth non-natural mutation in an endogenous nucleic acid molecule comprising a HAIRPLUS3 coding sequence or genomic sequence comprises an increased number of trichomes on at least one leaf as compared to a control tobacco plant lacking the non-natural mutations when grown under comparable conditions.

In an aspect, at least two leaves of a modified tobacco plant comprise an increased number of trichomes as compared to a control tobacco plant when grown under comparable conditions. In an aspect, at least three leaves of a modified tobacco plant comprise an increased number of trichomes as compared to a control tobacco plant when grown under comparable conditions. In an aspect, at least four leaves of a modified tobacco plant comprise an increased number of trichomes as compared to a control tobacco plant when grown under comparable conditions. In an aspect, at least five leaves of a modified tobacco plant comprise an increased number of trichomes as compared to a control tobacco plant when grown under comparable conditions. In an aspect, at least six leaves of a modified tobacco plant comprise an increased number of trichomes as compared to a control tobacco plant when grown under comparable conditions. In an aspect, at least seven leaves of a modified tobacco plant comprise an increased number of trichomes as compared to a control tobacco plant when grown under comparable conditions. In an aspect, at least eight leaves of a modified tobacco plant comprise an increased number of trichomes as compared to a control tobacco plant when grown under comparable conditions. In an aspect, at least nine leaves of a modified tobacco plant comprise an increased number of trichomes as compared to a control tobacco plant when grown under comparable conditions. In an aspect, at least 10 leaves of a modified tobacco plant comprise an increased number of trichomes as compared to a control tobacco plant when grown under comparable conditions.

In an aspect, at least 5% of the leaves of a modified tobacco plant comprise an increased number of trichomes as compared to a control tobacco plant when grown under comparable conditions. In an aspect, at least 10% of the leaves of a modified tobacco plant comprise an increased number of trichomes as compared to a control tobacco plant when grown under comparable conditions. In an aspect, at least 20% of the leaves of a modified tobacco plant comprise an increased number of trichomes as compared to a control tobacco plant when grown under comparable conditions. In an aspect, at least 30% of the leaves of a modified tobacco plant comprise an increased number of trichomes as compared to a control tobacco plant when grown under comparable conditions. In an aspect, at least 40% of the leaves of a modified tobacco plant comprise an increased number of trichomes as compared to a control tobacco plant when grown under comparable conditions. In an aspect, at least 50% of the leaves of a modified tobacco plant comprise an increased number of trichomes as compared to a control tobacco plant when grown under comparable conditions. In an aspect, at least 60% of the leaves of a modified tobacco plant comprise an increased number of trichomes as compared to a control tobacco plant when grown under comparable conditions. In an aspect, at least 70% of the leaves of a modified tobacco plant comprise an increased number of trichomes as compared to a control tobacco plant when grown under comparable conditions. In an aspect, at least 75% of the leaves of a modified tobacco plant comprise an increased number of trichomes as compared to a control tobacco plant when grown under comparable conditions. In an aspect, at least 80% of the leaves of a modified tobacco plant comprise an increased number of trichomes as compared to a control tobacco plant when grown under comparable conditions. In an aspect, at least 85% of the leaves of a modified tobacco plant comprise an increased number of trichomes as compared to a control tobacco plant when grown under comparable conditions. In an aspect, at least 90% of the leaves of a modified tobacco plant comprise an increased number of trichomes as compared to a control tobacco plant when grown under comparable conditions. In an aspect, at least 95% of the leaves of a modified tobacco plant comprise an increased number of trichomes as compared to a control tobacco plant when grown under comparable conditions. In an aspect, at least 97.5% of the leaves of a modified tobacco plant comprise an increased number of trichomes as compared to a control tobacco plant when grown under comparable conditions. In an aspect, 100% of the leaves of a modified tobacco plant comprise an increased number of trichomes as compared to a control tobacco plant when grown under comparable conditions.

In an aspect, a modified tobacco plant comprises an increased number of trichomes on the adaxial surface of one or more leaves as compared to a control tobacco plant when grown under comparable conditions. In an aspect, a modified tobacco plant comprises an increased number of trichomes on the abaxial surface of one or more leaves as compared to a control tobacco plant when grown under comparable conditions. In an aspect, a modified tobacco plant comprises an increased number of trichomes on the adaxial surface and the abaxial surface of one or more leaves as compared to a control tobacco plant when grown under comparable conditions.

In an aspect, a modified tobacco plant comprises an increased number of glandular trichomes on one or more leaves as compared to a control tobacco plant when grown under comparable conditions.

In an aspect, a leaf of a modified tobacco plant comprises at least 1% more trichomes as compared to a leaf from a control tobacco plant when grown under comparable conditions. In an aspect, a leaf of a modified tobacco plant comprises at least 5% more trichomes as compared to a leaf from a control tobacco plant when grown under comparable conditions. In an aspect, a leaf of a modified tobacco plant comprises at least 10% more trichomes as compared to a leaf from a control tobacco plant when grown under comparable conditions. In an aspect, a leaf of a modified tobacco plant comprises at least 15% more trichomes as compared to a leaf from a control tobacco plant when grown under comparable conditions. In an aspect, a leaf of a modified tobacco plant comprises at least 20% more trichomes as compared to a leaf from a control tobacco plant when grown under comparable conditions. In an aspect, a leaf of a modified tobacco plant comprises at least 25% more trichomes as compared to a leaf from a control tobacco plant when grown under comparable conditions. In an aspect, a leaf of a modified tobacco plant comprises at least 30% more trichomes as compared to a leaf from a control tobacco plant when grown under comparable conditions. In an aspect, a leaf of a modified tobacco plant comprises at least 40% more trichomes as compared to a leaf from a control tobacco plant when grown under comparable conditions. In an aspect, a leaf of a modified tobacco plant comprises at least 50% more trichomes as compared to a leaf from a control tobacco plant when grown under comparable conditions. In an aspect, a leaf of a modified tobacco plant comprises at least 60% more trichomes as compared to a leaf from a control tobacco plant when grown under comparable conditions. In an aspect, a leaf of a modified tobacco plant comprises at least 70% more trichomes as compared to a leaf from a control tobacco plant when grown under comparable conditions. In an aspect, a leaf of a modified tobacco plant comprises at least 80% more trichomes as compared to a leaf from a control tobacco plant when grown under comparable conditions. In an aspect, a leaf of a modified tobacco plant comprises at least 90% more trichomes as compared to a leaf from a control tobacco plant when grown under comparable conditions. In an aspect, a leaf of a modified tobacco plant comprises at least 100% more trichomes as compared to a leaf from a control tobacco plant when grown under comparable conditions. In an aspect, a leaf of a modified tobacco plant comprises at least 110% more trichomes as compared to a leaf from a control tobacco plant when grown under comparable conditions. In an aspect, a leaf of a modified tobacco plant comprises at least 125% more trichomes as compared to a leaf from a control tobacco plant when grown under comparable conditions. In an aspect, a leaf of a modified tobacco plant comprises at least 150% more trichomes as compared to a leaf from a control tobacco plant when grown under comparable conditions. In an aspect, a leaf of a modified tobacco plant comprises at least 175% more trichomes as compared to a leaf from a control tobacco plant when grown under comparable conditions. In an aspect, a leaf of a modified tobacco plant comprises at least 200% more trichomes as compared to a leaf from a control tobacco plant when grown under comparable conditions. In an aspect, a leaf of a modified tobacco plant comprises at least 250% more trichomes as compared to a leaf from a control tobacco plant when grown under comparable conditions. In an aspect, a leaf of a modified tobacco plant comprises at least 300% more trichomes as compared to a leaf from a control tobacco plant when grown under comparable conditions. In an aspect, a leaf of a modified tobacco plant comprises at least 400% more trichomes as compared to a leaf from a control tobacco plant when grown under comparable conditions. In an aspect, a leaf of a modified tobacco plant comprises at least 500% more trichomes as compared to a leaf from a control tobacco plant when grown under comparable conditions.

In an aspect, a modified tobacco plant comprising a non-natural mutation in an endogenous nucleic acid molecule encoding a HAIRPLUS1a protein comprises an increased total number of trichomes as compared to a control tobacco plant lacking the non-natural mutation when grown under comparable conditions. In an aspect, a modified tobacco plant comprising a non-natural mutation in an endogenous nucleic acid molecule encoding a HAIRPLUS1b protein comprises an increased total number of trichomes as compared to a control tobacco plant lacking the non-natural mutation when grown under comparable conditions. In an aspect, a modified tobacco plant comprising a non-natural mutation in an endogenous nucleic acid molecule encoding a HAIRPLUS2 protein comprises an increased total number of trichomes as compared to a control tobacco plant lacking the non-natural mutation when grown under comparable conditions. In an aspect, a modified tobacco plant comprising a non-natural mutation in an endogenous nucleic acid molecule encoding a HAIRPLUS3 protein comprises an increased total number of trichomes as compared to a control tobacco plant lacking the non-natural mutation when grown under comparable conditions.

In an aspect, a modified tobacco plant comprising a first non-natural mutation in an endogenous nucleic acid molecule encoding a HAIRPLUS1a protein and a second non-natural mutation in an endogenous nucleic acid molecule encoding a HAIRPLUS1b protein comprises an increased total number of trichomes as compared to a control tobacco plant lacking the non-natural mutations when grown under comparable conditions. In an aspect, a modified tobacco plant comprising a first non-natural mutation in an endogenous nucleic acid molecule encoding a HAIRPLUS1a protein and a second non-natural mutation in an endogenous nucleic acid molecule encoding a HAIRPLUS2 protein comprises an increased total number of trichomes as compared to a control tobacco plant lacking the non-natural mutations when grown under comparable conditions. In an aspect, a modified tobacco plant comprising a first non-natural mutation in an endogenous nucleic acid molecule encoding a HAIRPLUS1b protein and a second non-natural mutation in an endogenous nucleic acid molecule encoding a HAIRPLUS2 protein comprises an increased total number of trichomes as compared to a control tobacco plant lacking the non-natural mutations when grown under comparable conditions. In an aspect, a modified tobacco plant comprising a first non-natural mutation in an endogenous nucleic acid molecule encoding a HAIRPLUS1a protein and a second non-natural mutation in an endogenous nucleic acid molecule encoding a HAIRPLUS3 protein comprises an increased total number of trichomes as compared to a control tobacco plant lacking the non-natural mutations when grown under comparable conditions. In an aspect, a modified tobacco plant comprising a first non-natural mutation in an endogenous nucleic acid molecule encoding a HAIRPLUS1b protein and a second non-natural mutation in an endogenous nucleic acid molecule encoding a HAIRPLUS3 protein comprises an increased total number of trichomes as compared to a control tobacco plant lacking the non-natural mutations when grown under comparable conditions. In an aspect, a modified tobacco plant comprising a first non-natural mutation in an endogenous nucleic acid molecule encoding a HAIRPLUS2 protein and a second non-natural mutation in an endogenous nucleic acid molecule encoding a HAIRPLUS3 protein comprises an increased total number of trichomes as compared to a control tobacco plant lacking the non-natural mutations when grown under comparable conditions.

In an aspect, a modified tobacco plant comprising a first non-natural mutation in an endogenous nucleic acid molecule encoding a HAIRPLUS1a protein, a second non-natural mutation in an endogenous nucleic acid molecule encoding a HAIRPLUS1b protein, and a third non-natural mutation in an endogenous nucleic acid molecule encoding a HAIRPLUS2 protein comprises an increased total number of trichomes as compared to a control tobacco plant lacking the non-natural mutations when grown under comparable conditions. In an aspect, a modified tobacco plant comprising a first non-natural mutation in an endogenous nucleic acid molecule encoding a HAIRPLUS1a protein, a second non-natural mutation in an endogenous nucleic acid molecule encoding a HAIRPLUS1b protein, and a third non-natural mutation in an endogenous nucleic acid molecule encoding a HAIRPLUS3 protein comprises an increased total number of trichomes as compared to a control tobacco plant lacking the non-natural mutations when grown under comparable conditions. In an aspect, a modified tobacco plant comprising a first non-natural mutation in an endogenous nucleic acid molecule encoding a HAIRPLUS1a protein, a second non-natural mutation in an endogenous nucleic acid molecule encoding a HAIRPLUS2 protein, and a third non-natural mutation in an endogenous nucleic acid molecule encoding a HAIRPLUS3 protein comprises an increased total number of trichomes as compared to a control tobacco plant lacking the non-natural mutations when grown under comparable conditions. In an aspect, a modified tobacco plant comprising a first non-natural mutation in an endogenous nucleic acid molecule encoding a HAIRPLUS1b protein, a second non-natural mutation in an endogenous nucleic acid molecule encoding a HAIRPLUS2 protein, and a third non-natural mutation in an endogenous nucleic acid molecule encoding a HAIRPLUS3 protein comprises an increased total number of trichomes as compared to a control tobacco plant lacking the non-natural mutations when grown under comparable conditions.

In an aspect, a modified tobacco plant comprising a first non-natural mutation in an endogenous nucleic acid molecule encoding a HAIRPLUS1a protein, a second non-natural mutation in an endogenous nucleic acid molecule encoding a HAIRPLUS1b protein, a third non-natural mutation in an endogenous nucleic acid molecule encoding a HAIRPLUS2 protein, and a fourth non-natural mutation in an endogenous nucleic acid molecule encoding a HAIRPLUS3 protein comprises an increased total number of trichomes as compared to a control tobacco plant lacking the non-natural mutations when grown under comparable conditions.

In an aspect, a modified tobacco plant comprising a non-natural mutation in an endogenous nucleic acid molecule comprising a HAIRPLUS1a coding sequence or genomic sequence comprises an increased total number of trichomes as compared to a control tobacco plant lacking the non-natural mutation when grown under comparable conditions. In an aspect, a modified tobacco plant comprising a non-natural mutation in an endogenous nucleic acid molecule comprising a HAIRPLUS1b coding sequence or genomic sequence comprises an increased total number of trichomes as compared to a leaf of the control tobacco plant lacking the non-natural mutation when grown under comparable conditions. In an aspect, a modified tobacco plant comprising a non-natural mutation in an endogenous nucleic acid molecule comprising a HAIRPLUS2 coding sequence or genomic sequence comprises an increased total number of trichomes as compared to a control tobacco plant lacking the non-natural mutation when grown under comparable conditions. In an aspect, a modified tobacco plant comprising a non-natural mutation in an endogenous nucleic acid molecule comprising a HAIRPLUS3 coding sequence or genomic sequence comprises an increased total number of trichomes as compared to a control tobacco plant lacking the non-natural mutation when grown under comparable conditions.

In an aspect, a modified tobacco plant comprising a first non-natural mutation in an endogenous nucleic acid molecule comprising a HAIRPLUS1a coding sequence or genomic sequence and a second non-natural mutation in an endogenous nucleic acid molecule comprising a HAIRPLUS1b coding sequence or genomic sequence comprises an increased total number of trichomes as compared to a control tobacco plant lacking the non-natural mutations when grown under comparable conditions. In an aspect, a modified tobacco plant comprising a first non-natural mutation in an endogenous nucleic acid molecule comprising a HAIRPLUS1a coding sequence or genomic sequence and a second non-natural mutation in an endogenous nucleic acid molecule comprising a HAIRPLUS2 coding sequence or genomic sequence comprises an increased total number of trichomes as compared to a control tobacco plant lacking the non-natural mutations when grown under comparable conditions. In an aspect, a modified tobacco plant comprising a first non-natural mutation in an endogenous nucleic acid molecule comprising a HAIRPLUS1b coding sequence or genomic sequence and a second non-natural mutation in an endogenous nucleic acid molecule comprising a HAIRPLUS2 coding sequence or genomic sequence comprises an increased total number of trichomes as compared to a control tobacco plant lacking the non-natural mutations when grown under comparable conditions. In an aspect, a modified tobacco plant comprising a first non-natural mutation in an endogenous nucleic acid molecule comprising a HAIRPLUS1a coding sequence or genomic sequence and a second non-natural mutation in an endogenous nucleic acid molecule comprising a HAIRPLUS3 coding sequence or genomic sequence comprises an increased total number of trichomes as compared to a control tobacco plant lacking the non-natural mutations when grown under comparable conditions. In an aspect, a modified tobacco plant comprising a first non-natural mutation in an endogenous nucleic acid molecule comprising a HAIRPLUS1b coding sequence or genomic sequence and a second non-natural mutation in an endogenous nucleic acid molecule comprising a HAIRPLUS3 coding sequence or genomic sequence comprises an increased total number of trichomes as compared to a control tobacco plant lacking the non-natural mutations when grown under comparable conditions. In an aspect, a modified tobacco plant comprising a first non-natural mutation in an endogenous nucleic acid molecule comprising a HAIRPLUS2 coding sequence or genomic sequence and a second non-natural mutation in an endogenous nucleic acid molecule comprising a HAIRPLUS3 coding sequence or genomic sequence comprises an increased total number of trichomes as compared to a control tobacco plant lacking the non-natural mutations when grown under comparable conditions.

In an aspect, a modified tobacco plant comprising a first non-natural mutation in an endogenous nucleic acid molecule comprising a HAIRPLUS1a coding sequence or genomic sequence, a second non-natural mutation in an endogenous nucleic acid molecule comprising a HAIRPLUS1b coding sequence or genomic sequence, and a third non-natural mutation in an endogenous nucleic acid molecule comprising a HAIRPLUS2 coding sequence or genomic sequence comprises an increased total number of trichomes as compared to a control tobacco plant lacking the non-natural mutations when grown under comparable conditions. In an aspect, a modified tobacco plant comprising a first non-natural mutation in an endogenous nucleic acid molecule comprising a HAIRPLUS1a coding sequence or genomic sequence, a second non-natural mutation in an endogenous nucleic acid molecule comprising a HAIRPLUS1b coding sequence or genomic sequence, and a third non-natural mutation in an endogenous nucleic acid molecule comprising a HAIRPLUS3 coding sequence or genomic sequence comprises an increased total number of trichomes as compared to a control tobacco plant lacking the non-natural mutations when grown under comparable conditions. In an aspect, a modified tobacco plant comprising a first non-natural mutation in an endogenous nucleic acid molecule comprising a HAIRPLUS1a coding sequence or genomic sequence, a second non-natural mutation in an endogenous nucleic acid molecule comprising a HAIRPLUS2 coding sequence or genomic sequence, and a third non-natural mutation in an endogenous nucleic acid molecule comprising a HAIRPLUS3 coding sequence or genomic sequence comprises an increased total number of trichomes as compared to a control tobacco plant lacking the non-natural mutations when grown under comparable conditions. In an aspect, a modified tobacco plant comprising a first non-natural mutation in an endogenous nucleic acid molecule comprising a HAIRPLUS1b coding sequence or genomic sequence, a second non-natural mutation in an endogenous nucleic acid molecule comprising a HAIRPLUS2 coding sequence or genomic sequence, and a third non-natural mutation in an endogenous nucleic acid molecule comprising a HAIRPLUS3 coding sequence or genomic sequence comprises an increased total number of trichomes as compared to a control tobacco plant lacking the non-natural mutations when grown under comparable conditions.

In an aspect, a modified tobacco plant comprising a first non-natural mutation in an endogenous nucleic acid molecule comprising a HAIRPLUS1a coding sequence or genomic sequence, a second non-natural mutation in an endogenous nucleic acid molecule comprising a HAIRPLUS1b coding sequence or genomic sequence, a third non-natural mutation in an endogenous nucleic acid molecule comprising a HAIRPLUS2 coding sequence or genomic sequence, and a fourth non-natural mutation in an endogenous nucleic acid molecule comprising a HAIRPLUS3 coding sequence or genomic sequence comprises an increased total number of trichomes as compared to a control tobacco plant lacking the non-natural mutations when grown under comparable conditions.

It will be appreciated that the “total number of trichomes” on a plant refers to the total number of trichomes on all leaves (both surfaces of the blade and the petiole), the stems, and floral structures (e.g., all above-ground parts of the plant) of the plant being measured.

In an aspect, a modified tobacco plant comprises at least 1% more total trichomes as compared to a control tobacco plant when grown under comparable conditions. In an aspect, a modified tobacco plant comprises at least 5% more total trichomes as compared to a control tobacco plant when grown under comparable conditions. In an aspect, a modified tobacco plant comprises at least 10% more total trichomes as compared to a control tobacco plant when grown under comparable conditions. In an aspect, a modified tobacco plant comprises at least 20% more total trichomes as compared to a control tobacco plant when grown under comparable conditions. In an aspect, a modified tobacco plant comprises at least 30% more total trichomes as compared to a control tobacco plant when grown under comparable conditions. In an aspect, a modified tobacco plant comprises at least 40% more total trichomes as compared to a control tobacco plant when grown under comparable conditions. In an aspect, a modified tobacco plant comprises at least 50% more total trichomes as compared to a control tobacco plant when grown under comparable conditions. In an aspect, a modified tobacco plant comprises at least 60% more total trichomes as compared to a control tobacco plant when grown under comparable conditions. In an aspect, a modified tobacco plant comprises at least 70% more total trichomes as compared to a control tobacco plant when grown under comparable conditions. In an aspect, a modified tobacco plant comprises at least 80% more total trichomes as compared to a control tobacco plant when grown under comparable conditions. In an aspect, a modified tobacco plant comprises at least 90% more total trichomes as compared to a control tobacco plant when grown under comparable conditions. In an aspect, a modified tobacco plant comprises at least 100% more total trichomes as compared to a control tobacco plant when grown under comparable conditions. In an aspect, a modified tobacco plant comprises at least 200% more total trichomes as compared to a control tobacco plant when grown under comparable conditions. In an aspect, a modified tobacco plant comprises at least 300% more total trichomes as compared to a control tobacco plant when grown under comparable conditions. In an aspect, a modified tobacco plant comprises at least 400% more total trichomes as compared to a control tobacco plant when grown under comparable conditions. In an aspect, a modified tobacco plant comprises at least 500% more total trichomes as compared to a control tobacco plant when grown under comparable conditions. In an aspect, a modified tobacco plant comprises at least 600% more total trichomes as compared to a control tobacco plant when grown under comparable conditions. In an aspect, a modified tobacco plant comprises at least 700% more total trichomes as compared to a control tobacco plant when grown under comparable conditions. In an aspect, a modified tobacco plant comprises at least 800% more total trichomes as compared to a control tobacco plant when grown under comparable conditions. In an aspect, a modified tobacco plant comprises at least 900% more total trichomes as compared to a control tobacco plant when grown under comparable conditions. In an aspect, a modified tobacco plant comprises at least 1000% more total trichomes as compared to a control tobacco plant when grown under comparable conditions.

In an aspect, a modified tobacco plant comprising a non-natural mutation in an endogenous nucleic acid molecule encoding a HAIRPLUS1a protein comprises an increased average length of trichomes on at least one leaf as compared to a control tobacco plant lacking the non-natural mutation when grown under comparable conditions. In an aspect, a modified tobacco plant comprising a non-natural mutation in an endogenous nucleic acid molecule encoding a HAIRPLUS1b protein comprises an increased average length of trichomes on at least one leaf as compared to a control tobacco plant lacking the non-natural mutation when grown under comparable conditions. In an aspect, a modified tobacco plant comprising a non-natural mutation in an endogenous nucleic acid molecule encoding a HAIRPLUS2 protein comprises an increased average length of trichomes on at least one leaf as compared to a control tobacco plant lacking the non-natural mutation when grown under comparable conditions. In an aspect, a modified tobacco plant comprising a non-natural mutation in an endogenous nucleic acid molecule encoding a HAIRPLUS3 protein comprises an increased average length of trichomes on at least one leaf as compared to a control tobacco plant lacking the non-natural mutation when grown under comparable conditions.

In an aspect, a modified tobacco plant comprising a first non-natural mutation in an endogenous nucleic acid molecule encoding a HAIRPLUS1a protein and a second non-natural mutation in an endogenous nucleic acid molecule encoding a HAIRPLUS1b protein comprises an increased average length of trichomes on at least one leaf as compared to a control tobacco plant lacking the non-natural mutations when grown under comparable conditions. In an aspect, a modified tobacco plant comprising a first non-natural mutation in an endogenous nucleic acid molecule encoding a HAIRPLUS1a protein and a second non-natural mutation in an endogenous nucleic acid molecule encoding a HAIRPLUS2 protein comprises an increased average length of trichomes on at least one leaf as compared to a control tobacco plant lacking the non-natural mutations when grown under comparable conditions. In an aspect, a modified tobacco plant comprising a first non-natural mutation in an endogenous nucleic acid molecule encoding a HAIRPLUS1b protein and a second non-natural mutation in an endogenous nucleic acid molecule encoding a HAIRPLUS2 protein comprises an increased average length of trichomes on at least one leaf as compared to a control tobacco plant lacking the non-natural mutations when grown under comparable conditions. In an aspect, a modified tobacco plant comprising a first non-natural mutation in an endogenous nucleic acid molecule encoding a HAIRPLUS1a protein and a second non-natural mutation in an endogenous nucleic acid molecule encoding a HAIRPLUS3 protein comprises an increased average length of trichomes on at least one leaf as compared to a control tobacco plant lacking the non-natural mutations when grown under comparable conditions. In an aspect, a modified tobacco plant comprising a first non-natural mutation in an endogenous nucleic acid molecule encoding a HAIRPLUS1b protein and a second non-natural mutation in an endogenous nucleic acid molecule encoding a HAIRPLUS3 protein comprises an increased average length of trichomes on at least one leaf as compared to a control tobacco plant lacking the non-natural mutations when grown under comparable conditions. In an aspect, a modified tobacco plant comprising a first non-natural mutation in an endogenous nucleic acid molecule encoding a HAIRPLUS2 protein and a second non-natural mutation in an endogenous nucleic acid molecule encoding a HAIRPLUS3 protein comprises an increased average length of trichomes on at least one leaf as compared to a control tobacco plant lacking the non-natural mutations when grown under comparable conditions.

In an aspect, a modified tobacco plant comprising a first non-natural mutation in an endogenous nucleic acid molecule encoding a HAIRPLUS1a protein, a second non-natural mutation in an endogenous nucleic acid molecule encoding a HAIRPLUS1b protein, and a third non-natural mutation in an endogenous nucleic acid molecule encoding a HAIRPLUS2 protein comprises an increased average length of trichomes on at least one leaf as compared to a control tobacco plant lacking the non-natural mutations when grown under comparable conditions. In an aspect, a modified tobacco plant comprising a first non-natural mutation in an endogenous nucleic acid molecule encoding a HAIRPLUS1a protein, a second non-natural mutation in an endogenous nucleic acid molecule encoding a HAIRPLUS1b protein, and a third non-natural mutation in an endogenous nucleic acid molecule encoding a HAIRPLUS3 protein comprises an increased average length of trichomes on at least one leaf as compared to a control tobacco plant lacking the non-natural mutations when grown under comparable conditions. In an aspect, a modified tobacco plant comprising a first non-natural mutation in an endogenous nucleic acid molecule encoding a HAIRPLUS1a protein, a second non-natural mutation in an endogenous nucleic acid molecule encoding a HAIRPLUS2 protein, and a third non-natural mutation in an endogenous nucleic acid molecule encoding a HAIRPLUS3 protein comprises an increased average length of trichomes on at least one leaf as compared to a control tobacco plant lacking the non-natural mutations when grown under comparable conditions. In an aspect, a modified tobacco plant comprising a first non-natural mutation in an endogenous nucleic acid molecule encoding a HAIRPLUS1b protein, a second non-natural mutation in an endogenous nucleic acid molecule encoding a HAIRPLUS2 protein, and a third non-natural mutation in an endogenous nucleic acid molecule encoding a HAIRPLUS3 protein comprises an increased average length of trichomes on at least one leaf as compared to a control tobacco plant lacking the non-natural mutations when grown under comparable conditions.

In an aspect, a modified tobacco plant comprising a first non-natural mutation in an endogenous nucleic acid molecule encoding a HAIRPLUS1a protein, a second non-natural mutation in an endogenous nucleic acid molecule encoding a HAIRPLUS1b protein, a third non-natural mutation in an endogenous nucleic acid molecule encoding a HAIRPLUS2 protein comprises an increased average length of trichomes on at least one leaf as compared to a control tobacco plant lacking the non-natural mutations when grown under comparable conditions, and a fourth non-natural mutation in an endogenous nucleic acid molecule encoding a HAIRPLUS3 protein comprises an increased average length of trichomes on at least one leaf as compared to a control tobacco plant lacking the first, second, third, and fourth non-natural mutations when grown under comparable conditions.

In an aspect, a modified tobacco plant comprising a non-natural mutation in an endogenous nucleic acid molecule comprising a HAIRPLUS1a coding sequence or genomic sequence comprises an increased average length of trichomes on at least one leaf as compared to a control tobacco plant lacking the non-natural mutation when grown under comparable conditions. In an aspect, a modified tobacco plant comprising a non-natural mutation in an endogenous nucleic acid molecule comprising a HAIRPLUS1b coding sequence or genomic sequence comprises an increased average length of trichomes on at least one leaf as compared to a control tobacco plant lacking the non-natural mutation when grown under comparable conditions. In an aspect, a modified tobacco plant comprising a non-natural mutation in an endogenous nucleic acid molecule comprising a HAIRPLUS2 coding sequence or genomic sequence comprises an increased average length of trichomes on at least one leaf as compared to a control tobacco plant lacking the non-natural mutation when grown under comparable conditions. In an aspect, a modified tobacco plant comprising a non-natural mutation in an endogenous nucleic acid molecule comprising a HAIRPLUS3 coding sequence or genomic sequence comprises an increased average length of trichomes on at least one leaf as compared to a control tobacco plant lacking the non-natural mutation when grown under comparable conditions.

In an aspect, a modified tobacco plant comprising a first non-natural mutation in an endogenous nucleic acid molecule comprising a HAIRPLUS1a coding sequence or genomic sequence and a second non-natural mutation in an endogenous nucleic acid molecule comprising a HAIRPLUS1b coding sequence or genomic sequence comprises an increased average length of trichomes on at least one leaf as compared to a control tobacco plant lacking the non-natural mutations when grown under comparable conditions. In an aspect, a modified tobacco plant comprising a first non-natural mutation in an endogenous nucleic acid molecule comprising a HAIRPLUS1a coding sequence or genomic sequence and a second non-natural mutation in an endogenous nucleic acid molecule comprising a HAIRPLUS2 coding sequence or genomic sequence comprises an increased average length of trichomes on at least one leaf as compared to a control tobacco plant lacking the non-natural mutations when grown under comparable conditions. In an aspect, a modified tobacco plant comprising a first non-natural mutation in an endogenous nucleic acid molecule comprising a HAIRPLUS1b coding sequence or genomic sequence and a second non-natural mutation in an endogenous nucleic acid molecule comprising a HAIRPLUS2 coding sequence or genomic sequence comprises an increased average length of trichomes on at least one leaf as compared to a control tobacco plant lacking the non-natural mutations when grown under comparable conditions. In an aspect, a modified tobacco plant comprising a first non-natural mutation in an endogenous nucleic acid molecule comprising a HAIRPLUS1a coding sequence or genomic sequence and a second non-natural mutation in an endogenous nucleic acid molecule comprising a HAIRPLUS3 coding sequence or genomic sequence comprises an increased average length of trichomes on at least one leaf as compared to a control tobacco plant lacking the non-natural mutations when grown under comparable conditions. In an aspect, a modified tobacco plant comprising a first non-natural mutation in an endogenous nucleic acid molecule comprising a HAIRPLUS1b coding sequence or genomic sequence and a second non-natural mutation in an endogenous nucleic acid molecule comprising a HAIRPLUS3 coding sequence or genomic sequence comprises an increased average length of trichomes on at least one leaf as compared to a control tobacco plant lacking the non-natural mutations when grown under comparable conditions. In an aspect, a modified tobacco plant comprising a first non-natural mutation in an endogenous nucleic acid molecule comprising a HAIRPLUS2 coding sequence or genomic sequence and a second non-natural mutation in an endogenous nucleic acid molecule comprising a HAIRPLUS3 coding sequence or genomic sequence comprises an increased average length of trichomes on at least one leaf as compared to a control tobacco plant lacking the non-natural mutations when grown under comparable conditions.

In an aspect, a modified tobacco plant comprising a first non-natural mutation in an endogenous nucleic acid molecule comprising a HAIRPLUS1a coding sequence or genomic sequence, a second non-natural mutation in an endogenous nucleic acid molecule comprising a HAIRPLUS1b coding sequence or genomic sequence, and a third non-natural mutation in an endogenous nucleic acid molecule comprising a HAIRPLUS2 coding sequence or genomic sequence comprises an increased average length of trichomes on at least one leaf as compared to a control tobacco plant lacking the non-natural mutations when grown under comparable conditions. In an aspect, a modified tobacco plant comprising a first non-natural mutation in an endogenous nucleic acid molecule comprising a HAIRPLUS1a coding sequence or genomic sequence, a second non-natural mutation in an endogenous nucleic acid molecule comprising a HAIRPLUS1b coding sequence or genomic sequence, and a third non-natural mutation in an endogenous nucleic acid molecule comprising a HAIRPLUS3 coding sequence or genomic sequence comprises an increased average length of trichomes on at least one leaf as compared to a control tobacco plant lacking the non-natural mutations when grown under comparable conditions. In an aspect, a modified tobacco plant comprising a first non-natural mutation in an endogenous nucleic acid molecule comprising a HAIRPLUS1a coding sequence or genomic sequence, a second non-natural mutation in an endogenous nucleic acid molecule comprising a HAIRPLUS2 coding sequence or genomic sequence, and a third non-natural mutation in an endogenous nucleic acid molecule comprising a HAIRPLUS3 coding sequence or genomic sequence comprises an increased average length of trichomes on at least one leaf as compared to a control tobacco plant lacking the non-natural mutations when grown under comparable conditions. In an aspect, a modified tobacco plant comprising a first non-natural mutation in an endogenous nucleic acid molecule comprising a HAIRPLUS1b coding sequence or genomic sequence, a second non-natural mutation in an endogenous nucleic acid molecule comprising a HAIRPLUS2 coding sequence or genomic sequence, and a third non-natural mutation in an endogenous nucleic acid molecule comprising a HAIRPLUS3 coding sequence or genomic sequence comprises an increased average length of trichomes on at least one leaf as compared to a control tobacco plant lacking the non-natural mutations when grown under comparable conditions.

In an aspect, a modified tobacco plant comprising a first non-natural mutation in an endogenous nucleic acid molecule comprising a HAIRPLUS1a coding sequence or genomic sequence, a second non-natural mutation in an endogenous nucleic acid molecule comprising a HAIRPLUS1b coding sequence or genomic sequence, a third non-natural mutation in an endogenous nucleic acid molecule comprising a HAIRPLUS2 coding sequence or genomic sequence, and a fourth non-natural mutation in an endogenous nucleic acid molecule comprising a HAIRPLUS3 coding sequence or genomic sequence comprises an increased average length of trichomes on at least one leaf as compared to a control tobacco plant lacking the non-natural mutations when grown under comparable conditions.

In an aspect, at least two leaves of a modified tobacco plant comprise an increased average length of trichomes as compared to a control tobacco plant when grown under comparable conditions. In an aspect, at least three leaves of a modified tobacco plant comprise an increased average length of trichomes as compared to a control tobacco plant when grown under comparable conditions. In an aspect, at least four leaves of a modified tobacco plant comprise an increased average length of trichomes as compared to a control tobacco plant when grown under comparable conditions. In an aspect, at least five leaves of a modified tobacco plant comprise an increased average length of trichomes as compared to a control tobacco plant when grown under comparable conditions. In an aspect, at least six leaves of a modified tobacco plant comprise an increased average length of trichomes as compared to a control tobacco plant when grown under comparable conditions. In an aspect, at least seven leaves of a modified tobacco plant comprise an increased average length of trichomes as compared to a control tobacco plant when grown under comparable conditions. In an aspect, at least eight leaves of a modified tobacco plant comprise an increased average length of trichomes as compared to a control tobacco plant when grown under comparable conditions. In an aspect, at least nine leaves of a modified tobacco plant comprise an increased average length of trichomes as compared to a control tobacco plant when grown under comparable conditions. In an aspect, at least 10 leaves of a modified tobacco plant comprise an increased average length of trichomes as compared to a control tobacco plant when grown under comparable conditions.

In an aspect, at least 10% of the leaves of a modified tobacco plant comprise an increased average length of trichomes as compared to a control tobacco plant when grown under comparable conditions. In an aspect, at least 20% of the leaves of a modified tobacco plant comprise an increased average length of trichomes as compared to a control tobacco plant when grown under comparable conditions. In an aspect, at least 30% of the leaves of a modified tobacco plant comprise an increased average length of trichomes as compared to a control tobacco plant when grown under comparable conditions. In an aspect, at least 40% of the leaves of a modified tobacco plant comprise an increased average length of trichomes as compared to a control tobacco plant when grown under comparable conditions. In an aspect, at least 50% of the leaves of a modified tobacco plant comprise an increased average length of trichomes as compared to a control tobacco plant when grown under comparable conditions. In an aspect, at least 60% of the leaves of a modified tobacco plant comprise an increased average length of trichomes as compared to a control tobacco plant when grown under comparable conditions. In an aspect, at least 70% of the leaves of a modified tobacco plant comprise an increased average length of trichomes as compared to a control tobacco plant when grown under comparable conditions. In an aspect, at least 75% of the leaves of a modified tobacco plant comprise an increased average length of trichomes as compared to a control tobacco plant when grown under comparable conditions. In an aspect, at least 80% of the leaves of a modified tobacco plant comprise an increased average length of trichomes as compared to a control tobacco plant when grown under comparable conditions. In an aspect, at least 85% of the leaves of a modified tobacco plant comprise an increased average length of trichomes as compared to a control tobacco plant when grown under comparable conditions. In an aspect, at least 90% of the leaves of a modified tobacco plant comprise an increased average length of trichomes as compared to a control tobacco plant when grown under comparable conditions. In an aspect, at least 95% of the leaves of a modified tobacco plant comprise an increased average length of trichomes as compared to a control tobacco plant when grown under comparable conditions. In an aspect, at least 97.5% of the leaves of a modified tobacco plant comprise an increased average length of trichomes as compared to a control tobacco plant when grown under comparable conditions. In an aspect, 100% of the leaves of a modified tobacco plant comprise an increased average length of trichomes as compared to a control tobacco plant when grown under comparable conditions.

In an aspect, a modified tobacco plant comprises an increased average length of glandular trichomes on one or more leaves as compared to a control tobacco plant when grown under comparable conditions.

As used herein, the “average length” of trichomes refers to measuring the length of 20 to 30 trichomes from a randomly selected 1 cm² area on a leaf and taking the average length of those trichomes across three independent measurements (e.g., the average of 60 to 90 total measurements).

In an aspect, an amino acid sequence is at least 70% identical to an amino acid sequence selected from the group consisting of SEQ ID NOs: 1 to 3 and 538. In an aspect, an amino acid sequence is at least 75% identical to an amino acid sequence selected from the group consisting of SEQ ID NOs: 1 to 3 and 538. In an aspect, an amino acid sequence is at least 80% identical to an amino acid sequence selected from the group consisting of SEQ ID NOs: 1 to 3 and 538. In an aspect, an amino acid sequence is at least 85% identical to an amino acid sequence selected from the group consisting of SEQ ID NOs: 1 to 3 and 538. In an aspect, an amino acid sequence is at least 90% identical to an amino acid sequence selected from the group consisting of SEQ ID NOs: 1 to 3 and 538. In an aspect, an amino acid sequence is at least 92.5% identical to an amino acid sequence selected from the group consisting of SEQ ID NOs: 1 to 3 and 538. In an aspect, an amino acid sequence is at least 95% identical to an amino acid sequence selected from the group consisting of SEQ ID NOs: 1 to 3 and 538. In an aspect, an amino acid sequence is at least 97.5% identical to an amino acid sequence selected from the group consisting of SEQ ID NOs: 1 to 3 and 538. In an aspect, an amino acid sequence is at least 99% identical to an amino acid sequence selected from the group consisting of SEQ ID NOs: 1 to 3 and 538. In an aspect, an amino acid sequence is at least 99.5% identical to an amino acid sequence selected from the group consisting of SEQ ID NOs: 1 to 3 and 538. In an aspect, an amino acid sequence is 100% identical to an amino acid sequence selected from the group consisting of SEQ ID NOs: 1 to 3 and 538.

In an aspect, an amino acid sequence is at least 70% identical to an amino acid sequence selected from the group consisting of SEQ ID NOs: 547 to 588. In an aspect, an amino acid sequence is at least 75% identical to an amino acid sequence selected from the group consisting of SEQ ID NOs: 547 to 588. In an aspect, an amino acid sequence is at least 80% identical to an amino acid sequence selected from the group consisting of SEQ ID NOs: 547 to 588. In an aspect, an amino acid sequence is at least 85% identical to an amino acid sequence selected from the group consisting of SEQ ID NOs: 547 to 588. In an aspect, an amino acid sequence is at least 90% identical to an amino acid sequence selected from the group consisting of SEQ ID NOs: 547 to 588. In an aspect, an amino acid sequence is at least 92.5% identical to an amino acid sequence selected from the group consisting of SEQ ID NOs: 547 to 588. In an aspect, an amino acid sequence is at least 95% identical to an amino acid sequence selected from the group consisting of SEQ ID NOs: 547 to 588. In an aspect, an amino acid sequence is at least 97.5% identical to an amino acid sequence selected from the group consisting of SEQ ID NOs: 547 to 588. In an aspect, an amino acid sequence is at least 99% identical to an amino acid sequence selected from the group consisting of SEQ ID NOs: 547 to 588. In an aspect, an amino acid sequence is at least 99.5% identical to an amino acid sequence selected from the group consisting of SEQ ID NOs: 547 to 588. In an aspect, an amino acid sequence is 100% identical to an amino acid sequence selected from the group consisting of SEQ ID NOs: 547 to 588.

In an aspect, an amino acid sequence is at least 70% similar to an amino acid sequence selected from the group consisting of SEQ ID NOs: 1 to 3 and 538. In an aspect, an amino acid sequence is at least 75% similar to an amino acid sequence selected from the group consisting of SEQ ID NOs: 1 to 3 and 538. In an aspect, an amino acid sequence is at least 80% similar to an amino acid sequence selected from the group consisting of SEQ ID NOs: 1 to 3 and 538. In an aspect, an amino acid sequence is at least 85% similar to an amino acid sequence selected from the group consisting of SEQ ID NOs: 1 to 3 and 538. In an aspect, an amino acid sequence is at least 90% similar to an amino acid sequence selected from the group consisting of SEQ ID NOs: 1 to 3 and 538. In an aspect, an amino acid sequence is at least 92.5% similar to an amino acid sequence selected from the group consisting of SEQ ID NOs: 1 to 3 and 538. In an aspect, an amino acid sequence is at least 95% similar to an amino acid sequence selected from the group consisting of SEQ ID NOs: 1 to 3 and 538. In an aspect, an amino acid sequence is at least 97.5% similar to an amino acid sequence selected from the group consisting of SEQ ID NOs: 1 to 3 and 538. In an aspect, an amino acid sequence is at least 99% similar to an amino acid sequence selected from the group consisting of SEQ ID NOs: 1 to 3 and 538. In an aspect, an amino acid sequence is at least 99.5% similar to an amino acid sequence selected from the group consisting of SEQ ID NOs: 1 to 3 and 538. In an aspect, an amino acid sequence is 100% similar to an amino acid sequence selected from the group consisting of SEQ ID NOs: 1 to 3 and 538.

In an aspect, an amino acid sequence is at least 70% similar to an amino acid sequence selected from the group consisting of SEQ ID NOs: 547 to 588. In an aspect, an amino acid sequence is at least 75% similar to an amino acid sequence selected from the group consisting of SEQ ID NOs: 547 to 588. In an aspect, an amino acid sequence is at least 80% similar to an amino acid sequence selected from the group consisting of SEQ ID NOs: 547 to 588. In an aspect, an amino acid sequence is at least 85% similar to an amino acid sequence selected from the group consisting of SEQ ID NOs: 547 to 588. In an aspect, an amino acid sequence is at least 90% similar to an amino acid sequence selected from the group consisting of SEQ ID NOs: 547 to 588. In an aspect, an amino acid sequence is at least 92.5% similar to an amino acid sequence selected from the group consisting of SEQ ID NOs: 547 to 588. In an aspect, an amino acid sequence is at least 95% similar to an amino acid sequence selected from the group consisting of SEQ ID NOs: 547 to 588. In an aspect, an amino acid sequence is at least 97.5% similar to an amino acid sequence selected from the group consisting of SEQ ID NOs: 547 to 588. In an aspect, an amino acid sequence is at least 99% similar to an amino acid sequence selected from the group consisting of SEQ ID NOs: 547 to 588. In an aspect, an amino acid sequence is at least 99.5% similar to an amino acid sequence selected from the group consisting of SEQ ID NOs: 547 to 588. In an aspect, an amino acid sequence is 100% similar to an amino acid sequence selected from the group consisting of SEQ ID NOs: 547 to 588.

In an aspect, a nucleic acid sequence is at least 70% identical to a nucleic acid sequence selected from the group consisting of SEQ ID NOs: 4 to 9, 537, and 612. In an aspect, a nucleic acid sequence is at least 75% identical to a nucleic acid sequence selected from the group consisting of SEQ ID NOs: 4 to 9, 537, and 612. In an aspect, a nucleic acid sequence is at least 80% identical to a nucleic acid sequence selected from the group consisting of SEQ ID NOs: 4 to 9, 537, and 612. In an aspect, a nucleic acid sequence is at least 85% identical to a nucleic acid sequence selected from the group consisting of SEQ ID NOs: 4 to 9, 537, and 612. In an aspect, a nucleic acid sequence is at least 90% identical to a nucleic acid sequence selected from the group consisting of SEQ ID NOs: 4 to 9, 537, and 612. In an aspect, a nucleic acid sequence is at least 92.5% identical to a nucleic acid sequence selected from the group consisting of SEQ ID NOs: 4 to 9, 537, and 612. In an aspect, a nucleic acid sequence is at least 95% identical to a nucleic acid sequence selected from the group consisting of SEQ ID NOs: 4 to 9, 537, and 612. In an aspect, a nucleic acid sequence is at least 97.5% identical to a nucleic acid sequence selected from the group consisting of SEQ ID NOs: 4 to 9, 537, and 612. In an aspect, a nucleic acid sequence is at least 99% identical to a nucleic acid sequence selected from the group consisting of SEQ ID NOs: 4 to 9, 537, and 612. In an aspect, a nucleic acid sequence is at least 99.5% identical to a nucleic acid sequence selected from the group consisting of SEQ ID NOs: 4 to 9, 537, and 612. In an aspect, a nucleic acid sequence is 100% identical to a nucleic acid sequence selected from the group consisting of SEQ ID NOs: 4 to 9, 537, and 612.

In an aspect, a nucleic acid sequence encodes an amino acid sequence at least 70% identical to an amino acid sequence selected from the group consisting of SEQ ID NOs: 1 to 3 and 538. In an aspect, a nucleic acid sequence encodes an amino acid sequence at least 75% identical to an amino acid sequence selected from the group consisting of SEQ ID NOs: 1 to 3 and 538. In an aspect, a nucleic acid sequence encodes an amino acid sequence at least 80% identical to an amino acid sequence selected from the group consisting of SEQ ID NOs: 1 to 3 and 538. In an aspect, a nucleic acid sequence encodes an amino acid sequence at least 85% identical to an amino acid sequence selected from the group consisting of SEQ ID NOs: 1 to 3 and 538. In an aspect, a nucleic acid sequence encodes an amino acid sequence at least 90% identical to an amino acid sequence selected from the group consisting of SEQ ID NOs: 1 to 3 and 538. In an aspect, a nucleic acid sequence encodes an amino acid sequence at least 92.5% identical to an amino acid sequence selected from the group consisting of SEQ ID NOs: 1 to 3 and 538. In an aspect, a nucleic acid sequence encodes an amino acid sequence at least 95% identical to an amino acid sequence selected from the group consisting of SEQ ID NOs: 1 to 3 and 538. In an aspect, a nucleic acid sequence encodes an amino acid sequence at least 97.5% identical to an amino acid sequence selected from the group consisting of SEQ ID NOs: 1 to 3 and 538. In an aspect, a nucleic acid sequence encodes an amino acid sequence at least 99% identical to an amino acid sequence selected from the group consisting of SEQ ID NOs: 1 to 3 and 538. In an aspect, a nucleic acid sequence encodes an amino acid sequence at least 99.5% identical to an amino acid sequence selected from the group consisting of SEQ ID NOs: 1 to 3 and 538. In an aspect, a nucleic acid sequence encodes an amino acid sequence 100% identical to an amino acid sequence selected from the group consisting of SEQ ID NOs: 1 to 3 and 538.

In an aspect, a nucleic acid sequence encodes an amino acid sequence at least 70% similar to an amino acid sequence selected from the group consisting of SEQ ID NOs: 1 to 3 and 538. In an aspect, a nucleic acid sequence encodes an amino acid sequence at least 75% similar to an amino acid sequence selected from the group consisting of SEQ ID NOs: 1 to 3 and 538. In an aspect, a nucleic acid sequence encodes an amino acid sequence at least 80% similar to an amino acid sequence selected from the group consisting of SEQ ID NOs: 1 to 3 and 538. In an aspect, a nucleic acid sequence encodes an amino acid sequence at least 85% similar to an amino acid sequence selected from the group consisting of SEQ ID NOs: 1 to 3 and 538. In an aspect, a nucleic acid sequence encodes an amino acid sequence at least 90% similar to an amino acid sequence selected from the group consisting of SEQ ID NOs: 1 to 3 and 538. In an aspect, a nucleic acid sequence encodes an amino acid sequence at least 92.5% similar to an amino acid sequence selected from the group consisting of SEQ ID NOs: 1 to 3 and 538. In an aspect, a nucleic acid sequence encodes an amino acid sequence at least 95% similar to an amino acid sequence selected from the group consisting of SEQ ID NOs: 1 to 3 and 538. In an aspect, a nucleic acid sequence encodes an amino acid sequence at least 97.5% similar to an amino acid sequence selected from the group consisting of SEQ ID NOs: 1 to 3 and 538. In an aspect, a nucleic acid sequence encodes an amino acid sequence at least 99% similar to an amino acid sequence selected from the group consisting of SEQ ID NOs: 1 to 3 and 538. In an aspect, a nucleic acid sequence encodes an amino acid sequence at least 99.5% similar to an amino acid sequence selected from the group consisting of SEQ ID NOs: 1 to 3 and 538. In an aspect, a nucleic acid sequence encodes an amino acid sequence 100% similar to an amino acid sequence selected from the group consisting of SEQ ID NOs: 1 to 3 and 538.

The terms “percent identity” or “percent identical” as used herein in reference to two or more nucleotide or amino acid sequences is calculated by (i) comparing two optimally aligned sequences (nucleotide or amino acid) over a window of comparison (the “alignable” region or regions), (ii) determining the number of positions at which the identical nucleic acid base (for nucleotide sequences) or amino acid residue (for proteins and polypeptides) occurs in both sequences to yield the number of matched positions, (iii) dividing the number of matched positions by the total number of positions in the window of comparison, and then (iv) multiplying this quotient by 100% to yield the percent identity. If the “percent identity” is being calculated in relation to a reference sequence without a particular comparison window being specified, then the percent identity is determined by dividing the number of matched positions over the region of alignment by the total length of the reference sequence (e.g., a SEQ ID NO provided in the instant disclosure). Accordingly, for purposes of the present application, when two sequences (query and subject) are optimally aligned (with allowance for gaps in their alignment), the “percent identity” for the query sequence is equal to the number of identical positions between the two sequences divided by the total number of positions in the query sequence over its length (or a comparison window), which is then multiplied by 100%.

The terms “percent sequence complementarity” or “percent complementarity” as used herein in reference to two nucleotide sequences is similar to the concept of percent identity but refers to the percentage of nucleotides of a query sequence that optimally base-pair or hybridize to nucleotides a subject sequence when the query and subject sequences are linearly arranged and optimally base paired without secondary folding structures, such as loops, stems or hairpins. Such a percent complementarity can be between two DNA strands, two RNA strands, or a DNA strand and a RNA strand. The “percent complementarity” can be calculated by (i) optimally base-pairing or hybridizing the two nucleotide sequences in a linear and fully extended arrangement (i.e., without folding or secondary structures) over a window of comparison, (ii) determining the number of positions that base-pair between the two sequences over the window of comparison to yield the number of complementary positions, (iii) dividing the number of complementary positions by the total number of positions in the window of comparison, and (iv) multiplying this quotient by 100% to yield the percent complementarity of the two sequences. Optimal base pairing of two sequences can be determined based on the known pairings of nucleotide bases, such as G-C, A-T, and A-U, through hydrogen binding. If the “percent complementarity” is being calculated in relation to a reference sequence without specifying a particular comparison window, then the percent identity is determined by dividing the number of complementary positions between the two linear sequences by the total length of the reference sequence (e.g., a SEQ ID NO provided herein). Thus, for purposes of the present application, when two sequences (query and subject) are optimally base-paired (with allowance for mismatches or non-base-paired nucleotides), the “percent complementarity” for the query sequence is equal to the number of base-paired positions between the two sequences divided by the total number of positions in the query sequence over its length, which is then multiplied by 100%.

When percentage of sequence identity is used in reference to amino acids it is recognized that residue positions which are not identical often differ by conservative amino acid substitutions, where amino acid residues are substituted for other amino acid residues with similar chemical properties (e.g., charge or hydrophobicity) and therefore do not change the functional properties of the molecule. When sequences differ in conservative substitutions, the percent sequence identity can be adjusted upwards to correct for the conservative nature of the substitution. Sequences that differ by such conservative substitutions are said to have “sequence similarity” or “similarity.”

Without being limiting, two aliphatic (e.g., glycine, alanine, valine, leucine, isoleucine) amino acid residues can be substituted for each other in a conservative substitution; two hydroxyl (e.g., serine, cysteine, threonine, methionine) amino acid residues can be substituted for each other in a conservative substitution; two aromatic (e.g., phenylalanine, tyrosine, tryptophan) amino acid residues can be substituted for each other in a conservative substitution; two basic (e.g., histidine, lysine, arginine) amino acid residues can be substituted for each other in a conservative substitution; and two acid (e.g., aspartate, glutamate, asparagine, glutamine) amino acid residues can be substituted for each other in a conservative substitution.

For optimal alignment of sequences to calculate their percent identity, various pair-wise or multiple sequence alignment algorithms and programs are known in the art, such as ClustalW or Basic Local Alignment Search Tool® (BLAST™), etc., that can be used to compare the sequence identity or similarity between two or more nucleotide or amino acid sequences. Although other alignment and comparison methods are known in the art, the alignment and percent identity between two sequences (including the percent identity ranges described above) can be as determined by the ClustalW algorithm, see, e.g., Chenna et al., “Multiple sequence alignment with the Clustal series of programs,” Nucleic Acids Research 31:3497-3500 (2003); Thompson et al., “Clustal W: Improving the sensitivity of progressive multiple sequence alignment through sequence weighting, position-specific gap penalties and weight matrix choice,” Nucleic Acids Research 22:4673-4680 (1994); Larkin M A et al., “Clustal W and Clustal X version 2.0,” Bioinformatics 23:2947-48 (2007); and Altschul et al. “Basic local alignment search tool.” J. Mol. Biol. 215:403-410 (1990), the entire contents and disclosures of which are incorporated herein by reference.

The use of the term “polynucleotide” or “nucleic acid molecule” is not intended to limit the present disclosure to polynucleotides comprising deoxyribonucleic acid (DNA). For example, ribonucleic acid (RNA) molecules encoded by the DNA molecules provided herein are also envisioned. As a non-limiting example, a DNA sequence provided herein also provides the corresponding RNA sequence, where the thymine(s) (T) in the DNA sequence are replaced by uridine(s) (U) in the RNA sequence (e.g., the DNA sequence 5′-ATTG-3′ provides the RNA sequence 5′-AUUG-3′). Those of ordinary skill in the art will recognize that polynucleotides and nucleic acid molecules can comprise ribonucleotides and combinations of ribonucleotides and deoxyribonucleotides. Such deoxyribonucleotides and ribonucleotides include both naturally occurring molecules and synthetic analogues. The polynucleotides of the present disclosure also encompass all forms of sequences including, but not limited to, single-stranded forms, double-stranded forms, hairpins, stem-and-loop structures, and the like. In an aspect, a nucleic acid molecule provided herein is a DNA molecule. In an aspect, a nucleic acid molecule is an RNA molecule. In an aspect, a nucleic acid molecule is single-stranded. In an aspect, a nucleic acid molecule is double-stranded. In an aspect, a nucleic acid molecule encodes a polypeptide. In an aspect, a nucleic acid molecule encodes a non-coding RNA molecule.

Nucleic acid molecules can be isolated using techniques routine in the art. For example, nucleic acids can be isolated using any method including, without limitation, recombinant nucleic acid technology, and/or the polymerase chain reaction (PCR). General PCR techniques are described, for example in PCR Primer: A Laboratory Manual, Dieffenbach & Dveksler, Eds., Cold Spring Harbor Laboratory Press, 1995. Recombinant nucleic acid techniques include, for example, restriction enzyme digestion and ligation, which can be used to isolate a nucleic acid. Isolated nucleic acids also can be chemically synthesized, either as a single nucleic acid molecule or as a series of oligonucleotides. Polypeptides can be purified from natural sources (e.g., a biological sample) by known methods such as DEAE ion exchange, gel filtration, and hydroxyapatite chromatography. A polypeptide also can be purified, for example, by expressing a nucleic acid in an expression vector. In addition, a purified polypeptide can be obtained by chemical synthesis. The extent of purity of a polypeptide can be measured using any appropriate method, e.g., column chromatography, polyacrylamide gel electrophoresis, or HPLC analysis.

In one aspect, this disclosure provides methods of detecting recombinant nucleic acids and polypeptides in plant cells. Without being limiting, nucleic acids also can be detected using hybridization. Hybridization between nucleic acids is discussed in detail in Sambrook et al. (1989, Molecular Cloning: A Laboratory Manual, 2nd Ed., Cold Spring Harbor Laboratory Press, Cold Spring Harbor, NY).

As used herein, the term “polypeptide” refers to a chain of at least two covalently linked amino acids. Polypeptides can be encoded by polynucleotides provided herein. Proteins provided herein can be encoded by nucleic acid molecules provided herein. Proteins can comprise polypeptides provided herein. As used herein, a “protein” refers to a chain of amino acid residues that is capable of providing structure or enzymatic activity to a cell. As used herein, the “product” of an allele refers to a protein encoded by the allele.

Polypeptides can be detected using antibodies. Techniques for detecting polypeptides using antibodies include enzyme linked immunosorbent assays (ELISAs), Western blots, immunoprecipitations and immunofluorescence. An antibody provided herein can be a polyclonal antibody or a monoclonal antibody. An antibody having specific binding affinity for a polypeptide provided herein can be generated using methods well known in the art. An antibody provided herein can be attached to a solid support such as a microtiter plate using methods known in the art.

Detection (e.g., of an amplification product, of a hybridization complex, of a polypeptide) can be accomplished using detectable labels. The term “label” is intended to encompass the use of direct labels as well as indirect labels. Detectable labels include enzymes, prosthetic groups, fluorescent materials, luminescent materials, bioluminescent materials, and radioactive materials.

As used herein, “allele” refers to a form or variant of the sequence of nucleotides present at a specific locus on a DNA molecule. As used herein, a “reference allele” refers to a naturally occurring wild type sequence of a NtHAP1a, NtHAP1b, NtHAP2, or NtHAP3 gene. As used herein, a “mutant allele” refers to a non-naturally occurring nucleic acid sequence of a NtHAP1a, NtHAP1b, NtHAP2, or NtHAP3 gene that has been modified by humans to differ in sequence from a corresponding reference allele by at least one nucleotide (e.g., it contains at least one non-natural mutation). When comparing sequences of mutant and reference alleles, a mutant allele of NtHAP1a should only be compared to a reference allele of NtHAP1a, a mutant allele of NtHAP1b should only be compared to a reference allele of NtHAP1b; a mutant allele of NtHAP2 should only be compared to a reference allele of NtHAP2. As a non-limiting example, it is improper to compare a NtHAP1a reference allele to an NtHAP1b, NtHAP2, or NtHAP3 mutant allele sequence.

In an aspect, a modified tobacco plant, or part thereof, comprises mutant alleles of both a coding sequence and a promoter for any of NtHAP1a, NtHAP1b, NtHAP2, and NtHAP3.

In an aspect, a mutant allele comprises a nucleic acid sequence selected from the group consisting of SEQ ID NOs: 72 to 89 and 285 to 351. In an aspect, a mutant allele comprises a nucleic acid sequence selected from the group consisting of SEQ ID NOs: 34 to 71 and 135 to 284. In an aspect, a mutant allele comprises a nucleic acid sequence encoding an amino acid sequence selected from the group consisting of SEQ ID NOs: 97 to 134 or 385 to 533.

Examples of reference allele sequences for NtHAP1a, NtHAP1b, and NtHAP2 include those provided in Table 9. Table 13 and Table 14 provide non-limiting examples 4 and 385 to 533. In an aspect, a mutant allele comprises the nucleic acid sequence of SEQ ID NO: 96. In an aspect, a mutant allele comprises a deletion of a nucleic acid sequence selected from the group consisting of SEQ ID NOs: 90 to 95 and 352 to 384 as compared to a corresponding reference sequence.

In an aspect, a mutant allele of NtHAP1a comprises a nucleic acid sequence selected from the group consisting of SEQ ID NOs: 34, 35, 37, 40, 42, 44, 46, 47, 49, 50, 53, 57, 58, 61 to 71, 135 to 137, 140 to 142, 144, 145, 148, 149, 152, 153, 156, 157, 160, 161, 164, 166, 167, 169, 170, 173, 174, 177, 178, 184, 198, 202, 207, 209, 215, 216, 221, and 223 to 269. In an aspect, a mutant allele of NtHAP1a comprises a nucleic acid sequence selected from the group consisting of SEQ ID NOs: 72, 75, 77, 79, 80, 83, 86, 87, 286, 287, 290, 291, 294, 295, 297 to 299, 301, 303, 304, 307 to 310, 313, 316 to 318, 326, 327, 329, 331, 336, 339, 341, 343, 345, 346, 349, and 350. In an aspect, a mutant allele of NtHAP1a comprises a nucleic acid sequence encoding an amino acid sequence selected from the group consisting of SEQ ID NOs: 97, 98, 100, 103, 105, 107, 109, 110, 112, 113, 116, 120, 121, 124 to 134, 385 to 387, 390 to 392, 394, 395, 398, 399, 402, 403, 406, 407, 410, 411, 414, 416, 417, 419, 420, 423, 424, 427, 428, 434, 448, 452, 457, 459, 465, 466, 471, and 473 to 519.

In an aspect, a mutant allele of NtHAP1b comprises a nucleic acid sequence selected from the group consisting of SEQ ID NOs: 36, 38, 39, 41, 43, 45, 48, 138, 139, 143, 146, 147, 150, 151, 154, 155, 158, 159, 162, 163, 165, 168, 171, 172, 175, 176, 179 to 181, 185, 186, 191, 194, 195, 199, 203, 205, 210, 218, 222, and 270 to 277. In an aspect, a mutant allele of NtHAP1b comprises a nucleic acid sequence selected from the group consisting of SEQ ID NOs: 73, 74, 76, 78, 81, 82, 84, 85, 88, 89, 285, 288, 289, 292, 293, 296, 300, 302, 305, 306, 311, 312, 314, 315, 319, 320, 323 to 325, 328, 330, 332 to 335, 337, 338, 340, 342, 344, 347, 348, and 351. In an aspect, a mutant allele of NtHAP1b comprises a nucleic acid sequence encoding an amino acid sequence selected from the group consisting of SEQ ID NOs: 99, 101, 102, 104, 106, 108, 111, 117, 118, 388, 389, 393, 396, 397, 400, 401, 404, 405, 408, 409, 412, 413, 415, 418, 421, 422, 425, 426, 429 to 431, 435, 436, 441, 444, 445, 449, 453, 455, 460, 468, 472, and 520 to 526.

In an aspect, a mutant allele of NtHAP2 comprises a nucleic acid sequence selected from the group consisting of SEQ ID NOs: 51, 52, 56, 182, 183, 187 to 190, 192, 193, 196, 197, 200, 201, 204, 206, 208, 211 to 214, 217, 219, 220, and 278 to 284. In an aspect, a mutant allele of NtHAP2 comprises a nucleic acid sequence selected from the group consisting of SEQ ID NOs: 321 and 322. In an aspect, a mutant allele of NtHAP2 comprises a nucleic acid sequence encoding an amino acid sequence selected from the group consisting of SEQ ID NOs: 114, 115, 119, 432, 433, 437 to 440, 442, 443, 446, 447, 450, 451, 454, 456, 458, 461 to 464, 467, 469, 470, and 527 to 533.

In an aspect, a modified tobacco plant, or parts thereof, comprises any combination of NtHAP1a mutant alleles provided herein. In an aspect, a modified tobacco plant, or parts thereof, comprises any combination of NtHAP1b mutant alleles provided herein. In an aspect, a modified tobacco plant, or parts thereof, comprises any combination of NtHAP2 mutant alleles provided herein. In an aspect, a modified tobacco plant, or parts thereof, comprises any combination of NtHAP1a mutant alleles and NtHAP1b mutant alleles provided herein. In an aspect, a modified tobacco plant, or parts thereof, comprises any combination of NtHAP1a mutant alleles and NtHAP2 mutant alleles provided herein. In an aspect, a modified tobacco plant, or parts thereof, comprises any combination of NtHAP2 mutant alleles and NtHAP1b mutant alleles provided herein. In an aspect, a modified tobacco plant, or parts thereof, comprises any combination of NtHAP1a mutant alleles, NtHAP1b mutant alleles, and NtHAP2 mutant alleles provided herein.

In an aspect, a modified tobacco plant, or parts thereof, comprises two NtHAP1a mutant alleles, each mutant allele comprising a nucleic acid sequence independently selected from the group consisting of SEQ ID NOs: 34, 35, 37, 40, 42, 44, 46, 47, 49, 50, 53, 57, 58, 61 to 71, 135 to 137, 140 to 142, 144, 145, 148, 149, 152, 153, 156, 157, 160, 161, 164, 166, 167, 169, 170, 173, 174, 177, 178, 184, 198, 202, 207, 209, 215, 216, 221, and 223 to 269. In an aspect, a modified tobacco plant, or parts thereof, comprises two NtHAP1a mutant alleles, each mutant allele comprising a nucleic acid sequence independently selected from the group consisting of SEQ ID NOs: 72, 75, 77, 79, 80, 83, 86, 87, 286, 287, 290, 291, 294, 295, 297 to 299, 301, 303, 304, 307 to 310, 313, 316 to 318, 326, 327, 329, 331, 336, 339, 341, 343, 345, 346, 349, and 350. In an aspect, a modified tobacco plant, or parts thereof, comprises two NtHAP1a mutant alleles, each mutant allele comprising a nucleic acid sequence independently selected from the group consisting of SEQ ID NOs: 34, 35, 37, 40, 42, 44, 46, 47, 49, 50, 53, 57, 58, 61 to 71, 135 to 137, 140 to 142, 144, 145, 148, 149, 152, 153, 156, 157, 160, 161, 164, 166, 167, 169, 170, 173, 174, 177, 178, 184, 198, 202, 207, 209, 215, 216, 221, 223 to 269, 72, 75, 77, 79, 80, 83, 86, 87, 286, 287, 290, 291, 294, 295, 297 to 299, 301, 303, 304, 307 to 310, 313, 316 to 318, 326, 327, 329, 331, 336, 339, 341, 343, 345, 346, 349, and 350. In an aspect, a modified tobacco plant, or parts thereof, comprises two NtHAP1a mutant alleles, each mutant allele comprising a nucleic acid sequence encoding an amino acid sequence independently selected from the group consisting of SEQ ID NOs: 97, 98, 100, 103, 105, 107, 109, 110, 112, 113, 116, 120, 121, 124 to 134, 385 to 387, 390 to 392, 394, 395, 398, 399, 402, 403, 406, 407, 410, 411, 414, 416, 417, 419, 420, 423, 424, 427, 428, 434, 448, 452, 457, 459, 465, 466, 471, and 473 to 519.

In an aspect, a modified tobacco plant, or parts thereof, comprises two NtHAP1b mutant alleles, each mutant allele comprising a nucleic acid sequence independently selected from the group consisting of SEQ ID NOs: 36, 38, 39, 41, 43, 45, 48, 138, 139, 143, 146, 147, 150, 151, 154, 155, 158, 159, 162, 163, 165, 168, 171, 172, 175, 176, 179 to 181, 185, 186, 191, 194, 195, 199, 203, 205, 210, 218, 222, and 270 to 277. In an aspect, a modified tobacco plant, or parts thereof, comprises two NtHAP1b mutant alleles, each mutant allele comprising a nucleic acid sequence independently selected from the group consisting of SEQ ID NOs: 73, 74, 76, 78, 81, 82, 84, 85, 88, 89, 285, 288, 289, 292, 293, 296, 300, 302, 305, 306, 311, 312, 314, 315, 319, 320, 323 to 325, 328, 330, 332 to 335, 337, 338, 340, 342, 344, 347, 348, and 351. In an aspect, a modified tobacco plant, or parts thereof, comprises two NtHAP1b mutant alleles, each mutant allele comprising a nucleic acid sequence independently selected from the group consisting of SEQ ID NOs: 36, 38, 39, 41, 43, 45, 48, 138, 139, 143, 146, 147, 150, 151, 154, 155, 158, 159, 162, 163, 165, 168, 171, 172, 175, 176, 179 to 181, 185, 186, 191, 194, 195, 199, 203, 205, 210, 218, 222, and 270 to 277, 73, 74, 76, 78, 81, 82, 84, 85, 88, 89, 285, 288, 289, 292, 293, 296, 300, 302, 305, 306, 311, 312, 314, 315, 319, 320, 323 to 325, 328, 330, 332 to 335, 337, 338, 340, 342, 344, 347, 348, and 351. In an aspect, a modified tobacco plant, or parts thereof, comprises two NtHAP1b mutant alleles, each mutant allele comprising a nucleic acid sequence encoding an amino acid sequence independently selected from the group consisting of SEQ ID NOs: 99, 101, 102, 104, 106, 108, 111, 117, 118, 388, 389, 393, 396, 397, 400, 401, 404, 405, 408, 409, 412, 413, 415, 418, 421, 422, 425, 426, 429 to 431, 435, 436, 441, 444, 445, 449, 453, 455, 460, 468, 472, and 520 to 526.

In an aspect, a modified tobacco plant, or parts thereof, comprises two NtHAP2 mutant alleles, each mutant allele comprising a nucleic acid sequence independently selected from the group consisting of SEQ ID NOs: 51, 52, 56, 182, 183, 187 to 190, 192, 193, 196, 197, 200, 201, 204, 206, 208, 211 to 214, 217, 219, 220, and 278 to 284. In an aspect, a modified tobacco plant, or parts thereof, comprises two NtHAP2 mutant alleles, each mutant allele comprising a nucleic acid sequence independently selected from the group consisting of SEQ ID NOs: 321 and 322. In an aspect, a modified tobacco plant, or parts thereof, comprises two NtHAP2 mutant alleles, each mutant allele comprising a nucleic acid sequence independently selected from the group consisting of SEQ ID NOs: 51, 52, 56, 182, 183, 187 to 190, 192, 193, 196, 197, 200, 201, 204, 206, 208, 211 to 214, 217, 219, 220, 278 to 284, 321, and 322. In an aspect, a modified tobacco plant, or parts thereof, comprises two NtHAP2 mutant alleles, each mutant allele comprising a nucleic acid sequence encoding an amino acid sequence independently selected from the group consisting of SEQ ID NOs: 114, 115, 119, 432, 433, 437 to 440, 442, 443, 446, 447, 450, 451, 454, 456, 458, 461 to 464, 467, 469, 470, and 527 to 533.

In an aspect, a modified tobacco plant, or part thereof, comprises at least one mutant allele of an endogenous locus encoding a HAIRPLUS1a (NtHAP1a) protein, where the at least one mutant allele comprises a nucleic acid sequence selected from the group consisting of SEQ ID NOs: 34, 35, 37, 40, 42, 44, 46, 47, 49, 50, 53, 57, 58, 61 to 71, 135 to 137, 140 to 142, 144, 145, 148, 149, 152, 153, 156, 157, 160, 161, 164, 166, 167, 169, 170, 173, 174, 177, 178, 184, 198, 202, 207, 209, 215, 216, 221, and 223 to 269. In an aspect, a modified tobacco plant, or part thereof, comprises two mutant alleles of an endogenous locus encoding a HAIRPLUS1a (NtHAP1a) protein, where each of the two mutant alleles comprises a nucleic acid sequence independently selected from the group consisting of SEQ ID NOs: 34, 35, 37, 40, 42, 44, 46, 47, 49, 50, 53, 57, 58, 61 to 71, 135 to 137, 140 to 142, 144, 145, 148, 149, 152, 153, 156, 157, 160, 161, 164, 166, 167, 169, 170, 173, 174, 177, 178, 184, 198, 202, 207, 209, 215, 216, 221, and 223 to 269.

In an aspect, a modified tobacco plant, or part thereof, comprises at least one mutant allele of an endogenous locus encoding a HAIRPLUS1a (NtHAP1a) protein, where the at least one mutant allele comprises a nucleic acid sequence selected from the group consisting of SEQ ID NOs: 72, 75, 77, 79, 80, 83, 86, 87, 286, 287, 290, 291, 294, 295, 297 to 299, 301, 303, 304, 307 to 310, 313, 316 to 318, 326, 327, 329, 331, 336, 339, 341, 343, 345, 346, 349, and 350. In an aspect, a modified tobacco plant, or part thereof, comprises two mutant alleles of an endogenous locus encoding a HAIRPLUS1a (NtHAP1a) protein, where each of the two mutant alleles comprises a nucleic acid sequence independently selected from the group consisting of SEQ ID NOs: 72, 75, 77, 79, 80, 83, 86, 87, 286, 287, 290, 291, 294, 295, 297 to 299, 301, 303, 304, 307 to 310, 313, 316 to 318, 326, 327, 329, 331, 336, 339, 341, 343, 345, 346, 349, and 350.

In an aspect, a modified tobacco plant, or part thereof, comprises (a) a first mutant allele of an endogenous HAIRPLUS1a (NtHAP1a) protein, where the first mutant allele comprises a nucleic acid sequence selected from the group consisting of SEQ ID NOs: 34, 35, 37, 40, 42, 44, 46, 47, 49, 50, 53, 57, 58, 61 to 71, 135 to 137, 140 to 142, 144, 145, 148, 149, 152, 153, 156, 157, 160, 161, 164, 166, 167, 169, 170, 173, 174, 177, 178, 184, 198, 202, 207, 209, 215, 216, 221, and 223 to 269; and (b) a second mutant allele of the endogenous locus encoding a HAIRPLUS1a (NtHAP1a) protein, where the second mutant allele comprises a nucleic acid sequence selected from the group consisting of SEQ ID NOs: 72, 75, 77, 79, 80, 83, 86, 87, 286, 287, 290, 291, 294, 295, 297 to 299, 301, 303, 304, 307 to 310, 313, 316 to 318, 326, 327, 329, 331, 336, 339, 341, 343, 345, 346, 349, and 350. In an aspect, a modified tobacco plant, or part thereof, comprises (a) two mutant alleles of an endogenous HAIRPLUS1a (NtHAP1a) protein, where each of the two mutant alleles comprises a nucleic acid sequence independently selected from the group consisting of SEQ ID NOs: 34, 35, 37, 40, 42, 44, 46, 47, 49, 50, 53, 57, 58, 61 to 71, 135 to 137, 140 to 142, 144, 145, 148, 149, 152, 153, 156, 157, 160, 161, 164, 166, 167, 169, 170, 173, 174, 177, 178, 184, 198, 202, 207, 209, 215, 216, 221, and 223 to 269; and (b) two mutant promoter alleles of the endogenous locus encoding a HAIRPLUS1a (NtHAP1a) protein, where each of the two mutant promoter alleles comprises a nucleic acid sequence independently selected from the group consisting of SEQ ID NOs: 72, 75, 77, 79, 80, 83, 86, 87, 286, 287, 290, 291, 294, 295, 297 to 299, 301, 303, 304, 307 to 310, 313, 316 to 318, 326, 327, 329, 331, 336, 339, 341, 343, 345, 346, 349, and 350.

In an aspect, a modified tobacco plant, or part thereof, comprises at least one mutant allele of an endogenous locus encoding a HAIRPLUS1a (NtHAP1a) protein, where the at least one mutant allele comprises a nucleic acid sequence encoding an amino acid sequence selected from the group consisting of SEQ ID NOs: 97, 98, 100, 103, 105, 107, 109, 110, 112, 113, 116, 120, 121, 124 to 134, 385 to 387, 390 to 392, 394, 395, 398, 399, 402, 403, 406, 407, 410, 411, 414, 416, 417, 419, 420, 423, 424, 427, 428, 434, 448, 452, 457, 459, 465, 466, 471, and 473 to 519. In an aspect, a modified tobacco plant, or part thereof, comprises two mutant alleles of an endogenous locus encoding a HAIRPLUS1a (NtHAP1a) protein, where each of the two mutant alleles comprises a nucleic acid sequence encoding an amino acid sequence independently selected from the group consisting of SEQ ID NOs: 97, 98, 100, 103, 105, 107, 109, 110, 112, 113, 116, 120, 121, 124 to 134, 385 to 387, 390 to 392, 394, 395, 398, 399, 402, 403, 406, 407, 410, 411, 414, 416, 417, 419, 420, 423, 424, 427, 428, 434, 448, 452, 457, 459, 465, 466, 471, and 473 to 519.

In an aspect, a modified tobacco plant, or part thereof, comprises at least one mutant allele of an endogenous locus encoding a HAIRPLUS1b (NtHAP1b) protein, where the at least one mutant allele comprises a nucleic acid sequence selected from the group consisting of SEQ ID NOs: 36, 38, 39, 41, 43, 45, 48, 138, 139, 143, 146, 147, 150, 151, 154, 155, 158, 159, 162, 163, 165, 168, 171, 172, 175, 176, 179 to 181, 185, 186, 191, 194, 195, 199, 203, 205, 210, 218, 222, and 270 to 277. In an aspect, a modified tobacco plant, or part thereof, comprises two mutant alleles of an endogenous locus encoding a HAIRPLUS1b (NtHAP1b) protein, where each of the two mutant alleles comprises a nucleic acid sequence independently selected from the group consisting of SEQ ID NOs: 36, 38, 39, 41, 43, 45, 48, 138, 139, 143, 146, 147, 150, 151, 154, 155, 158, 159, 162, 163, 165, 168, 171, 172, 175, 176, 179 to 181, 185, 186, 191, 194, 195, 199, 203, 205, 210, 218, 222, and 270 to 277.

In an aspect, a modified tobacco plant, or part thereof, comprises at least one mutant allele of an endogenous locus encoding a HAIRPLUS1b (NtHAP1b) protein, where the at least one mutant allele comprises a nucleic acid sequence selected from the group consisting of SEQ ID NOs: 73, 74, 76, 78, 81, 82, 84, 85, 88, 89, 285, 288, 289, 292, 293, 296, 300, 302, 305, 306, 311, 312, 314, 315, 319, 320, 323 to 325, 328, 330, 332 to 335, 337, 338, 340, 342, 344, 347, 348, and 351. In an aspect, a modified tobacco plant, or part thereof, comprises two mutant alleles of an endogenous locus encoding a HAIRPLUS1b (NtHAP1b) protein, where each of the two mutant alleles comprises a nucleic acid sequence independently selected from the group consisting of SEQ ID NOs: 73, 74, 76, 78, 81, 82, 84, 85, 88, 89, 285, 288, 289, 292, 293, 296, 300, 302, 305, 306, 311, 312, 314, 315, 319, 320, 323 to 325, 328, 330, 332 to 335, 337, 338, 340, 342, 344, 347, 348, and 351.

In an aspect, a modified tobacco plant, or part thereof, comprises (a) a first mutant allele of an endogenous HAIRPLUS1b (NtHAP1b) protein, where the first mutant allele comprises a nucleic acid sequence selected from the group consisting of SEQ ID NOs: 36, 38, 39, 41, 43, 45, 48, 138, 139, 143, 146, 147, 150, 151, 154, 155, 158, 159, 162, 163, 165, 168, 171, 172, 175, 176, 179 to 181, 185, 186, 191, 194, 195, 199, 203, 205, 210, 218, 222, and 270 to 277; and (b) a second mutant allele of the endogenous locus encoding a HAIRPLUS1b (NtHAP1b) protein, where the second mutant allele comprises a nucleic acid sequence selected from the group consisting of SEQ ID NOs: 73, 74, 76, 78, 81, 82, 84, 85, 88, 89, 285, 288, 289, 292, 293, 296, 300, 302, 305, 306, 311, 312, 314, 315, 319, 320, 323 to 325, 328, 330, 332 to 335, 337, 338, 340, 342, 344, 347, 348, and 351. In an aspect, a modified tobacco plant, or part thereof, comprises (a) two mutant alleles of an endogenous HAIRPLUS1b (NtHAP1b) protein, where each of the two mutant alleles comprises a nucleic acid sequence independently selected from the group consisting of SEQ ID NOs: 36, 38, 39, 41, 43, 45, 48, 138, 139, 143, 146, 147, 150, 151, 154, 155, 158, 159, 162, 163, 165, 168, 171, 172, 175, 176, 179 to 181, 185, 186, 191, 194, 195, 199, 203, 205, 210, 218, 222, and 270 to 277; and (b) two mutant promoter alleles of the endogenous locus encoding a HAIRPLUS1b (NtHAP1b) protein, where each of the two mutant promoter alleles comprises a nucleic acid sequence independently selected from the group consisting of SEQ ID NOs: 73, 74, 76, 78, 81, 82, 84, 85, 88, 89, 285, 288, 289, 292, 293, 296, 300, 302, 305, 306, 311, 312, 314, 315, 319, 320, 323 to 325, 328, 330, 332 to 335, 337, 338, 340, 342, 344, 347, 348, and 351.

In an aspect, a modified tobacco plant, or part thereof, comprises at least one mutant allele of an endogenous locus encoding a HAIRPLUS1b (NtHAP1b) protein, where the at least one mutant allele comprises a nucleic acid sequence encoding an amino acid sequence selected from the group consisting of SEQ ID NOs: 99, 101, 102, 104, 106, 108, 111, 117, 118, 388, 389, 393, 396, 397, 400, 401, 404, 405, 408, 409, 412, 413, 415, 418, 421, 422, 425, 426, 429 to 431, 435, 436, 441, 444, 445, 449, 453, 455, 460, 468, 472, and 520 to 526. In an aspect, a modified tobacco plant, or part thereof, comprises two mutant alleles of an endogenous locus encoding a HAIRPLUS1b (NtHAP1b) protein, where each of the two mutant alleles comprises a nucleic acid sequence encoding an amino acid sequence independently selected from the group consisting of SEQ ID NOs: 99, 101, 102, 104, 106, 108, 111, 117, 118, 388, 389, 393, 396, 397, 400, 401, 404, 405, 408, 409, 412, 413, 415, 418, 421, 422, 425, 426, 429 to 431, 435, 436, 441, 444, 445, 449, 453, 455, 460, 468, 472, and 520 to 526.

In an aspect, a modified tobacco plant, or part thereof, comprises at least one mutant allele of an endogenous locus encoding a HAIRPLUS2 (NtHAP2) protein, where the at least one mutant allele comprises a nucleic acid sequence selected from the group consisting of SEQ ID NOs: 51, 52, 56, 182, 183, 187 to 190, 192, 193, 196, 197, 200, 201, 204, 206, 208, 211 to 214, 217, 219, 220, and 278 to 284. In an aspect, a modified tobacco plant, or part thereof, comprises two mutant alleles of an endogenous locus encoding a HAIRPLUS2 (NtHAP2) protein, where each of the two mutant alleles comprises a nucleic acid sequence independently selected from the group consisting of SEQ ID NOs: 51, 52, 56, 182, 183, 187 to 190, 192, 193, 196, 197, 200, 201, 204, 206, 208, 211 to 214, 217, 219, 220, and 278 to 284.

In an aspect, a modified tobacco plant, or part thereof, comprises at least one mutant allele of an endogenous locus encoding a HAIRPLUS2 (NtHAP2) protein, where the at least one mutant allele comprises a nucleic acid sequence selected from the group consisting of SEQ ID NOs: 321 and 322. In an aspect, a modified tobacco plant, or part thereof, comprises two mutant alleles of an endogenous locus encoding a HAIRPLUS2 (NtHAP2) protein, where each of the two mutant alleles comprises a nucleic acid sequence independently selected from the group consisting of SEQ ID NOs: 321 and 322.

In an aspect, a modified tobacco plant, or part thereof, comprises (a) a first mutant allele of an endogenous HAIRPLUS2 (NtHAP2) protein, where the first mutant allele comprises a nucleic acid sequence selected from the group consisting of SEQ ID NOs: 51, 52, 56, 182, 183, 187 to 190, 192, 193, 196, 197, 200, 201, 204, 206, 208, 211 to 214, 217, 219, 220, and 278 to 284; and (b) a second mutant allele of the endogenous locus encoding a HAIRPLUS2 (NtHAP2) protein, where the second mutant allele comprises a nucleic acid sequence selected from the group consisting of SEQ ID NOs: 321 and 322. In an aspect, a modified tobacco plant, or part thereof, comprises (a) two mutant alleles of an endogenous HAIRPLUS2 (NtHAP2) protein, where each of the two mutant alleles comprises a nucleic acid sequence independently selected from the group consisting of SEQ ID NOs: 51, 52, 56, 182, 183, 187 to 190, 192, 193, 196, 197, 200, 201, 204, 206, 208, 211 to 214, 217, 219, 220, and 278 to 284; and (b) two mutant promoter alleles of the endogenous locus encoding a HAIRPLUS2 (NtHAP2) protein, where each of the two mutant promoter alleles comprises a nucleic acid sequence independently selected from the group consisting of SEQ ID NOs: 321 and 322.

In an aspect, a modified tobacco plant, or part thereof, comprises at least one mutant allele of an endogenous locus encoding a HAIRPLUS2 (NtHAP2) protein, where the at least one mutant allele comprises a nucleic acid sequence encoding an amino acid sequence selected from the group consisting of SEQ ID NOs: 114, 115, 119, 432, 433, 437 to 440, 442, 443, 446, 447, 450, 451, 454, 456, 458, 461 to 464, 467, 469, 470, and 527 to 533. In an aspect, a modified tobacco plant, or part thereof, comprises two mutant alleles of an endogenous locus encoding a HAIRPLUS2 (NtHAP2) protein, where each of the two mutant alleles comprises a nucleic acid sequence encoding an amino acid sequence independently selected from the group consisting of SEQ ID NOs: 114, 115, 119, 432, 433, 437 to 440, 442, 443, 446, 447, 450, 451, 454, 456, 458, 461 to 464, 467, 469, 470, and 527 to 533.

In an aspect, a modified tobacco plant comprises at least one NtHAP1a mutant allele comprising a nucleic acid sequence selected from the group consisting of SEQ ID NOs: 34, 35, 37, 40, 42, 44, 46, 47, 49, 50, 53, 57, 58, 61 to 71, 135 to 137, 140 to 142, 144, 145, 148, 149, 152, 153, 156, 157, 160, 161, 164, 166, 167, 169, 170, 173, 174, 177, 178, 184, 198, 202, 207, 209, 215, 216, 221, 223 to 269, 72, 75, 77, 79, 80, 83, 86, 87, 286, 287, 290, 291, 294, 295, 297 to 299, 301, 303, 304, 307 to 310, 313, 316 to 318, 326, 327, 329, 331, 336, 339, 341, 343, 345, 346, 349, and 350 and at least one NtHAP1b mutant allele comprising a nucleic acid sequence selected from the group consisting of SEQ ID NOs: 36, 38, 39, 41, 43, 45, 48, 138, 139, 143, 146, 147, 150, 151, 154, 155, 158, 159, 162, 163, 165, 168, 171, 172, 175, 176, 179 to 181, 185, 186, 191, 194, 195, 199, 203, 205, 210, 218, 222, 270 to 277, 73, 74, 76, 78, 81, 82, 84, 85, 88, 89, 285, 288, 289, 292, 293, 296, 300, 302, 305, 306, 311, 312, 314, 315, 319, 320, 323 to 325, 328, 330, 332 to 335, 337, 338, 340, 342, 344, 347, 348, and 351.

In an aspect, a modified tobacco plant comprises at least one NtHAP1a mutant allele comprising a nucleic acid sequence selected from the group consisting of SEQ ID NOs: 34, 35, 37, 40, 42, 44, 46, 47, 49, 50, 53, 57, 58, 61 to 71, 135 to 137, 140 to 142, 144, 145, 148, 149, 152, 153, 156, 157, 160, 161, 164, 166, 167, 169, 170, 173, 174, 177, 178, 184, 198, 202, 207, 209, 215, 216, 221, 223 to 269, 72, 75, 77, 79, 80, 83, 86, 87, 286, 287, 290, 291, 294, 295, 297 to 299, 301, 303, 304, 307 to 310, 313, 316 to 318, 326, 327, 329, 331, 336, 339, 341, 343, 345, 346, 349, and 350 and at least one NtHAP2 mutant allele comprising a nucleic acid sequence selected from the group consisting of SEQ ID NOs: 51, 52, 56, 182, 183, 187 to 190, 192, 193, 196, 197, 200, 201, 204, 206, 208, 211 to 214, 217, 219, 220, 278 to 284, 321, and 322.

In an aspect, a modified tobacco plant comprises at least one NtHAP1b mutant allele comprising a nucleic acid sequence selected from the group consisting of SEQ ID NOs: 36, 38, 39, 41, 43, 45, 48, 138, 139, 143, 146, 147, 150, 151, 154, 155, 158, 159, 162, 163, 165, 168, 171, 172, 175, 176, 179 to 181, 185, 186, 191, 194, 195, 199, 203, 205, 210, 218, 222, 270 to 277, 73, 74, 76, 78, 81, 82, 84, 85, 88, 89, 285, 288, 289, 292, 293, 296, 300, 302, 305, 306, 311, 312, 314, 315, 319, 320, 323 to 325, 328, 330, 332 to 335, 337, 338, 340, 342, 344, 347, 348, and 351 and at least one NtHAP2 mutant allele comprising a nucleic acid sequence selected from the group consisting of SEQ ID NOs: 51, 52, 56, 182, 183, 187 to 190, 192, 193, 196, 197, 200, 201, 204, 206, 208, 211 to 214, 217, 219, 220, 278 to 284, 321, and 322.

In an aspect, a modified tobacco plant comprises at least one NtHAP1a mutant allele comprising a nucleic acid sequence selected from the group consisting of SEQ ID NOs: 34, 35, 37, 40, 42, 44, 46, 47, 49, 50, 53, 57, 58, 61 to 71, 135 to 137, 140 to 142, 144, 145, 148, 149, 152, 153, 156, 157, 160, 161, 164, 166, 167, 169, 170, 173, 174, 177, 178, 184, 198, 202, 207, 209, 215, 216, 221, 223 to 269, 72, 75, 77, 79, 80, 83, 86, 87, 286, 287, 290, 291, 294, 295, 297 to 299, 301, 303, 304, 307 to 310, 313, 316 to 318, 326, 327, 329, 331, 336, 339, 341, 343, 345, 346, 349, and 350, at least one NtHAP1b mutant allele comprising a nucleic acid sequence selected from the group consisting of SEQ ID NOs: 36, 38, 39, 41, 43, 45, 48, 138, 139, 143, 146, 147, 150, 151, 154, 155, 158, 159, 162, 163, 165, 168, 171, 172, 175, 176, 179 to 181, 185, 186, 191, 194, 195, 199, 203, 205, 210, 218, 222, 270 to 277, 73, 74, 76, 78, 81, 82, 84, 85, 88, 89, 285, 288, 289, 292, 293, 296, 300, 302, 305, 306, 311, 312, 314, 315, 319, 320, 323 to 325, 328, 330, 332 to 335, 337, 338, 340, 342, 344, 347, 348, and 351, and at least one NtHAP2 mutant allele comprising a nucleic acid sequence selected from the group consisting of SEQ ID NOs: 51, 52, 56, 182, 183, 187 to 190, 192, 193, 196, 197, 200, 201, 204, 206, 208, 211 to 214, 217, 219, 220, 278 to 284, 321, and 322.

In an aspect, a modified tobacco plant comprises at least two NtHAP1a mutant alleles, where each of the at least two NtHAP1a mutant alleles comprises a nucleic acid sequence independently selected from the group consisting of SEQ ID NOs: 34, 35, 37, 40, 42, 44, 46, 47, 49, 50, 53, 57, 58, 61 to 71, 135 to 137, 140 to 142, 144, 145, 148, 149, 152, 153, 156, 157, 160, 161, 164, 166, 167, 169, 170, 173, 174, 177, 178, 184, 198, 202, 207, 209, 215, 216, 221, 223 to 269, 72, 75, 77, 79, 80, 83, 86, 87, 286, 287, 290, 291, 294, 295, 297 to 299, 301, 303, 304, 307 to 310, 313, 316 to 318, 326, 327, 329, 331, 336, 339, 341, 343, 345, 346, 349, and 350 and at least two NtHAP1b mutant alleles, where each of the at least two NtHAP1b mutant alleles comprises a nucleic acid sequence independently selected from the group consisting of SEQ ID NOs: 36, 38, 39, 41, 43, 45, 48, 138, 139, 143, 146, 147, 150, 151, 154, 155, 158, 159, 162, 163, 165, 168, 171, 172, 175, 176, 179 to 181, 185, 186, 191, 194, 195, 199, 203, 205, 210, 218, 222, 270 to 277, 73, 74, 76, 78, 81, 82, 84, 85, 88, 89, 285, 288, 289, 292, 293, 296, 300, 302, 305, 306, 311, 312, 314, 315, 319, 320, 323 to 325, 328, 330, 332 to 335, 337, 338, 340, 342, 344, 347, 348, and 351.

In an aspect, a modified tobacco plant comprises at least two NtHAP1a mutant alleles, where each of the at least two NtHAP1a mutant alleles comprises a nucleic acid sequence independently selected from the group consisting of SEQ ID NOs: 34, 35, 37, 40, 42, 44, 46, 47, 49, 50, 53, 57, 58, 61 to 71, 135 to 137, 140 to 142, 144, 145, 148, 149, 152, 153, 156, 157, 160, 161, 164, 166, 167, 169, 170, 173, 174, 177, 178, 184, 198, 202, 207, 209, 215, 216, 221, 223 to 269, 72, 75, 77, 79, 80, 83, 86, 87, 286, 287, 290, 291, 294, 295, 297 to 299, 301, 303, 304, 307 to 310, 313, 316 to 318, 326, 327, 329, 331, 336, 339, 341, 343, 345, 346, 349, and 350 and at least two NtHAP2 mutant alleles, where each of the at least two NtHAP2 mutant alleles comprises a nucleic acid sequence independently selected from the group consisting of SEQ ID NOs: 51, 52, 56, 182, 183, 187 to 190, 192, 193, 196, 197, 200, 201, 204, 206, 208, 211 to 214, 217, 219, 220, 278 to 284, 321, and 322.

In an aspect, a modified tobacco plant comprises at least two NtHAP1b mutant alleles, where each of the at least two NtHAP1b mutant alleles comprises a nucleic acid sequence independently selected from the group consisting of SEQ ID NOs: 36, 38, 39, 41, 43, 45, 48, 138, 139, 143, 146, 147, 150, 151, 154, 155, 158, 159, 162, 163, 165, 168, 171, 172, 175, 176, 179 to 181, 185, 186, 191, 194, 195, 199, 203, 205, 210, 218, 222, 270 to 277, 73, 74, 76, 78, 81, 82, 84, 85, 88, 89, 285, 288, 289, 292, 293, 296, 300, 302, 305, 306, 311, 312, 314, 315, 319, 320, 323 to 325, 328, 330, 332 to 335, 337, 338, 340, 342, 344, 347, 348, and 351 and at least two NtHAP2 mutant alleles, where each of the at least two NtHAP2 mutant alleles comprises a nucleic acid sequence independently selected from the group consisting of SEQ ID NOs: 51, 52, 56, 182, 183, 187 to 190, 192, 193, 196, 197, 200, 201, 204, 206, 208, 211 to 214, 217, 219, 220, 278 to 284, 321, and 322.

In an aspect, a modified tobacco plant comprises at least two NtHAP1a mutant alleles, where each of the at least two NtHAP1a mutant alleles comprises a nucleic acid sequence independently selected from the group consisting of SEQ ID NOs: 34, 35, 37, 40, 42, 44, 46, 47, 49, 50, 53, 57, 58, 61 to 71, 135 to 137, 140 to 142, 144, 145, 148, 149, 152, 153, 156, 157, 160, 161, 164, 166, 167, 169, 170, 173, 174, 177, 178, 184, 198, 202, 207, 209, 215, 216, 221, 223 to 269, 72, 75, 77, 79, 80, 83, 86, 87, 286, 287, 290, 291, 294, 295, 297 to 299, 301, 303, 304, 307 to 310, 313, 316 to 318, 326, 327, 329, 331, 336, 339, 341, 343, 345, 346, 349, and 350, at least two NtHAP1b mutant alleles, where each of the at least two NtHAP1b mutant alleles comprises a nucleic acid sequence independently selected from the group consisting of SEQ ID NOs: 36, 38, 39, 41, 43, 45, 48, 138, 139, 143, 146, 147, 150, 151, 154, 155, 158, 159, 162, 163, 165, 168, 171, 172, 175, 176, 179 to 181, 185, 186, 191, 194, 195, 199, 203, 205, 210, 218, 222, 270 to 277, 73, 74, 76, 78, 81, 82, 84, 85, 88, 89, 285, 288, 289, 292, 293, 296, 300, 302, 305, 306, 311, 312, 314, 315, 319, 320, 323 to 325, 328, 330, 332 to 335, 337, 338, 340, 342, 344, 347, 348, and 351, and at least two NtHAP2 mutant alleles, where each of the at least two NtHAP2 mutant alleles comprises a nucleic acid sequence independently selected from the group consisting of SEQ ID NOs: 51, 52, 56, 182, 183, 187 to 190, 192, 193, 196, 197, 200, 201, 204, 206, 208, 211 to 214, 217, 219, 220, 278 to 284, 321, and 322.

In an aspect, a modified tobacco plant comprises the HAIRPLUS mutant alleles present in edited tobacco line KG12-05. In an aspect, a modified tobacco plant comprises the HAIRPLUS mutant alleles present in edited tobacco line KG12-09. In an aspect, a modified tobacco plant comprises the HAIRPLUS mutant alleles present in edited tobacco line KG12-13. In an aspect, a modified tobacco plant comprises the HAIRPLUS mutant alleles present in edited tobacco line KG12-24. In an aspect, a modified tobacco plant comprises the HAIRPLUS mutant alleles present in edited tobacco line KG12-29. In an aspect, a modified tobacco plant comprises the HAIRPLUS mutant alleles present in edited tobacco line KG12-31. In an aspect, a modified tobacco plant comprises the HAIRPLUS mutant alleles present in edited tobacco line KG1278-31. In an aspect, a modified tobacco plant comprises the HAIRPLUS mutant alleles present in edited tobacco line KG1278-44. In an aspect, a modified tobacco plant comprises the HAIRPLUS mutant alleles present in edited tobacco line KG1278-64. In an aspect, a modified tobacco plant comprises the HAIRPLUS mutant alleles present in edited tobacco line KG34-02. In an aspect, a modified tobacco plant comprises the HAIRPLUS mutant alleles present in edited tobacco line KG34-03. In an aspect, a modified tobacco plant comprises the HAIRPLUS mutant alleles present in edited tobacco line KG34-06. In an aspect, a modified tobacco plant comprises the HAIRPLUS mutant alleles present in edited tobacco line KG34-09. In an aspect, a modified tobacco plant comprises the HAIRPLUS mutant alleles present in edited tobacco line KG34-20. In an aspect, a modified tobacco plant comprises the HAIRPLUS mutant alleles present in edited tobacco line KG34-52. See Table 13 in this Specification for specific mutant allele information for the edited tobacco lines referenced in this paragraph. This disclosure also specifically provides self-pollinating any of the modified tobacco plants recited in this paragraph. This disclosure also specifically provides crossing any of the modified tobacco plants recited in this paragraph with any other tobacco plant.

In an aspect, a modified tobacco plant comprises the HAIRPLUS mutant alleles present in edited tobacco line IG12-08. In an aspect, a modified tobacco plant comprises the HAIRPLUS mutant alleles present in edited tobacco line IG12-09. In an aspect, a modified tobacco plant comprises the HAIRPLUS mutant alleles present in edited tobacco line IG12-13. In an aspect, a modified tobacco plant comprises the HAIRPLUS mutant alleles present in edited tobacco line IG12-14. In an aspect, a modified tobacco plant comprises the HAIRPLUS mutant alleles present in edited tobacco line IG12-15. In an aspect, a modified tobacco plant comprises the HAIRPLUS mutant alleles present in edited tobacco line IG12-17. In an aspect, a modified tobacco plant comprises the HAIRPLUS mutant alleles present in edited tobacco line IG12-30. In an aspect, a modified tobacco plant comprises the HAIRPLUS mutant alleles present in edited tobacco line IG12-35. In an aspect, a modified tobacco plant comprises the HAIRPLUS mutant alleles present in edited tobacco line IG12-39. In an aspect, a modified tobacco plant comprises the HAIRPLUS mutant alleles present in edited tobacco line IG12-41. In an aspect, a modified tobacco plant comprises the HAIRPLUS mutant alleles present in edited tobacco line IG12-42. In an aspect, a modified tobacco plant comprises the HAIRPLUS mutant alleles present in edited tobacco line IG12-44. In an aspect, a modified tobacco plant comprises the HAIRPLUS mutant alleles present in edited tobacco line IG12-53. In an aspect, a modified tobacco plant comprises the HAIRPLUS mutant alleles present in edited tobacco line IG12-58. In an aspect, a modified tobacco plant comprises the HAIRPLUS mutant alleles present in edited tobacco line IG1278-105. In an aspect, a modified tobacco plant comprises the mut In an aspect, a modified tobacco plant comprises the HAIRPLUS mutant alleles present in edited tobacco line IG1278-105.ant alleles present in edited tobacco line IG1278-107. In an aspect, a modified tobacco plant comprises the HAIRPLUS mutant alleles present in edited tobacco line IG1278-112. In an aspect, a modified tobacco plant comprises the HAIRPLUS mutant alleles present in edited tobacco line IG1278-117. In an aspect, a modified tobacco plant comprises the HAIRPLUS mutant alleles present in edited tobacco line IG1278-12. In an aspect, a modified tobacco plant comprises the HAIRPLUS mutant alleles present in edited tobacco line IG1278-13. In an aspect, a modified tobacco plant comprises the HAIRPLUS mutant alleles present in edited tobacco line IG1278-14. In an aspect, a modified tobacco plant comprises the HAIRPLUS mutant alleles present in edited tobacco line IG1278-32. In an aspect, a modified tobacco plant comprises the HAIRPLUS mutant alleles present in edited tobacco line IG1278-35. In an aspect, a modified tobacco plant comprises the HAIRPLUS mutant alleles present in edited tobacco line IG1278-47. In an aspect, a modified tobacco plant comprises the HAIRPLUS mutant alleles present in edited tobacco line IG1278-51. In an aspect, a modified tobacco plant comprises the HAIRPLUS mutant alleles present in edited tobacco line IG1278-59. In an aspect, a modified tobacco plant comprises the HAIRPLUS mutant alleles present in edited tobacco line IG1278-62. In an aspect, a modified tobacco plant comprises the HAIRPLUS mutant alleles present in edited tobacco line IG1278-69. In an aspect, a modified tobacco plant comprises the HAIRPLUS mutant alleles present in edited tobacco line IG1278-73. In an aspect, a modified tobacco plant comprises the HAIRPLUS mutant alleles present in edited tobacco line IG1278-76. In an aspect, a modified tobacco plant comprises the HAIRPLUS mutant alleles present in edited tobacco line IG1278-90. In an aspect, a modified tobacco plant comprises the HAIRPLUS mutant alleles present in edited tobacco line IG1278-97. In an aspect, a modified tobacco plant comprises the HAIRPLUS mutant alleles present in edited tobacco line IG34-14. In an aspect, a modified tobacco plant comprises the HAIRPLUS mutant alleles present in edited tobacco line IG34-15. In an aspect, a modified tobacco plant comprises the HAIRPLUS mutant alleles present in edited tobacco line IG34-19. In an aspect, a modified tobacco plant comprises the HAIRPLUS mutant alleles present in edited tobacco line IG34-29. In an aspect, a modified tobacco plant comprises the HAIRPLUS mutant alleles present in edited tobacco line IG34-42. In an aspect, a modified tobacco plant comprises the HAIRPLUS mutant alleles present in edited tobacco line IG34-48. In an aspect, a modified tobacco plant comprises the HAIRPLUS mutant alleles present in edited tobacco line IG34-65. In an aspect, a modified tobacco plant comprises the HAIRPLUS mutant alleles present in edited tobacco line IG34-71. In an aspect, a modified tobacco plant comprises the HAIRPLUS mutant alleles present in edited tobacco line IG34-72. In an aspect, a modified tobacco plant comprises the HAIRPLUS mutant alleles present in edited tobacco line IG34-74. In an aspect, a modified tobacco plant comprises the HAIRPLUS mutant alleles present in edited tobacco line IG34-94. In an aspect, a modified tobacco plant comprises the HAIRPLUS mutant alleles present in edited tobacco line IG56-02. In an aspect, a modified tobacco plant comprises the HAIRPLUS mutant alleles present in edited tobacco line IG56-12. In an aspect, a modified tobacco plant comprises the HAIRPLUS mutant alleles present in edited tobacco line IG56-20. In an aspect, a modified tobacco plant comprises the HAIRPLUS mutant alleles present in edited tobacco line IG56-75. In an aspect, a modified tobacco plant comprises the HAIRPLUS mutant alleles present in edited tobacco line IG78-11. In an aspect, a modified tobacco plant comprises the HAIRPLUS mutant alleles present in edited tobacco line IG78-13. In an aspect, a modified tobacco plant comprises the HAIRPLUS mutant alleles present in edited tobacco line IG78-33. In an aspect, a modified tobacco plant comprises the HAIRPLUS mutant alleles present in edited tobacco line IG78-35. In an aspect, a modified tobacco plant comprises the HAIRPLUS mutant alleles present in edited tobacco line IG78-47. See Table 14 in this Specification for specific mutant allele information for the edited tobacco lines referenced in this paragraph. This disclosure also specifically provides self-pollinating any of the modified tobacco plants recited in this paragraph. This disclosure also specifically provides crossing any of the modified tobacco plants recited in this paragraph with any other tobacco plant.

In an aspect, this disclosure provides a plant part, or plant parts, from any modified tobacco plant provided herein. In an aspect, this disclosure provides a seed from any modified tobacco plant provided herein. In an aspect, this disclosure provides a cell from any modified tobacco plant provided herein.

In an aspect, this disclosure provides the edited tobacco line KG12-05. In an aspect, this disclosure provides the edited tobacco line KG12-09. In an aspect, this disclosure provides the edited tobacco line KG12-13. In an aspect, this disclosure provides the edited tobacco line KG12-24. In an aspect, this disclosure provides the edited tobacco line KG12-29. In an aspect, this disclosure provides the edited tobacco line KG12-31. In an aspect, this disclosure provides the edited tobacco line KG1278-31. In an aspect, this disclosure provides the edited tobacco line KG1278-44. In an aspect, this disclosure provides the edited tobacco line KG1278-64. In an aspect, this disclosure provides the edited tobacco line KG34-02. In an aspect, this disclosure provides the edited tobacco line KG34-03. In an aspect, this disclosure provides the edited tobacco line KG34-06. In an aspect, this disclosure provides the edited tobacco line KG34-09. In an aspect, this disclosure provides the edited tobacco line KG34-20. In an aspect, this disclosure provides the edited tobacco line KG34-52. See Table 13 in this Specification for information regarding the edited tobacco lines referenced in this paragraph. This disclosure also specifically provides self-pollinating any of the edited tobacco lines recited in this paragraph. This disclosure also specifically provides crossing any of the edited tobacco lines recited in this paragraph with any other tobacco plant or line.

In an aspect, this disclosure provides the edited tobacco line IG12-08. In an aspect, this disclosure provides the edited tobacco line IG12-09. In an aspect, this disclosure provides the edited tobacco line IG12-13. In an aspect, this disclosure provides the edited tobacco line IG12-14. In an aspect, this disclosure provides the edited tobacco line IG12-15. In an aspect, this disclosure provides the edited tobacco line IG12-17. In an aspect, this disclosure provides the edited tobacco line IG12-30. In an aspect, this disclosure provides the edited tobacco line IG12-35. In an aspect, this disclosure provides the edited tobacco line IG12-39. In an aspect, this disclosure provides the edited tobacco line IG12-41. In an aspect, this disclosure provides the edited tobacco line IG12-42. In an aspect, this disclosure provides the edited tobacco line IG12-44. In an aspect, this disclosure provides the edited tobacco line IG12-53. In an aspect, this disclosure provides the edited tobacco line IG12-58. In an aspect, this disclosure provides the edited tobacco line IG1278-105. In an aspect, a modified tobacco plant comprises the mut In an aspect, this disclosure provides the edited tobacco line IG1278-105.ant alleles present in edited tobacco line IG1278-107. In an aspect, this disclosure provides the edited tobacco line IG1278-112. In an aspect, this disclosure provides the edited tobacco line IG1278-117. In an aspect, this disclosure provides the edited tobacco line IG1278-12. In an aspect, this disclosure provides the edited tobacco line IG1278-13. In an aspect, this disclosure provides the edited tobacco line IG1278-14. In an aspect, this disclosure provides the edited tobacco line IG1278-32. In an aspect, this disclosure provides the edited tobacco line IG1278-35. In an aspect, this disclosure provides the edited tobacco line IG1278-47. In an aspect, this disclosure provides the edited tobacco line IG1278-51. In an aspect, this disclosure provides the edited tobacco line IG1278-59. In an aspect, this disclosure provides the edited tobacco line IG1278-62. In an aspect, this disclosure provides the edited tobacco line IG1278-69. In an aspect, this disclosure provides the edited tobacco line IG1278-73. In an aspect, this disclosure provides the edited tobacco line IG1278-76. In an aspect, this disclosure provides the edited tobacco line IG1278-90. In an aspect, this disclosure provides the edited tobacco line IG1278-97. In an aspect, this disclosure provides the edited tobacco line IG34-14. In an aspect, this disclosure provides the edited tobacco line IG34-15. In an aspect, this disclosure provides the edited tobacco line IG34-19. In an aspect, this disclosure provides the edited tobacco line IG34-29. In an aspect, this disclosure provides the edited tobacco line IG34-42. In an aspect, this disclosure provides the edited tobacco line IG34-48. In an aspect, this disclosure provides the edited tobacco line IG34-65. In an aspect, this disclosure provides the edited tobacco line IG34-71. In an aspect, this disclosure provides the edited tobacco line IG34-72. In an aspect, this disclosure provides the edited tobacco line IG34-74. In an aspect, this disclosure provides the edited tobacco line IG34-94. In an aspect, this disclosure provides the edited tobacco line IG56-02. In an aspect, this disclosure provides the edited tobacco line IG56-12. In an aspect, this disclosure provides the edited tobacco line IG56-20. In an aspect, this disclosure provides the edited tobacco line IG56-75. In an aspect, this disclosure provides the edited tobacco line IG78-11. In an aspect, this disclosure provides the edited tobacco line IG78-13. In an aspect, this disclosure provides the edited tobacco line IG78-33. In an aspect, this disclosure provides the edited tobacco line IG78-35. In an aspect, this disclosure provides the edited tobacco line IG78-47. See Table 14 in this Specification for information regarding the edited tobacco lines referenced in this paragraph. This disclosure also specifically provides self-pollinating any of the edited tobacco lines recited in this paragraph. This disclosure also specifically provides crossing any of the edited tobacco lines recited in this paragraph with any other tobacco plant or line.

In an aspect, a modified tobacco plant comprises at least one NtHAP1a mutant allele. In an aspect, a modified tobacco plant comprises one NtHAP1a mutant allele. In an aspect, a modified tobacco plant comprises two NtHAP1a mutant alleles. In an aspect, a modified tobacco plant comprises at least one NtHAP1b mutant allele. In an aspect, a modified tobacco plant comprises one NtHAP1b mutant allele. In an aspect, a modified tobacco plant comprises two NtHAP1b mutant alleles. In an aspect, a modified tobacco plant comprises at least one NtHAP2 mutant allele. In an aspect, a modified tobacco plant comprises one NtHAP2 mutant allele. In an aspect, a modified tobacco plant comprises two NtHAP2 mutant alleles. In an aspect, a modified tobacco plant comprises at least one NtHAP3 mutant allele. In an aspect, a modified tobacco plant comprises one NtHAP3 mutant allele. In an aspect, a modified tobacco plant comprises two NtHAP3 mutant alleles.

In an aspect, a modified tobacco plant comprises at least one NtHAP1a mutant allele and at least one NtHAP1b mutant allele. In an aspect, a modified tobacco plant comprises one NtHAP1a mutant allele and one NtHAP1b mutant allele. In an aspect, a modified tobacco plant comprises two NtHAP1a mutant alleles and two NtHAP1b mutant alleles.

In an aspect, a modified tobacco plant comprises at least one NtHAP1a mutant allele and at least one NtHAP2 mutant allele. In an aspect, a modified tobacco plant comprises one NtHAP1a mutant allele and one NtHAP2 mutant allele. In an aspect, a modified tobacco plant comprises two NtHAP1a mutant alleles and two NtHAP2 mutant alleles.

In an aspect, a modified tobacco plant comprises at least one NtHAP1a mutant allele and at least one NtHAP3 mutant allele. In an aspect, a modified tobacco plant comprises one NtHAP1a mutant allele and one NtHAP3 mutant allele. In an aspect, a modified tobacco plant comprises two NtHAP1a mutant alleles and two NtHAP3 mutant alleles.

In an aspect, a modified tobacco plant comprises at least one NtHAP1b mutant allele and at least one NtHAP2 mutant allele. In an aspect, a modified tobacco plant comprises one NtHAP1b mutant allele and one NtHAP2 mutant allele. In an aspect, a modified tobacco plant comprises two NtHAP1b mutant alleles and two NtHAP2 mutant alleles.

In an aspect, a modified tobacco plant comprises at least one NtHAP1b mutant allele and at least one NtHAP3 mutant allele. In an aspect, a modified tobacco plant comprises one NtHAP1b mutant allele and one NtHAP3 mutant allele. In an aspect, a modified tobacco plant comprises two NtHAP1b mutant alleles and two NtHAP3 mutant alleles.

In an aspect, a modified tobacco plant comprises at least one NtHAP2 mutant allele and at least one NtHAP3 mutant allele. In an aspect, a modified tobacco plant comprises one NtHAP2 mutant allele and one NtHAP3 mutant allele. In an aspect, a modified tobacco plant comprises two NtHAP2 mutant alleles and two NtHAP3 mutant alleles.

In an aspect, a modified tobacco plant comprises at least one NtHAP1a mutant allele, at least one NtHAP1b mutant allele, and at least one NtHAP2 mutant allele. In an aspect, a modified tobacco plant comprises one NtHAP1a mutant allele, one NtHAP1b mutant allele, and one NtHAP2 mutant allele. In an aspect, a modified tobacco plant comprises two NtHAP1a mutant alleles, two NtHAP1b mutant alleles, and two NtHAP2 mutant alleles. In an aspect, a modified tobacco plant comprises at least one NtHAP1a mutant allele, at least one NtHAP1b mutant allele, and at least one NtHAP3 mutant allele. In an aspect, a modified tobacco plant comprises one NtHAP1a mutant allele, one NtHAP1b mutant allele, and one NtHAP3 mutant allele. In an aspect, a modified tobacco plant comprises two NtHAP1a mutant alleles, two NtHAP1b mutant alleles, and two NtHAP3 mutant alleles. In an aspect, a modified tobacco plant comprises at least one NtHAP1a mutant allele, at least one NtHAP2 mutant allele, and at least one NtHAP3 mutant allele. In an aspect, a modified tobacco plant comprises one NtHAP1a mutant allele, one NtHAP2 mutant allele, and one NtHAP3 mutant allele. In an aspect, a modified tobacco plant comprises two NtHAP1a mutant alleles, two NtHAP2 mutant alleles, and two NtHAP3 mutant alleles. In an aspect, a modified tobacco plant comprises at least one NtHAP1b mutant allele, at least one NtHAP2 mutant allele, and at least one NtHAP3 mutant allele. In an aspect, a modified tobacco plant comprises one NtHAP1b mutant allele, one NtHAP2 mutant allele, and one NtHAP3 mutant allele. In an aspect, a modified tobacco plant comprises two NtHAP1b mutant alleles, two NtHAP2 mutant alleles, and two NtHAP3 mutant alleles.

In an aspect, a modified tobacco plant comprises at least one NtHAP1a mutant allele, at least one NtHAP1b mutant allele, at least one NtHAP2, and at least one NtHAP3 mutant allele. In an aspect, a modified tobacco plant comprises one NtHAP1a mutant allele, one NtHAP2 mutant allele, one NtHAP2 mutant allele, and one NtHAP3 mutant allele. In an aspect, a modified tobacco plant comprises two NtHAP1a mutant alleles, two NtHAP1b mutant alleles, two NtHAP2 mutant alleles and two NtHAP3 mutant alleles.

In an aspect, a modified tobacco plant comprises: (a) one or two NtHAP1a mutant alleles; and (b) one or two NtHAP1b mutant alleles. In an aspect, a modified tobacco plant comprises: (a) one or two NtHAP1a mutant alleles; and (b) one or two NtHAP2 mutant alleles. In an aspect, a modified tobacco plant comprises: (a) one or two NtHAP1a mutant alleles; and (b) one or two NtHAP3 mutant alleles. In an aspect, a modified tobacco plant comprises: (a) one or two NtHAP1b mutant alleles; and (b) one or two NtHAP2 mutant alleles. In an aspect, a modified tobacco plant comprises: (a) one or two NtHAP1b mutant alleles; and (b) one or two NtHAP3 mutant alleles. In an aspect, a modified tobacco plant comprises: (a) one or two NtHAP2 mutant alleles; and (b) one or two NtHAP3 mutant alleles.

In an aspect, a modified tobacco plant comprises: (a) one or two NtHAP1a mutant alleles; (b) one or two NtHAP1b mutant alleles; and (c) one or two NtHAP2 mutant alleles. In an aspect, a modified tobacco plant comprises: (a) one or two NtHAP1a mutant alleles; (b) one or two NtHAP1b mutant alleles; and (c) one or two NtHAP3 mutant alleles. In an aspect, a modified tobacco plant comprises: (a) one or two NtHAP1a mutant alleles; (b) one or two NtHAP2 mutant alleles; and (c) one or two NtHAP3 mutant alleles. In an aspect, a modified tobacco plant comprises: (a) one or two NtHAP1b mutant alleles; (b) one or two NtHAP2 mutant alleles; and (c) one or two NtHAP3 mutant alleles.

In an aspect, a modified tobacco plant comprises: (a) one or two NtHAP1a mutant alleles; (b) one or two NtHAP1b mutant alleles; (c) one or two NtHAP2 mutant alleles; and (d) one or two NtHAP3 mutant alleles.

A “promoter” or “promoter sequence” is a DNA regulatory region capable of binding RNA polymerase and initiating transcription of a polynucleotide or polypeptide coding sequence such as messenger RNA, ribosomal RNA, small nuclear or nucleolar RNA, guide RNA, or any kind of RNA. A promoter can be an endogenous promoter, synthetically produced, varied, or derived from a known or naturally occurring promoter sequence or other promoter sequence. In an aspect, a promoter is a constitutive promoter. In an aspect, a promoter is an inducible promoter. In an aspect, a promoter is a heterologous promoter. In an aspect, a promoter is a tissue-preferred promoter. In an aspect, a promoter is a trichome-preferred promoter. In an aspect, a promoter is selected from the group consisting of a constitutive promoter, an inducible promoter, and a tissue-preferred promoter.

Promoters that drive expression in all or most tissues of the plant are referred to as “constitutive” promoters. In an aspect, a constitutive promoter is selected from the group consisting of a Cauliflower Mosaic Virus 35S promoter, a ubiquitin promoter, an actin promoter, an opine promoter, and an alcohol dehydrogenase promoter.

Promoters that drive enhanced expression in certain tissues of an organism relative to other tissues of the organism are referred to as “tissue-preferred” promoters. Thus, a “tissue-preferred” promoter causes relatively higher or preferential expression in a specific tissue(s) of a plant, but with lower levels of expression in other tissue(s) of the plant. As a non-limiting example, a “trichome-preferred” promoter exhibits higher activity in trichomes than any other plant tissue. Non-limiting examples of trichome-preferred promoters can be found in U.S. Patent Application Publication No. 2022/0243214.

An “inducible” promoter is a promoter that initiates transcription in response to an environmental stimulus such as heat, cold, drought, light, or other stimuli, such as wounding or chemical application.

As used herein, “operably linked” refers to a functional linkage between two or more elements. For example, an operable linkage between a polynucleotide of interest and a regulatory sequence (e.g., a promoter) is a functional link that allows for expression of the polynucleotide of interest. Operably linked elements may be contiguous or non-contiguous. In an aspect, a promoter provided herein is operably linked to a heterologous nucleic acid molecule.

As used herein, “heterologous” refers to a sequence (nucleic acid or amino acid) that originates from a foreign species, or, if from the same species, is substantially modified from its native form in composition and/or genomic locus by deliberate human intervention.

In an aspect, this disclosure provides a modified tobacco plant, or part thereof, comprising a recombinant nucleic acid construct comprising a heterologous promoter operably linked to a polynucleotide that encodes a non-coding RNA molecule, where the non-coding RNA molecule suppresses the transcription or translation of at least one RNA molecule encoding a protein comprising an amino acid sequence at least 90% identical or similar to an amino acid sequence selected from the group consisting of SEQ ID NOs: 1, 2, 3, and 538, where the transcription or translation is as compared to a control tobacco plant lacking the recombinant nucleic acid construct when grown under comparable conditions.

In an aspect, a non-coding RNA molecule suppresses the transcription or translation of at least one RNA molecule encoding a protein comprising an amino acid sequence at least 90% identical to an amino acid sequence selected from the group consisting of SEQ ID NOs: 1, 2, 3, and 538. In an aspect, a non-coding RNA molecule suppresses the transcription or translation of at least one RNA molecule encoding a protein comprising an amino acid sequence at least 95% identical to an amino acid sequence selected from the group consisting of SEQ ID NOs: 1, 2, 3, and 538. In an aspect, a non-coding RNA molecule suppresses the transcription or translation of at least one RNA molecule encoding a protein comprising an amino acid sequence at least 98% identical to an amino acid sequence selected from the group consisting of SEQ ID NOs: 1, 2, 3, and 538. In an aspect, a non-coding RNA molecule suppresses the transcription or translation of at least one RNA molecule encoding a protein comprising an amino acid sequence at least 99% identical to an amino acid sequence selected from the group consisting of SEQ ID NOs: 1, 2, 3, and 538. In an aspect, a non-coding RNA molecule suppresses the transcription or translation of at least one RNA molecule encoding a protein comprising an amino acid sequence 100% identical to an amino acid sequence selected from the group consisting of SEQ ID NOs: 1, 2, 3, and 538.

In an aspect, a non-coding RNA molecule suppresses the transcription or translation of a first RNA molecule encoding a protein comprising an amino acid sequence at least 90% identical to SEQ ID NO: 1 and a second RNA molecule encoding a protein comprising an amino acid sequence at least 90% identical to SEQ ID NO: 2. In an aspect, a non-coding RNA molecule suppresses the transcription or translation of a first RNA molecule encoding a protein comprising an amino acid sequence at least 90% identical to SEQ ID NO: 1 and a second RNA molecule encoding a protein comprising an amino acid sequence at least 90% identical to SEQ ID NO: 3. In an aspect, a non-coding RNA molecule suppresses the transcription or translation of a first RNA molecule encoding a protein comprising an amino acid sequence at least 90% identical to SEQ ID NO: 2 and a second RNA molecule encoding a protein comprising an amino acid sequence at least 90% identical to SEQ ID NO: 3. In an aspect, a non-coding RNA molecule suppresses the transcription or translation of a first RNA molecule encoding a protein comprising an amino acid sequence at least 90% identical to SEQ ID NO: 1 and a second RNA molecule encoding a protein comprising an amino acid sequence at least 90% identical to SEQ ID NO: 538. In an aspect, a non-coding RNA molecule suppresses the transcription or translation of a first RNA molecule encoding a protein comprising an amino acid sequence at least 90% identical to SEQ ID NO: 2 and a second RNA molecule encoding a protein comprising an amino acid sequence at least 90% identical to SEQ ID NO: 538. In an aspect, a non-coding RNA molecule suppresses the transcription or translation of a first RNA molecule encoding a protein comprising an amino acid sequence at least 90% identical to SEQ ID NO: 3 and a second RNA molecule encoding a protein comprising an amino acid sequence at least 90% identical to SEQ ID NO: 538.

In an aspect, a non-coding RNA molecule suppresses the transcription or translation of a first RNA molecule encoding a protein comprising an amino acid sequence at least 90% identical to SEQ ID NO: 1, a second RNA molecule encoding a protein comprising an amino acid sequence at least 90% identical to SEQ ID NO: 2, and a third RNA molecule encoding a protein comprising an amino acid sequence at least 90% identical to SEQ ID NO: 3. In an aspect, a non-coding RNA molecule suppresses the transcription or translation of a first RNA molecule encoding a protein comprising an amino acid sequence at least 90% identical to SEQ ID NO: 1, a second RNA molecule encoding a protein comprising an amino acid sequence at least 90% identical to SEQ ID NO: 2, a third RNA molecule encoding a protein comprising an amino acid sequence at least 90% identical to SEQ ID NO: 3, and a fourth RNA molecule encoding a protein comprising an amino acid sequence at least 90% identical to SEQ ID NO: 538.

In an aspect, a non-coding RNA molecule suppresses the transcription or translation of a first RNA molecule encoding a protein comprising an amino acid sequence at least 95% identical to SEQ ID NO: 1 and a second RNA molecule encoding a protein comprising an amino acid sequence at least 95% identical to SEQ ID NO: 2. In an aspect, a non-coding RNA molecule suppresses the transcription or translation of a first RNA molecule encoding a protein comprising an amino acid sequence at least 95% identical to SEQ ID NO: 1 and a second RNA molecule encoding a protein comprising an amino acid sequence at least 95% identical to SEQ ID NO: 3. In an aspect, a non-coding RNA molecule suppresses the transcription or translation of a first RNA molecule encoding a protein comprising an amino acid sequence at least 95% identical to SEQ ID NO: 2 and a second RNA molecule encoding a protein comprising an amino acid sequence at least 95% identical to SEQ ID NO: 3. In an aspect, a non-coding RNA molecule suppresses the transcription or translation of a first RNA molecule encoding a protein comprising an amino acid sequence at least 95% identical to SEQ ID NO: 1 and a second RNA molecule encoding a protein comprising an amino acid sequence at least 95% identical to SEQ ID NO: 538. In an aspect, a non-coding RNA molecule suppresses the transcription or translation of a first RNA molecule encoding a protein comprising an amino acid sequence at least 95% identical to SEQ ID NO: 2 and a second RNA molecule encoding a protein comprising an amino acid sequence at least 95% identical to SEQ ID NO: 538. In an aspect, a non-coding RNA molecule suppresses the transcription or translation of a first RNA molecule encoding a protein comprising an amino acid sequence at least 95% identical to SEQ ID NO: 3 and a second RNA molecule encoding a protein comprising an amino acid sequence at least 95% identical to SEQ ID NO: 538.

In an aspect, a non-coding RNA molecule suppresses the transcription or translation of a first RNA molecule encoding a protein comprising an amino acid sequence at least 95% identical to SEQ ID NO: 1, a second RNA molecule encoding a protein comprising an amino acid sequence at least 95% identical to SEQ ID NO: 2, and a third RNA molecule encoding a protein comprising an amino acid sequence at least 95% identical to SEQ ID NO: 3. In an aspect, a non-coding RNA molecule suppresses the transcription or translation of a first RNA molecule encoding a protein comprising an amino acid sequence at least 95% identical to SEQ ID NO: 1, a second RNA molecule encoding a protein comprising an amino acid sequence at least 95% identical to SEQ ID NO: 2, a third RNA molecule encoding a protein comprising an amino acid sequence at least 95% identical to SEQ ID NO: 3, and a fourth RNA molecule encoding a protein comprising an amino acid sequence at least 95% identical to SEQ ID NO: 538.

In an aspect, a non-coding RNA molecule suppresses the transcription or translation of a first RNA molecule encoding a protein comprising an amino acid sequence at least 99% identical to SEQ ID NO: 1 and a second RNA molecule encoding a protein comprising an amino acid sequence at least 99% identical to SEQ ID NO: 2. In an aspect, a non-coding RNA molecule suppresses the transcription or translation of a first RNA molecule encoding a protein comprising an amino acid sequence at least 99% identical to SEQ ID NO: 1 and a second RNA molecule encoding a protein comprising an amino acid sequence at least 99% identical to SEQ ID NO: 3. In an aspect, a non-coding RNA molecule suppresses the transcription or translation of a first RNA molecule encoding a protein comprising an amino acid sequence at least 99% identical to SEQ ID NO: 2 and a second RNA molecule encoding a protein comprising an amino acid sequence at least 99% identical to SEQ ID NO: 3. In an aspect, a non-coding RNA molecule suppresses the transcription or translation of a first RNA molecule encoding a protein comprising an amino acid sequence at least 99% identical to SEQ ID NO: 1 and a second RNA molecule encoding a protein comprising an amino acid sequence at least 99% identical to SEQ ID NO: 538. In an aspect, a non-coding RNA molecule suppresses the transcription or translation of a first RNA molecule encoding a protein comprising an amino acid sequence at least 99% identical to SEQ ID NO: 2 and a second RNA molecule encoding a protein comprising an amino acid sequence at least 99% identical to SEQ ID NO: 538. In an aspect, a non-coding RNA molecule suppresses the transcription or translation of a first RNA molecule encoding a protein comprising an amino acid sequence at least 99% identical to SEQ ID NO: 3 and a second RNA molecule encoding a protein comprising an amino acid sequence at least 99% identical to SEQ ID NO: 538.

In an aspect, a non-coding RNA molecule suppresses the transcription or translation of a first RNA molecule encoding a protein comprising an amino acid sequence at least 99% identical to SEQ ID NO: 1, a second RNA molecule encoding a protein comprising an amino acid sequence at least 99% identical to SEQ ID NO: 2, and a third RNA molecule encoding a protein comprising an amino acid sequence at least 99% identical to SEQ ID NO: 3. In an aspect, a non-coding RNA molecule suppresses the transcription or translation of a first RNA molecule encoding a protein comprising an amino acid sequence at least 99% identical to SEQ ID NO: 1, a second RNA molecule encoding a protein comprising an amino acid sequence at least 99% identical to SEQ ID NO: 2, a third RNA molecule encoding a protein comprising an amino acid sequence at least 99% identical to SEQ ID NO: 3, and a fourth RNA molecule encoding a protein comprising an amino acid sequence at least 99% identical to SEQ ID NO: 538.

In an aspect, a non-coding RNA molecule suppresses the transcription or translation of a first RNA molecule encoding a protein comprising an amino acid sequence 100% identical to SEQ ID NO: 1 and a second RNA molecule encoding a protein comprising an amino acid sequence 100% identical to SEQ ID NO: 2. In an aspect, a non-coding RNA molecule suppresses the transcription or translation of a first RNA molecule encoding a protein comprising an amino acid sequence 100% identical to SEQ ID NO: 1 and a second RNA molecule encoding a protein comprising an amino acid sequence 100% identical to SEQ ID NO: 3. In an aspect, a non-coding RNA molecule suppresses the transcription or translation of a first RNA molecule encoding a protein comprising an amino acid sequence 100% identical to SEQ ID NO: 2 and a second RNA molecule encoding a protein comprising an amino acid sequence 100% identical to SEQ ID NO: 3. In an aspect, a non-coding RNA molecule suppresses the transcription or translation of a first RNA molecule encoding a protein comprising an amino acid sequence 100% identical to SEQ ID NO: 1 and a second RNA molecule encoding a protein comprising an amino acid sequence 100% identical to SEQ ID NO: 538. In an aspect, a non-coding RNA molecule suppresses the transcription or translation of a first RNA molecule encoding a protein comprising an amino acid sequence 100% identical to SEQ ID NO: 2 and a second RNA molecule encoding a protein comprising an amino acid sequence 100% identical to SEQ ID NO: 538. In an aspect, a non-coding RNA molecule suppresses the transcription or translation of a first RNA molecule encoding a protein comprising an amino acid sequence 100% identical to SEQ ID NO: 3 and a second RNA molecule encoding a protein comprising an amino acid sequence 100% identical to SEQ ID NO: 538.

In an aspect, a non-coding RNA molecule suppresses the transcription or translation of a first RNA molecule encoding a protein comprising an amino acid sequence 100% identical to SEQ ID NO: 1, a second RNA molecule encoding a protein comprising an amino acid sequence 100% identical to SEQ ID NO: 2, and a third RNA molecule encoding a protein comprising an amino acid sequence 100% identical to SEQ ID NO: 3. In an aspect, a non-coding RNA molecule suppresses the transcription or translation of a first RNA molecule encoding a protein comprising an amino acid sequence 100% identical to SEQ ID NO: 1, a second RNA molecule encoding a protein comprising an amino acid sequence 100% identical to SEQ ID NO: 2, a third RNA molecule encoding a protein comprising an amino acid sequence 100% identical to SEQ ID NO: 3, and a fourth RNA molecule encoding a protein comprising an amino acid sequence 100% identical to SEQ ID NO: 538.

As used herein, a “non-coding RNA molecule” refers to an RNA molecule that is not translated into a protein. Non-limiting examples of non-coding RNA molecules include microRNAs (miRNAs), small interfering RNAs (siRNAs), Piwi-interacting RNAs (piRNAs), small nucleolar RNAs (snoRNAs), extracellular RNAs (exRNAs), guide RNAs (gRNAs), prime editing gRNAs (pegRNAs), ribosomal RNAs (rRNAs), and others. In some aspects, a non-coding RNA molecule is a miRNA. In some aspects, a non-coding RNA molecule is an siRNA. In some aspects, a non-coding RNA molecule is a piRNA. In some aspects, a non-coding RNA molecule is a snoRNA. In some aspects, a non-coding RNA molecule is an exRNA. In some aspects, a non-coding RNA molecule is a gRNA. In some aspects, a non-coding RNA molecule is a pegRNA. In some aspects, a non-coding RNA molecule is an rRNA.

In an aspect, a non-coding RNA molecule is an siRNA molecule. In an aspect, a non-coding RNA molecule is a miRNA molecule. In an aspect, a non-coding RNA molecule is a trans-acting siRNA molecule. In an aspect, a non-coding RNA molecule is a double-stranded RNA molecule. In an aspect, a non-coding RNA molecule is a partially double-stranded RNA molecule.

miRNAs are generally of between about 19 to about 25 nucleotides (commonly about 20-24 nucleotides in plants), that guide cleavage in trans of target transcripts, negatively regulating the expression of genes involved in various regulation and development pathways. In some cases, miRNAs serve to guide in-phase processing of siRNA primary transcripts.

Many microRNA genes (MIR genes) have been identified and made publicly available in a database (“miRBase,” available online at microrna[dot]sanger[dot]ac[dot]uk/sequences; also see Griffiths-Jones et al. (2003) Nucleic Acids Res., 31:439-441). MIR genes have been reported to occur in intergenic regions, both isolated and in clusters in the genome, but can also be located entirely or partially within introns of other genes (both protein-coding and non-protein-coding). For a review of miRNA biogenesis, see Kim (2005) Nature Rev. Mol. Cell. Biol., 6:376-385. Transcription of MIR genes can be, at least in some cases, under promotional control of a MIR gene's own promoter. The primary transcript, termed a “pri-miRNA,” can be quite large (several kilobases) and can be polycistronic, containing one or more pre-miRNAs (fold-back structures containing a stem-loop arrangement that is processed to the mature miRNA) as well as the usual 5′ “cap” and polyadenylated tail of an mRNA. In an aspect, a tobacco plant comprises a pre-miRNA encoding a miRNA that suppresses the expression or accumulation of NtHAP1a, NtHAP1b, NtHAP2, and/or NtHAP3.

Maturation of a mature miRNA from its corresponding precursors (pri-miRNAs and pre-miRNAs) differs significantly between animals and plants. For example, in plant cells, microRNA precursor molecules are believed to be largely processed to the mature miRNA entirely in the nucleus, whereas in animal cells, the pri-miRNA transcript is processed in the nucleus by the animal-specific enzyme Drosha, followed by export of the pre-miRNA to the cytoplasm where it is further processed to the mature miRNA. Mature miRNAs in plants are typically 21 nucleotides in length.

Transgenic expression of miRNAs (whether a naturally occurring sequence or an artificial sequence) can be employed to regulate expression of the miRNA's target gene or genes. Inclusion of a miRNA recognition site in a transgenically expressed transcript is also useful in regulating expression of the transcript. Recognition sites of miRNAs have been validated in all regions of an mRNA, including the 5′ untranslated region, coding region, and 3′ untranslated region, indicating that the position of the miRNA target site relative to the coding sequence may not necessarily affect suppression. Because miRNAs are important regulatory elements in eukaryotes, transgenic suppression of miRNAs is useful for manipulating biological pathways and responses. Finally, promoters of MIR genes can have very specific expression patterns (e.g., cell-specific, tissue-specific, temporally specific, or inducible), and thus are useful in recombinant constructs to induce such specific transcription of a DNA sequence to which they are operably linked. Various utilities of miRNAs, their precursors, their recognition sites, and their promoters are described in detail in U.S. Patent Application Publication 2006/0200878 A1, incorporated by reference herein. Non-limiting examples of these utilities include: (1) the expression of a native miRNA or miRNA precursor sequence to suppress a target gene; (2) the expression of an artificial miRNA or miRNA precursor sequence to suppress a target gene; (3) expression of a transgene with a miRNA recognition site, where the transgene is suppressed when the mature miRNA is expressed; (4) expression of a transgene driven by a miRNA promoter.

Designing an artificial miRNA sequence can be as simple as substituting sequence that is complementary to the intended target for nucleotides in the miRNA stem region of the miRNA precursor, as demonstrated by Zeng et al. (2002) Mol. Cell, 9:1327-1333. One non-limiting example of a general method for determining nucleotide changes in the native miRNA sequence to produce the engineered miRNA precursor includes the following steps: (a) Selecting a unique target sequence of at least 18 nucleotides specific to the target gene, e.g., by using sequence alignment tools such as BLAST (see, for example, Altschul et al. (1990) J. Mol. Biol., 215:403-410; Altschul et al. (1997) Nucleic Acids Res., 25:3389-3402), for example, of both tobacco cDNA and genomic DNA databases, to identify target transcript orthologues and any potential matches to unrelated genes, thereby avoiding unintentional silencing of non-target sequences; (b) Analyzing the target gene for undesirable sequences (e.g., matches to sequences from non-target species), and score each potential 19-mer segment for GC content, Reynolds score (see Reynolds et al. (2004) Nature Biotechnol., 22:326-330), and functional asymmetry characterized by a negative difference in free energy (“DELTA . . . DELTA.G” or “ΔΔG”) (see Khvorova et al. (2003) Cell, 115:209-216). Preferably 19-mers are selected that have all or most of the following characteristics: (1) a Reynolds score>4, (2) a GC content between about 40% to about 60%, (3) a negative ΔΔG, (4) a terminal adenosine, (5) lack of a consecutive run of 4 or more of the same nucleotide; (6) a location near the 3′ terminus of the target gene; (7) minimal differences from the miRNA precursor transcript. Positions at every third nucleotide in an siRNA have been reported to be especially important in influencing RNAi efficacy and an algorithm, “siExplorer” is publicly available at rna[dot]chem[dot]t[dot]u-tokyo[dot]ac[dot]jp/siexplorer.htm (see Katoh and Suzuki (2007) Nucleic Acids Res., 10.1093/nar/gk11120); (c) Determining the reverse complement of the selected 19-mers to use in making a modified mature miRNA. The additional nucleotide at position 20 is preferably matched to the selected target sequence, and the nucleotide at position 21 is preferably chosen to either be unpaired to prevent spreading of silencing on the target transcript or paired to the target sequence to promote spreading of silencing on the target transcript; and (d) transforming the artificial miRNA into a plant.

Without being limited by any scientific theory, it is appreciated in the art that an RNAi knockdown of a candidate gene (e.g., via the use of an artificial miRNA or an siRNA) or a mutation (e.g., missense or nonsense mutations) in the same candidate gene can both cause reduction of expression and/or decreased protein activity and can manifest as identical or similar phenotypes in plants. See, for example, Agrawal et al., Microbiology and Molecular Biology Reviews, 67:657-685 (2003).

In an aspect, a non-coding RNA molecule comprises at least 15 nucleotides. In an aspect, a non-coding RNA molecule comprises at least 16 nucleotides. In an aspect, a non-coding RNA molecule comprises at least 17 nucleotides. In an aspect, a non-coding RNA molecule comprises at least 18 nucleotides. In an aspect, a non-coding RNA molecule comprises at least 19 nucleotides. In an aspect, a non-coding RNA molecule comprises at least 20 nucleotides. In an aspect, a non-coding RNA molecule comprises at least 21 nucleotides. In an aspect, a non-coding RNA molecule comprises at least 22 nucleotides. In an aspect, a non-coding RNA molecule comprises at least 23 nucleotides. In an aspect, a non-coding RNA molecule comprises at least 24 nucleotides. In an aspect, a non-coding RNA molecule comprises at least 25 nucleotides. In an aspect, a non-coding RNA molecule comprises at least 26 nucleotides. In an aspect, a non-coding RNA molecule comprises at least 27 nucleotides. In an aspect, a non-coding RNA molecule comprises at least 28 nucleotides. In an aspect, a non-coding RNA molecule comprises at least 29 nucleotides. In an aspect, a non-coding RNA molecule comprises at least 30 nucleotides. In an aspect, a non-coding RNA molecule comprises at least 40 nucleotides. In an aspect, a non-coding RNA molecule comprises at least 50 nucleotides.

In an aspect, a non-coding RNA molecule comprises between 15 nucleotides and 500 nucleotides. In an aspect, a non-coding RNA molecule comprises between 15 nucleotides and 400 nucleotides. In an aspect, a non-coding RNA molecule comprises between 15 nucleotides and 300 nucleotides. In an aspect, a non-coding RNA molecule comprises between 15 nucleotides and 200 nucleotides. In an aspect, a non-coding RNA molecule comprises between 15 nucleotides and 100 nucleotides. In an aspect, a non-coding RNA molecule comprises between 15 nucleotides and 75 nucleotides. In an aspect, a non-coding RNA molecule comprises between 15 nucleotides and 50 nucleotides. In an aspect, a non-coding RNA molecule comprises between 15 nucleotides and 30 nucleotides. In an aspect, a non-coding RNA molecule comprises between 15 nucleotides and 25 nucleotides. In an aspect, a non-coding RNA molecule comprises between 18 nucleotides and 30 nucleotides. In an aspect, a non-coding RNA molecule comprises between 18 nucleotides and 25 nucleotides. In an aspect, a non-coding RNA molecule comprises between 19 nucleotides and 25 nucleotides. In an aspect, a non-coding RNA molecule comprises between 20 nucleotides and 30 nucleotides. In an aspect, a non-coding RNA molecule comprises between 20 nucleotides and 25 nucleotides. In an aspect, a non-coding RNA molecule comprises between 20 nucleotides and 24 nucleotides.

In an aspect, a non-coding RNA molecule is at least 80% complementary to a nucleic acid sequence selected from the group consisting of SEQ ID NOs: 4 to 9, 537, and 612. In an aspect, a non-coding RNA molecule is at least 85% complementary to a nucleic acid sequence selected from the group consisting of SEQ ID NOs: 4 to 9, 537, and 612. In an aspect, a non-coding RNA molecule is at least 90% complementary to a nucleic acid sequence selected from the group consisting of SEQ ID NOs: 4 to 9, 537, and 612. In an aspect, a non-coding RNA molecule is at least 92.5% complementary to a nucleic acid sequence selected from the group consisting of SEQ ID NOs: 4 to 9, 537, and 612. In an aspect, a non-coding RNA molecule is at least 95% complementary to a nucleic acid sequence selected from the group consisting of SEQ ID NOs: 4 to 9, 537, and 612. In an aspect, a non-coding RNA molecule is at least 97.5% complementary to a nucleic acid sequence selected from the group consisting of SEQ ID NOS: 4 to 9, 537, and 612. In an aspect, a non-coding RNA molecule is at least 99% complementary to a nucleic acid sequence selected from the group consisting of SEQ ID NOs: 4 to 9, 537, and 612. In an aspect, a non-coding RNA molecule is 100% complementary to a nucleic acid sequence selected from the group consisting of SEQ ID NOs: 4 to 9, 537, and 612.

In an aspect, a non-coding RNA sequence comprises no more than 1 mismatch over 21 consecutive nucleotides when compared to any one of SEQ ID NOs: 4 to 9, 537, and 612. In an aspect, a non-coding RNA sequence comprises no more than 2 mismatches over 21 consecutive nucleotides when compared to any one of SEQ ID NOs: 4 to 9, 537, and 612. In an aspect, a non-coding RNA sequence comprises no more than 3 mismatches over 21 consecutive nucleotides when compared to any one of SEQ ID NOs: 4 to 9, 537, and 612. In an aspect, a non-coding RNA sequence comprises no more than 4 mismatches over 21 consecutive nucleotides when compared to any one of SEQ ID NOs: 4 to 9, 537, and 612.

In an aspect, a non-coding RNA sequence comprises no more than 1 mismatch over 20 consecutive nucleotides when compared to any one of SEQ ID NOs: 4 to 9, 537, and 612. In an aspect, a non-coding RNA sequence comprises no more than 2 mismatches over 20 consecutive nucleotides when compared to any one of SEQ ID NOs: 4 to 9, 537, and 612. In an aspect, a non-coding RNA sequence comprises no more than 3 mismatches over 20 consecutive nucleotides when compared to any one of SEQ ID NOs: 4 to 9, 537, and 612. In an aspect, a non-coding RNA sequence comprises no more than 4 mismatches over 20 consecutive nucleotides when compared to any one of SEQ ID NOs: 4 to 9, 537, and 612.

In an aspect, a non-coding RNA sequence comprises no more than 1 mismatch over 19 consecutive nucleotides when compared to any one of SEQ ID NOs: 4 to 9, 537, and 612. In an aspect, a non-coding RNA sequence comprises no more than 2 mismatches over 19 consecutive nucleotides when compared to any one of SEQ ID NOs: 4 to 9, 537, and 612. In an aspect, a non-coding RNA sequence comprises no more than 3 mismatches over 19 consecutive nucleotides when compared to any one of SEQ ID NOs: 4 to 9, 537, and 612. In an aspect, a non-coding RNA sequence comprises no more than 4 mismatches over 19 consecutive nucleotides when compared to any one of SEQ ID NOs: 4 to 9, 537, and 612.

In an aspect, a non-coding RNA sequence comprises no more than 1 mismatch over 18 consecutive nucleotides when compared to any one of SEQ ID NOs: 4 to 9, 537, and 612. In an aspect, a non-coding RNA sequence comprises no more than 2 mismatches over 18 consecutive nucleotides when compared to any one of SEQ ID NOs: 4 to 9, 537, and 612. In an aspect, a non-coding RNA sequence comprises no more than 3 mismatches over 18 consecutive nucleotides when compared to any one of SEQ ID NOs: 4 to 9, 537, and 612. In an aspect, a non-coding RNA sequence comprises no more than 4 mismatches over 18 consecutive nucleotides when compared to any one of SEQ ID NOs: 4 to 9, 537, and 612.

In an aspect, a non-coding RNA molecule is at least 80% complementary to a nucleic acid sequence selected from the group consisting of SEQ ID NOs: 18 and 19. In an aspect, a non-coding RNA molecule is at least 85% complementary to a nucleic acid sequence selected from the group consisting of SEQ ID NOs: 18 and 19. In an aspect, a non-coding RNA molecule is at least 90% complementary to a nucleic acid sequence selected from the group consisting of SEQ ID NOs: 18 and 19. In an aspect, a non-coding RNA molecule is at least 92.5% complementary to a nucleic acid sequence selected from the group consisting of SEQ ID NOs: 18 and 19. In an aspect, a non-coding RNA molecule is at least 95% complementary to a nucleic acid sequence selected from the group consisting of SEQ ID NOs: 18 and 19. In an aspect, a non-coding RNA molecule is at least 97.5% complementary to a nucleic acid sequence selected from the group consisting of SEQ ID NOs: 18 and 19. In an aspect, a non-coding RNA molecule is at least 99% complementary to a nucleic acid sequence selected from the group consisting of SEQ ID NOs: 18 and 19. In an aspect, a non-coding RNA molecule is 100% complementary to a nucleic acid sequence selected from the group consisting of SEQ ID NOs: 18 and 19.

In an aspect, a non-coding RNA sequence comprises no more than 1 mismatch over 21 consecutive nucleotides when compared to any one of SEQ ID NOs: 18 and 19. In an aspect, a non-coding RNA sequence comprises no more than 2 mismatches over 21 consecutive nucleotides when compared to any one of SEQ ID NOs: 18 and 19. In an aspect, a non-coding RNA sequence comprises no more than 3 mismatches over 21 consecutive nucleotides when compared to any one of SEQ ID NOs: 18 and 19. In an aspect, a non-coding RNA sequence comprises no more than 4 mismatches over 21 consecutive nucleotides when compared to any one of SEQ ID NOs: 18 and 19.

In an aspect, a non-coding RNA sequence comprises no more than 1 mismatch over 20 consecutive nucleotides when compared to any one of SEQ ID NOs: 18 and 19. In an aspect, a non-coding RNA sequence comprises no more than 2 mismatches over 20 consecutive nucleotides when compared to any one of SEQ ID NOs: 18 and 19. In an aspect, a non-coding RNA sequence comprises no more than 3 mismatches over 20 consecutive nucleotides when compared to any one of SEQ ID NOs: 18 and 19. In an aspect, a non-coding RNA sequence comprises no more than 4 mismatches over 20 consecutive nucleotides when compared to any one of SEQ ID NOs: 18 and 19.

In an aspect, a non-coding RNA sequence comprises no more than 1 mismatch over 19 consecutive nucleotides when compared to any one of SEQ ID NOs: 18 and 19. In an aspect, a non-coding RNA sequence comprises no more than 2 mismatches over 19 consecutive nucleotides when compared to any one of SEQ ID NOs: 18 and 19. In an aspect, a non-coding RNA sequence comprises no more than 3 mismatches over 19 consecutive nucleotides when compared to any one of SEQ ID NOs: 18 and 19. In an aspect, a non-coding RNA sequence comprises no more than 4 mismatches over 19 consecutive nucleotides when compared to any one of SEQ ID NOs: 18 and 19.

In an aspect, a non-coding RNA sequence comprises no more than 1 mismatch over 18 consecutive nucleotides when compared to any one of SEQ ID NOs: 18 and 19. In an aspect, a non-coding RNA sequence comprises no more than 2 mismatches over 18 consecutive nucleotides when compared to any one of SEQ ID NOs: 18 and 19. In an aspect, a non-coding RNA sequence comprises no more than 3 mismatches over 18 consecutive nucleotides when compared to any one of SEQ ID NOs: 18 and 19. In an aspect, a non-coding RNA sequence comprises no more than 4 mismatches over 18 consecutive nucleotides when compared to any one of SEQ ID NOs: 18 and 19.

Hybridization (e.g., binding of a non-coding RNA molecule to a target nucleic acid molecule) requires that two nucleic acids contain complementary sequences, although mismatches between bases are possible. The conditions appropriate for hybridization between two nucleic acids depend on the length of the nucleic acids and the degree of complementation, variables well known in the art. The greater the degree of complementation between two nucleotide sequences, the greater the value of the melting temperature (Tm) for hybrids of nucleic acids having those sequences. For hybridizations between nucleic acids with short stretches of complementarity (e.g. complementarity over 35 or fewer nucleotides) the position of mismatches becomes important (see Sambrook et al). Typically, the length for a hybridizable nucleic acid is at least about 10 nucleotides. Illustrative minimum lengths for a hybridizable nucleic acid are: at least about 15 nucleotides; at least about 20 nucleotides; at least about 22 nucleotides; at least about 25 nucleotides; and at least about 30 nucleotides). Furthermore, the skilled artisan will recognize that the temperature and wash solution salt concentration may be adjusted as necessary according to factors such as length of the region of complementation and the degree of complementation.

It is understood in the art that the sequence of polynucleotide need not be 100% complementary to that of its target nucleic acid to be specifically hybridizable or hybridizable (e.g., able to bind). Moreover, a polynucleotide may hybridize over one or more segments such that intervening or adjacent segments are not involved in the hybridization (binding) event (e.g., a loop structure or hairpin structure). For example, an antisense nucleic acid in which 18 of 20 nucleotides of the antisense compound are complementary to a target region, and would therefore specifically hybridize, would represent 90 percent complementarity. In this example, the remaining noncomplementary nucleotides may be clustered or interspersed with complementary nucleotides and need not be contiguous to each other or to complementary nucleotides.

Without being limited by any scientific theory, it is appreciated in the art that the binding of a non-coding RNA molecule to a target RNA molecule allows the non-coding RNA molecule to suppress the transcription or translation of the target RNA molecule.

In an aspect, the presence of a non-coding RNA molecule in a modified tobacco plant suppresses transcription or translation of NtHAP1a by at least 1% as compared to a control tobacco plant lacking the non-coding RNA molecule. In an aspect, the presence of a non-coding RNA molecule in a modified tobacco plant suppresses transcription or translation of NtHAP1a by at least 5% as compared to a control tobacco plant lacking the non-coding RNA molecule. In an aspect, the presence of a non-coding RNA molecule in a modified tobacco plant suppresses transcription or translation of NtHAP1a by at least 10% as compared to a control tobacco plant lacking the non-coding RNA molecule. In an aspect, the presence of a non-coding RNA molecule in a modified tobacco plant suppresses transcription or translation of NtHAP1a by at least 20% as compared to a control tobacco plant lacking the non-coding RNA molecule. In an aspect, the presence of a non-coding RNA molecule in a modified tobacco plant suppresses transcription or translation of NtHAP1a by at least 30% as compared to a control tobacco plant lacking the non-coding RNA molecule. In an aspect, the presence of a non-coding RNA molecule in a modified tobacco plant suppresses transcription or translation of NtHAP1a by at least 40% as compared to a control tobacco plant lacking the non-coding RNA molecule. In an aspect, the presence of a non-coding RNA molecule in a modified tobacco plant suppresses transcription or translation of NtHAP1a by at least 50% as compared to a control tobacco plant lacking the non-coding RNA molecule. In an aspect, the presence of a non-coding RNA molecule in a modified tobacco plant suppresses transcription or translation of NtHAP1a by at least 60% as compared to a control tobacco plant lacking the non-coding RNA molecule. In an aspect, the presence of a non-coding RNA molecule in a modified tobacco plant suppresses transcription or translation of NtHAP1a by at least 70% as compared to a control tobacco plant lacking the non-coding RNA molecule. In an aspect, the presence of a non-coding RNA molecule in a modified tobacco plant suppresses transcription or translation of NtHAP1a by at least 80% as compared to a control tobacco plant lacking the non-coding RNA molecule. In an aspect, the presence of a non-coding RNA molecule in a modified tobacco plant suppresses transcription or translation of NtHAP1a by at least 90% as compared to a control tobacco plant lacking the non-coding RNA molecule. In an aspect, the presence of a non-coding RNA molecule in a modified tobacco plant suppresses transcription or translation of NtHAP1a by at least 95% as compared to a control tobacco plant lacking the non-coding RNA molecule. In an aspect, the presence of a non-coding RNA molecule in a modified tobacco plant suppresses transcription or translation of NtHAP1a by at least 99% as compared to a control tobacco plant lacking the non-coding RNA molecule. In an aspect, the presence of a non-coding RNA molecule in a modified tobacco plant suppresses transcription or translation of NtHAP1a by 100% as compared to a control tobacco plant lacking the non-coding RNA molecule.

In an aspect, the presence of a non-coding RNA molecule in a modified tobacco plant suppresses transcription or translation of NtHAP1b by at least 1% as compared to a control tobacco plant lacking the non-coding RNA molecule. In an aspect, the presence of a non-coding RNA molecule in a modified tobacco plant suppresses transcription or translation of NtHAP1b by at least 5% as compared to a control tobacco plant lacking the non-coding RNA molecule. In an aspect, the presence of a non-coding RNA molecule in a modified tobacco plant suppresses transcription or translation of NtHAP1b by at least 10% as compared to a control tobacco plant lacking the non-coding RNA molecule. In an aspect, the presence of a non-coding RNA molecule in a modified tobacco plant suppresses transcription or translation of NtHAP1b by at least 20% as compared to a control tobacco plant lacking the non-coding RNA molecule. In an aspect, the presence of a non-coding RNA molecule in a modified tobacco plant suppresses transcription or translation of NtHAP1b by at least 30% as compared to a control tobacco plant lacking the non-coding RNA molecule. In an aspect, the presence of a non-coding RNA molecule in a modified tobacco plant suppresses transcription or translation of NtHAP1b by at least 40% as compared to a control tobacco plant lacking the non-coding RNA molecule. In an aspect, the presence of a non-coding RNA molecule in a modified tobacco plant suppresses transcription or translation of NtHAP1b by at least 50% as compared to a control tobacco plant lacking the non-coding RNA molecule. In an aspect, the presence of a non-coding RNA molecule in a modified tobacco plant suppresses transcription or translation of NtHAP1b by at least 60% as compared to a control tobacco plant lacking the non-coding RNA molecule. In an aspect, the presence of a non-coding RNA molecule in a modified tobacco plant suppresses transcription or translation of NtHAP1b by at least 70% as compared to a control tobacco plant lacking the non-coding RNA molecule. In an aspect, the presence of a non-coding RNA molecule in a modified tobacco plant suppresses transcription or translation of NtHAP1b by at least 80% as compared to a control tobacco plant lacking the non-coding RNA molecule. In an aspect, the presence of a non-coding RNA molecule in a modified tobacco plant suppresses transcription or translation of NtHAP1b by at least 90% as compared to a control tobacco plant lacking the non-coding RNA molecule. In an aspect, the presence of a non-coding RNA molecule in a modified tobacco plant suppresses transcription or translation of NtHAP1b by at least 95% as compared to a control tobacco plant lacking the non-coding RNA molecule. In an aspect, the presence of a non-coding RNA molecule in a modified tobacco plant suppresses transcription or translation of NtHAP1b by at least 99% as compared to a control tobacco plant lacking the non-coding RNA molecule. In an aspect, the presence of a non-coding RNA molecule in a modified tobacco plant suppresses transcription or translation of NtHAP1b by 100% as compared to a control tobacco plant lacking the non-coding RNA molecule.

In an aspect, the presence of a non-coding RNA molecule in a modified tobacco plant suppresses transcription or translation of NtHAP2 by at least 1% as compared to a control tobacco plant lacking the non-coding RNA molecule. In an aspect, the presence of a non-coding RNA molecule in a modified tobacco plant suppresses transcription or translation of NtHAP2 by at least 5% as compared to a control tobacco plant lacking the non-coding RNA molecule. In an aspect, the presence of a non-coding RNA molecule in a modified tobacco plant suppresses transcription or translation of NtHAP2 by at least 10% as compared to a control tobacco plant lacking the non-coding RNA molecule. In an aspect, the presence of a non-coding RNA molecule in a modified tobacco plant suppresses transcription or translation of NtHAP2 by at least 20% as compared to a control tobacco plant lacking the non-coding RNA molecule. In an aspect, the presence of a non-coding RNA molecule in a modified tobacco plant suppresses transcription or translation of NtHAP2 by at least 30% as compared to a control tobacco plant lacking the non-coding RNA molecule. In an aspect, the presence of a non-coding RNA molecule in a modified tobacco plant suppresses transcription or translation of NtHAP2 by at least 40% as compared to a control tobacco plant lacking the non-coding RNA molecule. In an aspect, the presence of a non-coding RNA molecule in a modified tobacco plant suppresses transcription or translation of NtHAP2 by at least 50% as compared to a control tobacco plant lacking the non-coding RNA molecule. In an aspect, the presence of a non-coding RNA molecule in a modified tobacco plant suppresses transcription or translation of NtHAP2 by at least 60% as compared to a control tobacco plant lacking the non-coding RNA molecule. In an aspect, the presence of a non-coding RNA molecule in a modified tobacco plant suppresses transcription or translation of NtHAP2 by at least 70% as compared to a control tobacco plant lacking the non-coding RNA molecule. In an aspect, the presence of a non-coding RNA molecule in a modified tobacco plant suppresses transcription or translation of NtHAP2 by at least 80% as compared to a control tobacco plant lacking the non-coding RNA molecule. In an aspect, the presence of a non-coding RNA molecule in a modified tobacco plant suppresses transcription or translation of NtHAP2 by at least 90% as compared to a control tobacco plant lacking the non-coding RNA molecule. In an aspect, the presence of a non-coding RNA molecule in a modified tobacco plant suppresses transcription or translation of NtHAP2 by at least 95% as compared to a control tobacco plant lacking the non-coding RNA molecule. In an aspect, the presence of a non-coding RNA molecule in a modified tobacco plant suppresses transcription or translation of NtHAP2 by at least 99% as compared to a control tobacco plant lacking the non-coding RNA molecule. In an aspect, the presence of a non-coding RNA molecule in a modified tobacco plant suppresses transcription or translation of NtHAP2 by 100% as compared to a control tobacco plant lacking the non-coding RNA molecule.

In an aspect, the presence of a non-coding RNA molecule in a modified tobacco plant suppresses transcription or translation of NtHAP3 by at least 1% as compared to a control tobacco plant lacking the non-coding RNA molecule. In an aspect, the presence of a non-coding RNA molecule in a modified tobacco plant suppresses transcription or translation of NtHAP3 by at least 5% as compared to a control tobacco plant lacking the non-coding RNA molecule. In an aspect, the presence of a non-coding RNA molecule in a modified tobacco plant suppresses transcription or translation of NtHAP3 by at least 10% as compared to a control tobacco plant lacking the non-coding RNA molecule. In an aspect, the presence of a non-coding RNA molecule in a modified tobacco plant suppresses transcription or translation of NtHAP3 by at least 20% as compared to a control tobacco plant lacking the non-coding RNA molecule. In an aspect, the presence of a non-coding RNA molecule in a modified tobacco plant suppresses transcription or translation of NtHAP3 by at least 30% as compared to a control tobacco plant lacking the non-coding RNA molecule. In an aspect, the presence of a non-coding RNA molecule in a modified tobacco plant suppresses transcription or translation of NtHAP3 by at least 40% as compared to a control tobacco plant lacking the non-coding RNA molecule. In an aspect, the presence of a non-coding RNA molecule in a modified tobacco plant suppresses transcription or translation of NtHAP3 by at least 50% as compared to a control tobacco plant lacking the non-coding RNA molecule. In an aspect, the presence of a non-coding RNA molecule in a modified tobacco plant suppresses transcription or translation of NtHAP3 by at least 60% as compared to a control tobacco plant lacking the non-coding RNA molecule. In an aspect, the presence of a non-coding RNA molecule in a modified tobacco plant suppresses transcription or translation of NtHAP3 by at least 70% as compared to a control tobacco plant lacking the non-coding RNA molecule. In an aspect, the presence of a non-coding RNA molecule in a modified tobacco plant suppresses transcription or translation of NtHAP3 by at least 80% as compared to a control tobacco plant lacking the non-coding RNA molecule. In an aspect, the presence of a non-coding RNA molecule in a modified tobacco plant suppresses transcription or translation of NtHAP3 by at least 90% as compared to a control tobacco plant lacking the non-coding RNA molecule. In an aspect, the presence of a non-coding RNA molecule in a modified tobacco plant suppresses transcription or translation of NtHAP3 by at least 95% as compared to a control tobacco plant lacking the non-coding RNA molecule. In an aspect, the presence of a non-coding RNA molecule in a modified tobacco plant suppresses transcription or translation of NtHAP3 by at least 99% as compared to a control tobacco plant lacking the non-coding RNA molecule. In an aspect, the presence of a non-coding RNA molecule in a modified tobacco plant suppresses transcription or translation of NtHAP3 by 100% as compared to a control tobacco plant lacking the non-coding RNA molecule.

In an aspect, a modified tobacco plant comprises a transgene comprising a heterologous promoter operably linked to a nucleic acid molecule encoding a polypeptide at least 80% identical or similar to an amino acid sequence selected from the group consisting of SEQ ID NOs: 1 to 3 and 538. In an aspect, a modified tobacco plant comprises a transgene comprising a heterologous promoter operably linked to a nucleic acid molecule encoding a polypeptide at least 85% identical or similar to an amino acid sequence selected from the group consisting of SEQ ID NOs: 1 to 3 and 538. In an aspect, a modified tobacco plant comprises a transgene comprising a heterologous promoter operably linked to a nucleic acid molecule encoding a polypeptide at least 90% identical or similar to an amino acid sequence selected from the group consisting of SEQ ID NOs: 1 to 3 and 538. In an aspect, a modified tobacco plant comprises a transgene comprising a heterologous promoter operably linked to a nucleic acid molecule encoding a polypeptide at least 95% identical or similar to an amino acid sequence selected from the group consisting of SEQ ID NOs: 1 to 3 and 538. In an aspect, a modified tobacco plant comprises a transgene comprising a heterologous promoter operably linked to a nucleic acid molecule encoding a polypeptide at least 97.5% identical or similar to an amino acid sequence selected from the group consisting of SEQ ID NOs: 1 to 3 and 538. In an aspect, a modified tobacco plant comprises a transgene comprising a heterologous promoter operably linked to a nucleic acid molecule encoding a polypeptide at least 99% identical or similar to an amino acid sequence selected from the group consisting of SEQ ID NOs: 1 to 3 and 538. In an aspect, a modified tobacco plant comprises a transgene comprising a heterologous promoter operably linked to a nucleic acid molecule encoding a polypeptide 100% identical or similar to an amino acid sequence selected from the group consisting of SEQ ID NOs: 1 to 3 and 538.

In an aspect, a modified tobacco plant comprises a transgene comprising a heterologous promoter operably linked to a nucleic acid molecule encoding a polypeptide at least 80% identical or similar to an amino acid sequence selected from the group consisting of SEQ ID NOs: 539 to 546. In an aspect, a modified tobacco plant comprises a transgene comprising a heterologous promoter operably linked to a nucleic acid molecule encoding a polypeptide at least 85% identical or similar to an amino acid sequence selected from the group consisting of SEQ ID NOs: 539 to 546. In an aspect, a modified tobacco plant comprises a transgene comprising a heterologous promoter operably linked to a nucleic acid molecule encoding a polypeptide at least 90% identical or similar to an amino acid sequence selected from the group consisting of SEQ ID NOs: 539 to 546. In an aspect, a modified tobacco plant comprises a transgene comprising a heterologous promoter operably linked to a nucleic acid molecule encoding a polypeptide at least 95% identical or similar to an amino acid sequence selected from the group consisting of SEQ ID NOs: 539 to 546. In an aspect, a modified tobacco plant comprises a transgene comprising a heterologous promoter operably linked to a nucleic acid molecule encoding a polypeptide at least 97.5% identical or similar to an amino acid sequence selected from the group consisting of SEQ ID NOs: 539 to 546. In an aspect, a modified tobacco plant comprises a transgene comprising a heterologous promoter operably linked to a nucleic acid molecule encoding a polypeptide at least 99% identical or similar to an amino acid sequence selected from the group consisting of SEQ ID NOs: 539 to 546. In an aspect, a modified tobacco plant comprises a transgene comprising a heterologous promoter operably linked to a nucleic acid molecule encoding a polypeptide 100% identical or similar to an amino acid sequence selected from the group consisting of SEQ ID NOs: 539 to 546.

In an aspect, a modified tobacco plant comprises a transgene comprising a heterologous promoter operably linked to a nucleic acid molecule encoding a polypeptide at least 80% identical or similar to an amino acid sequence selected from the group consisting of SEQ ID NOs: 547 to 589. In an aspect, a modified tobacco plant comprises a transgene comprising a heterologous promoter operably linked to a nucleic acid molecule encoding a polypeptide at least 85% identical or similar to an amino acid sequence selected from the group consisting of SEQ ID NOs: 547 to 589. In an aspect, a modified tobacco plant comprises a transgene comprising a heterologous promoter operably linked to a nucleic acid molecule encoding a polypeptide at least 90% identical or similar to an amino acid sequence selected from the group consisting of SEQ ID NOs: 547 to 589. In an aspect, a modified tobacco plant comprises a transgene comprising a heterologous promoter operably linked to a nucleic acid molecule encoding a polypeptide at least 95% identical or similar to an amino acid sequence selected from the group consisting of SEQ ID NOs: 547 to 589. In an aspect, a modified tobacco plant comprises a transgene comprising a heterologous promoter operably linked to a nucleic acid molecule encoding a polypeptide at least 97.5% identical or similar to an amino acid sequence selected from the group consisting of SEQ ID NOs: 547 to 589. In an aspect, a modified tobacco plant comprises a transgene comprising a heterologous promoter operably linked to a nucleic acid molecule encoding a polypeptide at least 99% identical or similar to an amino acid sequence selected from the group consisting of SEQ ID NOs: 547 to 589. In an aspect, a modified tobacco plant comprises a transgene comprising a heterologous promoter operably linked to a nucleic acid molecule encoding a polypeptide 100% identical or similar to an amino acid sequence selected from the group consisting of SEQ ID NOs: 547 to 589.

In an aspect, a modified tobacco plant comprises an non-natural mutation in an endogenous nucleic acid molecule encoding a HAIRPLUS (HAP) protein, wherein the endogenous nucleic acid molecule encodes an amino acid sequence selected from the group consisting of SEQ ID NOs: 539 to 546, and where expression or activity of the HAP protein is reduced in the modified tobacco plant as compared to a control tobacco plant lacking the non-natural mutation when grown under comparable conditions.

In an aspect, a modified tobacco plant, or part thereof, comprising a recombinant nucleic acid construct comprising a heterologous promoter operably linked to a polynucleotide that encodes a non-coding RNA molecule, wherein the non-coding RNA molecule suppresses the transcription or translation of at least one RNA molecule encoding a protein comprising an amino acid sequence at least 90% identical or similar to an amino acid sequence selected from the group consisting of SEQ ID NOs: 539 to 546, and wherein the transcription or translation is as compared to a control tobacco plant lacking the recombinant nucleic acid construct when grown under comparable conditions. In an aspect, a modified tobacco plant, or part thereof, comprising a recombinant nucleic acid construct comprising a heterologous promoter operably linked to a polynucleotide that encodes a non-coding RNA molecule, wherein the non-coding RNA molecule suppresses the transcription or translation of at least one RNA molecule encoding a protein comprising an amino acid sequence at least 95% identical or similar to an amino acid sequence selected from the group consisting of SEQ ID NOs: 539 to 546, and wherein the transcription or translation is as compared to a control tobacco plant lacking the recombinant nucleic acid construct when grown under comparable conditions. In an aspect, a modified tobacco plant, or part thereof, comprising a recombinant nucleic acid construct comprising a heterologous promoter operably linked to a polynucleotide that encodes a non-coding RNA molecule, wherein the non-coding RNA molecule suppresses the transcription or translation of at least one RNA molecule encoding a protein comprising an amino acid sequence at least 98% identical or similar to an amino acid sequence selected from the group consisting of SEQ ID NOs: 539 to 546, and wherein the transcription or translation is as compared to a control tobacco plant lacking the recombinant nucleic acid construct when grown under comparable conditions. In an aspect, a modified tobacco plant, or part thereof, comprising a recombinant nucleic acid construct comprising a heterologous promoter operably linked to a polynucleotide that encodes a non-coding RNA molecule, wherein the non-coding RNA molecule suppresses the transcription or translation of at least one RNA molecule encoding a protein comprising an amino acid sequence 100% identical or similar to an amino acid sequence selected from the group consisting of SEQ ID NOs: 539 to 546, and wherein the transcription or translation is as compared to a control tobacco plant lacking the recombinant nucleic acid construct when grown under comparable conditions.

In an aspect, a modified tobacco plant comprising a transgene described in this paragraph exhibits increased expression of the nucleic acid molecule and/or increased accumulation of the polypeptide as compared to a control tobacco plant lacking the transgene when grown under comparable conditions.

In an aspect, a modified tobacco plant comprises a transgene comprising a heterologous promoter operably linked to a nucleic acid molecule comprising a nucleic acid sequence at least 80% identical to a nucleic acid sequence selected from the group consisting of SEQ ID NOs: 4 to 9, 537, and 612. In an aspect, a modified tobacco plant comprises a transgene comprising a heterologous promoter operably linked to a nucleic acid molecule comprising a nucleic acid sequence at least 85% identical to a nucleic acid sequence selected from the group consisting of SEQ ID NOs: 4 to 9, 537, and 612. In an aspect, a modified tobacco plant comprises a transgene comprising a heterologous promoter operably linked to a nucleic acid molecule comprising a nucleic acid sequence at least 90% identical to a nucleic acid sequence selected from the group consisting of SEQ ID NOs: 4 to 9, 537, and 612. In an aspect, a modified tobacco plant comprises a transgene comprising a heterologous promoter operably linked to a nucleic acid molecule comprising a nucleic acid sequence at least 95% identical to a nucleic acid sequence selected from the group consisting of SEQ ID NOs: 4 to 9, 537, and 612. In an aspect, a modified tobacco plant comprises a transgene comprising a heterologous promoter operably linked to a nucleic acid molecule comprising a nucleic acid sequence at least 97.5% identical to a nucleic acid sequence selected from the group consisting of SEQ ID NOs: 4 to 9, 537, and 612. In an aspect, a modified tobacco plant comprises a transgene comprising a heterologous promoter operably linked to a nucleic acid molecule comprising a nucleic acid sequence at least 99% identical to a nucleic acid sequence selected from the group consisting of SEQ ID NOs: 4 to 9, 537, and 612. In an aspect, a modified tobacco plant comprises a transgene comprising a heterologous promoter operably linked to a nucleic acid molecule comprising a nucleic acid sequence 100% identical to a nucleic acid sequence selected from the group consisting of SEQ ID NOs: 4 to 9, 537, and 612. In an aspect, a modified tobacco plant comprising a transgene described in this paragraph exhibits increased expression of the nucleic acid molecule as compared to a control tobacco plant lacking the transgene when grown under comparable conditions.

In an aspect, this disclosure provides a progeny tobacco plant derived from a modified tobacco plant provided herein. As used herein, a “progeny plant” or “progeny seed” can be from any filial generation, e.g., F₁, F₂, F₃, F₄, F₅, F₆, F₇, etc.

In an aspect, a tobacco plant, seed, or plant part, is of a tobacco variety selected from the group consisting of a flue-cured variety, a bright variety, a Burley variety, a Virginia variety, a Maryland variety, a dark variety, a Galpão variety, an Oriental variety, and a Turkish variety.

In an aspect, a tobacco cell is of a tobacco variety selected from the group consisting of a flue cured variety, a bright variety, a Burley variety, a Virginia variety, a Maryland variety, a dark variety, a Galpão variety, an Oriental variety, and a Turkish variety.

In an aspect, a tobacco leaf is of a tobacco variety selected from the group consisting of a flue cured variety, a bright variety, a Burley variety, a Virginia variety, a Maryland variety, a dark variety, a Galpão variety, an Oriental variety, and a Turkish variety.

In an aspect, a cured tobacco leaf or plant part is of a tobacco variety selected from the group consisting of a flue cured variety, a bright variety, a Burley variety, a Virginia variety, a Maryland variety, a dark variety, a Galpão variety, an Oriental variety, and a Turkish variety. Skilled artisans further understand that cured tobacco does not constitute a living organism and is not capable of growth or reproduction. However, it is appreciated in the art that nucleic acid molecules can be extracted from cured tobacco material, which allows one to identify the genetic background of the cured tobacco material. See, for example, Biswas et al., Int J Anal Chem., 2016:4352308 (2016).

Flue-cured tobaccos (also called “Virginia” or “bright” tobaccos) amount to approximately 40% of world tobacco production. Flue-cured tobaccos are often also referred to as “bright tobacco” because of the golden-yellow to deep-orange color it reaches during curing. Flue-cured tobaccos have a light, bright aroma and taste. Flue-cured tobaccos are generally high in sugar and low in oils. Major flue-cured tobacco growing countries are Argentina, Brazil, China, India, Tanzania and the United States of America. In one aspect, tobacco plants, seeds, or plant parts provided herein are of a flue-cured tobacco variety selected from the group consisting of the varieties listed in Table 2, and any variety essentially derived from any one of the foregoing varieties. See WO 2004/041006 A1. In a further aspect, tobacco plants, seeds, or plant parts provided herein are in a flue-cured variety selected from the group consisting of K326, K346, and NC196.

TABLE 2
Flue-cured Tobacco Varieties

| | | |
|---|---|---|
| 400 (TC 225) | K 346 | Reams 134 |
| 401 (TC 226) | K 346 (TC 569) | Reams 158 |
| 401 Cherry Red (TC 227) | K 358 | Reams 713 |
| 401 Cherry Red Free (TC 228) | K 394 (TC 321) | Reams 744 |
| Cash (TC 250) | K 399 | Reams M1 |
| Cash (TI 278) | K 399 (TC 322) | RG 11 (TC 600) |
| CC 101 | K 730 | RG 13 (TC 601) |
| CC 1063 | Lonibow (TI 1573) | RG 17 (TC 627) |
| CC 13 | Lonibow (TI 1613) | RG 22 (TC 584) |
| CC 143 | McNair 10 (TC 330) | RG 8 (TC 585) |
| CC 200 | McNair 135 (TC 337) | RG 81 (TC 618) |
| CC 27 | McNair 30 (TC 334) | RG H51 |
| CC 301 | McNair 373 (TC 338) | RG4H 217 |
| CC 33 | McNair 944 (TC 339) | RGH 12 |
| CC 35 | MK94 (TI 1512) | RGH 4 |
| CC 37 | MS K 326 | RGH 51 |
| CC 400 | MS NC 71 | RGH 61 |
| CC 500 | MS NC 72 | SC 58 (TC 400) |
| CC 600 | NC 100 | SC 72 (TC 403) |
| CC 65 | NC 102 | Sp. G-168 |
| CC 67 | NC 1071 (TC 364) | SPEIGHT 168 |
| CC 700 | NC 1125-2 | Speight 168 (TC 633) |
| CC 800 | NC 12 (TC 346) | Speight 172 (TC 634) |
| CC 900 | NC 1226 | Speight 178 |
| Coker 139 (TC 259) | NC 196 | Speight 179 |
| Coker 139 ybl, yb2 | NC 2326 (TC 365) | Speight 190 |
| Coker 140 (TC 260) | NC 27 NF (TC 349) | Speight 196 |
| Coker 176 (TC 262) | NC 291 | SPEIGHT 220 |
| Coker 187 (TC 263) | NC 297 | SPEIGHT 225 |
| Coker 187-Hicks (TC 265) | NC 299 | SPEIGHT 227 |
| Coker 209 (TC 267) | NC 37 NF (TC 350) | SPEIGHT 236 |
| Coker 258 (TC 270) | NC 471 | Speight G-10 (TC 416) |
| Coker 298 (TC 272) | NC 55 | Speight G-102 |
| Coker 316 (TC 273) | NC 567 (TC 362) | Speight G-108 |
| Coker 319 (TC 274) | NC 60 (TC 352) | Speight G-111 |
| Coker 347 (TC 275) | NC 606 | Speight G-117 |
| Coker 371-Gold (TC 276) | NC 6140 | Speight G-126 |
| Coker 411 (TC 277) | NC 71 | Speight G-15 (TC 418) |
| Coker 48 (TC 253) | NC 72 | Speight G-23 |
| Coker 51 (TC 254) | NC 729 (TC 557) | Speight G-28 (TC 420) |
| Coker 86 (TC 256) | NC 810 (TC 659) | Speight G-33 |
| CU 263 (TC 619) | NC 82 (TC 356) | Speight G-41 |
| CU 561 | NC 8640 | Speight G-5 |
| DH95-1562-1 | NC 89 (TC 359) | Speight G-52 |
| Dixie Bright 101 (TC 290) | NC 92 | Speight G-58 |
| Dixie Bright 102 (TC 291) | NC 925 | Speight G-70 |
| Dixie Bright 244 (TC 292) | NC 95 (TC 360) | Speight G-70 (TC 426) |
| Dixie Bright 27 (TC 288) | NC 98 (TC 361) | Speight G-80 (TC 427) |
| Dixie Bright 28 (TC 289) | NC EX 24 | Speight NF3 (TC 629) |
| GF 157 | NC PY 10 (TC 367) | STNCB |
| GF 318 | NC TG 61 | VA 182 |
| GL 26H | Oxford 1 (TC 369) | VA 45 (TC 559) |
| GL 338 | Oxford 1-181 (TC 370) | Vesta 30 (TC 439) |
| GL 350 | Oxford 2 (TC 371) | Vesta 33 (TC 440) |
| GL 368 | Oxford 207 (TC 632) | Vesta 5 (TC 438) |
| GL 395 | Oxford 26 (TC 373) | Vesta 62 (TC 441) |
| GL 600 | Oxford 3 (TC 372) | Virginia (TI 220) |
| GL 737 | Oxford 414 NF | Virginia (TI 273) |
| GL 939 | PD 611 (TC 387) | Virginia (TI 877) |
| GL 939 (TC 628) | PVH 03 | Virginia 115 (TC 444) |
| Hicks (TC 310) | PVH 09 | Virginia 21 (TC 443) |
| Hicks Broadleaf (TC 311) | PVH 1118 | Virginia Bright (TI 964) |
| K 149 (TC 568) | PVH 1452 | Virginia Bright Leaf (TC 446) |
| K 317 | PVH 1600 | Virginia Gold (TC 447) |
| K 326 | PVH 2110 | White Stem Orinoco (TC 451) |
| K 326 (TC 319) | PVH 2275 | |
| K 340 (TC 320) | R 83 (Line 256-1) (TI 1400) | |

Air-cured tobaccos include “Burley,” “Maryland,” and “dark” tobaccos. The common factor linking air-cured tobaccos is that curing occurs primarily without artificial sources of heat and humidity. Burley tobaccos are light to dark brown in color, high in oil, and low in sugar. Burley tobaccos are typically air-cured in barns. Major Burley growing countries include Argentina, Brazil, Italy, Malawi, and the United States of America. Maryland tobaccos are extremely fluffy, have good burning properties, low nicotine and a neutral aroma. Major Maryland growing countries include the United States of America and Italy.

In one aspect, tobacco plants, seeds, or plant parts provided herein are of a Burley tobacco variety selected from the group consisting of the tobacco varieties listed in Table 3, and any variety essentially derived from any one of the foregoing varieties. In a further aspect, tobacco plants, seeds, or plant parts provided herein are in a Burley variety selected from the group consisting of TN 90, KT 209, KT 206, KT212, and HB 4488.

TABLE 3
Burley Tobacco Varieties

| | | |
|---|---|---|
| 4407 LC | HB 4108P | KY 54 (TC 71) |
| AA-37-1 | HB 4151P | KY 56 (TC 72) |
| Burley 21 (TC 7) | HB 4192P | KY 56 (TC 72) |
| Burley 49 (TC 10) | HB 4194P | KY 57 (TC 73) |
| Burley 64 (TC 11) | HB 4196 | KY 58 (TC 74) |
| Burley Mammoth KY 16 (TC 12) | HB 4488 | KY 8654 (TC 77) |
| Clay 402 | HB 4488P | KY 8959 |
| Clay 403 | HB04P | KY 9 (TC 54) |
| Clay 502 | HB 4488 LC | KY 907 LC |
| Clays 403 | HIB 21 | KY 908 (TC 630) |
| GR 10 (TC 19) | HPB 21 | NBH 98 (Screened) |
| GR 10 (TC 19) | HY 403 | NC 1206 |
| GR 10A (TC 20) | Hybrid 403 LC | NC 129 |
| GR 13 (TC 21) | Hybrid 404 LC | NC 2000 LC |
| GR 14 (TC 22) | Hybrid 501 LC | NC 2002 LC |
| GR 149 LC | KDH-959 (TC 576) | NC 3 LC |
| GR 153 | KDH-960 (TC 577) | NC 5 LC |
| GR 17 (TC 23) | KT 200 LC | NC 6 LC |
| GR 17B (TC 24) | KT 204 LC | NC 7 LC |
| GR 18 (TC 25) | KT 206 LC | NC BH 129 LC |
| GR 19 (TC 26) | KT 209 LC | NC03-42-2 |
| GR 2 (TC 15) | KT 210 LC | Newton 98 |
| GR 24 (TC 27) | KT 212 LC | R 610 LC |
| GR 36 (TC 28) | KT 215 LC | R 630 LC |
| GR 38 (TC 29) | KY 1 (TC 52) | R 7-11 |
| GR 38A (TC 30) | KY 10 (TC 55) | R 7-12 LC |
| GR 40 (TC 31) | KY 12 (TC 56) | RG 17 |
| GR 42 (TC 32) | KY 14 (TC 57) | TKF 1801 LC |
| GR 42C (TC 33) | KY 14 x L8 LC | TKF 2002 LC |
| GR 43 (TC 34) | KY 15 (TC 58) | TKF 4024 LC |
| GR 44 (TC 35) | KY 16 (TC 59) | TKF 4028 LC |
| GR 45 (TC 36) | KY 17 (TC 60) | TKF 6400 LC |
| GR 46 (TC 37) | KY 19 (TC 61) | TKF 7002 LC |
| GR 48 (TC 38) | KY 21 (TC 62) | TKS 2002 LC |
| GR 5 (TC 16) | KY 22 (TC 63) | TN 86 (TC 82) |
| GR 53 (TC 39) | KY 24 (TC 64) | TN 90 LC |
| GR 6 (TC 17) | KY 26 (TC 65) | TN 97 Hybrid LC |
| GR 9 (TC 18) | KY 33 (TC 66) | TN 97 LC |
| GR139 NS | KY 34 (TC 67) | VA 116 |
| GR139 S | KY 35 (TC 68) | VA 119 |
| HB 04P | KY 41A (TC 69) | Virgin A Mutante (TI 1406) |
| HB 04P LC | KY 5 (TC 53) | Virginia 509 (TC 84) |
| HB 3307P LC | KY 52 (TC 70) | |

In another aspect, tobacco plants, seeds, or plant parts provided herein are of a Maryland tobacco variety selected from the group consisting of the tobacco varieties listed in Table 4, and any variety essentially derived from any one of the foregoing varieties.

TABLE 4
Maryland Tobacco Varieties

Maryland 10 (TC 498)
Maryland 14 D2 (TC 499)
Maryland 201 (TC 503)
Maryland 21 (TC 500)
Maryland 341 (TC 504)
Maryland 40
Maryland 402
Maryland 59 (TC 501)
Maryland 601
Maryland 609 (TC 505)
Maryland 64 (TC 502)
Maryland 872 (TC 506)
Maryland Mammoth (TC 507)

Dark air-cured tobaccos are distinguished from other tobacco types primarily by its curing process, which gives dark air-cured tobacco its medium-brown to dark-brown color and a distinct aroma. Dark air-cured tobaccos are mainly used in the production of chewing tobacco and snuff. In one aspect, tobacco plants, seeds, or plant parts provided herein are of a dark air-cured tobacco variety selected from the group consisting of Sumatra, Jatim, Dominican Cubano, Besuki, One sucker, Green River, Virginia sun-cured, and Paraguayan Passado, and any variety essentially derived from any one of the foregoing varieties.

Dark fire-cured tobaccos are generally cured with low-burning wood fires on the floors of closed curing barns. Dark fire-cured tobaccos are typically used for making pipe blends, cigarettes, chewing tobacco, snuff, and strong-tasting cigars. Major growing regions for dark fire-cured tobaccos are Tennessee, Kentucky, and Virginia in the United States of America. In one aspect, tobacco plants, seeds, or plant parts provided herein are of a dark fire-cured tobacco variety selected from the group consisting of the tobacco varieties listed in Table 5, and any variety essentially derived from any one of the foregoing varieties.

TABLE 5
Dark Fire-Cured Tobacco Varieties

| | | |
|---|---|---|
| Black Mammoth (TC 461) | KY 171 (TC 475) | PD 7309 LC |
| Black Mammoth Small Stalk (TC 641) | KY 171 LC | PD 7312 LC |
| Certified Madole (TC 463) | KY 171 NS | PD 7318 LC |
| D-534-A-1 (TC 464) | KY 180 (TC 573) | PD 7319 LC |
| DAC ULT 302 | KY 190 (TC 574) | Petico M PG04 |
| DAC ULT 303 | Little Crittenden | PY KY 160 (TC 612) |
| DAC ULT 306 | Little Crittenden (TC 476) | PY KY 171 (TC 613) |
| DAC ULT 308 | Little Crittenden LC | Shirey |
| | (certified) | |
| DAC ULT 312 | Little Crittenden PhPh | TI 1372 |
| DF 300 (TC 465) | Lizard Tail Turtle Foot | TN D94 |
| DF 485 (TC 466) | Madole (TC 478) | TN D94 (TC 621) |
| DF 516 (TC 467) | Madole (TC 479) | TN D950 |
| DF 911 (TC 468) | MS KY 171 | TN D950 (PhPh) |
| DT 508 | MS NL Madole LC | TN D950 |
| DT 518 (Screened) | MS TN D950 LC | TN D950 (TC 622) |
| DT 538 LC | Nance (TC 616) | TR Madole (TC 486) |
| DT 592 | Narrow Leaf Madole LC | VA 309 |
| | (certified) | |
| Improved Madole (TC 471) | Neal Smith Madole (TC 646) | VA 309 (TC 560) |
| Jernigan's Madole (TC 472) | Newtons VH Madole | VA 309 LC (certified) |
| KT 14LC | NL Madole | VA 310 (TC 487) |
| KT D17LC | NL Madole (PhPh) | VA 331 (TC 592) |
| KT D4 LC | NL Madole (TC 484) | VA 355 (TC 638) |
| KT D6 LC | NL Madole LC | VA 359 |
| KT D8 LC | NL Madole LC (PhPh) | VA 359 (Screened) |
| KY 153 (TC 216) | NL Madole NS | VA 359 (TC 639) |
| KY 157 (TC 217) | One Sucker (TC 224) | VA 359 LC (certified) |
| KY 160 | OS 400 | VA 403 (TC 580) |
| KY 160 (TC 218) | PD 302H | VA 405 (TC 581) |
| KY 163 (TC 219) | PD 312H | VA 409 (TC 562) |
| KY 165 (TC 220) | PD 318H | VA 510 (TC 572) |
| KY 170 (TC 474) | PD 7302 LC | |
| KY 171 (PhPh) | PD 7305 | |

Oriental tobaccos are also referred to as Greek, aroma and Turkish tobaccos due to the fact that they are typically grown in eastern Mediterranean regions such as Turkey, Greece, Bulgaria, Macedonia, Syria, Lebanon, Italy, and Romania. The small plant size, small leaf size, and unique aroma properties of Oriental tobacco varieties are a result of their adaptation to the poor soil and stressful climatic conditions in which they have been developed. In one aspect, tobacco plants, seeds, or plant parts provided herein are of an Oriental tobacco variety selected from the group consisting of the tobacco varieties listed in Table 6, and any variety essentially derived from any one of the foregoing varieties.

TABLE 6
Oriental Tobacco Varieties

| | | |
|---|---|---|
| Bafra (TI 1641) | Edirne (TI 1671) | Samsun (TC 536) |
| Bahce (TI 1730) | Ege (TI 1642) | Samsun 959 (TI 1570) |
| Bahia (TI 1416) | Ege-64 (TI 1672) | Samsun Evkaf (TI 1723) |
| Bahia (TI 1455) | Izmir (Akhisar) (TI 1729) | Samsun Holmes NN (TC 540) |
| Baiano (TI 128) | Izmir (Gavurkoy) (TI 1727) | Samsun Maden (TI 1647) |
| Basma | Izmir Ege 64 | Samsun NO 15 (TC 541) |
| Basma (TI 1666) | Izmir-Incekara (TI 1674) | Samsun-BLK SHK Tol (TC 542) |
| Basma Drama | Izmir-Ozbas (TI 1675) | Samsun-Canik (TI 1678) |
| Basma Hybrid (PhPh) | Jaka Dzebel (TI 1326) | Samsun-Maden (TI 1679) |
| Basma Zihna I | Kaba-Kulak | Saribaptar 407 - Izmir Region |
| Bitlis (TI 1667) | Kagoshima Maruba (TI 158) | Smyrna (TC 543) |
| Bitlis (TI 1725) | Katerini | Smyrna No. 23 (TC 545) |
| Bubalovac (TI 1282) | Katerini S53 | Smyrna No. 9 (TC 544) |
| Bursa (TI 1650) | Krumovgrad 58 | Smyrna-Blk Shk Tol (TC 546) |
| Bursa (TI 1668) | MS Basma | Trabzon (TI 1649) |
| Canik (TI 1644) | MS Katerini S53 | Trabzon (TI 1682) |
| Djebel 174 (TI 1492) | Nevrokop 1146 | Trapezund 161 (TI 1407) |
| Djebel 359 (TI 1493) | Ozbas (TI 1645) | Turkish (TC 548) |
| Djebel 81 | Perustitza (TI 980) | Turkish Angshit (TI 90) |
| Dubec 566 (TI 1409) | Prilep (TI 1291) | Turkish Samsum (TI 92) |
| Dubec 7 (TI 1410) | Prilep (TI 1325) | Turkish Tropizoid (TI 93) |
| Dubek 566 (TI 1567) | Prilep 12-2/1 | Turkish Varotic (TI 89) |
| Duzce (TI 1670) | Prilep 23 | Xanthi (TI 1662) |

In an aspect, tobacco plants, seeds, or plant parts provided herein are of a cigar tobacco variety selected from the group consisting of the tobacco varieties listed in Table 7, and any variety essentially derived from any one of the foregoing varieties.

TABLE 7
Cigar Tobacco Varieties

| | | |
|---|---|---|
| Bahai (TI 62) | Castillo Negro, Blanco, Pina (TI 449) | Enshu (TI 1586) |
| Beinhart 1000 | Caujaro (TI 893) | Florida 301 |
| Beinhart 1000 (TI 1562) | Chocoa (TI 289) | Florida 301 (TC 195) |
| Beinhart 1000-1 (TI 1561) | Chocoa (TI 313) | PA Broadleaf (TC 119) |
| Bergerac C | Connecticut 15 (TC 183) | Pennsylvania Broadleaf |
| Bergerac C (TI 1529) | Connecticut Broadleaf | Pennsylvania Broadleaf (TC 119) |
| Big Cuban (TI 1565) | Connecticut Broadleaf (TC 186) | Petite Havana SR1 |
| Castillo Negro, Blanco, | Connecticut Shade (TC 188) | Petite Havana SR1 (TC 105) |
| Pina (TI 448) | | |
| Castillo Negro, Blanco, | Criollo, Colorado (TI 1093) | |
| Pina (TI 448A) | | |

In an aspect, tobacco plants, seeds, or plant parts provided herein are of a tobacco variety selected from the group consisting of the tobacco varieties listed in Table 8, and any variety essentially derived from any one of the foregoing varieties.

TABLE 8
Other Tobacco Varieties

Chocoa (TI 319)
Hoja Parada (TI 1089)
Hoja Parado (Galpão) (TI 1068)
Perique (St. James Parrish)
Perique (TC 556)
Perique (TI 1374)
Sylvestris (TI 984)
TI 179

In an aspect, a tobacco plant or plant part is from a variety selected from the group consisting of the tobacco varieties listed in Table 2, Table 3, Table 4, Table 5, Table 6, Table 7, and Table 8. In another aspect, a tobacco plant or plant part is from a variety listed in Table 2. In another aspect, a tobacco plant or plant part is from a variety listed in Table 3. In another aspect, a tobacco plant or plant part is from a variety listed in Table 4. In another aspect, a tobacco plant or plant part is from a variety listed in Table 5. In another aspect, a tobacco plant or plant part is from a variety listed in Table 6. In another aspect, a tobacco plant or plant part is from a variety listed in Table 7. In another aspect, a tobacco plant or plant part is from a variety listed in Table 8.

In an aspect, a tobacco seed is from a variety selected from the group consisting of the tobacco varieties listed in Table 2, Table 3, Table 4, Table 5, Table 6, Table 7, and Table 8. In another aspect, a tobacco seed is from a variety listed in Table 2. In another aspect, a tobacco seed is from a variety listed in Table 3. In another aspect, a tobacco seed is from a variety listed in Table 4. In another aspect, a tobacco seed is from a variety listed in Table 5. In another aspect, a tobacco seed is from a variety listed in Table 6. In another aspect, a tobacco seed is from a variety listed in Table 7. In another aspect, a tobacco seed is from a variety listed in Table 8.

In an aspect, a tobacco cell is from a variety selected from the group consisting of the tobacco varieties listed in Table 2, Table 3, Table 4, Table 5, Table 6, Table 7, and Table 8. In another aspect, a tobacco cell is from a variety listed in Table 2. In another aspect, a tobacco cell is from a variety listed in Table 3. In another aspect, a tobacco cell is from a variety listed in Table 4. In another aspect, a tobacco cell is from a variety listed in Table 5. In another aspect, a tobacco cell is from a variety listed in Table 6. In another aspect, a tobacco cell is from a variety listed in Table 7. In another aspect, a tobacco cell is from a variety listed in Table 8.

All foregoing mentioned specific varieties of flue-cured, dark air-cured, Burley, Maryland, dark fire-cured, cigar, or Oriental type are listed only for exemplary purposes. Any additional flue-cured, dark air-cured, Burley, Maryland, dark fire-cured, cigar, or Oriental varieties are also contemplated in the present application.

In an aspect, a plant or variety provided herein is an inbred plant or variety. As used herein, an “inbred” variety is a variety that has been bred for genetic homogeneity.

In an aspect, a modified tobacco plant provided herein is an inbred tobacco plant. In an aspect, a modified tobacco seed provided herein is an inbred tobacco seed.

As used herein, a “hybrid” is created by crossing two plants from different varieties or species, such that the progeny comprises genetic material from each parent. Skilled artisans recognize that higher order hybrids can be generated as well. For example, a first hybrid can be made by crossing Variety C with Variety D to create a C×D hybrid, and a second hybrid can be made by crossing Variety E with Variety F to create an Ex F hybrid. The first and second hybrids can be further crossed to create the higher order hybrid (C×D)×(E×F) comprising genetic information from all four parent varieties. In an aspect, a modified tobacco plant is a hybrid tobacco plant. In an aspect, a modified tobacco seed is a hybrid tobacco seed.

In an aspect, a modified tobacco plant is a male sterile tobacco plant. In an aspect, a modified tobacco plant is a cytoplasmic male sterile (CMS) tobacco plant. Male sterile plants can be produced by any method known in the art. Methods of producing male sterile tobacco are described in Wernsman, E. A., and Rufty, R. C. 1987. Chapter Seventeen. Tobacco. Pages 669-698 in: Cultivar Development. Crop Species. W. H. Fehr (ed.), MacMillan Publishing Go., Inc., New York, N.Y. 761 pp.

In an aspect, a modified tobacco plant provided herein is used to pollinate a male sterile tobacco plant. In an aspect, a modified tobacco plant provided herein is used to pollinate a CMS tobacco plant.

In another aspect, a modified tobacco plant is female sterile. As a non-limiting example, female sterile plants can be made by mutating the STIG1 gene. See, for example, Goldman et al. 1994, EMBO Journal 13:2976-2984.

In an aspect, a modified tobacco plant provided herein is pollinated by a female sterile tobacco plant.

As used herein, the term “crossing” refers to the deliberate mating of two plants. In an aspect, crossing comprises pollination and/or fertilization of a first tobacco plant by a second tobacco plant. The two tobacco plants being crossed can be distantly related, closely related, or identical. In an aspect, the two tobacco plants being crossed are both modified tobacco plants. In an aspect, the two tobacco plants being crossed are of the same tobacco variety. In an aspect, the two tobacco plants being crossed are of two different tobacco varieties.

In an aspect, this disclosure provides a method for producing a tobacco plant, the method comprising: (a) crossing a modified tobacco plant with a second tobacco plant to produce at least one progeny tobacco seed, where the modified tobacco plant comprises at least one non-natural mutation in at least one endogenous nucleic acid molecule encoding an amino acid sequence selected from the group consisting of SEQ ID NOs: 1, 2, 3, and 538; and (b) selecting at least one progeny tobacco seed, or a tobacco plant germinated therefrom, comprising the at least one non-natural mutation. In an aspect, the second tobacco plant comprises the at least one non-natural mutation. In an aspect, the modified tobacco plant comprises at least one non-natural mutation in a first endogenous nucleic acid molecule encoding the amino acid sequence of SEQ ID NO: 1 and at least one non-natural mutation in a second endogenous nucleic acid molecule encoding the amino acid sequence of SEQ ID NO: 2. In an aspect, the modified tobacco plant comprises at least one non-natural mutation in a first endogenous nucleic acid molecule encoding the amino acid sequence of SEQ ID NO: 1 and at least one non-natural mutation in a second endogenous nucleic acid molecule encoding the amino acid sequence of SEQ ID NO: 3. In an aspect, the modified tobacco plant comprises at least one non-natural mutation in a first endogenous nucleic acid molecule encoding the amino acid sequence of SEQ ID NO: 1 and at least one non-natural mutation in a second endogenous nucleic acid molecule encoding the amino acid sequence of SEQ ID NO: 538. In an aspect, the modified tobacco plant comprises at least one non-natural mutation in a first endogenous nucleic acid molecule encoding the amino acid sequence of SEQ ID NO: 2 and at least one non-natural mutation in a second endogenous nucleic acid molecule encoding the amino acid sequence of SEQ ID NO: 3. In an aspect, the modified tobacco plant comprises at least one non-natural mutation in a first endogenous nucleic acid molecule encoding the amino acid sequence of SEQ ID NO: 2 and at least one non-natural mutation in a second endogenous nucleic acid molecule encoding the amino acid sequence of SEQ ID NO: 538. In an aspect, the modified tobacco plant comprises at least one non-natural mutation in a first endogenous nucleic acid molecule encoding the amino acid sequence of SEQ ID NO: 3 and at least one non-natural mutation in a second endogenous nucleic acid molecule encoding the amino acid sequence of SEQ ID NO: 538. In an aspect, the modified tobacco plant comprises at least one non-natural mutation in a first endogenous nucleic acid molecule encoding the amino acid sequence of SEQ ID NO: 1, at least one non-natural mutation in a second endogenous nucleic acid molecule encoding the amino acid sequence of SEQ ID NO: 2, and at least one non-natural mutation in a third endogenous nucleic acid molecule encoding the amino acid sequence of SEQ ID NO: 3. In an aspect, the modified tobacco plant comprises at least one non-natural mutation in a first endogenous nucleic acid molecule encoding the amino acid sequence of SEQ ID NO: 1, at least one non-natural mutation in a second endogenous nucleic acid molecule encoding the amino acid sequence of SEQ ID NO: 2, and at least one non-natural mutation in a third endogenous nucleic acid molecule encoding the amino acid sequence of SEQ ID NO: 583. In an aspect, the modified tobacco plant comprises at least one non-natural mutation in a first endogenous nucleic acid molecule encoding the amino acid sequence of SEQ ID NO: 1, at least one non-natural mutation in a second endogenous nucleic acid molecule encoding the amino acid sequence of SEQ ID NO: 3, and at least one non-natural mutation in a third endogenous nucleic acid molecule encoding the amino acid sequence of SEQ ID NO: 538. In an aspect, the modified tobacco plant comprises at least one non-natural mutation in a first endogenous nucleic acid molecule encoding the amino acid sequence of SEQ ID NO: 2, at least one non-natural mutation in a second endogenous nucleic acid molecule encoding the amino acid sequence of SEQ ID NO: 3, and at least one non-natural mutation in a third endogenous nucleic acid molecule encoding the amino acid sequence of SEQ ID NO: 538. In an aspect, the modified tobacco plant comprises at least one non-natural mutation in a first endogenous nucleic acid molecule encoding the amino acid sequence of SEQ ID NO: 1, at least one non-natural mutation in a second endogenous nucleic acid molecule encoding the amino acid sequence of SEQ ID NO: 2, at least one non-natural mutation in a third endogenous nucleic acid molecule encoding the amino acid sequence of SEQ ID NO: 3, and at least one non-natural mutation in a fourth endogenous nucleic acid molecule encoding the amino acid sequence of SEQ ID NO: 538.

In an aspect, this disclosure provides a method for producing a tobacco plant, the method comprising: (a) crossing a modified tobacco plant with a second tobacco plant to produce at least one progeny tobacco seed, where the modified tobacco plant comprises at least one non-natural mutation in at least one endogenous nucleic acid molecule encoding an amino acid sequence selected from the group consisting of SEQ ID NOs: 539 to 546; and (b) selecting at least one progeny tobacco seed, or a tobacco plant germinated therefrom, comprising the at least one non-natural mutation.

In an aspect, this disclosure provides a method for producing a tobacco plant, the method comprising: (a) crossing a modified tobacco plant with a second tobacco plant to produce at least one progeny tobacco seed, where the modified tobacco plant comprises at least one non-natural mutation in at least one endogenous nucleic acid molecule comprising a nucleic acid sequence selected from the group consisting of SEQ ID NOs: 4, 5, 6, and 612; and (b) selecting at least one progeny tobacco seed, or a tobacco plant germinated therefrom, comprising the at least one non-natural mutation. In an aspect, the second tobacco plant comprises the at least one non-natural mutation. In an aspect, the modified tobacco plant comprises at least one non-natural mutation in a first endogenous nucleic acid molecule comprising the nucleic acid sequence of SEQ ID NO: 4 and at least one non-natural mutation in a second endogenous nucleic acid molecule comprising the nucleic acid sequence of SEQ ID NO: 5. In an aspect, the modified tobacco plant comprises at least one non-natural mutation in a first endogenous nucleic acid molecule comprising the nucleic acid sequence of SEQ ID NO: 4 and at least one non-natural mutation in a second endogenous nucleic acid molecule comprising the nucleic acid sequence of SEQ ID NO: 6. In an aspect, the modified tobacco plant comprises at least one non-natural mutation in a first endogenous nucleic acid molecule comprising the nucleic acid sequence of SEQ ID NO: 5 and at least one non-natural mutation in a second endogenous nucleic acid molecule comprising the nucleic acid sequence of SEQ ID NO: 6. In an aspect, the modified tobacco plant comprises at least one non-natural mutation in a first endogenous nucleic acid molecule comprising the nucleic acid sequence of SEQ ID NO: 4 and at least one non-natural mutation in a second endogenous nucleic acid molecule comprising the nucleic acid sequence of SEQ ID NO: 612. In an aspect, the modified tobacco plant comprises at least one non-natural mutation in a first endogenous nucleic acid molecule comprising the nucleic acid sequence of SEQ ID NO: 5 and at least one non-natural mutation in a second endogenous nucleic acid molecule comprising the nucleic acid sequence of SEQ ID NO: 612. In an aspect, the modified tobacco plant comprises at least one non-natural mutation in a first endogenous nucleic acid molecule comprising the nucleic acid sequence of SEQ ID NO: 6 and at least one non-natural mutation in a second endogenous nucleic acid molecule comprising the nucleic acid sequence of SEQ ID NO: 612. In an aspect, the modified tobacco plant comprises at least one non-natural mutation in a first endogenous nucleic acid molecule comprising the nucleic acid sequence of SEQ ID NO: 4, at least one non-natural mutation in a second endogenous nucleic acid molecule comprising the nucleic acid sequence of SEQ ID NO: 5, and at least one non-natural mutation in a third endogenous nucleic acid molecule comprising the nucleic acid sequence of SEQ ID NO: 6. In an aspect, the modified tobacco plant comprises at least one non-natural mutation in a first endogenous nucleic acid molecule comprising the nucleic acid sequence of SEQ ID NO: 4, at least one non-natural mutation in a second endogenous nucleic acid molecule comprising the nucleic acid sequence of SEQ ID NO: 5, and at least one non-natural mutation in a third endogenous nucleic acid molecule comprising the nucleic acid sequence of SEQ ID NO: 612. In an aspect, the modified tobacco plant comprises at least one non-natural mutation in a first endogenous nucleic acid molecule comprising the nucleic acid sequence of SEQ ID NO: 4, at least one non-natural mutation in a second endogenous nucleic acid molecule comprising the nucleic acid sequence of SEQ ID NO: 6, and at least one non-natural mutation in a third endogenous nucleic acid molecule comprising the nucleic acid sequence of SEQ ID NO: 612. In an aspect, the modified tobacco plant comprises at least one non-natural mutation in a first endogenous nucleic acid molecule comprising the nucleic acid sequence of SEQ ID NO: 6, at least one non-natural mutation in a second endogenous nucleic acid molecule comprising the nucleic acid sequence of SEQ ID NO: 6, and at least one non-natural mutation in a third endogenous nucleic acid molecule comprising the nucleic acid sequence of SEQ ID NO: 612. In an aspect, the modified tobacco plant comprises at least one non-natural mutation in a first endogenous nucleic acid molecule comprising the nucleic acid sequence of SEQ ID NO: 4, at least one non-natural mutation in a second endogenous nucleic acid molecule comprising the nucleic acid sequence of SEQ ID NO: 5, at least one non-natural mutation in a third endogenous nucleic acid molecule comprising the nucleic acid sequence of SEQ ID NO: 6, and at least one non-natural mutation in a fourth endogenous nucleic acid molecule comprising the nucleic acid sequence of SEQ ID NO: 612.

In an aspect, this disclosure provides a method for producing a tobacco plant, the method comprising: (a) crossing a modified tobacco plant with a second tobacco plant to produce at least one progeny tobacco seed, where the modified tobacco plant comprises at least one non-natural mutation in at least one endogenous nucleic acid molecule comprising a nucleic acid sequence selected from the group consisting of SEQ ID NOs: 7, 8, 9, and 537; and (b) selecting at least one progeny tobacco seed, or a tobacco plant germinated therefrom, comprising the at least one non-natural mutation. In an aspect, the second tobacco plant comprises the at least one non-natural mutation. In an aspect, the modified tobacco plant comprises at least one non-natural mutation in a first endogenous nucleic acid molecule comprising the nucleic acid sequence of SEQ ID NO: 7 and at least one non-natural mutation in a second endogenous nucleic acid molecule comprising the nucleic acid sequence of SEQ ID NO: 8. In an aspect, the modified tobacco plant comprises at least one non-natural mutation in a first endogenous nucleic acid molecule comprising the nucleic acid sequence of SEQ ID NO: 7 and at least one non-natural mutation in a second endogenous nucleic acid molecule comprising the nucleic acid sequence of SEQ ID NO: 9. In an aspect, the modified tobacco plant comprises at least one non-natural mutation in a first endogenous nucleic acid molecule comprising the nucleic acid sequence of SEQ ID NO: 8 and at least one non-natural mutation in a second endogenous nucleic acid molecule comprising the nucleic acid sequence of SEQ ID NO: 9. In an aspect, the modified tobacco plant comprises at least one non-natural mutation in a first endogenous nucleic acid molecule comprising the nucleic acid sequence of SEQ ID NO: 7 and at least one non-natural mutation in a second endogenous nucleic acid molecule comprising the nucleic acid sequence of SEQ ID NO: 537. In an aspect, the modified tobacco plant comprises at least one non-natural mutation in a first endogenous nucleic acid molecule comprising the nucleic acid sequence of SEQ ID NO: 8 and at least one non-natural mutation in a second endogenous nucleic acid molecule comprising the nucleic acid sequence of SEQ ID NO: 537. In an aspect, the modified tobacco plant comprises at least one non-natural mutation in a first endogenous nucleic acid molecule comprising the nucleic acid sequence of SEQ ID NO: 9 and at least one non-natural mutation in a second endogenous nucleic acid molecule comprising the nucleic acid sequence of SEQ ID NO: 537. In an aspect, the modified tobacco plant comprises at least one non-natural mutation in a first endogenous nucleic acid molecule comprising the nucleic acid sequence of SEQ ID NO: 7, at least one non-natural mutation in a second endogenous nucleic acid molecule comprising the nucleic acid sequence of SEQ ID NO: 8, and at least one non-natural mutation in a third endogenous nucleic acid molecule comprising the nucleic acid sequence of SEQ ID NO: 9. In an aspect, the modified tobacco plant comprises at least one non-natural mutation in a first endogenous nucleic acid molecule comprising the nucleic acid sequence of SEQ ID NO: 7, at least one non-natural mutation in a second endogenous nucleic acid molecule comprising the nucleic acid sequence of SEQ ID NO: 8, and at least one non-natural mutation in a third endogenous nucleic acid molecule comprising the nucleic acid sequence of SEQ ID NO: 537. In an aspect, the modified tobacco plant comprises at least one non-natural mutation in a first endogenous nucleic acid molecule comprising the nucleic acid sequence of SEQ ID NO: 7, at least one non-natural mutation in a second endogenous nucleic acid molecule comprising the nucleic acid sequence of SEQ ID NO: 9, and at least one non-natural mutation in a third endogenous nucleic acid molecule comprising the nucleic acid sequence of SEQ ID NO: 537. In an aspect, the modified tobacco plant comprises at least one non-natural mutation in a first endogenous nucleic acid molecule comprising the nucleic acid sequence of SEQ ID NO: 8, at least one non-natural mutation in a second endogenous nucleic acid molecule comprising the nucleic acid sequence of SEQ ID NO: 9, and at least one non-natural mutation in a third endogenous nucleic acid molecule comprising the nucleic acid sequence of SEQ ID NO: 537. In an aspect, the modified tobacco plant comprises at least one non-natural mutation in a first endogenous nucleic acid molecule comprising the nucleic acid sequence of SEQ ID NO: 7, at least one non-natural mutation in a second endogenous nucleic acid molecule comprising the nucleic acid sequence of SEQ ID NO: 8, at least one non-natural mutation in a third endogenous nucleic acid molecule comprising the nucleic acid sequence of SEQ ID NO: 9, and at least one non-natural mutation in a fourth endogenous nucleic acid molecule comprising the nucleic acid sequence of SEQ ID NO: 537.

In an aspect, this disclosure provides a method for producing a tobacco plant, the method comprising: (a) crossing a first tobacco plant with a second tobacco plant to produce at least one progeny tobacco seed, where the first tobacco plant comprises a recombinant nucleic acid construct comprising a heterologous promoter operably linked to a polynucleotide that encodes a non-coding RNA molecule that binds to and suppresses the transcription or translation of at least one RNA molecule encoding an amino acid sequence at least 90% identical or similar to an amino acid sequence selected from the group consisting of SEQ ID NOs: 1, 2, 3, and 538; and (b) selecting at least one progeny tobacco seed, or a tobacco plant germinated therefrom, comprising the recombinant nucleic acid construct. In an aspect, the non-coding RNA molecule suppresses the transcription of a first RNA molecule encoding an amino acid sequence at least 90% identical or similar to SEQ ID NO: 1 and a second RNA molecule encoding an amino acid sequence at least 90% identical or similar to SEQ ID NO: 2. In an aspect, the non-coding RNA molecule suppresses the transcription of a first RNA molecule encoding an amino acid sequence at least 90% identical or similar to SEQ ID NO: 1 and a second RNA molecule encoding an amino acid sequence at least 90% identical or similar to SEQ ID NO: 3. In an aspect, the non-coding RNA molecule suppresses the transcription of a first RNA molecule encoding an amino acid sequence at least 90% identical or similar to SEQ ID NO: 2 and a second RNA molecule encoding an amino acid sequence at least 90% identical or similar to SEQ ID NO: 3. In an aspect, the non-coding RNA molecule suppresses the transcription of a first RNA molecule encoding an amino acid sequence at least 90% identical or similar to SEQ ID NO: 1 and a second RNA molecule encoding an amino acid sequence at least 90% identical or similar to SEQ ID NO: 538. In an aspect, the non-coding RNA molecule suppresses the transcription of a first RNA molecule encoding an amino acid sequence at least 90% identical or similar to SEQ ID NO: 2 and a second RNA molecule encoding an amino acid sequence at least 90% identical or similar to SEQ ID NO: 538. In an aspect, the non-coding RNA molecule suppresses the transcription of a first RNA molecule encoding an amino acid sequence at least 90% identical or similar to SEQ ID NO: 3 and a second RNA molecule encoding an amino acid sequence at least 90% identical or similar to SEQ ID NO: 538. In an aspect, the non-coding RNA molecule suppresses the transcription of a first RNA molecule encoding an amino acid sequence at least 90% identical or similar to SEQ ID NO: 1, a second RNA molecule encoding an amino acid sequence at least 90% identical or similar to SEQ ID NO: 2, and a third RNA molecule encoding an amino acid sequence at least 90% identical or similar to SEQ ID NO: 3. In an aspect, the non-coding RNA molecule suppresses the transcription of a first RNA molecule encoding an amino acid sequence at least 90% identical or similar to SEQ ID NO: 1, a second RNA molecule encoding an amino acid sequence at least 90% identical or similar to SEQ ID NO: 2, and a third RNA molecule encoding an amino acid sequence at least 90% identical or similar to SEQ ID NO: 538. In an aspect, the non-coding RNA molecule suppresses the transcription of a first RNA molecule encoding an amino acid sequence at least 90% identical or similar to SEQ ID NO: 1, a second RNA molecule encoding an amino acid sequence at least 90% identical or similar to SEQ ID NO: 3, and a third RNA molecule encoding an amino acid sequence at least 90% identical or similar to SEQ ID NO: 538. In an aspect, the non-coding RNA molecule suppresses the transcription of a first RNA molecule encoding an amino acid sequence at least 90% identical or similar to SEQ ID NO: 2, a second RNA molecule encoding an amino acid sequence at least 90% identical or similar to SEQ ID NO: 3, and a third RNA molecule encoding an amino acid sequence at least 90% identical or similar to SEQ ID NO: 538. In an aspect, the non-coding RNA molecule suppresses the transcription of a first RNA molecule encoding an amino acid sequence at least 90% identical or similar to SEQ ID NO: 1, a second RNA molecule encoding an amino acid sequence at least 90% identical or similar to SEQ ID NO: 2, a third RNA molecule encoding an amino acid sequence at least 90% identical or similar to SEQ ID NO: 3, and a fourth RNA molecule encoding an amino acid sequence at least 90% identical or similar to SEQ ID NO: 538. In an aspect, the non-coding RNA molecule suppresses the transcription of a first RNA molecule encoding an amino acid sequence at least 95% identical or similar to SEQ ID NO: 1 and a second RNA molecule encoding an amino acid sequence at least 95% identical or similar to SEQ ID NO: 2. In an aspect, the non-coding RNA molecule suppresses the transcription of a first RNA molecule encoding an amino acid sequence at least 95% identical or similar to SEQ ID NO: 1 and a second RNA molecule encoding an amino acid sequence at least 95% identical or similar to SEQ ID NO: 3. In an aspect, the non-coding RNA molecule suppresses the transcription of a first RNA molecule encoding an amino acid sequence at least 95% identical or similar to SEQ ID NO: 2 and a second RNA molecule encoding an amino acid sequence at least 95% identical or similar to SEQ ID NO: 3. In an aspect, the non-coding RNA molecule suppresses the transcription of a first RNA molecule encoding an amino acid sequence at least 95% identical or similar to SEQ ID NO: 1 and a second RNA molecule encoding an amino acid sequence at least 95% identical or similar to SEQ ID NO: 538. In an aspect, the non-coding RNA molecule suppresses the transcription of a first RNA molecule encoding an amino acid sequence at least 95% identical or similar to SEQ ID NO: 2 and a second RNA molecule encoding an amino acid sequence at least 95% identical or similar to SEQ ID NO: 538. In an aspect, the non-coding RNA molecule suppresses the transcription of a first RNA molecule encoding an amino acid sequence at least 95% identical or similar to SEQ ID NO: 3 and a second RNA molecule encoding an amino acid sequence at least 95% identical or similar to SEQ ID NO: 538. In an aspect, the non-coding RNA molecule suppresses the transcription of a first RNA molecule encoding an amino acid sequence at least 95% identical or similar to SEQ ID NO: 1, a second RNA molecule encoding an amino acid sequence at least 95% identical or similar to SEQ ID NO: 2, and a third RNA molecule encoding an amino acid sequence at least 95% identical or similar to SEQ ID NO: 3. In an aspect, the non-coding RNA molecule suppresses the transcription of a first RNA molecule encoding an amino acid sequence at least 95% identical or similar to SEQ ID NO: 1, a second RNA molecule encoding an amino acid sequence at least 95% identical or similar to SEQ ID NO: 2, and a third RNA molecule encoding an amino acid sequence at least 95% identical or similar to SEQ ID NO: 538. In an aspect, the non-coding RNA molecule suppresses the transcription of a first RNA molecule encoding an amino acid sequence at least 95% identical or similar to SEQ ID NO: 1, a second RNA molecule encoding an amino acid sequence at least 95% identical or similar to SEQ ID NO: 3, and a third RNA molecule encoding an amino acid sequence at least 95% identical or similar to SEQ ID NO: 538. In an aspect, the non-coding RNA molecule suppresses the transcription of a first RNA molecule encoding an amino acid sequence at least 95% identical or similar to SEQ ID NO: 2, a second RNA molecule encoding an amino acid sequence at least 95% identical or similar to SEQ ID NO: 3, and a third RNA molecule encoding an amino acid sequence at least 95% identical or similar to SEQ ID NO: 538. In an aspect, the non-coding RNA molecule suppresses the transcription of a first RNA molecule encoding an amino acid sequence at least 95% identical or similar to SEQ ID NO: 1, a second RNA molecule encoding an amino acid sequence at least 95% identical or similar to SEQ ID NO: 2, a third RNA molecule encoding an amino acid sequence at least 95% identical or similar to SEQ ID NO: 3, and a fourth RNA molecule encoding an amino acid sequence at least 95% identical or similar to SEQ ID NO: 538. In an aspect, the non-coding RNA molecule suppresses the transcription of a first RNA molecule encoding an amino acid sequence 100% identical or similar to SEQ ID NO: 1 and a second RNA molecule encoding an amino acid sequence 100% identical or similar to SEQ ID NO: 2. In an aspect, the non-coding RNA molecule suppresses the transcription of a first RNA molecule encoding an amino acid sequence 100% identical or similar to SEQ ID NO: 1 and a second RNA molecule encoding an amino acid sequence 100% identical or similar to SEQ ID NO: 3. In an aspect, the non-coding RNA molecule suppresses the transcription of a first RNA molecule encoding an amino acid sequence 100% identical or similar to SEQ ID NO: 2 and a second RNA molecule encoding an amino acid sequence 100% identical or similar to SEQ ID NO: 3. In an aspect, the non-coding RNA molecule suppresses the transcription of a first RNA molecule encoding an amino acid sequence 100% identical or similar to SEQ ID NO: 1 and a second RNA molecule encoding an amino acid sequence 100% identical or similar to SEQ ID NO: 538. In an aspect, the non-coding RNA molecule suppresses the transcription of a first RNA molecule encoding an amino acid sequence 100% identical or similar to SEQ ID NO: 2 and a second RNA molecule encoding an amino acid sequence 100% identical or similar to SEQ ID NO: 538. In an aspect, the non-coding RNA molecule suppresses the transcription of a first RNA molecule encoding an amino acid sequence 100% identical or similar to SEQ ID NO: 3 and a second RNA molecule encoding an amino acid sequence 100% identical or similar to SEQ ID NO: 538. In an aspect, the non-coding RNA molecule suppresses the transcription of a first RNA molecule encoding an amino acid sequence 100% identical or similar to SEQ ID NO: 1, a second RNA molecule encoding an amino acid sequence 100% identical or similar to SEQ ID NO: 2, and a third RNA molecule encoding an amino acid sequence 100% identical or similar to SEQ ID NO: 3. In an aspect, the non-coding RNA molecule suppresses the transcription of a first RNA molecule encoding an amino acid sequence 100% identical or similar to SEQ ID NO: 1, a second RNA molecule encoding an amino acid sequence 100% identical or similar to SEQ ID NO: 2, and a third RNA molecule encoding an amino acid sequence 100% identical or similar to SEQ ID NO: 538. In an aspect, the non-coding RNA molecule suppresses the transcription of a first RNA molecule encoding an amino acid sequence 100% identical or similar to SEQ ID NO: 1, a second RNA molecule encoding an amino acid sequence 100% identical or similar to SEQ ID NO: 3, and a third RNA molecule encoding an amino acid sequence 100% identical or similar to SEQ ID NO: 538. In an aspect, the non-coding RNA molecule suppresses the transcription of a first RNA molecule encoding an amino acid sequence 100% identical or similar to SEQ ID NO: 2, a second RNA molecule encoding an amino acid sequence 100% identical or similar to SEQ ID NO: 3, and a third RNA molecule encoding an amino acid sequence 100% identical or similar to SEQ ID NO: 538. In an aspect, the non-coding RNA molecule suppresses the transcription of a first RNA molecule encoding an amino acid sequence 100% identical or similar to SEQ ID NO: 1, a second RNA molecule encoding an amino acid sequence 100% identical or similar to SEQ ID NO: 2, a third RNA molecule encoding an amino acid sequence 100% identical or similar to SEQ ID NO: 3, and a fourth RNA molecule encoding an amino acid sequence 100% identical or similar to SEQ ID NO: 538.

In an aspect, this disclosure provides a method for producing a tobacco plant, the method comprising: (a) crossing a first tobacco plant with a second tobacco plant to produce at least one progeny tobacco seed, where the first tobacco plant comprises a recombinant nucleic acid construct comprising a heterologous promoter operably linked to a polynucleotide that encodes a non-coding RNA molecule that binds to and suppresses the transcription or translation of at least one RNA molecule comprising a nucleic acid sequence at least 90% identical to a nucleic acid sequence selected from the group consisting of SEQ ID NOs: 4, 5, 6, and 612; and (b) selecting at least one progeny tobacco seed, or a tobacco plant germinated therefrom, comprising the recombinant nucleic acid construct. In an aspect, the non-coding RNA molecule suppresses the transcription or translation of a first RNA molecule comprising a nucleic acid sequence at least 90% identical to SEQ ID NO: 4 and a second RNA molecule comprising a nucleic acid sequence at least 90% identical to SEQ ID NO: 5. In an aspect, the non-coding RNA molecule suppresses the transcription or translation of a first RNA molecule comprising a nucleic acid sequence at least 90% identical to SEQ ID NO: 4 and a second RNA molecule comprising a nucleic acid sequence at least 90% identical to SEQ ID NO: 6. In an aspect, the non-coding RNA molecule suppresses the transcription or translation of a first RNA molecule comprising a nucleic acid sequence at least 90% identical to SEQ ID NO: 5 and a second RNA molecule comprising a nucleic acid sequence at least 90% identical to SEQ ID NO: 6. In an aspect, the non-coding RNA molecule suppresses the transcription or translation of a first RNA molecule comprising a nucleic acid sequence at least 90% identical to SEQ ID NO: 4 and a second RNA molecule comprising a nucleic acid sequence at least 90% identical to SEQ ID NO: 612. In an aspect, the non-coding RNA molecule suppresses the transcription or translation of a first RNA molecule comprising a nucleic acid sequence at least 90% identical to SEQ ID NO: 5 and a second RNA molecule comprising a nucleic acid sequence at least 90% identical to SEQ ID NO: 612. In an aspect, the non-coding RNA molecule suppresses the transcription or translation of a first RNA molecule comprising a nucleic acid sequence at least 90% identical to SEQ ID NO: 6 and a second RNA molecule comprising a nucleic acid sequence at least 90% identical to SEQ ID NO: 612. In an aspect, the non-coding RNA molecule suppresses the transcription or translation of a first RNA molecule comprising a nucleic acid sequence at least 90% identical to SEQ ID NO: 4, a second RNA molecule comprising a nucleic acid sequence at least 90% identical to SEQ ID NO: 5, and a third RNA molecule comprising a nucleic acid sequence at least 90% identical to SEQ ID NO: 6. In an aspect, the non-coding RNA molecule suppresses the transcription or translation of a first RNA molecule comprising a nucleic acid sequence at least 90% identical to SEQ ID NO: 4, a second RNA molecule comprising a nucleic acid sequence at least 90% identical to SEQ ID NO: 5, and a third RNA molecule comprising a nucleic acid sequence at least 90% identical to SEQ ID NO: 612. In an aspect, the non-coding RNA molecule suppresses the transcription or translation of a first RNA molecule comprising a nucleic acid sequence at least 90% identical to SEQ ID NO: 4, a second RNA molecule comprising a nucleic acid sequence at least 90% identical to SEQ ID NO: 6, and a third RNA molecule comprising a nucleic acid sequence at least 90% identical to SEQ ID NO: 612. In an aspect, the non-coding RNA molecule suppresses the transcription or translation of a first RNA molecule comprising a nucleic acid sequence at least 90% identical to SEQ ID NO: 5, a second RNA molecule comprising a nucleic acid sequence at least 90% identical to SEQ ID NO: 6, and a third RNA molecule comprising a nucleic acid sequence at least 90% identical to SEQ ID NO: 612. In an aspect, the non-coding RNA molecule suppresses the transcription or translation of a first RNA molecule comprising a nucleic acid sequence at least 90% identical to SEQ ID NO: 4, a second RNA molecule comprising a nucleic acid sequence at least 90% identical to SEQ ID NO: 5, a third RNA molecule comprising a nucleic acid sequence at least 90% identical to SEQ ID NO: 6, and a fourth RNA molecule comprising a nucleic acid sequence at least 90% identical to SEQ ID NO: 612. In an aspect, the non-coding RNA molecule suppresses the transcription or translation of a first RNA molecule comprising a nucleic acid sequence at least 95% identical to SEQ ID NO: 4 and a second RNA molecule comprising a nucleic acid sequence at least 95% identical to SEQ ID NO: 5. In an aspect, the non-coding RNA molecule suppresses the transcription or translation of a first RNA molecule comprising a nucleic acid sequence at least 95% identical to SEQ ID NO: 4 and a second RNA molecule comprising a nucleic acid sequence at least 95% identical to SEQ ID NO: 6. In an aspect, the non-coding RNA molecule suppresses the transcription or translation of a first RNA molecule comprising a nucleic acid sequence at least 95% identical to SEQ ID NO: 5 and a second RNA molecule comprising a nucleic acid sequence at least 95% identical to SEQ ID NO: 6. In an aspect, the non-coding RNA molecule suppresses the transcription or translation of a first RNA molecule comprising a nucleic acid sequence at least 95% identical to SEQ ID NO: 4 and a second RNA molecule comprising a nucleic acid sequence at least 95% identical to SEQ ID NO: 612. In an aspect, the non-coding RNA molecule suppresses the transcription or translation of a first RNA molecule comprising a nucleic acid sequence at least 95% identical to SEQ ID NO: 5 and a second RNA molecule comprising a nucleic acid sequence at least 95% identical to SEQ ID NO: 612. In an aspect, the non-coding RNA molecule suppresses the transcription or translation of a first RNA molecule comprising a nucleic acid sequence at least 95% identical to SEQ ID NO: 6 and a second RNA molecule comprising a nucleic acid sequence at least 95% identical to SEQ ID NO: 612. In an aspect, the non-coding RNA molecule suppresses the transcription or translation of a first RNA molecule comprising a nucleic acid sequence at least 95% identical to SEQ ID NO: 4, a second RNA molecule comprising a nucleic acid sequence at least 95% identical to SEQ ID NO: 5, and a third RNA molecule comprising a nucleic acid sequence at least 95% identical to SEQ ID NO: 6. In an aspect, the non-coding RNA molecule suppresses the transcription or translation of a first RNA molecule comprising a nucleic acid sequence at least 95% identical to SEQ ID NO: 4, a second RNA molecule comprising a nucleic acid sequence at least 95% identical to SEQ ID NO: 5, and a third RNA molecule comprising a nucleic acid sequence at least 95% identical to SEQ ID NO: 612. In an aspect, the non-coding RNA molecule suppresses the transcription or translation of a first RNA molecule comprising a nucleic acid sequence at least 95% identical to SEQ ID NO: 4, a second RNA molecule comprising a nucleic acid sequence at least 95% identical to SEQ ID NO: 6, and a third RNA molecule comprising a nucleic acid sequence at least 95% identical to SEQ ID NO: 612. In an aspect, the non-coding RNA molecule suppresses the transcription or translation of a first RNA molecule comprising a nucleic acid sequence at least 95% identical to SEQ ID NO: 5, a second RNA molecule comprising a nucleic acid sequence at least 95% identical to SEQ ID NO: 6, and a third RNA molecule comprising a nucleic acid sequence at least 95% identical to SEQ ID NO: 612. In an aspect, the non-coding RNA molecule suppresses the transcription or translation of a first RNA molecule comprising a nucleic acid sequence at least 95% identical to SEQ ID NO: 4, a second RNA molecule comprising a nucleic acid sequence at least 95% identical to SEQ ID NO: 5, a third RNA molecule comprising a nucleic acid sequence at least 95% identical to SEQ ID NO: 6, and a fourth RNA molecule comprising a nucleic acid sequence at least 95% identical to SEQ ID NO: 612. In an aspect, the non-coding RNA molecule suppresses the transcription or translation of a first RNA molecule comprising a nucleic acid sequence 100% identical to SEQ ID NO: 4 and a second RNA molecule comprising a nucleic acid sequence 100% identical to SEQ ID NO: 5. In an aspect, the non-coding RNA molecule suppresses the transcription or translation of a first RNA molecule comprising a nucleic acid sequence 100% identical to SEQ ID NO: 4 and a second RNA molecule comprising a nucleic acid sequence 100% identical to SEQ ID NO: 6. In an aspect, the non-coding RNA molecule suppresses the transcription or translation of a first RNA molecule comprising a nucleic acid sequence 100% identical to SEQ ID NO: 5 and a second RNA molecule comprising a nucleic acid sequence 100% identical to SEQ ID NO: 6. In an aspect, the non-coding RNA molecule suppresses the transcription or translation of a first RNA molecule comprising a nucleic acid sequence 100% identical to SEQ ID NO: 4 and a second RNA molecule comprising a nucleic acid sequence 100% identical to SEQ ID NO: 612. In an aspect, the non-coding RNA molecule suppresses the transcription or translation of a first RNA molecule comprising a nucleic acid sequence 100% identical to SEQ ID NO: 5 and a second RNA molecule comprising a nucleic acid sequence 100% identical to SEQ ID NO: 612. In an aspect, the non-coding RNA molecule suppresses the transcription or translation of a first RNA molecule comprising a nucleic acid sequence 100% identical to SEQ ID NO: 6 and a second RNA molecule comprising a nucleic acid sequence 100% identical to SEQ ID NO: 612. In an aspect, the non-coding RNA molecule suppresses the transcription or translation of a first RNA molecule comprising a nucleic acid sequence 100% identical to SEQ ID NO: 4, a second RNA molecule comprising a nucleic acid sequence 100% identical to SEQ ID NO: 5, and a third RNA molecule comprising a nucleic acid sequence 100% identical to SEQ ID NO: 6. In an aspect, the non-coding RNA molecule suppresses the transcription or translation of a first RNA molecule comprising a nucleic acid sequence 100% identical to SEQ ID NO: 4, a second RNA molecule comprising a nucleic acid sequence 100% identical to SEQ ID NO: 5, and a third RNA molecule comprising a nucleic acid sequence 100% identical to SEQ ID NO: 612. In an aspect, the non-coding RNA molecule suppresses the transcription or translation of a first RNA molecule comprising a nucleic acid sequence 100% identical to SEQ ID NO: 4, a second RNA molecule comprising a nucleic acid sequence 100% identical to SEQ ID NO: 6, and a third RNA molecule comprising a nucleic acid sequence 100% identical to SEQ ID NO: 612. In an aspect, the non-coding RNA molecule suppresses the transcription or translation of a first RNA molecule comprising a nucleic acid sequence 100% identical to SEQ ID NO: 5, a second RNA molecule comprising a nucleic acid sequence 100% identical to SEQ ID NO: 6, and a third RNA molecule comprising a nucleic acid sequence 100% identical to SEQ ID NO: 612. In an aspect, the non-coding RNA molecule suppresses the transcription or translation of a first RNA molecule comprising a nucleic acid sequence 100% identical to SEQ ID NO: 4, a second RNA molecule comprising a nucleic acid sequence 100% identical to SEQ ID NO: 5, a third RNA molecule comprising a nucleic acid sequence 100% identical to SEQ ID NO: 6, and a fourth RNA molecule comprising a nucleic acid sequence 100% identical to SEQ ID NO: 612.

In an aspect, a modified tobacco plant comprising a recombinant nucleic acid construct is the male parent when used in a method of crossing. In an aspect, a modified tobacco plant comprising a recombinant nucleic acid construct is the female parent when used in a method of crossing. In an aspect, a modified tobacco plant comprising a non-natural mutation is the male parent when used in a method of crossing. In an aspect, a modified tobacco plant comprising a non-natural mutation is the female parent when used in a method of crossing. It will be appreciated that a “male parent” provides pollen and a “female parent” receives the pollen in a crossing event.

“Curing” is the aging process that reduces moisture and brings about the destruction of chlorophyll giving tobacco leaves a golden color and by which starch is converted to sugar. Cured tobacco therefore has a higher reducing sugar content and a lower starch content compared to harvested green leaf. Cured tobacco is not considered living tissue. Cured tobacco material does not include tobacco seeds. Cured tobacco is not capable of giving rise to a tobacco plant. In one aspect, tobacco plants or plant components provided herein can be cured using conventional means, e.g., flue-cured, barn-cured, fire-cured, air-cured or sun-cured. See, for example, Tso (1999, Chapter 1 in Tobacco, Production, Chemistry and Technology, Davis & Nielsen, eds., Blackwell Publishing, Oxford) for a description of different types of curing methods. Cured tobacco is usually aged in a wooden drum (e.g., a hogshead) or cardboard cartons in compressed conditions for several years (e.g., two to five years), at a moisture content ranging from 10% to about 25%. See, U.S. Pat. Nos. 4,516,590 and 5,372,149. Cured and aged tobacco then can be further processed. Further processing includes conditioning the tobacco under vacuum with or without the introduction of steam at various temperatures, pasteurization, and fermentation.

Information regarding the harvesting of burley and dark tobacco varieties can be found in the 2019-2020 Burley and Dark Tobacco Production Guide (December 2018) published by the University of Kentucky, The University of Tennessee, Virginia Tech, and North Carolina State University, which is incorporated herein by reference in its entirety.

As used herein, “cured tobacco material” refers to tobacco plant tissue that has been subjected to curing. Non-limiting examples of tobacco plant tissues that can be subjected to curing include leaf tissue, stem tissue, bud tissue, floral tissue, and root tissue. In an aspect, tobacco seeds are not subjected to curing, and do not form a component of cured tobacco material. In an aspect, this disclosure provides cured tobacco material from any modified tobacco plant, or part thereof, provided herein. In an aspect, this disclosure provides a method comprising preparing a tobacco product using cured tobacco material from any modified tobacco plant, or part thereof, provided herein.

In an aspect, cured tobacco material comprises tobacco material selected from the group selected from cured leaf material, cured stem material, cured bud material, cured flower material, and cured root material. In another aspect, cured tobacco material comprises cured leaf material, cured stem material, or both. In a further aspect, cured tobacco material comprises cured leaf material. In yet another aspect, cured tobacco material comprises cured stem material. In an aspect, cured tobacco material does not contain tobacco seed.

In an aspect, cured tobacco material comprises flue-cured tobacco material. In another aspect, cured tobacco material comprises air-cured tobacco material. In another aspect, cured tobacco material comprises fire-cured tobacco material. In another aspect, cured tobacco material comprises sun-cured tobacco material. In another aspect, cured tobacco material provided herein is selected from the group consisting of air-cured tobacco material, fire-cured tobacco material, sun-cured tobacco material, and flue-cured tobacco material. In another aspect, cured tobacco material is from a tobacco variety selected from the group consisting of a flue-cured variety, a bright variety, a Burley variety, a Virginia variety, a Maryland variety, a dark variety, an Oriental variety, and a Turkish variety.

In an aspect, cured tobacco leaf provided herein is selected from the group consisting of air-cured tobacco leaf, fire-cured tobacco leaf, sun-cured tobacco leaf, and flue-cured tobacco leaf. In an aspect, cured tobacco leaf is from a tobacco variety selected from the group consisting of a flue-cured variety, a bright variety, a Burley variety, a Virginia variety, a Maryland variety, a dark variety, an Oriental variety, and a Turkish variety.

In an aspect, cured tobacco stem provided herein is selected from the group consisting of air-cured tobacco stem, fire-cured tobacco stem, sun-cured tobacco stem, and flue-cured tobacco stem. In an aspect, cured tobacco stem is from a tobacco variety selected from the group consisting of a flue-cured variety, a bright variety, a Burley variety, a Virginia variety, a Maryland variety, a dark variety, an Oriental variety, and a Turkish variety.

In an aspect, this disclosure provides fermented tobacco material from any modified tobacco plant, or part therefrom, provided herein. Fermentation typically is characterized by high initial moisture content, heat generation, and a 10 to 20% loss of dry weight. See, for example, U.S. Pat. Nos. 4,528,993, 4,660,577, 4,848,373, 5,372,149; U.S. Publication No. 2005/0178398; and Tso (1999, Chapter 1 in Tobacco, Production, Chemistry and Technology, Davis & Nielsen, eds., Blackwell Publishing, Oxford).

In an aspect, this disclosure provides cured and fermented tobacco material from any modified tobacco plant, or part thereof, provided herein.

Cured and/or fermented tobacco can be further processed (e.g., cut, shredded, expanded, blended). See, for example, U.S. Pat. Nos. 4,528,993; 4,660,577; and 4,987,907. In an aspect, cured tobacco material is cut. In an aspect, cured tobacco material is shredded. In an aspect, cured tobacco material is expanded. In an aspect, cured tobacco material is blended. In an aspect, fermented tobacco material is cut. In an aspect, fermented tobacco material is shredded. In an aspect, fermented tobacco material is expanded. In an aspect, fermented tobacco material is blended.

Tobacco material obtained from the modified tobacco plants, and parts thereof, of the present disclosure can be used to make tobacco products. As used herein, “tobacco product” is defined as any product made or derived from tobacco that is intended for human use or consumption. In an aspect, a tobacco product does not comprise a tobacco seed. In an aspect, a tobacco product does not comprise living tobacco material.

In an aspect, a tobacco product comprises plant material from a modified tobacco plant provided herein. In an aspect, a tobacco product comprises cured tobacco material. In an aspect, a tobacco product comprises fermented tobacco material. In an aspect, a tobacco product comprises a tobacco blend.

Tobacco products include, without limitation, cigarette products (e.g., cigarettes and bidi cigarettes), cigar products (e.g., cigar wrapping tobacco and cigarillos), pipe tobacco products, products derived from tobacco, tobacco-derived nicotine products, smokeless tobacco products (e.g., moist snuff, dry snuff, and chewing tobacco), films, chewables, tabs, shaped parts, gels, consumable units, insoluble matrices, hollow shapes, reconstituted tobacco, expanded tobacco, and the like. See, e.g., U.S. Patent Publication No. US 2006/0191548.

As used herein, “cigarette” refers a tobacco product having a “rod” and “filler”. The cigarette “rod” includes the cigarette paper, filter, plug wrap (used to contain filtration materials), tipping paper that holds the cigarette paper (including the filler) to the filter, and all glues that hold these components together. The “filler” includes (1) all tobaccos, including but not limited to reconstituted and expanded tobacco, (2) non-tobacco substitutes (including but not limited to herbs, non-tobacco plant materials and other spices that may accompany tobaccos rolled within the cigarette paper), (3) casings, (4) flavorings, and (5) all other additives (that are mixed into tobaccos and substitutes and rolled into the cigarette).

In an aspect, a tobacco product comprises reconstituted tobacco. In an aspect, reconstituted tobacco comprises cured tobacco material. As used herein, “reconstituted tobacco” refers to a part of tobacco filler made from tobacco dust and other tobacco scrap material, processed into sheet form and cut into strips to resemble tobacco. In addition to the cost savings, reconstituted tobacco is very important for its contribution to cigarette taste from processing flavor development using reactions between ammonia and sugars.

In an aspect, a tobacco product comprises expanded tobacco. As used herein, “expanded tobacco” refers to a part of tobacco filler which is processed through expansion of suitable gases so that the tobacco is “puffed” resulting in reduced density and greater filling capacity. It reduces the weight of tobacco used in cigarettes.

Tobacco products derived from plants of the present disclosure also include cigarettes and other smoking articles, particularly those smoking articles including filter elements, where the rod of smokable material includes cured tobacco within a tobacco blend.

In an aspect, a tobacco product is selected from the group consisting of a cigarillo, a non-ventilated recess filter cigarette, a vented recess filter cigarette, a cigar, snuff, pipe tobacco, cigar tobacco, cigarette tobacco, chewing tobacco, leaf tobacco, hookah tobacco, shredded tobacco, and cut tobacco. In an aspect, a tobacco product is selected from the group consisting of a cigarette, a heated tobacco product, a kretek, a bidi cigarette, a cigar, a cigarillo, a non-ventilated cigarette, a vented recess filter cigarette, pipe tobacco, snuff, snus, chewing tobacco, moist smokeless tobacco, fine cut chewing tobacco, long cut chewing tobacco, pouched chewing tobacco product, gum, a tablet, a lozenge, and a dissolving strip. In an aspect, a tobacco product is selected from the group consisting of a cigarette, a heated tobacco product, a cigar, pipe tobacco, snuff, snus, chewing tobacco, moist smokeless tobacco, fine cut chewing tobacco, long cut chewing tobacco, pouched chewing tobacco product, gum, a tablet, a lozenge, and a dissolving strip. In an aspect, a tobacco product is selected from the group consisting of a cigarette, a heated tobacco product, a cigar, pipe tobacco, snuff, snus, chewing tobacco, moist smokeless tobacco, fine cut chewing tobacco, long cut chewing tobacco, and pouched chewing tobacco product.

In an aspect, a cigarette is selected from the group consisting of a kretek, a bidi cigarette, a non-ventilated cigarette, and a vented recess filter cigarette. In an aspect, a cigar is a cigarillo.

In an aspect, a tobacco product is a heated tobacco product. As used herein, a “heated tobacco product” is a tobacco product that is heated to a lower temperature (e.g., about 600° C.) than a conventional cigarette. Heated tobacco products generate an aerosol or smoke that can be inhaled upon heating to an appropriate temperature. Heated tobacco products are also referred to as “heat-not-burn” tobacco products. Heated tobacco products are often used in electronic devices that use a battery to heat the heated tobacco product or a lit carbon ember that heats the heated tobacco product. In an aspect, a heated tobacco product is a film. In an aspect, a heated tobacco product does not combust when heated.

In an aspect, a tobacco product comprises a reconstituted tobacco film. In an aspect, a tobacco product comprises a humectant. In an aspect, a humectant is glycerin.

In an aspect, a tobacco product is a smokeless tobacco product. In an aspect, a smokeless tobacco product is selected from the group consisting of loose leaf chewing tobacco, plug chewing tobacco, moist snuff, nasal snuff, dry snuff, and snus.

Smokeless tobacco products are not combusted and include, but not limited to, chewing tobacco, moist smokeless tobacco, snus, and dry snuff. Chewing tobacco is coarsely divided tobacco leaf that is typically packaged in a large pouch-like package and used in a plug or twist. Moist smokeless tobacco is a moist, more finely divided tobacco that is provided in loose form or in pouch form and is typically packaged in round cans and used as a pinch or in a pouch placed between an adult tobacco consumer's cheek and gum. Snus is a heat-treated smokeless tobacco. Dry snuff is finely ground tobacco that is placed in the mouth or used nasally.

In yet another aspect, a tobacco product of the present disclosure is selected from the group consisting of an electronically heated cigarette, an e-cigarette, an electronic vaporing device.

In an aspect, a tobacco product is a blended tobacco product.

In an aspect, a tobacco blend comprises cured tobacco material. A tobacco blend can comprise any combination of cured tobacco, uncured tobacco, fermented tobacco, unfermented tobacco, expanded tobacco, and reconstituted tobacco.

In an aspect, a tobacco blend comprises at least 5% cured tobacco by weight. In an aspect, a tobacco blend comprises at least 10% cured tobacco by weight. In an aspect, a tobacco blend comprises at least 15% cured tobacco by weight. In an aspect, a tobacco blend comprises at least 20% cured tobacco by weight. In an aspect, a tobacco blend comprises at least 25% cured tobacco by weight. In an aspect, a tobacco blend comprises at least 30% cured tobacco by weight. In an aspect, a tobacco blend comprises at least 35% cured tobacco by weight. In an aspect, a tobacco blend comprises at least 40% cured tobacco by weight. In an aspect, a tobacco blend comprises at least 45% cured tobacco by weight. In an aspect, a tobacco blend comprises at least 50% cured tobacco by weight. In an aspect, a tobacco blend comprises at least 55% cured tobacco by weight. In an aspect, a tobacco blend comprises at least 60% cured tobacco by weight. In an aspect, a tobacco blend comprises at least 65% cured tobacco by weight. In an aspect, a tobacco blend comprises at least 70% cured tobacco by weight. In an aspect, a tobacco blend comprises at least 75% cured tobacco by weight. In an aspect, a tobacco blend comprises at least 80% cured tobacco by weight. In an aspect, a tobacco blend comprises at least 85% cured tobacco by weight. In an aspect, a tobacco blend comprises at least 90% cured tobacco by weight. In an aspect, a tobacco blend comprises at least 95% cured tobacco by weight.

In an aspect, a tobacco blend comprises at least 5% cured tobacco by volume. In an aspect, a tobacco blend comprises at least 10% cured tobacco by volume. In an aspect, a tobacco blend comprises at least 15% cured tobacco by volume. In an aspect, a tobacco blend comprises at least 20% cured tobacco by volume. In an aspect, a tobacco blend comprises at least 25% cured tobacco by volume. In an aspect, a tobacco blend comprises at least 30% cured tobacco by volume. In an aspect, a tobacco blend comprises at least 35% cured tobacco by volume. In an aspect, a tobacco blend comprises at least 40% cured tobacco by volume. In an aspect, a tobacco blend comprises at least 45% cured tobacco by volume. In an aspect, a tobacco blend comprises at least 50% cured tobacco by volume. In an aspect, a tobacco blend comprises at least 55% cured tobacco by volume. In an aspect, a tobacco blend comprises at least 60% cured tobacco by volume. In an aspect, a tobacco blend comprises at least 65% cured tobacco by volume. In an aspect, a tobacco blend comprises at least 70% cured tobacco by volume. In an aspect, a tobacco blend comprises at least 75% cured tobacco by volume. In an aspect, a tobacco blend comprises at least 80% cured tobacco by volume. In an aspect, a tobacco blend comprises at least 85% cured tobacco by volume. In an aspect, a tobacco blend comprises at least 90% cured tobacco by volume. In an aspect, a tobacco blend comprises at least 95% cured tobacco by volume.

In an aspect, a method comprises producing a tobacco product comprising any cured tobacco material provided herein.

In an aspect, a method comprises: (a) obtaining any cured tobacco material provide herein; and (b) producing a tobacco product comprising the cured tobacco material.

In an aspect, this disclosure provides a method of producing a tobacco product, the method comprising: (a) curing tobacco material to produce cured tobacco material, where the tobacco material is from a modified tobacco plant comprising a non-natural mutation in an endogenous nucleic acid molecule encoding a HAIRPLUS1a (NtHAP1a) protein, where the endogenous NtHAP1a protein comprises the amino acid sequence of SEQ ID NO: 1, and where expression or activity of the NtHAP1a protein is reduced in the modified tobacco plant as compared to a control tobacco plant lacking the non-natural mutation when grown under comparable conditions; and (b) producing a tobacco product comprising the cured tobacco material from step (a). In an aspect, the modified tobacco plant further comprises a non-natural mutation in an endogenous nucleic acid molecule encoding a HAIRPLUS1b (NtHAP1b) protein, where the endogenous NtHAP1b protein comprises the amino acid sequence of SEQ ID NO: 2, and where expression or activity of the NtHAP1b protein is reduced in the modified tobacco plant as compared to a control tobacco plant lacking the non-natural mutation when grown under comparable conditions. In an aspect, the modified tobacco plant further comprises a non-natural mutation in an endogenous nucleic acid molecule encoding a HAIRPLUS2 (NtHAP2) protein, where the endogenous NtHAP2 protein comprises the amino acid sequence of SEQ ID NO: 3, and where expression or activity of the NtHAP2 protein is reduced in the modified tobacco plant as compared to a control tobacco plant lacking the non-natural mutation when grown under comparable conditions. In an aspect, the modified tobacco plant further comprises a non-natural mutation in an endogenous nucleic acid molecule encoding a HAIRPLUS3 (NtHAP3) protein, where the endogenous NtHAP3 protein comprises the amino acid sequence of SEQ ID NO: 538, and where expression or activity of the NtHAP3 protein is reduced in the modified tobacco plant as compared to a control tobacco plant lacking the non-natural mutation when grown under comparable conditions.

In an aspect, this disclosure provides a method of producing a tobacco product, the method comprising: (a) curing tobacco material to produce cured tobacco material, where the tobacco material is from a modified tobacco plant comprising a non-natural mutation in an endogenous nucleic acid molecule encoding a HAIRPLUS1b (NtHAP1b) protein, where the endogenous NtHAP1b protein comprises the amino acid sequence of SEQ ID NO: 2, and where expression or activity of the NtHAP1b protein is reduced in the modified tobacco plant as compared to a control tobacco plant lacking the non-natural mutation when grown under comparable conditions; and (b) producing a tobacco product comprising the cured tobacco material from step (a). In an aspect, the modified tobacco plant further comprises a non-natural mutation in an endogenous nucleic acid molecule encoding a HAIRPLUS1a (NtHAP1a) protein, where the endogenous NtHAP1a protein comprises the amino acid sequence of SEQ ID NO: 1, and where expression or activity of the NtHAP1a protein is reduced in the modified tobacco plant as compared to a control tobacco plant lacking the non-natural mutation when grown under comparable conditions. In an aspect, the modified tobacco plant further comprises a non-natural mutation in an endogenous nucleic acid molecule encoding a HAIRPLUS2 (NtHAP2) protein, where the endogenous NtHAP2 protein comprises the amino acid sequence of SEQ ID NO: 3, and where expression or activity of the NtHAP2 protein is reduced in the modified tobacco plant as compared to a control tobacco plant lacking the non-natural mutation when grown under comparable conditions. In an aspect, the modified tobacco plant further comprises a non-natural mutation in an endogenous nucleic acid molecule encoding a HAIRPLUS3 (NtHAP3) protein, where the endogenous NtHAP3 protein comprises the amino acid sequence of SEQ ID NO: 538, and where expression or activity of the NtHAP3 protein is reduced in the modified tobacco plant as compared to a control tobacco plant lacking the non-natural mutation when grown under comparable conditions.

In an aspect, this disclosure provides a method of producing a tobacco product, the method comprising: (a) curing tobacco material to produce cured tobacco material, where the tobacco material is from a modified tobacco plant comprising a non-natural mutation in an endogenous nucleic acid molecule encoding a HAIRPLUS2 (NtHAP2) protein, where the endogenous NtHAP2 protein comprises the amino acid sequence of SEQ ID NO: 3, and where expression or activity of the NtHAP2 protein is reduced in the modified tobacco plant as compared to a control tobacco plant lacking the non-natural mutation when grown under comparable conditions; (b) producing a tobacco product comprising the cured tobacco material from step (a). In an aspect, the modified tobacco plant further comprises a non-natural mutation in an endogenous nucleic acid molecule encoding a HAIRPLUS1b (NtHAP1b) protein, where the endogenous NtHAP1b protein comprises the amino acid sequence of SEQ ID NO: 2, and where expression or activity of the NtHAP1b protein is reduced in the modified tobacco plant as compared to a control tobacco plant lacking the non-natural mutation when grown under comparable conditions. In an aspect, the modified tobacco plant further comprises a non-natural mutation in an endogenous nucleic acid molecule encoding a HAIRPLUS1a (NtHAP1a) protein, where the endogenous NtHAP1a protein comprises the amino acid sequence of SEQ ID NO: 1, and where expression or activity of the NtHAP1a protein is reduced in the modified tobacco plant as compared to a control tobacco plant lacking the non-natural mutation when grown under comparable conditions. In an aspect, the modified tobacco plant further comprises a non-natural mutation in an endogenous nucleic acid molecule encoding a HAIRPLUS3 (NtHAP3) protein, where the endogenous NtHAP3 protein comprises the amino acid sequence of SEQ ID NO: 538, and where expression or activity of the NtHAP3 protein is reduced in the modified tobacco plant as compared to a control tobacco plant lacking the non-natural mutation when grown under comparable conditions.

In an aspect, this disclosure provides a method of producing a tobacco product, the method comprising: (a) curing tobacco material to produce cured tobacco material, where the tobacco material is from a modified tobacco plant comprising a non-natural mutation in an endogenous nucleic acid molecule encoding a HAIRPLUS3 (NtHAP3) protein, where the endogenous NtHAP3 protein comprises the amino acid sequence of SEQ ID NO: 538, and where expression or activity of the NtHAP3 protein is reduced in the modified tobacco plant as compared to a control tobacco plant lacking the non-natural mutation when grown under comparable conditions; (b) producing a tobacco product comprising the cured tobacco material from step (a). In an aspect, the modified tobacco plant further comprises a non-natural mutation in an endogenous nucleic acid molecule encoding a HAIRPLUS1b (NtHAP1b) protein, where the endogenous NtHAP1b protein comprises the amino acid sequence of SEQ ID NO: 2, and where expression or activity of the NtHAP1b protein is reduced in the modified tobacco plant as compared to a control tobacco plant lacking the non-natural mutation when grown under comparable conditions. In an aspect, the modified tobacco plant further comprises a non-natural mutation in an endogenous nucleic acid molecule encoding a HAIRPLUS1a (NtHAP1a) protein, where the endogenous NtHAP1a protein comprises the amino acid sequence of SEQ ID NO: 1, and where expression or activity of the NtHAP1a protein is reduced in the modified tobacco plant as compared to a control tobacco plant lacking the non-natural mutation when grown under comparable conditions. In an aspect, the modified tobacco plant further comprises a non-natural mutation in an endogenous nucleic acid molecule encoding a HAIRPLUS2 (NtHAP2) protein, where the endogenous NtHAP2 protein comprises the amino acid sequence of SEQ ID NO: 3, and where expression or activity of the NtHAP2 protein is reduced in the modified tobacco plant as compared to a control tobacco plant lacking the non-natural mutation when grown under comparable conditions.

In an aspect, this disclosure provides a method of producing a tobacco product, the method comprising: (a) curing tobacco material to produce cured tobacco material, where the tobacco material is from a modified tobacco plant comprising a non-natural mutation in an endogenous nucleic acid molecule encoding a HAIRPLUS (HAP) protein, where the endogenous HAP protein comprises an amino acid sequence selected from the group consisting of SEQ ID NOs: 539 to 546, and where expression or activity of the NtHAP3 protein is reduced in the modified tobacco plant as compared to a control tobacco plant lacking the non-natural mutation when grown under comparable conditions; (b) producing a tobacco product comprising the cured tobacco material from step (a).

In an aspect, this disclosure provides a method of producing a tobacco product, the method comprising: (a) curing tobacco material to produce cured tobacco material, where the tobacco material is from a modified tobacco plant comprising a non-natural mutation in an endogenous nucleic acid molecule comprising the nucleic acid sequence of SEQ ID NO: 4 or 7, and where expression or activity of the endogenous nucleic acid molecule is reduced in the modified tobacco plant as compared to a control tobacco plant lacking the non-natural mutation when grown under comparable conditions; and (b) producing a tobacco product comprising the cured tobacco material from step (a). In an aspect, the modified tobacco plant further comprises a non-natural mutation in an endogenous nucleic acid molecule comprising the nucleic acid sequence of SEQ ID NO: 5 or 8, where expression or activity of the endogenous nucleic acid molecule is reduced in the modified tobacco plant as compared to a control tobacco plant lacking the non-natural mutation when grown under comparable conditions. In an aspect, the modified tobacco plant further comprises a non-natural mutation in an endogenous nucleic acid molecule comprising the nucleic acid sequence of SEQ ID NO: 6 or 9, where expression or activity of the endogenous nucleic acid molecule is reduced in the modified tobacco plant as compared to a control tobacco plant lacking the non-natural mutation when grown under comparable conditions. In an aspect, the modified tobacco plant further comprises a non-natural mutation in an endogenous nucleic acid molecule comprising the nucleic acid sequence of SEQ ID NO: 537 or 612, where expression or activity of the endogenous nucleic acid molecule is reduced in the modified tobacco plant as compared to a control tobacco plant lacking the non-natural mutation when grown under comparable conditions.

In an aspect, this disclosure provides a method of producing a tobacco product, the method comprising: (a) curing tobacco material to produce cured tobacco material, where the tobacco material is from a modified tobacco plant comprising a non-natural mutation in an endogenous nucleic acid molecule comprising the nucleic acid sequence of SEQ ID NO: 5 or 8, and where expression or activity of the endogenous nucleic acid molecule is reduced in the modified tobacco plant as compared to a control tobacco plant lacking the non-natural mutation when grown under comparable conditions; and (b) producing a tobacco product comprising the cured tobacco material from step (a). In an aspect, the modified tobacco plant further comprises a non-natural mutation in an endogenous nucleic acid molecule comprising the nucleic acid sequence of SEQ ID NO: 4 or 7, where expression or activity of the endogenous nucleic acid molecule is reduced in the modified tobacco plant as compared to a control tobacco plant lacking the non-natural mutation when grown under comparable conditions. In an aspect, the modified tobacco plant further comprises a non-natural mutation in an endogenous nucleic acid molecule comprising the nucleic acid sequence of SEQ ID NO: 6 or 9, where expression or activity of the endogenous nucleic acid molecule is reduced in the modified tobacco plant as compared to a control tobacco plant lacking the non-natural mutation when grown under comparable conditions. In an aspect, the modified tobacco plant further comprises a non-natural mutation in an endogenous nucleic acid molecule comprising the nucleic acid sequence of SEQ ID NO: 537 or 612, where expression or activity of the endogenous nucleic acid molecule is reduced in the modified tobacco plant as compared to a control tobacco plant lacking the non-natural mutation when grown under comparable conditions.

In an aspect, this disclosure provides a method of producing a tobacco product, the method comprising: (a) curing tobacco material to produce cured tobacco material, where the tobacco material is from a modified tobacco plant comprising a non-natural mutation in an endogenous nucleic acid molecule comprising the nucleic acid sequence of SEQ ID NO: 6 or 9, and where expression or activity of the endogenous nucleic acid molecule is reduced in the modified tobacco plant as compared to a control tobacco plant lacking the non-natural mutation when grown under comparable conditions; and (b) producing a tobacco product comprising the cured tobacco material from step (a). In an aspect, the modified tobacco plant further comprises a non-natural mutation in an endogenous nucleic acid molecule comprising the nucleic acid sequence of SEQ ID NO: 5 or 8, where expression or activity of the endogenous nucleic acid molecule is reduced in the modified tobacco plant as compared to a control tobacco plant lacking the non-natural mutation when grown under comparable conditions. In an aspect, the modified tobacco plant further comprises a non-natural mutation in an endogenous nucleic acid molecule comprising the nucleic acid sequence of SEQ ID NO: 4 or 7, where expression or activity of the endogenous nucleic acid molecule is reduced in the modified tobacco plant as compared to a control tobacco plant lacking the non-natural mutation when grown under comparable conditions. In an aspect, the modified tobacco plant further comprises a non-natural mutation in an endogenous nucleic acid molecule comprising the nucleic acid sequence of SEQ ID NO: 537 or 612, where expression or activity of the endogenous nucleic acid molecule is reduced in the modified tobacco plant as compared to a control tobacco plant lacking the non-natural mutation when grown under comparable conditions.

In an aspect, this disclosure provides a method of producing a tobacco product, the method comprising: (a) curing tobacco material to produce cured tobacco material, where the tobacco material is from a modified tobacco plant comprising a non-natural mutation in an endogenous nucleic acid molecule comprising the nucleic acid sequence of SEQ ID NO: 537 or 612, and where expression or activity of the endogenous nucleic acid molecule is reduced in the modified tobacco plant as compared to a control tobacco plant lacking the non-natural mutation when grown under comparable conditions; and (b) producing a tobacco product comprising the cured tobacco material from step (a). In an aspect, the modified tobacco plant further comprises a non-natural mutation in an endogenous nucleic acid molecule comprising the nucleic acid sequence of SEQ ID NO: 5 or 8, where expression or activity of the endogenous nucleic acid molecule is reduced in the modified tobacco plant as compared to a control tobacco plant lacking the non-natural mutation when grown under comparable conditions. In an aspect, the modified tobacco plant further comprises a non-natural mutation in an endogenous nucleic acid molecule comprising the nucleic acid sequence of SEQ ID NO: 4 or 7, where expression or activity of the endogenous nucleic acid molecule is reduced in the modified tobacco plant as compared to a control tobacco plant lacking the non-natural mutation when grown under comparable conditions. In an aspect, the modified tobacco plant further comprises a non-natural mutation in an endogenous nucleic acid molecule comprising the nucleic acid sequence of SEQ ID NO: 6 or 9, where expression or activity of the endogenous nucleic acid molecule is reduced in the modified tobacco plant as compared to a control tobacco plant lacking the non-natural mutation when grown under comparable conditions.

In an aspect, this disclosure provides a method of producing a modified tobacco plant, the method comprising: (a) inducing at least one non-natural mutation in at least one endogenous nucleic acid molecule encoding an amino acid sequence selected from the group consisting of SEQ ID NOs: 1, 2, 3, and 538 in at least one tobacco cell; and (b) regenerating a modified tobacco plant from the at least one tobacco cell of step (a), where the modified tobacco plant comprises the at least one non-natural mutation, and where the modified tobacco plant exhibits reduced expression or activity of the at least one endogenous nucleic acid molecule as compared to a control tobacco plant lacking the at least one non-natural mutation when grown under comparable conditions. In an aspect, a method produces a non-natural mutation in an endogenous nucleic acid molecule encoding the amino acid sequence of SEQ ID NO: 1. In an aspect, a method produces a non-natural mutation in an endogenous nucleic acid molecule encoding the amino acid sequence of SEQ ID NO: 2. In an aspect, a method produces a non-natural mutation in an endogenous nucleic acid molecule encoding the amino acid sequence of SEQ ID NO: 3. In an aspect, a method produces a non-natural mutation in an endogenous nucleic acid molecule encoding the amino acid sequence of SEQ ID NO: 538. In an aspect, a method produces a first non-natural mutation in an endogenous nucleic acid molecule encoding the amino acid sequence of SEQ ID NO: 1 and a second non-natural mutation in an endogenous nucleic acid molecule encoding the amino acid sequence of SEQ ID NO: 2. In an aspect, a method produces a first non-natural mutation in an endogenous nucleic acid molecule encoding the amino acid sequence of SEQ ID NO: 1 and a second non-natural mutation in an endogenous nucleic acid molecule encoding the amino acid sequence of SEQ ID NO: 3. In an aspect, a method produces a first non-natural mutation in an endogenous nucleic acid molecule encoding the amino acid sequence of SEQ ID NO: 2 and a second non-natural mutation in an endogenous nucleic acid molecule encoding the amino acid sequence of SEQ ID NO: 3. In an aspect, a method produces a first non-natural mutation in an endogenous nucleic acid molecule encoding the amino acid sequence of SEQ ID NO: 1 and a second non-natural mutation in an endogenous nucleic acid molecule encoding the amino acid sequence of SEQ ID NO: 538. In an aspect, a method produces a first non-natural mutation in an endogenous nucleic acid molecule encoding the amino acid sequence of SEQ ID NO: 2 and a second non-natural mutation in an endogenous nucleic acid molecule encoding the amino acid sequence of SEQ ID NO: 538. In an aspect, a method produces a first non-natural mutation in an endogenous nucleic acid molecule encoding the amino acid sequence of SEQ ID NO: 3 and a second non-natural mutation in an endogenous nucleic acid molecule encoding the amino acid sequence of SEQ ID NO: 538. In an aspect, a method produces a first non-natural mutation in an endogenous nucleic acid molecule encoding the amino acid sequence of SEQ ID NO: 1, a second non-natural mutation in an endogenous nucleic acid molecule encoding the amino acid sequence of SEQ ID NO: 2, and a third non-natural mutation in an endogenous nucleic acid molecule encoding the amino acid sequence of SEQ ID NO: 3. In an aspect, a method produces a first non-natural mutation in an endogenous nucleic acid molecule encoding the amino acid sequence of SEQ ID NO: 1, a second non-natural mutation in an endogenous nucleic acid molecule encoding the amino acid sequence of SEQ ID NO: 2, and a third non-natural mutation in an endogenous nucleic acid molecule encoding the amino acid sequence of SEQ ID NO: 538. In an aspect, a method produces a first non-natural mutation in an endogenous nucleic acid molecule encoding the amino acid sequence of SEQ ID NO: 1, a second non-natural mutation in an endogenous nucleic acid molecule encoding the amino acid sequence of SEQ ID NO: 3, and a third non-natural mutation in an endogenous nucleic acid molecule encoding the amino acid sequence of SEQ ID NO: 538. In an aspect, a method produces a first non-natural mutation in an endogenous nucleic acid molecule encoding the amino acid sequence of SEQ ID NO: 2, a second non-natural mutation in an endogenous nucleic acid molecule encoding the amino acid sequence of SEQ ID NO: 3, and a third non-natural mutation in an endogenous nucleic acid molecule encoding the amino acid sequence of SEQ ID NO: 538. In an aspect, a method produces a first non-natural mutation in an endogenous nucleic acid molecule encoding the amino acid sequence of SEQ ID NO: 1, a second non-natural mutation in an endogenous nucleic acid molecule encoding the amino acid sequence of SEQ ID NO: 2, a third non-natural mutation in an endogenous nucleic acid molecule encoding the amino acid sequence of SEQ ID NO: 3, and a fourth non-natural mutation in an endogenous nucleic acid molecule encoding the amino acid sequence of SEQ ID NO: 538.

In an aspect, this disclosure provides a method of producing a modified tobacco plant, the method comprising: (a) inducing at least one non-natural mutation in at least one endogenous nucleic acid molecule encoding an amino acid sequence selected from the group consisting of SEQ ID NOs: 539 to 546 in at least one tobacco cell; and (b) regenerating a modified tobacco plant from the at least one tobacco cell of step (a), where the modified tobacco plant comprises the at least one non-natural mutation, and where the modified tobacco plant exhibits reduced expression or activity of the at least one endogenous nucleic acid molecule as compared to a control tobacco plant lacking the at least one non-natural mutation when grown under comparable conditions.

In an aspect, this disclosure provides a method of producing a modified tobacco plant, the method comprising: (a) inducing at least one non-natural mutation in at least one endogenous nucleic acid molecule comprising a nucleic acid sequence selected from the group consisting of SEQ ID NOs: 4, 5, 6, and 612 in at least one tobacco cell; and (b) regenerating a modified tobacco plant from the at least one tobacco cell of step (a), where the modified tobacco plant comprises the at least one non-natural mutation, and where the modified tobacco plant exhibits reduced expression or activity of the at least one endogenous nucleic acid molecule as compared to a control tobacco plant lacking the at least one non-natural mutation when grown under comparable conditions. In an aspect, a method produces a non-natural mutation in an endogenous nucleic acid molecule comprising the nucleic acid sequence of SEQ ID NO: 4. In an aspect, a method produces a non-natural mutation in an endogenous nucleic acid molecule comprising the nucleic acid sequence of SEQ ID NO: 5. In an aspect, a method produces a non-natural mutation in an endogenous nucleic acid molecule comprising the nucleic acid sequence of SEQ ID NO: 6. In an aspect, a method produces a non-natural mutation in an endogenous nucleic acid molecule comprising the nucleic acid sequence of SEQ ID NO: 612. In an aspect, a method produces a first non-natural mutation in an endogenous nucleic acid molecule comprising the nucleic acid sequence of SEQ ID NO: 4 and a second non-natural mutation in an endogenous nucleic acid molecule comprising the nucleic acid sequence of SEQ ID NO: 5. In an aspect, a method produces a first non-natural mutation in an endogenous nucleic acid molecule comprising the nucleic acid sequence of SEQ ID NO: 4 and a second non-natural mutation in an endogenous nucleic acid molecule comprising the nucleic acid sequence of SEQ ID NO: 6. In an aspect, a method produces a first non-natural mutation in an endogenous nucleic acid molecule comprising the nucleic acid sequence of SEQ ID NO: 5 and a second non-natural mutation in an endogenous nucleic acid molecule comprising the nucleic acid sequence of SEQ ID NO: 6. In an aspect, a method produces a first non-natural mutation in an endogenous nucleic acid molecule comprising the nucleic acid sequence of SEQ ID NO: 4 and a second non-natural mutation in an endogenous nucleic acid molecule comprising the nucleic acid sequence of SEQ ID NO: 612. In an aspect, a method produces a first non-natural mutation in an endogenous nucleic acid molecule comprising the nucleic acid sequence of SEQ ID NO: 5 and a second non-natural mutation in an endogenous nucleic acid molecule comprising the nucleic acid sequence of SEQ ID NO: 612. In an aspect, a method produces a first non-natural mutation in an endogenous nucleic acid molecule comprising the nucleic acid sequence of SEQ ID NO: 6 and a second non-natural mutation in an endogenous nucleic acid molecule comprising the nucleic acid sequence of SEQ ID NO: 612. In an aspect, a method produces a first non-natural mutation in an endogenous nucleic acid molecule comprising the nucleic acid sequence of SEQ ID NO: 4, a second non-natural mutation in an endogenous nucleic acid molecule comprising the nucleic acid sequence of SEQ ID NO: 5, and a third non-natural mutation in an endogenous nucleic acid molecule comprising the nucleic acid sequence of SEQ ID NO: 6. In an aspect, a method produces a first non-natural mutation in an endogenous nucleic acid molecule comprising the nucleic acid sequence of SEQ ID NO: 4, a second non-natural mutation in an endogenous nucleic acid molecule comprising the nucleic acid sequence of SEQ ID NO: 5, and a third non-natural mutation in an endogenous nucleic acid molecule comprising the nucleic acid sequence of SEQ ID NO: 612. In an aspect, a method produces a first non-natural mutation in an endogenous nucleic acid molecule comprising the nucleic acid sequence of SEQ ID NO: 4, a second non-natural mutation in an endogenous nucleic acid molecule comprising the nucleic acid sequence of SEQ ID NO: 6, and a third non-natural mutation in an endogenous nucleic acid molecule comprising the nucleic acid sequence of SEQ ID NO: 612. In an aspect, a method produces a first non-natural mutation in an endogenous nucleic acid molecule comprising the nucleic acid sequence of SEQ ID NO: 5, a second non-natural mutation in an endogenous nucleic acid molecule comprising the nucleic acid sequence of SEQ ID NO: 6, and a third non-natural mutation in an endogenous nucleic acid molecule comprising the nucleic acid sequence of SEQ ID NO: 612. In an aspect, a method produces a first non-natural mutation in an endogenous nucleic acid molecule comprising the nucleic acid sequence of SEQ ID NO: 4, a second non-natural mutation in an endogenous nucleic acid molecule comprising the nucleic acid sequence of SEQ ID NO: 5, a third non-natural mutation in an endogenous nucleic acid molecule comprising the nucleic acid sequence of SEQ ID NO: 6, and a fourth non-natural mutation in an endogenous nucleic acid molecule comprising the nucleic acid sequence of SEQ ID NO: 612.

In an aspect, this disclosure provides a method of producing a modified tobacco plant, the method comprising: (a) inducing at least one non-natural mutation in at least one endogenous nucleic acid molecule comprising a nucleic acid sequence selected from the group consisting of SEQ ID NOs: 7, 8, 9, and 537 in at least one tobacco cell; and (b) regenerating a modified tobacco plant from the at least one tobacco cell of step (a), where the modified tobacco plant comprises the at least one non-natural mutation, and where the modified tobacco plant exhibits reduced expression or activity of the at least one endogenous nucleic acid molecule as compared to a control tobacco plant lacking the at least one non-natural mutation when grown under comparable conditions. In an aspect, a method produces a non-natural mutation in an endogenous nucleic acid molecule comprising the nucleic acid sequence of SEQ ID NO: 7. In an aspect, a method produces a non-natural mutation in an endogenous nucleic acid molecule comprising the nucleic acid sequence of SEQ ID NO: 8. In an aspect, a method produces a non-natural mutation in an endogenous nucleic acid molecule comprising the nucleic acid sequence of SEQ ID NO: 9. In an aspect, a method produces a non-natural mutation in an endogenous nucleic acid molecule comprising the nucleic acid sequence of SEQ ID NO: 537. In an aspect, a method produces a first non-natural mutation in an endogenous nucleic acid molecule comprising the nucleic acid sequence of SEQ ID NO: 7 and a second non-natural mutation in an endogenous nucleic acid molecule comprising the nucleic acid sequence of SEQ ID NO: 8. In an aspect, a method produces a first non-natural mutation in an endogenous nucleic acid molecule comprising the nucleic acid sequence of SEQ ID NO: 7 and a second non-natural mutation in an endogenous nucleic acid molecule comprising the nucleic acid sequence of SEQ ID NO: 9. In an aspect, a method produces a first non-natural mutation in an endogenous nucleic acid molecule comprising the nucleic acid sequence of SEQ ID NO: 8 and a second non-natural mutation in an endogenous nucleic acid molecule comprising the nucleic acid sequence of SEQ ID NO: 9. In an aspect, a method produces a first non-natural mutation in an endogenous nucleic acid molecule comprising the nucleic acid sequence of SEQ ID NO: 7 and a second non-natural mutation in an endogenous nucleic acid molecule comprising the nucleic acid sequence of SEQ ID NO: 537. In an aspect, a method produces a first non-natural mutation in an endogenous nucleic acid molecule comprising the nucleic acid sequence of SEQ ID NO: 8 and a second non-natural mutation in an endogenous nucleic acid molecule comprising the nucleic acid sequence of SEQ ID NO: 537. In an aspect, a method produces a first non-natural mutation in an endogenous nucleic acid molecule comprising the nucleic acid sequence of SEQ ID NO: 9 and a second non-natural mutation in an endogenous nucleic acid molecule comprising the nucleic acid sequence of SEQ ID NO: 537. In an aspect, a method produces a first non-natural mutation in an endogenous nucleic acid molecule comprising the nucleic acid sequence of SEQ ID NO: 7, a second non-natural mutation in an endogenous nucleic acid molecule comprising the nucleic acid sequence of SEQ ID NO: 8, and a third non-natural mutation in an endogenous nucleic acid molecule comprising the nucleic acid sequence of SEQ ID NO: 9. In an aspect, a method produces a first non-natural mutation in an endogenous nucleic acid molecule comprising the nucleic acid sequence of SEQ ID NO: 7, a second non-natural mutation in an endogenous nucleic acid molecule comprising the nucleic acid sequence of SEQ ID NO: 8, and a third non-natural mutation in an endogenous nucleic acid molecule comprising the nucleic acid sequence of SEQ ID NO: 537. In an aspect, a method produces a first non-natural mutation in an endogenous nucleic acid molecule comprising the nucleic acid sequence of SEQ ID NO: 7, a second non-natural mutation in an endogenous nucleic acid molecule comprising the nucleic acid sequence of SEQ ID NO: 9, and a third non-natural mutation in an endogenous nucleic acid molecule comprising the nucleic acid sequence of SEQ ID NO: 537. In an aspect, a method produces a first non-natural mutation in an endogenous nucleic acid molecule comprising the nucleic acid sequence of SEQ ID NO: 8, a second non-natural mutation in an endogenous nucleic acid molecule comprising the nucleic acid sequence of SEQ ID NO: 9, and a third non-natural mutation in an endogenous nucleic acid molecule comprising the nucleic acid sequence of SEQ ID NO: 537. In an aspect, a method produces a first non-natural mutation in an endogenous nucleic acid molecule comprising the nucleic acid sequence of SEQ ID NO: 7, a second non-natural mutation in an endogenous nucleic acid molecule comprising the nucleic acid sequence of SEQ ID NO: 8, a third non-natural mutation in an endogenous nucleic acid molecule comprising the nucleic acid sequence of SEQ ID NO: 9, and a fourth non-natural mutation in an endogenous nucleic acid molecule comprising the nucleic acid sequence of SEQ ID NO: 9.

As used herein, “inducing” a non-natural mutation refers to generating a mutation in a polynucleotide sequence via human intervention. Many suitable methods for inducing mutations in tobacco are known in the art. Non-limiting examples of such methods include use of chemical mutagens, use of irradiation, use of nucleases, use of transposons, and use of Agrobacterium. In an aspect, inducing a non-natural mutation comprises the use of an agent selected from the group consisting of a chemical mutagen, irradiation, a transposon, Agrobacterium, and a nuclease.

In an aspect, inducing a non-natural mutation comprises the use of a chemical mutagen. In an aspect, a chemical mutagen comprises ethyl methanesulfonate (EMS). In an aspect, a chemical mutagen is methyl methanesulfonate (MMS). In an aspect, a chemical mutagen is hydrogen fluoride (HF). In an aspect, a chemical mutagen is sodium azide (SA). In an aspect, a chemical mutagen is N-methyl-N-nitrosourea (MNU). In an aspect, a chemical mutagen is hydroxylamine (H₃NO). In an aspect, a chemical mutagen is selected from the group consisting of EMS, MMS, HF, SA, MNU, and H₃NO.

In another aspect, inducing a mutation comprises the use of irradiation. In an aspect, irradiation is selected from the group consisting of gamma rays, X-rays, ionizing radiation, or fast neutrons. In an aspect, irradiation comprises the use of gamma rays. In an aspect, irradiation comprises the use of X-rays. In an aspect, irradiation comprises the use of ionizing radiation. In an aspect, irradiation comprises the use of fast neutrons.

In an aspect, inducing a mutation comprises the use of a transposon. In another aspect, inducing a mutation comprises the use of Agrobacterium.

In a further aspect, inducing a mutation comprises the use of a nuclease. In an aspect, a nuclease is a nickase. As used herein, a “nickase” refers to a nuclease that generates a single-stranded break rather than a double-stranded break in a targeted nucleic acid molecule. In an aspect, a nuclease is selected from the group consisting of a meganuclease, a zinc-finger nuclease, a transcription activator-like effector nuclease (TALEN), a CRISPR/Cas9 nuclease, a CRISPR/Cpf1 nuclease (also known as a CRISPR/Cas12a nuclease), a CRISPR/CasX nuclease, a CRISPR/CasY nuclease, and a Csm1 nuclease. In an aspect, inducing a mutation comprises the use of a CRISPR/Cas9 nuclease. In an aspect, inducing a mutation comprises the use of a CRISPR/Cpf1 nuclease. In an aspect, inducing a mutation comprises the use of a CRISPR/CasX nuclease. In an aspect, inducing a mutation comprises the use of a CRISPR/CasY nuclease. In an aspect, inducing a mutation comprises the use of a Csm1 nuclease. In an aspect, inducing a mutation comprises the use of a meganuclease. In an aspect, inducing a mutation comprises the use of a zinc-finger nuclease. In an aspect, inducing a mutation comprises the use of a TALEN.

In an aspect, inducing a mutation comprises the use of a base editor. As used herein, a “base editor” refers to a catalytically impaired Cas nuclease fused to a nucleotide deaminase. In some aspects, base editors further comprise DNA repair proteins. In an aspect, a base editor is a cytosine base editor. A cytosine base editor enables C-G to T-A transitions. In an aspect, a base editor is an adenine base editor. An adenine base editor enables A-T to G-C conversion. In an aspect, a base editor is a C-to-G base editor.

In an aspect, inducing a mutation comprises the use of a prime editor. As used herein, a “prime editor” refers to a Cas nickase fused to an engineered reverse transcriptase. Prime editors can introduce all 12 transition and transversion mutations and small insertions or deletions, as well as combinations thereof. Prime editors use a prime editing guide RNA (pegRNA) that specifies the target site for editing and encodes the desired edit. In an aspect, a pegRNA is provided to a tobacco cell. In an aspect, a pegRNA comprises a nucleic acid sequence that binds to a nucleic acid sequence selected from the group consisting of SEQ ID NOs: 4 to 9, 537, and 612.

Additional information about base editors and prime editors, and their use in plants, can be found in Molla et al., Nature Plants, 7:1166-1187 (2021). See also Anzalone et al., Nature, 576:149-157 (2019); Komor et al., Nature, 533:420-424 (2016); and Gaudelli et al., Nature, 551:464-471 (2017).

In an aspect, this disclosure provides a method of producing a modified tobacco plant, the method comprising: (a) introducing a recombinant nucleic acid construct to at least one tobacco cell, where the recombinant nucleic acid construct comprises a heterologous promoter operably linked to a polynucleotide that encodes a non-coding RNA molecule; and (b) regenerating a modified tobacco plant from the at least one tobacco cell of step (a), where the modified tobacco plant comprises the recombinant nucleic acid construct, and where the non-coding RNA molecule suppresses the transcription or translation of at least one RNA molecule encoding an amino acid sequence at least 90% identical or similar to an amino acid sequence selected from the group consisting of SEQ ID NOs: 1, 2, 3, and 538 as compared to a control tobacco plant lacking the recombinant nucleic acid construct when grown under comparable conditions. In an aspect, the non-coding RNA molecule suppresses the transcription or translation of a first RNA molecule encoding an amino acid sequence at least 90% identical or similar to SEQ ID NO: 1 and a second RNA molecule encoding an amino acid sequence at least 90% identical or similar to SEQ ID NO: 2. In an aspect, the non-coding RNA molecule suppresses the transcription or translation of a first RNA molecule encoding an amino acid sequence at least 90% identical or similar to SEQ ID NO: 1 and a second RNA molecule encoding an amino acid sequence at least 90% identical or similar to SEQ ID NO: 3. In an aspect, the non-coding RNA molecule suppresses the transcription or translation of a first RNA molecule encoding an amino acid sequence at least 90% identical or similar to SEQ ID NO: 2 and a second RNA molecule encoding an amino acid sequence at least 90% identical or similar to SEQ ID NO: 3. In an aspect, the non-coding RNA molecule suppresses the transcription or translation of a first RNA molecule encoding an amino acid sequence at least 90% identical or similar to SEQ ID NO: 1 and a second RNA molecule encoding an amino acid sequence at least 90% identical or similar to SEQ ID NO: 538. In an aspect, the non-coding RNA molecule suppresses the transcription or translation of a first RNA molecule encoding an amino acid sequence at least 90% identical or similar to SEQ ID NO: 2 and a second RNA molecule encoding an amino acid sequence at least 90% identical or similar to SEQ ID NO: 538. In an aspect, the non-coding RNA molecule suppresses the transcription or translation of a first RNA molecule encoding an amino acid sequence at least 90% identical or similar to SEQ ID NO: 3 and a second RNA molecule encoding an amino acid sequence at least 90% identical or similar to SEQ ID NO: 538. In an aspect, the non-coding RNA molecule suppresses the transcription or translation of a first RNA molecule encoding an amino acid sequence at least 90% identical or similar to SEQ ID NO: 1, a second RNA molecule encoding an amino acid sequence at least 90% identical or similar to SEQ ID NO: 2, and a third RNA molecule encoding an amino acid sequence at least 90% identical or similar to SEQ ID NO: 3. In an aspect, the non-coding RNA molecule suppresses the transcription or translation of a first RNA molecule encoding an amino acid sequence at least 90% identical or similar to SEQ ID NO: 1, a second RNA molecule encoding an amino acid sequence at least 90% identical or similar to SEQ ID NO: 2, and a third RNA molecule encoding an amino acid sequence at least 90% identical or similar to SEQ ID NO: 538. In an aspect, the non-coding RNA molecule suppresses the transcription or translation of a first RNA molecule encoding an amino acid sequence at least 90% identical or similar to SEQ ID NO: 1, a second RNA molecule encoding an amino acid sequence at least 90% identical or similar to SEQ ID NO: 3, and a third RNA molecule encoding an amino acid sequence at least 90% identical or similar to SEQ ID NO: 538. In an aspect, the non-coding RNA molecule suppresses the transcription or translation of a first RNA molecule encoding an amino acid sequence at least 90% identical or similar to SEQ ID NO: 2, a second RNA molecule encoding an amino acid sequence at least 90% identical or similar to SEQ ID NO: 3, and a third RNA molecule encoding an amino acid sequence at least 90% identical or similar to SEQ ID NO: 538. In an aspect, the non-coding RNA molecule suppresses the transcription or translation of a first RNA molecule encoding an amino acid sequence at least 90% identical or similar to SEQ ID NO: 1, a second RNA molecule encoding an amino acid sequence at least 90% identical or similar to SEQ ID NO: 2, a third RNA molecule encoding an amino acid sequence at least 90% identical or similar to SEQ ID NO: 3, and a fourth RNA molecule encoding an amino acid sequence at least 90% identical or similar to SEQ ID NO: 538. In an aspect, the non-coding RNA molecule suppresses the transcription or translation of a first RNA molecule encoding an amino acid sequence at least 95% identical or similar to SEQ ID NO: 1 and a second RNA molecule encoding an amino acid sequence at least 95% identical or similar to SEQ ID NO: 2. In an aspect, the non-coding RNA molecule suppresses the transcription or translation of a first RNA molecule encoding an amino acid sequence at least 95% identical or similar to SEQ ID NO: 1 and a second RNA molecule encoding an amino acid sequence at least 95% identical or similar to SEQ ID NO: 3. In an aspect, the non-coding RNA molecule suppresses the transcription or translation of a first RNA molecule encoding an amino acid sequence at least 95% identical or similar to SEQ ID NO: 2 and a second RNA molecule encoding an amino acid sequence at least 95% identical or similar to SEQ ID NO: 3. In an aspect, the non-coding RNA molecule suppresses the transcription or translation of a first RNA molecule encoding an amino acid sequence at least 95% identical or similar to SEQ ID NO: 1 and a second RNA molecule encoding an amino acid sequence at least 95% identical or similar to SEQ ID NO: 538. In an aspect, the non-coding RNA molecule suppresses the transcription or translation of a first RNA molecule encoding an amino acid sequence at least 95% identical or similar to SEQ ID NO: 2 and a second RNA molecule encoding an amino acid sequence at least 95% identical or similar to SEQ ID NO: 538. In an aspect, the non-coding RNA molecule suppresses the transcription or translation of a first RNA molecule encoding an amino acid sequence at least 95% identical or similar to SEQ ID NO: 3 and a second RNA molecule encoding an amino acid sequence at least 95% identical or similar to SEQ ID NO: 538. In an aspect, the non-coding RNA molecule suppresses the transcription or translation of a first RNA molecule encoding an amino acid sequence at least 95% identical or similar to SEQ ID NO: 1, a second RNA molecule encoding an amino acid sequence at least 95% identical or similar to SEQ ID NO: 2, and a third RNA molecule encoding an amino acid sequence at least 95% identical or similar to SEQ ID NO: 3. In an aspect, the non-coding RNA molecule suppresses the transcription or translation of a first RNA molecule encoding an amino acid sequence at least 95% identical or similar to SEQ ID NO: 1, a second RNA molecule encoding an amino acid sequence at least 95% identical or similar to SEQ ID NO: 2, and a third RNA molecule encoding an amino acid sequence at least 95% identical or similar to SEQ ID NO: 538. In an aspect, the non-coding RNA molecule suppresses the transcription or translation of a first RNA molecule encoding an amino acid sequence at least 95% identical or similar to SEQ ID NO: 1, a second RNA molecule encoding an amino acid sequence at least 95% identical or similar to SEQ ID NO: 3, and a third RNA molecule encoding an amino acid sequence at least 95% identical or similar to SEQ ID NO: 538. In an aspect, the non-coding RNA molecule suppresses the transcription or translation of a first RNA molecule encoding an amino acid sequence at least 95% identical or similar to SEQ ID NO: 2, a second RNA molecule encoding an amino acid sequence at least 95% identical or similar to SEQ ID NO: 3, and a third RNA molecule encoding an amino acid sequence at least 95% identical or similar to SEQ ID NO: 538. In an aspect, the non-coding RNA molecule suppresses the transcription or translation of a first RNA molecule encoding an amino acid sequence at least 95% identical or similar to SEQ ID NO: 1, a second RNA molecule encoding an amino acid sequence at least 95% identical or similar to SEQ ID NO: 2, a third RNA molecule encoding an amino acid sequence at least 95% identical or similar to SEQ ID NO: 3, and a fourth RNA molecule encoding an amino acid sequence at least 95% identical or similar to SEQ ID NO: 538. In an aspect, the non-coding RNA molecule suppresses the transcription or translation of a first RNA molecule encoding an amino acid sequence 100% identical or similar to SEQ ID NO: 1 and a second RNA molecule encoding an amino acid sequence 100% identical or similar to SEQ ID NO: 2. In an aspect, the non-coding RNA molecule suppresses the transcription or translation of a first RNA molecule encoding an amino acid sequence 100% identical or similar to SEQ ID NO: 1 and a second RNA molecule encoding an amino acid sequence 100% identical or similar to SEQ ID NO: 3. In an aspect, the non-coding RNA molecule suppresses the transcription or translation of a first RNA molecule encoding an amino acid sequence 100% identical or similar to SEQ ID NO: 2 and a second RNA molecule encoding an amino acid sequence 100% identical or similar to SEQ ID NO: 3. In an aspect, the non-coding RNA molecule suppresses the transcription or translation of a first RNA molecule encoding an amino acid sequence 100% identical or similar to SEQ ID NO: 1 and a second RNA molecule encoding an amino acid sequence 100% identical or similar to SEQ ID NO: 538. In an aspect, the non-coding RNA molecule suppresses the transcription or translation of a first RNA molecule encoding an amino acid sequence 100% identical or similar to SEQ ID NO: 2 and a second RNA molecule encoding an amino acid sequence 100% identical or similar to SEQ ID NO: 538. In an aspect, the non-coding RNA molecule suppresses the transcription or translation of a first RNA molecule encoding an amino acid sequence 100% identical or similar to SEQ ID NO: 3 and a second RNA molecule encoding an amino acid sequence 100% identical or similar to SEQ ID NO: 538. In an aspect, the non-coding RNA molecule suppresses the transcription or translation of a first RNA molecule encoding an amino acid sequence 100% identical or similar to SEQ ID NO: 1, a second RNA molecule encoding an amino acid sequence 100% identical or similar to SEQ ID NO: 2, and a third RNA molecule encoding an amino acid sequence 100% identical or similar to SEQ ID NO: 3. In an aspect, the non-coding RNA molecule suppresses the transcription or translation of a first RNA molecule encoding an amino acid sequence 100% identical or similar to SEQ ID NO: 1, a second RNA molecule encoding an amino acid sequence 100% identical or similar to SEQ ID NO: 2, and a third RNA molecule encoding an amino acid sequence 100% identical or similar to SEQ ID NO: 538. In an aspect, the non-coding RNA molecule suppresses the transcription or translation of a first RNA molecule encoding an amino acid sequence 100% identical or similar to SEQ ID NO: 1, a second RNA molecule encoding an amino acid sequence 100% identical or similar to SEQ ID NO: 3, and a third RNA molecule encoding an amino acid sequence 100% identical or similar to SEQ ID NO: 538. In an aspect, the non-coding RNA molecule suppresses the transcription or translation of a first RNA molecule encoding an amino acid sequence 100% identical or similar to SEQ ID NO: 2, a second RNA molecule encoding an amino acid sequence 100% identical or similar to SEQ ID NO: 3, and a third RNA molecule encoding an amino acid sequence 100% identical or similar to SEQ ID NO: 538. In an aspect, the non-coding RNA molecule suppresses the transcription or translation of a first RNA molecule encoding an amino acid sequence 100% identical or similar to SEQ ID NO: 1, a second RNA molecule encoding an amino acid sequence 100% identical or similar to SEQ ID NO: 2, a third RNA molecule encoding an amino acid sequence 100% identical or similar to SEQ ID NO: 3, and a fourth RNA molecule encoding an amino acid sequence 100% identical or similar to SEQ ID NO: 538.

In an aspect, this disclosure provides a method of producing a modified tobacco plant, the method comprising: (a) introducing a recombinant nucleic acid construct to at least one tobacco cell, where the recombinant nucleic acid construct comprises a heterologous promoter operably linked to a polynucleotide that encodes a non-coding RNA molecule; and (b) regenerating a modified tobacco plant from the at least one tobacco cell of step (a), where the modified tobacco plant comprises the recombinant nucleic acid construct, and where the non-coding RNA molecule suppresses the transcription or translation of at least one RNA molecule encoding an amino acid sequence at least 90% identical or similar to an amino acid sequence selected from the group consisting of SEQ ID NOs: 539 to 546 as compared to a control tobacco plant lacking the recombinant nucleic acid construct when grown under comparable conditions. In an aspect, this disclosure provides a method of producing a modified tobacco plant, the method comprising: (a) introducing a recombinant nucleic acid construct to at least one tobacco cell, where the recombinant nucleic acid construct comprises a heterologous promoter operably linked to a polynucleotide that encodes a non-coding RNA molecule; and (b) regenerating a modified tobacco plant from the at least one tobacco cell of step (a), where the modified tobacco plant comprises the recombinant nucleic acid construct, and where the non-coding RNA molecule suppresses the transcription or translation of at least one RNA molecule encoding an amino acid sequence at least 95% identical or similar to an amino acid sequence selected from the group consisting of SEQ ID NOs: 539 to 546 as compared to a control tobacco plant lacking the recombinant nucleic acid construct when grown under comparable conditions. In an aspect, this disclosure provides a method of producing a modified tobacco plant, the method comprising: (a) introducing a recombinant nucleic acid construct to at least one tobacco cell, where the recombinant nucleic acid construct comprises a heterologous promoter operably linked to a polynucleotide that encodes a non-coding RNA molecule; and (b) regenerating a modified tobacco plant from the at least one tobacco cell of step (a), where the modified tobacco plant comprises the recombinant nucleic acid construct, and where the non-coding RNA molecule suppresses the transcription or translation of at least one RNA molecule encoding an amino acid sequence 100% identical or similar to an amino acid sequence selected from the group consisting of SEQ ID NOs: 539 to 546 as compared to a control tobacco plant lacking the recombinant nucleic acid construct when grown under comparable conditions.

In an aspect, this disclosure provides a method of producing a modified tobacco plant, the method comprising: (a) introducing a recombinant nucleic acid construct to at least one tobacco cell, where the recombinant nucleic acid construct comprises a heterologous promoter operably linked to a polynucleotide that encodes a non-coding RNA molecule; and (b) regenerating a modified tobacco plant from the at least one tobacco cell of step (a), where the modified tobacco plant comprises the recombinant nucleic acid construct, and where the non-coding RNA molecule suppresses the transcription or translation of at least one RNA molecule comprising a nucleic acid sequence at least 90% identical to a nucleic acid sequence selected from the group consisting of SEQ ID NOs: 4, 5, 6, and 612 as compared to a control tobacco plant lacking the recombinant nucleic acid construct when grown under comparable conditions. In an aspect, the non-coding RNA molecule suppresses the transcription or translation of a first RNA molecule comprising a nucleic acid sequence at least 90% identical to SEQ ID NO: 4 and a second RNA molecule comprising a nucleic acid sequence at least 90% identical to SEQ ID NO: 5. In an aspect, the non-coding RNA molecule suppresses the transcription or translation of a first RNA molecule comprising a nucleic acid sequence at least 90% identical to SEQ ID NO: 4 and a second RNA molecule comprising a nucleic acid sequence at least 90% identical to SEQ ID NO: 6. In an aspect, the non-coding RNA molecule suppresses the transcription or translation of a first RNA molecule comprising a nucleic acid sequence at least 90% identical to SEQ ID NO: 5 and a second RNA molecule comprising a nucleic acid sequence at least 90% identical to SEQ ID NO: 6. In an aspect, the non-coding RNA molecule suppresses the transcription or translation of a first RNA molecule comprising a nucleic acid sequence at least 90% identical to SEQ ID NO: 4 and a second RNA molecule comprising a nucleic acid sequence at least 90% identical to SEQ ID NO: 612. In an aspect, the non-coding RNA molecule suppresses the transcription or translation of a first RNA molecule comprising a nucleic acid sequence at least 90% identical to SEQ ID NO: 5 and a second RNA molecule comprising a nucleic acid sequence at least 90% identical to SEQ ID NO: 612. In an aspect, the non-coding RNA molecule suppresses the transcription or translation of a first RNA molecule comprising a nucleic acid sequence at least 90% identical to SEQ ID NO: 6 and a second RNA molecule comprising a nucleic acid sequence at least 90% identical to SEQ ID NO: 612. In an aspect, the non-coding RNA molecule suppresses the transcription or translation of a first RNA molecule comprising a nucleic acid sequence at least 90% identical to SEQ ID NO: 4, a second RNA molecule comprising a nucleic acid sequence at least 90% identical to SEQ ID NO: 5, and a third RNA molecule comprising a nucleic acid sequence at least 90% identical to SEQ ID NO: 6. In an aspect, the non-coding RNA molecule suppresses the transcription or translation of a first RNA molecule comprising a nucleic acid sequence at least 90% identical to SEQ ID NO: 4, a second RNA molecule comprising a nucleic acid sequence at least 90% identical to SEQ ID NO: 5, and a third RNA molecule comprising a nucleic acid sequence at least 90% identical to SEQ ID NO: 612. In an aspect, the non-coding RNA molecule suppresses the transcription or translation of a first RNA molecule comprising a nucleic acid sequence at least 90% identical to SEQ ID NO: 4, a second RNA molecule comprising a nucleic acid sequence at least 90% identical to SEQ ID NO: 6, and a third RNA molecule comprising a nucleic acid sequence at least 90% identical to SEQ ID NO: 612. In an aspect, the non-coding RNA molecule suppresses the transcription or translation of a first RNA molecule comprising a nucleic acid sequence at least 90% identical to SEQ ID NO: 5, a second RNA molecule comprising a nucleic acid sequence at least 90% identical to SEQ ID NO: 6, and a third RNA molecule comprising a nucleic acid sequence at least 90% identical to SEQ ID NO: 612. In an aspect, the non-coding RNA molecule suppresses the transcription or translation of a first RNA molecule comprising a nucleic acid sequence at least 90% identical to SEQ ID NO: 4, a second RNA molecule comprising a nucleic acid sequence at least 90% identical to SEQ ID NO: 5, a third RNA molecule comprising a nucleic acid sequence at least 90% identical to SEQ ID NO: 6, and a fourth RNA molecule comprising a nucleic acid sequence at least 90% identical to SEQ ID NO: 612. In an aspect, the non-coding RNA molecule suppresses the transcription or translation of a first RNA molecule comprising a nucleic acid sequence at least 95% identical to SEQ ID NO: 4 and a second RNA molecule comprising a nucleic acid sequence at least 95% identical to SEQ ID NO: 5. In an aspect, the non-coding RNA molecule suppresses the transcription or translation of a first RNA molecule comprising a nucleic acid sequence at least 95% identical to SEQ ID NO: 4 and a second RNA molecule comprising a nucleic acid sequence at least 95% identical to SEQ ID NO: 6. In an aspect, the non-coding RNA molecule suppresses the transcription or translation of a first RNA molecule comprising a nucleic acid sequence at least 95% identical to SEQ ID NO: 5 and a second RNA molecule comprising a nucleic acid sequence at least 95% identical to SEQ ID NO: 6. In an aspect, the non-coding RNA molecule suppresses the transcription or translation of a first RNA molecule comprising a nucleic acid sequence at least 95% identical to SEQ ID NO: 4 and a second RNA molecule comprising a nucleic acid sequence at least 95% identical to SEQ ID NO: 612. In an aspect, the non-coding RNA molecule suppresses the transcription or translation of a first RNA molecule comprising a nucleic acid sequence at least 95% identical to SEQ ID NO: 5 and a second RNA molecule comprising a nucleic acid sequence at least 95% identical to SEQ ID NO: 612. In an aspect, the non-coding RNA molecule suppresses the transcription or translation of a first RNA molecule comprising a nucleic acid sequence at least 95% identical to SEQ ID NO: 6 and a second RNA molecule comprising a nucleic acid sequence at least 95% identical to SEQ ID NO: 612. In an aspect, the non-coding RNA molecule suppresses the transcription or translation of a first RNA molecule comprising a nucleic acid sequence at least 95% identical to SEQ ID NO: 4, a second RNA molecule comprising a nucleic acid sequence at least 95% identical to SEQ ID NO: 5, and a third RNA molecule comprising a nucleic acid sequence at least 95% identical to SEQ ID NO: 6. In an aspect, the non-coding RNA molecule suppresses the transcription or translation of a first RNA molecule comprising a nucleic acid sequence at least 95% identical to SEQ ID NO: 4, a second RNA molecule comprising a nucleic acid sequence at least 95% identical to SEQ ID NO: 5, and a third RNA molecule comprising a nucleic acid sequence at least 95% identical to SEQ ID NO: 612. In an aspect, the non-coding RNA molecule suppresses the transcription or translation of a first RNA molecule comprising a nucleic acid sequence at least 95% identical to SEQ ID NO: 4, a second RNA molecule comprising a nucleic acid sequence at least 95% identical to SEQ ID NO: 6, and a third RNA molecule comprising a nucleic acid sequence at least 95% identical to SEQ ID NO: 612. In an aspect, the non-coding RNA molecule suppresses the transcription or translation of a first RNA molecule comprising a nucleic acid sequence at least 95% identical to SEQ ID NO: 5, a second RNA molecule comprising a nucleic acid sequence at least 95% identical to SEQ ID NO: 6, and a third RNA molecule comprising a nucleic acid sequence at least 95% identical to SEQ ID NO: 612. In an aspect, the non-coding RNA molecule suppresses the transcription or translation of a first RNA molecule comprising a nucleic acid sequence at least 95% identical to SEQ ID NO: 4, a second RNA molecule comprising a nucleic acid sequence at least 95% identical to SEQ ID NO: 5, a third RNA molecule comprising a nucleic acid sequence at least 95% identical to SEQ ID NO: 6, and a fourth RNA molecule comprising a nucleic acid sequence at least 95% identical to SEQ ID NO: 612. In an aspect, the non-coding RNA molecule suppresses the transcription or translation of a first RNA molecule comprising a nucleic acid sequence 100% identical to SEQ ID NO: 4 and a second RNA molecule comprising a nucleic acid sequence 100% identical to SEQ ID NO: 5. In an aspect, the non-coding RNA molecule suppresses the transcription or translation of a first RNA molecule comprising a nucleic acid sequence 100% identical to SEQ ID NO: 4 and a second RNA molecule comprising a nucleic acid sequence 100% identical to SEQ ID NO: 6. In an aspect, the non-coding RNA molecule suppresses the transcription or translation of a first RNA molecule comprising a nucleic acid sequence 100% identical to SEQ ID NO: 5 and a second RNA molecule comprising a nucleic acid sequence 100% identical to SEQ ID NO: 6. In an aspect, the non-coding RNA molecule suppresses the transcription or translation of a first RNA molecule comprising a nucleic acid sequence 100% identical to SEQ ID NO: 4 and a second RNA molecule comprising a nucleic acid sequence 100% identical to SEQ ID NO: 612. In an aspect, the non-coding RNA molecule suppresses the transcription or translation of a first RNA molecule comprising a nucleic acid sequence 100% identical to SEQ ID NO: 5 and a second RNA molecule comprising a nucleic acid sequence 100% identical to SEQ ID NO: 612. In an aspect, the non-coding RNA molecule suppresses the transcription or translation of a first RNA molecule comprising a nucleic acid sequence 100% identical to SEQ ID NO: 6 and a second RNA molecule comprising a nucleic acid sequence 100% identical to SEQ ID NO: 612. In an aspect, the non-coding RNA molecule suppresses the transcription or translation of a first RNA molecule comprising a nucleic acid sequence 100% identical to SEQ ID NO: 4, a second RNA molecule comprising a nucleic acid sequence 100% identical to SEQ ID NO: 5, and a third RNA molecule comprising a nucleic acid sequence 100% identical to SEQ ID NO: 6. In an aspect, the non-coding RNA molecule suppresses the transcription or translation of a first RNA molecule comprising a nucleic acid sequence 100% identical to SEQ ID NO: 4, a second RNA molecule comprising a nucleic acid sequence 100% identical to SEQ ID NO: 5, and a third RNA molecule comprising a nucleic acid sequence 100% identical to SEQ ID NO: 612. In an aspect, the non-coding RNA molecule suppresses the transcription or translation of a first RNA molecule comprising a nucleic acid sequence 100% identical to SEQ ID NO: 4, a second RNA molecule comprising a nucleic acid sequence 100% identical to SEQ ID NO: 6, and a third RNA molecule comprising a nucleic acid sequence 100% identical to SEQ ID NO: 612. In an aspect, the non-coding RNA molecule suppresses the transcription or translation of a first RNA molecule comprising a nucleic acid sequence 100% identical to SEQ ID NO: 5, a second RNA molecule comprising a nucleic acid sequence 100% identical to SEQ ID NO: 6, and a third RNA molecule comprising a nucleic acid sequence 100% identical to SEQ ID NO: 612. In an aspect, the non-coding RNA molecule suppresses the transcription or translation of a first RNA molecule comprising a nucleic acid sequence 100% identical to SEQ ID NO: 4, a second RNA molecule comprising a nucleic acid sequence 100% identical to SEQ ID NO: 5, a third RNA molecule comprising a nucleic acid sequence 100% identical to SEQ ID NO: 6, and a fourth RNA molecule comprising a nucleic acid sequence 100% identical to SEQ ID NO: 612.

Numerous methods for introducing a recombinant nucleic acid construct to a plant cell are known in the art, which can be used according to methods of the present application to produce a modified tobacco cell and/or a modified tobacco plant. Any suitable method or technique for transformation of a plant cell known in the art can be used according to present methods. Effective methods for transformation of plants include bacterially mediated transformation, such as Agrobacterium-mediated or Rhizobium-mediated transformation and microprojectile bombardment-mediated transformation. A variety of methods are known in the art for transforming explants with a transformation vector via bacterially-mediated transformation or microprojectile bombardment and then subsequently culturing, etc., those explants to regenerate or develop transgenic plants. Other methods for plant transformation, such as microinjection, electroporation, vacuum infiltration, pressure, sonication, silicon carbide fiber agitation, polyethylene glycol (PEG)-mediated transformation, etc., are also known in the art. Modified tobacco plants produced by these transformation (“introduction”) methods can be chimeric or non-chimeric for the transformation event depending on the methods and explants used.

Methods of transforming plant cells are well known by persons of ordinary skill in the art. For instance, specific instructions for transforming plant cells by microprojectile bombardment with particles coated with recombinant DNA (e.g., biolistic transformation) are found in U.S. Pat. Nos. 5,550,318; 5,538,880 6,160,208; 6,399,861; and 6,153,812 and Agrobacterium-mediated transformation is described in U.S. Pat. Nos. 5,159,135; 5,824,877; 5,591,616; 6,384,301; 5,750,871; 5,463,174; and 5,188,958, all of which are incorporated herein by reference. Additional methods for transforming plants can be found in, for example, Compendium of Transgenic Crop Plants (2009) Blackwell Publishing. Any appropriate method known to those skilled in the art can be used to transform a tobacco cell with any of the nucleic acid molecules provided herein.

In an aspect, a method of introducing a nucleic acid molecule to a tobacco cell comprises Agrobacterium-mediated transformation. In another aspect, a method of introducing a nucleic acid molecule to a tobacco cell comprises PEG-mediated transformation. In another aspect, a method of introducing a nucleic acid molecule to a tobacco cell comprises biolistic transformation. In another aspect, a method of introducing a nucleic acid molecule to a tobacco cell comprises liposome-mediated transfection (lipofection). In another aspect, a method of introducing a nucleic acid molecule to a tobacco cell comprises lentiviral transfection.

Lipofection is described in e.g., U.S. Pat. Nos. 5,049,386, 4,946,787; and 4,897,355) and lipofection reagents are sold commercially (e.g., Transfectam™ and Lipofectin™). Cationic and neutral lipids that are suitable for efficient receptor-recognition lipofection of polynucleotides include those of WO 91/17424 and WO 91/16024. Delivery can be to cells (e.g. in vitro or ex vivo administration) or target tissues (e.g. in vivo administration).

Any tobacco cell from which a fertile tobacco plant can be regenerated is contemplated as a useful recipient cell for practice of this disclosure. In an aspect, a recombinant nucleic acid construct is introduced to a tobacco cell. In an aspect, a recombinant nucleic acid construct is introduced to a tobacco protoplast cell. In another aspect, a recombinant nucleic acid construct is introduced to a tobacco callus cell. In an aspect, a recombinant nucleic acid construct is introduced to a tobacco cell selected from the group consisting of a seed cell, a fruit cell, a leaf cell, a cotyledon cell, a hypocotyl cell, a meristem cell, an embryo cell, an endosperm cell, a root cell, a shoot cell, a stem cell, a trichome cell, a flower cell, an inflorescence cell, a stalk cell, a pedicel cell, a style cell, a stigma cell, a receptacle cell, a petal cell, a sepal cell, a pollen cell, an anther cell, a filament cell, an ovary cell, an ovule cell, a pericarp cell, and a phloem cell.

Callus can be initiated from various tissue sources, including, but not limited to, immature embryos or parts of embryos, seedling apical meristems, microspores, and the like. Those cells which are capable of proliferating as callus can serve as recipient cells for transformation. Practical transformation methods and materials for making modified tobacco plants of this disclosure (e.g., various media and recipient target cells, transformation of immature embryos, and subsequent regeneration of fertile transgenic plants) are disclosed, for example, in U.S. Pat. Nos. 6,194,636 and 6,232,526 and U. S. Patent Application Publication 2004/0216189, all of which are incorporated herein by reference. Additional, non-limiting, information regarding regeneration of modified tobacco plants following transformation can be found in Example 2 of this disclosure.

In some aspects, a modified tobacco plant provided herein comprises a statistically significant increase in neophytadiene as compared to a control tobacco plant grown under comparable conditions. In some aspects, a modified tobacco plant provided herein comprises a statistically significant decrease in cis-abienol as compared to a control tobacco plant grown under comparable conditions. In some aspects, a modified tobacco plant provided herein comprises a statistically significant decrease in 4,8,13-duvatriene-1,3-diol as compared to a control tobacco plant grown under comparable conditions. In some aspects, a modified tobacco plant provided herein comprises a statistically significant decrease in thunbergol as compared to a control tobacco plant grown under comparable conditions.

The Following Embodiments are Specifically Envisioned, Although they are not Intended to be Limiting:
1. A modified tobacco plant, or part thereof, comprising a non-natural mutation in an endogenous nucleic acid molecule, wherein the endogenous nucleic acid molecule encodes a HAIRPLUS1a (NtHAP1a) protein comprising the amino acid sequence of SEQ ID NO: 1, and wherein expression or activity of the NtHAP1a protein is reduced in the modified tobacco plant as compared to a control tobacco plant lacking the non-natural mutation when grown under comparable conditions.
2. A modified tobacco plant, or part thereof, comprising a non-natural mutation in an endogenous nucleic acid molecule, wherein the endogenous nucleic acid molecule encodes a HAIRPLUS1b (NtHAP1b) protein comprising the amino acid sequence of SEQ ID NO: 2, and wherein expression or activity of the NtHAP1b protein is reduced in the modified tobacco plant as compared to a control tobacco plant lacking the non-natural mutation when grown under comparable conditions.
3. A modified tobacco plant, or part thereof, comprising a non-natural mutation in an endogenous nucleic acid molecule, wherein the endogenous nucleic acid molecule encodes a HAIRPLUS2 (NtHAP2) protein comprising the amino acid sequence of SEQ ID NO: 3, and wherein expression or activity of the NtHAP2 protein is reduced in the modified tobacco plant as compared to a control tobacco plant lacking the non-natural mutation when grown under comparable conditions.
4. The modified tobacco plant, or part thereof, of embodiment 1, wherein the endogenous nucleic acid molecule is a coding sequence comprising SEQ ID NO: 4.
5. The modified tobacco plant, or part thereof, of embodiment 2, wherein the endogenous nucleic acid molecule is a coding sequence comprising SEQ ID NO: 5.
6. The modified tobacco plant, or part thereof, of embodiment 3, wherein the endogenous nucleic acid molecule is a coding sequence comprising SEQ ID NO: 6.
7. The modified tobacco plant, or part thereof, of embodiment 1, wherein the endogenous nucleic acid molecule is a genomic sequence comprising SEQ ID NO: 7.
8. The modified tobacco plant, or part thereof, of embodiment 2, wherein the endogenous nucleic acid molecule is a genomic sequence comprising SEQ ID NO: 8.
9. The modified tobacco plant, or part thereof, of embodiment 3, wherein the endogenous nucleic acid molecule is a genomic sequence comprising SEQ ID NO: 9.
10. The modified tobacco plant of embodiment 1, wherein the modified tobacco plant further comprises a second non-natural mutation in an endogenous nucleic acid molecule encoding a HAIRPLUS1b (NtHAP1b) protein, wherein the endogenous NtHAP1b protein comprises the amino acid sequence of SEQ ID NO: 2, and wherein expression or activity of NtHAP1b is reduced in the modified tobacco plant as compared to a control tobacco plant lacking the non-natural mutation when grown under comparable conditions.
11. The modified tobacco plant of embodiment 10, wherein the modified tobacco plant further comprises a third non-natural mutation in an endogenous nucleic acid molecule encoding a HAIRPLUS2 (NtHAP2) protein, wherein the endogenous NtHAP2 protein comprises the amino acid sequence of SEQ ID NO: 3, and wherein expression or activity of NtHAP2 is reduced in the modified tobacco plant as compared to a control tobacco plant lacking the non-natural mutation when grown under comparable conditions.
12. The modified tobacco plant of embodiment 1, wherein the modified tobacco plant further comprises a second non-natural mutation in an endogenous nucleic acid molecule encoding a HAIRPLUS2 (NtHAP2) protein, wherein the endogenous NtHAP2 protein comprises the amino acid sequence of SEQ ID NO: 3, and wherein expression or activity of NtHAP2 is reduced in the modified tobacco plant as compared to a control tobacco plant lacking the non-natural mutation when grown under comparable conditions.
13. The modified tobacco plant of embodiment 2, wherein the modified tobacco plant further comprises a second non-natural mutation in an endogenous nucleic acid molecule encoding a HAIRPLUS2 (NtHAP2) protein, wherein the endogenous NtHAP2 protein comprises the amino acid sequence of SEQ ID NO: 3, and wherein expression or activity of NtHAP2 is reduced in the modified tobacco plant as compared to a control tobacco plant lacking the non-natural mutation when grown under comparable conditions.
14. The modified tobacco plant, or part thereof, of any one of embodiments 1 to 3, wherein the non-natural mutation is a null mutation.
15. The modified tobacco plant, or part thereof, of any one of embodiments 1 to 3, wherein the non-natural mutation comprises a premature stop codon as compared to the endogenous nucleic acid molecule.
16. The modified tobacco plant, or part thereof, of embodiment 1, wherein the non-natural mutation results in a truncated NtHAP1a protein as compared to SEQ ID NO: 1.
17. The modified tobacco plant, or part thereof, of embodiment 2, wherein the non-natural mutation results in a truncated NtHAP1b protein as compared to SEQ ID NO: 2.
18. The modified tobacco plant, or part thereof, of embodiment 3, wherein the non-natural mutation results in a truncated NtHAP2 protein as compared to SEQ ID NO: 3.
19. The modified tobacco plant, or part thereof, of any one of embodiments 1 to 3, wherein the non-natural mutation comprises the deletion of at least one nucleotide as compared to the endogenous nucleic acid molecule.
20. The modified tobacco plant, or part thereof, of any one of embodiments 1 to 3, wherein the non-natural mutation comprises the insertion of at least one nucleotide as compared to the endogenous nucleic acid molecule.
21. The modified tobacco plant, or part thereof, of any one of embodiments 1 to 3, wherein the non-natural mutation comprises the substitution of at least one nucleotide as compared to the endogenous nucleic acid molecule.
22. The modified tobacco plant, or part thereof, of any one of embodiments 1 to 3, wherein the non-natural mutation comprises the inversion of at least two nucleotides as compared to the endogenous nucleic acid molecule.
23. The modified tobacco plant, or part thereof, of any one of embodiments 1 to 3, wherein the non-natural mutation comprises at least one mutation selected from the group consisting of a nonsense mutation, a missense mutation, a frameshift mutation, and a splice-site mutation.
24. The modified tobacco plant, or part thereof, of any one of embodiments 1 to 3, wherein the non-natural mutation is positioned within a promoter of the endogenous nucleic acid molecule.
25. The modified tobacco plant, or part thereof, of any one of embodiments 1 to 3, wherein the non-natural mutation is positioned within a 5′-untranslated region of the endogenous nucleic acid molecule.
26. The modified tobacco plant, or part thereof, of any one of embodiments 1 to 3, wherein the non-natural mutation is positioned within a 3′-untranslated region of the endogenous nucleic acid molecule.
27. The modified tobacco plant, or part thereof, of any one of embodiments 1 to 3, wherein the non-natural mutation is positioned within an exon of the endogenous nucleic acid molecule.
28. The modified tobacco plant, or part thereof, of embodiment 1, wherein the non-natural mutation comprises a nucleic acid sequence selected from the group consisting of SEQ ID NOs: 34, 35, 37, 40, 42, 44, 46, 47, 49, 50, 53, 57, 58, 61 to 71, 135 to 137, 140 to 142, 144, 145, 148, 149, 152, 153, 156, 157, 160, 161, 164, 166, 167, 169, 170, 173, 174, 177, 178, 184, 198, 202, 207, 209, 215, 216, 221, and 223 to 269.
29. The modified tobacco plant, or part thereof, of embodiment 1, wherein the non-natural mutation comprises a nucleic acid sequence selected from the group consisting of SEQ ID NOs: 72, 75, 77, 79, 80, 83, 86, 87, 286, 287, 290, 291, 294, 295, 297 to 299, 301, 303, 304, 307 to 310, 313, 316 to 318, 326, 327, 329, 331, 336, 339, 341, 343, 345, 346, 349, and 350.
30. The modified tobacco plant, or part thereof, of embodiment 1, wherein the non-natural mutation comprises a nucleic acid sequence encoding an amino acid sequence selected from the group consisting of SEQ ID NOs: 97, 98, 100, 103, 105, 107, 109, 110, 112, 113, 116, 120, 121, 124 to 134, 385 to 387, 390 to 392, 394, 395, 398, 399, 402, 403, 406, 407, 410, 411, 414, 416, 417, 419, 420, 423, 424, 427, 428, 434, 448, 452, 457, 459, 465, 466, 471, and 473 to 519.
31. The modified tobacco plant, or part thereof, of embodiment 2, wherein the non-natural mutation comprises a nucleic acid sequence selected from the group consisting of SEQ ID NOs: 36, 38, 39, 41, 43, 45, 48, 138, 139, 143, 146, 147, 150, 151, 154, 155, 158, 159, 162, 163, 165, 168, 171, 172, 175, 176, 179 to 181, 185, 186, 191, 194, 195, 199, 203, 205, 210, 218, 222, and 270 to 277.
32. The modified tobacco plant, or part thereof, of embodiment 2, wherein the non-natural mutation comprises a nucleic acid sequence selected from the group consisting of SEQ ID NOs: 73, 74, 76, 78, 81, 82, 84, 85, 88, 89, 285, 288, 289, 292, 293, 296, 300, 302, 305, 306, 311, 312, 314, 315, 319, 320, 323 to 325, 328, 330, 332 to 335, 337, 338, 340, 342, 344, 347, 348, and 351.
33. The modified tobacco plant, or part thereof, of embodiment 2, wherein the non-natural mutation comprises a nucleic acid sequence encoding an amino acid sequence selected from the group consisting of SEQ ID NOs: 99, 101, 102, 104, 106, 108, 111, 117, 118, 388, 389, 393, 396, 397, 400, 401, 404, 405, 408, 409, 412, 413, 415, 418, 421, 422, 425, 426, 429 to 431, 435, 436, 441, 444, 445, 449, 453, 455, 460, 468, 472, and 520 to 526.
34. The modified tobacco plant, or part thereof, of embodiment 3, wherein the non-natural mutation comprises a nucleic acid sequence selected from the group consisting of SEQ ID NOs: 51, 52, 56, 182, 183, 187 to 190, 192, 193, 196, 197, 200, 201, 204, 206, 208, 211 to 214, 217, 219, 220, and 278 to 284.
35. The modified tobacco plant, or part thereof, of embodiment 3, wherein the non-natural mutation comprises a nucleic acid sequence selected from the group consisting of SEQ ID NOs: 321 and 322.
36. The modified tobacco plant, or part thereof, of embodiment 3, wherein the non-natural mutation comprises a nucleic acid sequence encoding an amino acid sequence selected from the group consisting of SEQ ID NOs: 114, 115, 119, 432, 433, 437 to 440, 442, 443, 446, 447, 450, 451, 454, 456, 458, 461 to 464, 467, 469, 470, and 527 to 533.
37. A modified tobacco plant, or part thereof, comprising a recombinant nucleic acid construct comprising a heterologous promoter operably linked to a polynucleotide that encodes a non-coding RNA molecule, wherein the non-coding RNA molecule suppresses the transcription or translation of at least one RNA molecule encoding a protein comprising an amino acid sequence at least 90% identical or similar to an amino acid sequence selected from the group consisting of SEQ ID NOs: 1, 2, 3, and 538, and wherein the transcription or translation is as compared to a control tobacco plant lacking the recombinant nucleic acid construct when grown under comparable conditions.
38. The modified tobacco plant, or part thereof, of embodiment 37, wherein the non-coding RNA molecule suppresses the transcription or translation of a first RNA molecule encoding a protein comprising the amino acid sequence of SEQ ID NO: 1 and a second RNA molecule encoding a protein comprising the amino acid sequence of SEQ ID NO: 2.
39. The modified tobacco plant, or part thereof, of embodiment 37, wherein the non-coding RNA molecule suppresses the transcription or translation of a first RNA molecule encoding a protein comprising the amino acid sequence of SEQ ID NO: 1 and a second RNA molecule encoding a protein comprising the amino acid sequence of SEQ ID NO: 3.
40. The modified tobacco plant, or part thereof, of embodiment 37, wherein the non-coding RNA molecule suppresses the transcription or translation of a first RNA molecule encoding a protein comprising the amino acid sequence of SEQ ID NO: 2 and a second RNA molecule encoding a protein comprising the amino acid sequence of SEQ ID NO: 3.
41. The modified tobacco plant, or part thereof, of embodiment 37, wherein the non-coding RNA molecule suppresses the transcription or translation of a first RNA molecule encoding a protein comprising the amino acid sequence of SEQ ID NO: 1, a second RNA molecule encoding a protein comprising the amino acid sequence of SEQ ID NO: 2, and a third RNA molecule encoding a protein comprising the amino acid sequence of SEQ ID NO: 3.
42. The modified tobacco plant, or part thereof, of any one of embodiments 37 to 41, wherein the non-coding RNA molecule is a microRNA (miRNA) molecule.
43. A modified tobacco plant, or part thereof, comprising a non-natural mutation in a nucleic acid molecule encoding a HAIRPLUS1a (NtHAP1a) protein, wherein the nucleic acid molecule comprises a nucleic acid sequence selected from the group consisting of SEQ ID NOs: 20 to 23.
44. The modified tobacco plant, or part thereof, of embodiment 43, wherein expression or activity of the NtHAP1a protein is reduced in the modified tobacco plant as compared to a control tobacco plant lacking the non-natural mutation when grown under comparable conditions.
45. A modified tobacco plant, or part thereof, comprising a mutated HAIRPLUS1a (NtHAP1a) protein, wherein the mutated NtHAP1a protein comprises an amino acid sequence selected from the group consisting of SEQ ID NOs: 27 to 30.
46. The modified tobacco plant, or part thereof, of embodiment 45, wherein expression or activity of the mutated NtHAP1a protein is reduced in the modified tobacco plant as compared to a control tobacco plant comprising a wild type NtHAP1a protein when grown under comparable conditions.
47. A modified tobacco plant, or part thereof, comprising a non-natural mutation in a nucleic acid molecule encoding a HAIRPLUS1b (NtHAP1b) protein, wherein the nucleic acid molecule comprises a nucleic acid sequence selected from the group consisting of SEQ ID NOs: 24 and 25.
48. The modified tobacco plant, or part thereof, of embodiment 47, wherein expression or activity of the NtHAP1b protein is reduced in the modified tobacco plant as compared to a control tobacco plant lacking the non-natural mutation when grown under comparable conditions.
49. A modified tobacco plant, or part thereof, comprising a mutated HAIRPLUS1b (NtHAP1b) protein, wherein the mutated NtHAP1b protein comprises an amino acid sequence selected from the group consisting of SEQ ID NOs: 31 and 32.
50. The modified tobacco plant, or part thereof, of embodiment 49, wherein expression or activity of the mutated NtHAP1b protein is reduced in the modified tobacco plant as compared to a control tobacco plant comprising a wild type NtHAP1b protein when grown under comparable conditions.
51. A modified tobacco plant, or part thereof, comprising a non-natural mutation in a nucleic acid molecule encoding a HAIRPLUS2 (NtHAP2) protein, wherein the nucleic acid molecule comprises the nucleic acid sequence of SEQ ID NO: 26.
52. The modified tobacco plant, or part thereof, of embodiment 51, wherein expression or activity of the NtHAP2 protein is reduced in the modified tobacco plant as compared to a control tobacco plant lacking the non-natural mutation when grown under comparable conditions.
53. A modified tobacco plant, or part thereof, comprising a mutated HAIRPLUS2 (NtHAP2) protein, wherein the mutated NtHAP2 protein comprises the amino acid sequence of SEQ ID NO: 33.
54. The modified tobacco plant, or part thereof, of embodiment 53, wherein expression or activity of the mutated NtHAP2 protein is reduced in the modified tobacco plant as compared to a control tobacco plant comprising a wild type NtHAP2 protein when grown under comparable conditions.
55. A modified tobacco plant, or part thereof, comprising at least one mutant allele of an endogenous locus encoding a HAIRPLUS1a (NtHAP1a) protein, wherein the at least one mutant allele comprises a nucleic acid sequence selected from the group consisting of SEQ ID NOs: 34, 35, 37, 40, 42, 44, 46, 47, 49, 50, 53, 57, 58, 61 to 71, 135 to 137, 140 to 142, 144, 145, 148, 149, 152, 153, 156, 157, 160, 161, 164, 166, 167, 169, 170, 173, 174, 177, 178, 184, 198, 202, 207, 209, 215, 216, 221, and 223 to 269.
56. A modified tobacco plant, or part thereof, comprising at least one mutant allele of an endogenous locus encoding a HAIRPLUS1a (NtHAP1a) protein, wherein the at least one mutant allele comprises a nucleic acid sequence selected from the group consisting of SEQ ID NOs: 72, 75, 77, 79, 80, 83, 86, 87, 286, 287, 290, 291, 294, 295, 297 to 299, 301, 303, 304, 307 to 310, 313, 316 to 318, 326, 327, 329, 331, 336, 339, 341, 343, 345, 346, 349, and 350.
57. A modified tobacco plant, or part thereof, comprising at least one mutant allele of an endogenous locus encoding a HAIRPLUS1a (NtHAP1a) protein, wherein the at least one mutant allele comprises a nucleic acid sequence encoding an amino acid sequence selected from the group consisting of SEQ ID NOs: 97, 98, 100, 103, 105, 107, 109, 110, 112, 113, 116, 120, 121, 124 to 134, 385 to 387, 390 to 392, 394, 395, 398, 399, 402, 403, 406, 407, 410, 411, 414, 416, 417, 419, 420, 423, 424, 427, 428, 434, 448, 452, 457, 459, 465, 466, 471, and 473 to 519.
58. A modified tobacco plant, or part thereof, comprising at least one mutant allele of an endogenous locus encoding a HAIRPLUS1b (NtHAP1b) protein, wherein the at least one mutant allele comprises a nucleic acid sequence selected from the group consisting of SEQ ID NOs: 36, 38, 39, 41, 43, 45, 48, 138, 139, 143, 146, 147, 150, 151, 154, 155, 158, 159, 162, 163, 165, 168, 171, 172, 175, 176, 179 to 181, 185, 186, 191, 194, 195, 199, 203, 205, 210, 218, 222, and 270 to 277.
59. A modified tobacco plant, or part thereof, comprising at least one mutant allele of an endogenous locus encoding a HAIRPLUS1b (NtHAP1b) protein, wherein the at least one mutant allele comprises a nucleic acid sequence selected from the group consisting of SEQ ID NOs: 73, 74, 76, 78, 81, 82, 84, 85, 88, 89, 285, 288, 289, 292, 293, 296, 300, 302, 305, 306, 311, 312, 314, 315, 319, 320, 323 to 325, 328, 330, 332 to 335, 337, 338, 340, 342, 344, 347, 348, and 351.
60. A modified tobacco plant, or part thereof, comprising at least one mutant allele of an endogenous locus encoding a HAIRPLUS1b (NtHAP1b) protein, wherein the at least one mutant allele comprises a nucleic acid sequence encoding an amino acid sequence selected from the group consisting of SEQ ID NOs: 99, 101, 102, 104, 106, 108, 111, 117, 118, 388, 389, 393, 396, 397, 400, 401, 404, 405, 408, 409, 412, 413, 415, 418, 421, 422, 425, 426, 429 to 431, 435, 436, 441, 444, 445, 449, 453, 455, 460, 468, 472, and 520 to 526.
61. A modified tobacco plant, or part thereof, comprising at least one mutant allele of an endogenous locus encoding a HAIRPLUS2 (NtHAP2) protein, wherein the at least one mutant allele comprises a nucleic acid sequence selected from the group consisting of SEQ ID NOs: 51, 52, 56, 182, 183, 187 to 190, 192, 193, 196, 197, 200, 201, 204, 206, 208, 211 to 214, 217, 219, 220, and 278 to 284.
62. A modified tobacco plant, or part thereof, comprising at least one mutant allele of an endogenous locus encoding a HAIRPLUS2 (NtHAP2) protein, wherein the at least one mutant allele comprises a nucleic acid sequence selected from the group consisting of SEQ ID NOs: 321 and 322.
63. A modified tobacco plant, or part thereof, comprising at least one mutant allele of an endogenous locus encoding a HAIRPLUS2 (NtHAP2) protein, wherein the at least one mutant allele comprises a nucleic acid sequence encoding an amino acid sequence selected from the group consisting of SEQ ID NOs: 114, 115, 119, 432, 433, 437 to 440, 442, 443, 446, 447, 450, 451, 454, 456, 458, 461 to 464, 467, 469, 470, and 527 to 533.
64. The modified tobacco plant, or part thereof, of any one of embodiments 55 to 57 or 61 to 63, wherein the modified tobacco plant further comprises at least one mutant allele of an endogenous locus encoding a HAIRPLUS1b (NtHAP1b) protein, wherein the at least one mutant allele comprises a nucleic acid sequence selected from the group consisting of SEQ ID NOs: 36, 38, 39, 41, 43, 45, 48, 138, 139, 143, 146, 147, 150, 151, 154, 155, 158, 159, 162, 163, 165, 168, 171, 172, 175, 176, 179 to 181, 185, 186, 191, 194, 195, 199, 203, 205, 210, 218, 222, and 270 to 277.
65. The modified tobacco plant, or part thereof, of any one of embodiments 55 to 57 or 61 to 63, wherein the modified tobacco plant further comprises at least one mutant allele of an endogenous locus encoding a HAIRPLUS1b (NtHAP1b) protein, wherein the at least one mutant allele comprises a nucleic acid sequence selected from the group consisting of SEQ ID NOs: 73, 74, 76, 78, 81, 82, 84, 85, 88, 89, 285, 288, 289, 292, 293, 296, 300, 302, 305, 306, 311, 312, 314, 315, 319, 320, 323 to 325, 328, 330, 332 to 335, 337, 338, 340, 342, 344, 347, 348, and 351.
66. The modified tobacco plant, or part thereof, of any one of embodiments 55 to 57 or 61 to 63, wherein the modified tobacco plant further comprises at least one mutant allele of an endogenous locus encoding a HAIRPLUS1b (NtHAP1b) protein, wherein the at least one mutant allele comprises a nucleic acid sequence encoding an amino acid sequence selected from the group consisting of SEQ ID NOs: 99, 101, 102, 104, 106, 108, 111, 117, 118, 388, 389, 393, 396, 397, 400, 401, 404, 405, 408, 409, 412, 413, 415, 418, 421, 422, 425, 426, 429 to 431, 435, 436, 441, 444, 445, 449, 453, 455, 460, 468, 472, and 520 to 526.
67. The modified tobacco plant, or part thereof, of any one of embodiments 55 to 57, wherein the modified tobacco plant further comprises: (a) at least one mutant allele of an endogenous locus encoding a HAIRPLUS1b (NtHAP1b) protein, wherein the at least one mutant allele comprises a nucleic acid sequence selected from the group consisting of SEQ ID NOs: 36, 38, 39, 41, 43, 45, 48, 138, 139, 143, 146, 147, 150, 151, 154, 155, 158, 159, 162, 163, 165, 168, 171, 172, 175, 176, 179 to 181, 185, 186, 191, 194, 195, 199, 203, 205, 210, 218, 222, and 270 to 277; and (b) at least one mutant allele of an endogenous locus encoding a HAIRPLUS2 (NtHAP2) protein, wherein the at least one mutant allele comprises a nucleic acid sequence selected from the group consisting of SEQ ID NOs: 51, 52, 56, 182, 183, 187 to 190, 192, 193, 196, 197, 200, 201, 204, 206, 208, 211 to 214, 217, 219, 220, and 278 to 284.
68. The modified tobacco plant, or part thereof, of any one of embodiments 55 to 57, wherein the modified tobacco plant further comprises: (a) at least one mutant allele of an endogenous locus encoding a HAIRPLUS1b (NtHAP1b) protein, wherein the at least one mutant allele comprises a nucleic acid sequence selected from the group consisting of SEQ ID NOs: 36, 38, 39, 41, 43, 45, 48, 138, 139, 143, 146, 147, 150, 151, 154, 155, 158, 159, 162, 163, 165, 168, 171, 172, 175, 176, 179 to 181, 185, 186, 191, 194, 195, 199, 203, 205, 210, 218, 222, and 270 to 277; and (b) at least one mutant allele of an endogenous locus encoding a HAIRPLUS2 (NtHAP2) protein, wherein the at least one mutant allele comprises a nucleic acid sequence selected from the group consisting of SEQ ID NOs: 321 and 322.
69. The modified tobacco plant, or part thereof, of any one of embodiments 55 to 57, wherein the modified tobacco plant further comprises: (a) at least one mutant allele of an endogenous locus encoding a HAIRPLUS1b (NtHAP1b) protein, wherein the at least one mutant allele comprises a nucleic acid sequence selected from the group consisting of SEQ ID NOs: 36, 38, 39, 41, 43, 45, 48, 138, 139, 143, 146, 147, 150, 151, 154, 155, 158, 159, 162, 163, 165, 168, 171, 172, 175, 176, 179 to 181, 185, 186, 191, 194, 195, 199, 203, 205, 210, 218, 222, and 270 to 277; and (b) at least one mutant allele of an endogenous locus encoding a HAIRPLUS2 (NtHAP2) protein, wherein the at least one mutant allele comprises a nucleic acid sequence encoding an amino acid sequence selected from the group consisting of SEQ ID NOs: 114, 115, 119, 432, 433, 437 to 440, 442, 443, 446, 447, 450, 451, 454, 456, 458, 461 to 464, 467, 469, 470, and 527 to 533.
70. The modified tobacco plant, or part thereof, of any one of embodiments 55 to 57, wherein the modified tobacco plant further comprises: (a) at least one mutant allele of an endogenous locus encoding a HAIRPLUS1b (NtHAP1b) protein, wherein the at least one mutant allele comprises a nucleic acid sequence selected from the group consisting of SEQ ID NOs: 73, 74, 76, 78, 81, 82, 84, 85, 88, 89, 285, 288, 289, 292, 293, 296, 300, 302, 305, 306, 311, 312, 314, 315, 319, 320, 323 to 325, 328, 330, 332 to 335, 337, 338, 340, 342, 344, 347, 348, and 351; and (b) at least one mutant allele of an endogenous locus encoding a HAIRPLUS2 (NtHAP2) protein, wherein the at least one mutant allele comprises a nucleic acid sequence selected from the group consisting of SEQ ID NOs: 51, 52, 56, 182, 183, 187 to 190, 192, 193, 196, 197, 200, 201, 204, 206, 208, 211 to 214, 217, 219, 220, and 278 to 284.
71. The modified tobacco plant, or part thereof, of any one of embodiments 55 to 57, wherein the modified tobacco plant further comprises: (a) at least one mutant allele of an endogenous locus encoding a HAIRPLUS1b (NtHAP1b) protein, wherein the at least one mutant allele comprises a nucleic acid sequence selected from the group consisting of SEQ ID NOs: 73, 74, 76, 78, 81, 82, 84, 85, 88, 89, 285, 288, 289, 292, 293, 296, 300, 302, 305, 306, 311, 312, 314, 315, 319, 320, 323 to 325, 328, 330, 332 to 335, 337, 338, 340, 342, 344, 347, 348, and 351; and (b) at least one mutant allele of an endogenous locus encoding a HAIRPLUS2 (NtHAP2) protein, wherein the at least one mutant allele comprises a nucleic acid sequence selected from the group consisting of SEQ ID NOs: 321 and 322.
72. The modified tobacco plant, or part thereof, of any one of embodiments 55 to 57, wherein the modified tobacco plant further comprises: (a) at least one mutant allele of an endogenous locus encoding a HAIRPLUS1b (NtHAP1b) protein, wherein the at least one mutant allele comprises a nucleic acid sequence selected from the group consisting of SEQ ID NOs: 73, 74, 76, 78, 81, 82, 84, 85, 88, 89, 285, 288, 289, 292, 293, 296, 300, 302, 305, 306, 311, 312, 314, 315, 319, 320, 323 to 325, 328, 330, 332 to 335, 337, 338, 340, 342, 344, 347, 348, and 351; and (b) at least one mutant allele of an endogenous locus encoding a HAIRPLUS2 (NtHAP2) protein, wherein the at least one mutant allele comprises a nucleic acid sequence encoding an amino acid sequence selected from the group consisting of SEQ ID NOs: 114, 115, 119, 432, 433, 437 to 440, 442, 443, 446, 447, 450, 451, 454, 456, 458, 461 to 464, 467, 469, 470, and 527 to 533.
73. The modified tobacco plant, or part thereof, of any one of embodiments 55 to 57, wherein the modified tobacco plant further comprises: (a) at least one mutant allele of an endogenous locus encoding a HAIRPLUS1b (NtHAP1b) protein, wherein the at least one mutant allele comprises a nucleic acid sequence encoding an amino acid sequence selected from the group consisting of SEQ ID NOs: 99, 101, 102, 104, 106, 108, 111, 117, 118, 388, 389, 393, 396, 397, 400, 401, 404, 405, 408, 409, 412, 413, 415, 418, 421, 422, 425, 426, 429 to 431, 435, 436, 441, 444, 445, 449, 453, 455, 460, 468, 472, and 520 to 526; and (b) at least one mutant allele of an endogenous locus encoding a HAIRPLUS2 (NtHAP2) protein, wherein the at least one mutant allele comprises a nucleic acid sequence selected from the group consisting of SEQ ID NOs: 51, 52, 56, 182, 183, 187 to 190, 192, 193, 196, 197, 200, 201, 204, 206, 208, 211 to 214, 217, 219, 220, and 278 to 284.
74. The modified tobacco plant, or part thereof, of any one of embodiments 55 to 57, wherein the modified tobacco plant further comprises: (a) at least one mutant allele of an endogenous locus encoding a HAIRPLUS1b (NtHAP1b) protein, wherein the at least one mutant allele comprises a nucleic acid sequence encoding an amino acid sequence selected from the group consisting of SEQ ID NOs: 99, 101, 102, 104, 106, 108, 111, 117, 118, 388, 389, 393, 396, 397, 400, 401, 404, 405, 408, 409, 412, 413, 415, 418, 421, 422, 425, 426, 429 to 431, 435, 436, 441, 444, 445, 449, 453, 455, 460, 468, 472, and 520 to 526; and (b) at least one mutant allele of an endogenous locus encoding a HAIRPLUS2 (NtHAP2) protein, wherein the at least one mutant allele comprises a nucleic acid sequence selected from the group consisting of SEQ ID NOs: 321 and 322.
75. The modified tobacco plant, or part thereof, of any one of embodiments 55 to 57, wherein the modified tobacco plant further comprises: (a) at least one mutant allele of an endogenous locus encoding a HAIRPLUS1b (NtHAP1b) protein, wherein the at least one mutant allele comprises a nucleic acid sequence encoding an amino acid sequence selected from the group consisting of SEQ ID NOs: 99, 101, 102, 104, 106, 108, 111, 117, 118, 388, 389, 393, 396, 397, 400, 401, 404, 405, 408, 409, 412, 413, 415, 418, 421, 422, 425, 426, 429 to 431, 435, 436, 441, 444, 445, 449, 453, 455, 460, 468, 472, and 520 to 526; and (b) at least one mutant allele of an endogenous locus encoding a HAIRPLUS2 (NtHAP2) protein, wherein the at least one mutant allele comprises a nucleic acid sequence encoding an amino acid sequence selected from the group consisting of SEQ ID NOs: 114, 115, 119, 432, 433, 437 to 440, 442, 443, 446, 447, 450, 451, 454, 456, 458, 461 to 464, 467, 469, 470, and 527 to 533.
76. The modified tobacco plant, or part thereof, of any one of embodiments 1 to 75, wherein the modified tobacco plant is selected from the group consisting of a flue-cured tobacco plant, a bright tobacco plant, a Burley tobacco plant, a Virginia tobacco plant, a Maryland tobacco plant, a dark tobacco plant, a Galpão tobacco plant, an Oriental tobacco plant, and a Turkish tobacco plant.
77. The modified tobacco plant, or part thereof, of any one of embodiments 1 to 75, wherein the modified tobacco plant is selected from the group consisting of the tobacco plants listed in Table 2, Table 3, Table 4, Table 5, Table 6, Table 7, and Table 8.
78. The modified tobacco plant, or part thereof, of any one of embodiments 1 to 75, wherein the modified tobacco plant is a hybrid tobacco plant.
79. The modified tobacco plant, or part thereof, of any one of embodiments 1 to 75, wherein the modified tobacco plant is male sterile or cytoplasmically male sterile.
80. The modified tobacco plant, or part thereof, of any one of embodiments 1 to 79, wherein the modified tobacco plant comprises an increased density of trichomes on at least one leaf as compared to the control tobacco plant lacking the non-natural mutation when grown under comparable conditions.
81. The modified tobacco plant, or part thereof, of any one of embodiments 1 to 79, wherein the modified tobacco plant comprises an increased number of trichomes on at least one leaf as compared to the control tobacco plant lacking the non-natural mutation when grown under comparable conditions.
82. The modified tobacco plant, or part thereof, of any one of embodiments 1 to 79, wherein the modified tobacco plant comprises an increased total number of trichomes as compared to the control tobacco plant lacking the non-natural mutation when grown under comparable conditions.
83. A seed obtained from the modified tobacco plant, or part thereof, of any one of embodiments 1 to 82.
84. Cured tobacco material from the modified tobacco plant, or part thereof, of any one of embodiments 1 to 82.
85. The cured tobacco material of embodiment 84, wherein the cured tobacco material comprises leaf material.
86. The cured tobacco material of embodiment 84, wherein the cured tobacco material comprises stem material.
87. The cured tobacco material of any one of embodiments 84 to 86, wherein the cured tobacco material is selected from the group consisting of flue-cured tobacco material, air-cured tobacco material, fire-cured tobacco material, and sun-cured tobacco material.
88. A tobacco blend comprising the cured tobacco material of any one of embodiments 84 to 87.
89. The tobacco blend of embodiment 88, wherein the cured tobacco material comprises at least 10% of cured tobacco in the tobacco blend by weight.
90. The tobacco blend of embodiment 88, wherein the cured tobacco material comprises at least 10% of cured tobacco in the tobacco blend by volume.
91. A tobacco product comprising the cured tobacco material of any one of embodiments 84 to 87.
92. A tobacco product comprising the tobacco blend of any one of embodiments 88 to 90.
93. The tobacco product of embodiment 91 or 92, wherein the tobacco product is selected from the group consisting of a cigarillo, non-ventilated recess filter cigarette, vented recess filter cigarette, cigar, snuff, pipe tobacco, cigar tobacco, cigarette tobacco, chewing tobacco, leaf tobacco, hookah tobacco, shredded tobacco, and cut tobacco.
94. The tobacco product of embodiment 91 or 92, wherein the tobacco product is a smokeless tobacco product.
95. The tobacco product of embodiment 94, wherein the smokeless tobacco product is selected from the group consisting of loose leaf chewing tobacco, plug chewing tobacco, moist snuff, nasal snuff, dry snuff, and snus.
96. The tobacco product of embodiment 91 or 92, wherein the tobacco product is a heated tobacco product.
97. A tobacco product comprising tobacco material from the tobacco plant, or part thereof, of any one of embodiments 1 to 82.
98. Reconstituted tobacco comprising tobacco material from the modified tobacco plant, or part thereof, of any one of embodiments 1 to 82.
99. A tobacco product comprising the reconstituted tobacco of embodiment 98.
100. Fermented tobacco comprising tobacco material from the modified tobacco plant, or part thereof, of any one of embodiments 1 to 82.
101. A tobacco product comprising the reconstituted tobacco of embodiment 100.
102. A method of producing a modified tobacco plant, the method comprising:
   (a) inducing at least one non-natural mutation in at least one endogenous nucleic acid molecule encoding an amino acid sequence selected from the group consisting of SEQ ID NOs: 1, 2, 3, and 538 in at least one tobacco cell; and
   (b) regenerating a modified tobacco plant from the at least one tobacco cell of step (a), wherein the tobacco plant comprises the at least one non-natural mutation, and wherein the modified tobacco plant exhibits reduced expression or activity of the at least one endogenous nucleic acid molecule as compared to a control tobacco plant lacking the at least one non-natural mutation when grown under comparable conditions.
103. The method of embodiment 102, wherein the endogenous nucleic acid molecule comprises a sequence selected from the group consisting of SEQ ID NOs: 4 to 8.
104. The method of embodiment 102, wherein the at least one non-natural mutation comprises a first non-natural mutation in the endogenous nucleic acid molecule encoding SEQ ID NO: 1 and a second non-natural mutation in the endogenous nucleic acid molecule encoding SEQ ID NO: 2.
105. The method of embodiment 104, wherein the at least one non-natural mutation further comprises a third non-natural mutation in the endogenous nucleic acid molecule encoding SEQ ID NO: 3.
106. The method of embodiment 102, wherein the at least one non-natural mutation comprises a first non-natural mutation in the endogenous nucleic acid molecule encoding SEQ ID NO: 1 and a second non-natural mutation in the endogenous nucleic acid molecule encoding SEQ ID NO: 3.
107. The method of embodiment 102, wherein the at least one non-natural mutation comprises a first non-natural mutation in the endogenous nucleic acid molecule encoding SEQ ID NO: 2 and a second non-natural mutation in the endogenous nucleic acid molecule encoding SEQ ID NO: 3.
108. The method of embodiment 102, wherein the modified tobacco plant is homozygous for the at least one non-natural mutation.
109. The method of embodiment 102, wherein the inducing comprises the use of a chemical mutagen.
110. The method of embodiment 109, wherein the chemical mutagen is ethyl methylsulfate.
111. The method of embodiment 102, wherein the inducing comprises the use of irradiation.
112. The method of embodiment 111, wherein the irradiation is selected from the group consisting of ultraviolet irradiation, X-rays, and fast neutron irradiation.
113. The method of embodiment 102, wherein the inducing comprises the use of a nuclease.
114. The method of embodiment 113, wherein the nuclease is selected from the group consisting of a Cas9 nuclease, a Cas12a/Cpf1 nuclease, a Csm1 nuclease, a CasX nuclease, a CasY nuclease, a zinc-finger nuclease, a meganuclease, and a transcription activator-like nuclease.
115. A method of producing a modified tobacco plant, the method comprising:
   (a) introducing a recombinant nucleic acid construct to at least one tobacco cell, wherein the recombinant nucleic acid construct comprises a heterologous promoter operably linked to a polynucleotide that encodes a non-coding RNA molecule; and
   (b) regenerating a modified tobacco plant from the at least one tobacco cell of step (a), wherein the tobacco plant comprises the recombinant nucleic acid construct, and wherein the non-coding RNA molecule suppresses the transcription or translation of at least one RNA molecule encoding an amino acid sequence at least 90% identical or similar to an amino acid sequence selected from the group consisting of SEQ ID NOs: 1, 2, 3, and 538 as compared to a control tobacco plant lacking the recombinant nucleic acid construct when grown under comparable conditions.
116. The method of embodiment 115, wherein the non-coding RNA molecule suppresses the transcription or translation of SEQ ID NO: 1 and SEQ ID NO: 2.
117. The method of embodiment 116, wherein the non-coding RNA molecule further suppresses the transcription or translation of SEQ ID NO: 3.
118. The method of embodiment 115, wherein the non-coding RNA molecule suppresses the transcription or translation of SEQ ID NO: 1 and SEQ ID NO: 3.
119. The method of embodiment 115, wherein the non-coding RNA molecule suppresses the transcription or translation of SEQ ID NO: 2 and SEQ ID NO: 3.
120. The method of embodiment 115, wherein the at least one RNA molecule comprises a sequence selected from the group consisting of SEQ ID NOs: 4 to 6.
121. The method of embodiment 115, wherein the non-coding RNA molecule is at least 90% complementary to at least 19 consecutive nucleotides of a nucleic acid sequence selected from the group consisting of SEQ ID NOs: 4 to 8.
122. The method of embodiment 115, wherein the introducing comprises a technique selected from the group consisting of Agrobacterium-mediated transformation, microprojectile bombardment, and electroporation.
123. A method for producing a tobacco plant, the method comprising:
   (a) crossing a modified tobacco plant with a second tobacco plant to produce at least one progeny tobacco seed, wherein the modified tobacco plant comprises at least one non-natural mutation in at least one endogenous nucleic acid molecule encoding an amino acid sequence selected from the group consisting of SEQ ID NOs: 1, 2, 3, and 538; and
   (b) selecting at least one progeny tobacco seed, or a tobacco plant germinated therefrom, comprising the at least one non-natural mutation.
124. The method of embodiment 123, wherein the second tobacco plant comprises the at least one non-natural mutation.
125. The method of embodiment 123, wherein the modified tobacco plant comprises at least one non-natural mutation in a first endogenous nucleic acid molecule encoding the amino acid sequence of SEQ ID NO: 1 and at least one non-natural mutation in a second endogenous nucleic acid molecule encoding the amino acid sequence of SEQ ID NO: 2.
126. The method of embodiment 125, wherein the modified tobacco plant further comprises at least one non-natural mutation in a third endogenous nucleic acid molecule encoding the amino acid sequence of SEQ ID NO: 3.
127. The method of embodiment 123, wherein the modified tobacco plant comprises at least one non-natural mutation in a first endogenous nucleic acid molecule encoding the amino acid sequence of SEQ ID NO: 1 and at least one non-natural mutation in a second endogenous nucleic acid molecule encoding the amino acid sequence of SEQ ID NO: 3.
128. The method of embodiment 123, wherein the modified tobacco plant comprises at least one non-natural mutation in a first endogenous nucleic acid molecule encoding the amino acid sequence of SEQ ID NO: 2 and at least one non-natural mutation in a second endogenous nucleic acid molecule encoding the amino acid sequence of SEQ ID NO: 3.
129. A method for producing a tobacco plant, the method comprising:
   (a) crossing a first tobacco plant with a second tobacco plant to produce at least one progeny tobacco seed, wherein the first tobacco plant comprises a recombinant nucleic acid construct comprising a heterologous promoter operably linked to a polynucleotide that encodes a non-coding RNA molecule that binds to and suppresses the transcription or translation of at least one RNA molecule encoding an amino acid sequence at least 90% identical or similar to an amino acid sequence selected from the group consisting of SEQ ID NOs: 1, 2, 3, and 538; and
   (b) selecting at least one progeny tobacco seed, or a tobacco plant germinated therefrom, comprising the recombinant nucleic acid construct.
130. The method of embodiment 129, wherein the non-coding RNA molecule suppresses the transcription or translation of SEQ ID NO: 1 and SEQ ID NO: 2.
131. The method of embodiment 130, wherein the non-coding RNA molecule further suppresses the transcription or translation of SEQ ID NO: 3.
132. The method of embodiment 129, wherein the non-coding RNA molecule suppresses the transcription or translation of SEQ ID NO: 1 and SEQ ID NO: 3.
133. The method of embodiment 129, wherein the non-coding RNA molecule suppresses the transcription or translation of SEQ ID NO: 2 and SEQ ID NO: 3.
134. The method of any one of embodiments 102 to 133, wherein the first tobacco plant is a male parent.
135. The method of any one of embodiments 102 to 103, wherein the first tobacco plant is a female parent.
136. The method of any one of embodiments 129 to 135, wherein the first tobacco plant or the tobacco plant germinated from the at least one progeny tobacco seed comprises an increased density of trichomes on at least one leaf as compared to the control tobacco plant lacking the non-natural mutation when grown under comparable conditions.
137. The method of any one of embodiments 129 to 135, wherein the first tobacco plant or the tobacco plant germinated from the at least one progeny tobacco seed comprises an increased number of trichomes on at least one leaf as compared to the control tobacco plant lacking the non-natural mutation when grown under comparable conditions.
138. The method of any one of embodiments 129 to 135, wherein the first tobacco plant or the or the tobacco plant germinated from the at least one progeny tobacco seed tobacco plant germinated from the at least one progeny tobacco seed comprises an increased total number of trichomes as compared to the control tobacco plant lacking the non-natural mutation when grown under comparable conditions.
139. A method of producing a tobacco product, the method comprising:
   (a) obtaining the cured tobacco material of any one of embodiments 84 to 87; and
   (b) producing a tobacco product comprising the cured tobacco material.
140. A method of producing a tobacco product, the method comprising:
   (a) obtaining tobacco material from the modified tobacco plant of any one of embodiments 1 to 75;
   (b) curing the tobacco material to produce cured tobacco material; and
   (c) producing the tobacco product, wherein the tobacco product comprises the cured tobacco material.
141. A method of producing a tobacco product, the method comprising:
   (a) curing tobacco material to produce cured tobacco material, wherein the tobacco material is from a modified tobacco plant comprising a non-natural mutation in an endogenous nucleic acid molecule encoding a HAIRPLUS1a (NtHAP1a) protein, wherein the endogenous NtHAP1a protein comprises the amino acid sequence of SEQ ID NO: 1, and wherein expression or activity of the NtHAP1a protein is reduced in the modified tobacco plant as compared to a control tobacco plant lacking the non-natural mutation when grown under comparable conditions; and
   (b) producing a tobacco product comprising the cured tobacco material from step (a).
142. A method of producing a tobacco product, the method comprising:
   (a) curing tobacco material to produce cured tobacco material, wherein the tobacco material is from a modified tobacco plant comprising a non-natural mutation in an endogenous nucleic acid molecule encoding a HAIRPLUS1b (NtHAP1b) protein, wherein the endogenous NtHAP1a protein comprises the amino acid sequence of SEQ ID NO: 2, and wherein expression or activity of the NtHAP1b protein is reduced in the modified tobacco plant as compared to a control tobacco plant lacking the non-natural mutation when grown under comparable conditions; and
   (b) producing a tobacco product comprising the cured tobacco material from step (a).
143. A method of producing a tobacco product, the method comprising:
   (a) curing tobacco material to produce cured tobacco material, wherein the tobacco material is from a modified tobacco plant comprising a non-natural mutation in an endogenous nucleic acid molecule encoding a HAIRPLUS2 (NtHAP2) protein, wherein the endogenous NtHAP2 protein comprises the amino acid sequence of SEQ ID NO: 3, and wherein expression or activity of the NtHAP2 protein is reduced in the modified tobacco plant as compared to a control tobacco plant lacking the non-natural mutation when grown under comparable conditions; and
   (b) producing a tobacco product comprising the cured tobacco material from step (a).
144. The method of any one of embodiments 102 to 128 or 141 to 143, wherein the modified tobacco plant comprises an increased density of trichomes on at least one leaf as compared to the control tobacco plant lacking the non-natural mutation when grown under comparable conditions.
145. The method of any one of embodiments 102 to 128 or 141 to 143, wherein the modified tobacco plant comprises an increased number of trichomes on at least one leaf as compared to the control tobacco plant lacking the non-natural mutation when grown under comparable conditions.
146. The method of any one of embodiments 102 to 128 or 141 to 143, wherein the modified tobacco plant comprises an increased total number of trichomes as compared to the control tobacco plant lacking the non-natural mutation when grown under comparable conditions.
147. The method of any one of embodiments 102 to 128 or 141 to 146, wherein the modified tobacco plant comprises an increased average length of trichomes as compared to the control tobacco plant lacking the non-natural mutation when grown under comparable conditions.
148. The method of any one of embodiments 102 to 128 or 141 to 147, wherein the modified tobacco plant exhibits a statistically significant increase in neophytadiene as compared to the control tobacco plant lacking the non-natural mutation when grown under comparable conditions.
149. The method of any one of embodiments 102 to 128 or 141 to 148, wherein the modified tobacco plant exhibits a statistically significant decrease in a terpene selected from the group consisting of cis-abienol, 4,8,13-duvatriene-1,3-diol, and thunbergol as compared to the control tobacco plant lacking the non-natural mutation when grown under comparable conditions.
150. The modified tobacco plant, or part thereof, of any one of embodiments 1 to 79, wherein the modified tobacco plant comprises an increased average length of trichomes as compared to the control tobacco plant lacking the non-natural mutation when grown under comparable conditions.
151. The modified tobacco plant, or part thereof, of any one of embodiments 1 to 79 or 150, wherein the modified tobacco plant exhibits a statistically significant increase in neophytadiene as compared to the control tobacco plant lacking the non-natural mutation when grown under comparable conditions.
152. The modified tobacco plant, or part thereof, of any one of embodiments 1 to 79, 150, or 151, wherein the modified tobacco plant exhibits a statistically significant decrease in a terpene selected from the group consisting of cis-abienol, 4,8,13-duvatriene-1,3-diol, and thunbergol as compared to the control tobacco plant lacking the non-natural mutation when grown under comparable conditions.

Having now generally described the disclosure, the same will be more readily understood through reference to the following examples that are provided by way of illustration, and are not intended to be limiting of the present disclosure, unless specified.

### EXAMPLES

### Example 1. Identification of Tobacco Homologues of HAIRPLUS

The HAIRPLUS (HAP) gene was previously identified in tomato as being involved in trichome formation. Prior to this disclosure, no tobacco homologues of HAP had been identified.

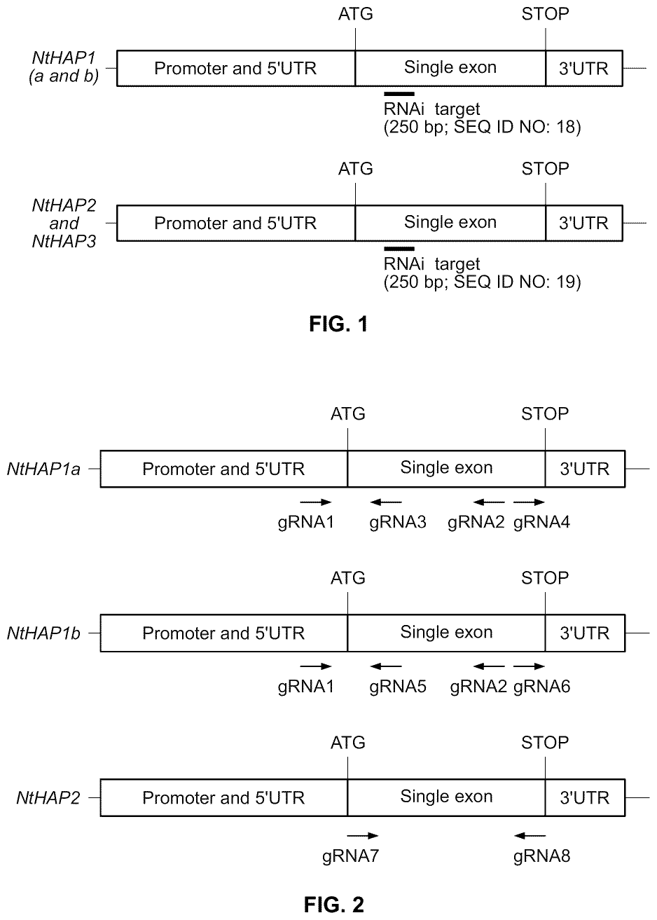

Four tobacco HAP homologues are identified in the Nicotiana tabacum genome, as shown in Table 9. FIG. 1 provides the structure of four NtHAP genes and the position of RNAi targets within the genes (see Example 3). The start codon for NtHAP1a is at nucleotide position 2001 of SEQ ID NO: 7 or nucleotide position 1 of SEQ ID NO: 4. The start codon for NtHAP1b is at nucleotide position 2001 of SEQ ID NO: 8, or nucleotide position 1 of SEQ ID NO: 5. The start codon for NtHAP2 is at nucleotide position 2001 of SEQ ID NO: 9, or nucleotide position 1 of SEQ ID NO: 6. The start codon for NtHAP3 is at nucleotide position 157 of SEQ ID NO: 537, or nucleotide position 1 of SEQ ID NO: 612.

TABLE 9
Nicotiana tabacum HAP homologues

| | | Coding | |
|---|---|---|---|
| | Amino Acid | DNA | Genomic DNA |
| NtHAP gene | SEQ ID NO | SEQ ID NO | SEQ ID NO |
| NtHAP1a/G23609 | 1 | 4 | 7 |
| NtHAP1b/G95893 | 2 | 5 | 8 |
| NtHAP2/G68445 | 3 | 6 | 9 |
| NtHAP3/ | 538 | 612 | 537 |
| NM_001324736.1 | | | |

In addition to the four HAP homologs identified in Nicotiana tabacum, there are eight HAP homologs in other Nicotiana species, with two in Nicotiana attenuate, two in Nicotiana benthamiana, two in Nicotiana sylvestris, and two in Nicotiana tomentosiformis.

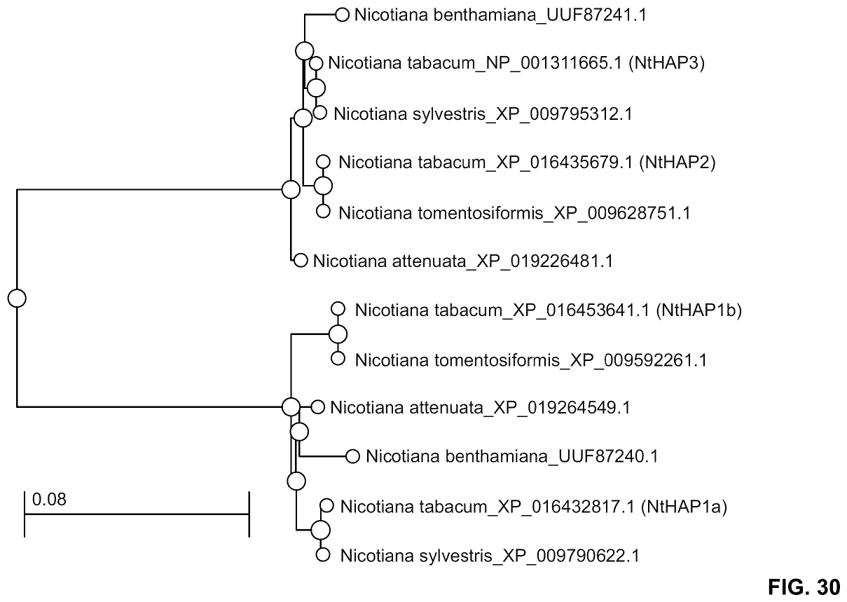

The phylogenetic tree for the HAP homologs in Nicotiana clearly reveals the lineage of NtHAP1 and NtHAP2, which are descended from Nicotiana sylvestris (for NtHAP1a and 3) and Nicotiana tomentosiformis (for NtHAP1b and 2), the commonly believed ancestral species of tobacco. FIG. 30 depicts the phylogenetic relationship between NtHAP1a, NtHAP1b, NtHAP2, and NtHAP3 amino acid sequences with the related amino acid sequences from other Nicotiana species.

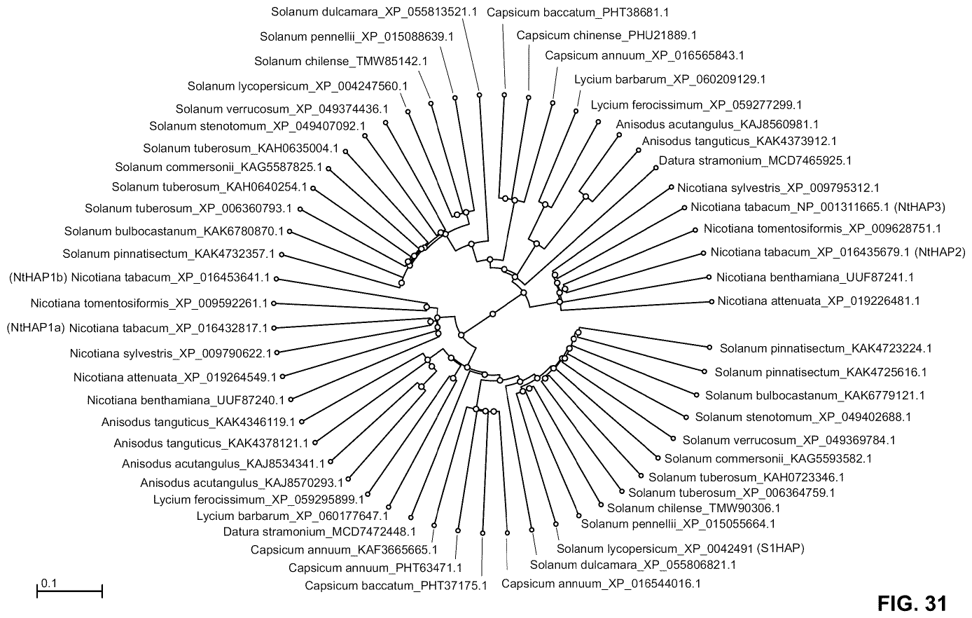

A total of 55 HAP homologues are identified in other members of Family Solanaceae, including the original HAP gene from tomato (SIHAP; SEQ ID NO: 589). FIG. 31 depicts the phylogenetic relationship between Nicotiana HAP amino acid sequences and amino acid sequences of HAP homologues from other Family Solanaceae species.

Table 10 shows the percent identities between the tobacco HAP genes and SIHAP.

TABLE 10
Comparison between N. tabacum HAP genes and tomato HAP gene
(Accession Solyc10g077070.1.1 from solgenomics[dot]net)
using BLASTN (coding nucleic acid) or BLASTP (amino acid; SEQ ID
NO: 589) pairwise comparisons with the tomato sequence as the Query
and the N. tabacum sequences as the subject. Total identity
(shown as a percent) is calculated by multiplying the Percent
Coverage by Percent Identity for each comparison.

| | BLASTN | | | BLASTP | | |
|---|---|---|---|---|---|---|
| NtHAP | Percent | Percent | Total | Percent | Percent | Total |
| gene | Coverage | Identity | Identity | Coverage | Identity | Identity |
| NtHAP1a | 100 | 89.47 | 89.47 | 100 | 88.15 | 88.15 |
| NtHAP1b | 100 | 89.09 | 89.09 | 100 | 87.73 | 87.73 |
| NtHAP2 | 91 | 79.73 | 72.55 | 100 | 73.56 | 73.56 |
| NtHAP3 | 91 | 79.92 | 72.72 | 100 | 73.84 | 73.84 |

### Example 2. Transformation and Regeneration of Modified Tobacco Plants

Tobacco cells are transformed with desired transformation vectors, and the transformed cells are used to regenerate transformed tobacco plants comprising desired transgenes and/or desired edits.

Tobacco plants (for example, without being limiting, NL Madole LC tobacco plants) are grown in Magenta™ GA-7 boxes and leaf discs are cut and placed into Petri plates. Agrobacterium tumefaciens cells comprising a transformation vector are collected by centrifuging a 20 mL cell suspension in a 50 mL centrifuge tube at 3500 RPM for 10 minutes. The supernatant is removed and the Agrobacterium tumefaciens cell pellet is re-suspended in 40 mL liquid re-suspension medium. Tobacco leaves, avoiding the midrib, are cut into eight 0.6 cm discs with a #15 razor blade and placed upside down in a Petri plate. A thin layer of Murashige & Skoog (MS) with B5 vitamins liquid re-suspension medium is added to the Petri plate and the leaf discs are poked uniformly with a fine point needle. About 25 mL of the Agrobacterium tumefaciens suspension is added to the Petri plate and the leaf discs are incubated in the suspension for 10 minutes.

Leaf discs are transferred to co-cultivation Petri plates (½ MS medium) and discs are placed upside down in contact with filter paper overlaid on the co-cultivation TOM medium (MS medium with 30 g/L sucrose; 0.1 mg/L 1-naphthaleneacetic acid (NAA); and 1 mg/L 6-benzyl aminopurine (BAP)). The Petri plate is sealed with parafilm prior to incubation in the dark for two days.

After incubation, leaf discs are transferred to regeneration/selection TOM-Hyg medium Petri plates (TOM medium plus 200 mg/L cefotaxime and 50 mg/L hygromycin). Calli formed from leaf discs are sub-cultured bi-weekly to fresh TOM-Hyg medium in dim light (between 60 mE/ms and 80 mE/ms) with photoperiods of 18 hours light, 6 hours dark at 24° C. until shoots (plantlets) become excisable. Plantlets formed from calli are removed with forceps and subcultured into MS rooting medium (MS medium with 3 g/L sucrose; 7 g/L dextrose; 200 mg/L cefotaxime; 50 mg/L hygromycin). Shoots on MS rooting medium are incubated at 24° C. with dim light and photoperiods of 18 hours light, 6 hours dark to induce rooting

When plantlets comprising both shoots and roots grow large enough (e.g., over half the height of a Magenta™ GA-7 box), they are transferred Jiffy peat pellets for acclimatization in the growth room. Once established, seedlings are transferred to a greenhouse for further growth, breeding, and analysis.

### Example 3. Generation of Tobacco Plants with NtHAP Knockouts

NtHAP knockout mutants (e.g., non-natural mutations) are produced by editing various NtHAP genes. Tobacco protoplasts are transfected using polyethylene glycol (PEG) with plasmids encoding a genome editing technology protein and specific guide RNAs (gRNAs) to target specific NtHAP genes at desired positions. FIG. 2 provides a schematic showing which gRNAs are used to specifically knockout each of NtHAP1a, NtHAP1b, and NtHAP2. Table 11 provides the SEQ ID NO for each gRNA shown in FIG. 2. Various knockout lines are produced, including: NtHAP1a knockout; NtHAP1b knockout; NtHAP2 knockout; NtHAP1a and NtHAP1b knockout; NtHAP1a and NtHAP2 knockout; NtHAP1b and NtHAP2 knockout, and NtHAP1a, NtHAP1b, and NtHAP2 knockout. Table 12 provides gRNA combinations that can be used to knock out selected combinations of NtHAP genes.

TABLE 11
gRNAs used for NtHAP editing

| gRNA | SEQ ID NO |
|---|---|
| gRNA1 | 10 |
| gRNA2 | 11 |
| gRNA3 | 12 |
| gRNA4 | 13 |
| gRNA5 | 14 |
| gRNA6 | 15 |
| gRNA7 | 16 |
| gRNA8 | 17 |

TABLE 12
gRNA combinations used to generate NtHAP knockouts

| NtHAP genes targeted | gRNAs used |
|---|---|
| NtHAP1a | gRNA3 and gRNA4 |
| NtHAP1b | gRNA5 and gRNA6 |
| NtHAP2 | gRNA7 and gRNA8 |
| NtHAP1a and NtHAP1b | gRNA1 and gRNA2 |
| NtHAP1a, NtHAP1b, | gRNA1, gRNA2, |
| and NtHAP2 | gRNA7, and gRNA8 |
| NtHAP1a and NtHAP2 | gRNA3, gRNA4, |
| | gRNA7, and gRNA8 |
| NtHAP1b and NtHAP2 | gRNA5, gRNA6, |
| | gRNA7, and gRNA8 |

Transfected protoplasts are then immobilized in 1% agarose beads and subjected to tissue culture. When calli grow to approximately 1 mm in diameter, they are spread on TOM2 plates. Calli are screened for non-natural mutations in the appropriate NtHAP gene(s). Candidates, showing an insertion, substitution, deletion, or inversion in the targeted gene(s), are selected for further culture and the consequent shoots are tested again to confirm the presence of edits. Rooted shoots are potted and sequenced for the target positions to determine the exact sequences of the NtHAP gene(s).

NtHAP genes are mutated in the tobacco lines K326 and Izmir. Table 13 provides information related to K326 mutants, and Table 14 provides information related to Izmir mutants. Primers comprising the nucleic acid sequences of SEQ ID NOs: 590 to 605 are used to amplify and sequence mutations in NtHAP1a, NtHAP1b, and NtHAP2.

TABLE 13
K326 NtHAP mutants. Mutation position is measured from the start codon for each gene (e.g.,
the first nucleotide of the start codon is position 1). “nt” refers to “nucleotide(s)”.
Reference allele provides the nucleotides that are deleted, when applicable. Mutant allele provides
the nucleotides that are inserted, when applicable. In some instances, a SEQ ID NO is provided
instead of individual nucleotides. The Mutant SEQ ID NO columns provide sequence references
for individual mutant alleles. A “—” symbol indicates “not applicable.”

| | | | | Number of | | | | | |
|---|---|---|---|---|---|---|---|---|---|
| Edited | | | | nt deleted | | | Mutant SEQ ID NO | | |
| Line | Edited | Mutation | Mutation | or | Reference | Mutant | | | Amino |
| Name | Gene | Position | Type | inserted | Allele | Allele | Promoter | Coding | Acid |
| KG12-05 | NtHAP1a | 1932 | Insertion | 1 | — | A | — | 34 | 97 |
| | | 1932 | Insertion | 1 | — | T | — | 35 | 98 |
| | NtHAP1b | 1932 | Insertion | 1 | — | T | — | 36 | 99 |
| KG12-09 | NtHAP1a | −101 | Insertion | 1 | — | A | 72 | — | — |
| | | 1932 | Deletion | 1 | T | — | — | 37 | 100 |
| | NtHAP1b | 1889 | Deletion | 1 | G | — | — | 38 | 101 |
| | | 1932 | Insertion | 1 | — | T | — | 39 | 102 |
| KG12-13 | NtHAP1a | 1932 | Insertion | 1 | — | T | — | 40 | 103 |
| | NtHAP1b | −100 | Insertion | 1 | — | T | 73 | — | — |
| | | 1932 | Insertion | 1 | — | C | — | 41 | 104 |
| KG12-24 | NtHAP1a | 1932 | Deletion | 1 | T | — | — | 42 | 105 |
| | NtHAP1b | −101 | Insertion | 1 | — | A | 74 | — | — |
| | | 1932 | Insertion | 1 | — | T | — | 43 | 106 |
| KG12-29 | NtHAP1a | −100 | Insertion | 1 | | T | 75 | — | — |
| | | 1932 | Insertion | 1 | — | T | — | 44 | 107 |
| | NtHAP1b | −101 | Insertion | 1 | — | A | 76 | — | |
| | | 1932 | Deletion | 1 | T | — | — | 45 | 108 |
| KG12-31 | NtHAP1a | −100 | Insertion | 1 | — | C | 77 | — | — |
| | | 1932 | Insertion | 1 | — | A | — | 46 | 109 |
| | | 1932 | Insertion | 1 | — | T | — | 47 | 110 |
| | NtHAP1b | −112 | Deletion | 30 | SEQ ID NO: | — | 78 | — | — |
| | | | | | 90 | | | | |
| | | 1932 | Insertion | 1 | — | C | — | 48 | 111 |
| KG1278- | NtHAP1a | −103 | Deletion | 3 | GGA | — | 79 | — | — |
| 31 | | −100 | Insertion | 1 | — | T | 80 | — | — |
| | | 1932 | Insertion | 1 | | A | — | 49 | 112 |
| | | 1932 | Insertion | 1 | — | T | — | 50 | 113 |
| | NtHAP1b | −109 | Deletion | 30 | SEQ ID NO: | — | 81 | — | — |
| | | | | | 91 | | | | |
| | | −100 | Insertion | 1 | — | T | 82 | — | — |
| | NtHAP2 | 21 | Insertion | 1 | — | A | — | 51 | 114 |
| | | 21 | Insertion | 1 | — | T | — | 52 | 115 |
| KG1278- | NtHAP1a | −101 | Insertion | 1 | — | A | 83 | — | — |
| 44 | | 1932 | Deletion | 2 | TT | — | — | 53 | 116 |
| | NtHAP1b | −101 | Insertion | 1 | — | A | 84 | — | — |
| | | −100 | Insertion | 1 | — | T | 85 | — | — |
| | | 1932 | Insertion | 1 | — | A | — | 54 | 117 |
| | | 1932 | Insertion | 1 | — | T | — | 55 | 118 |
| | NtHAP2 | 20 | Deletion | 1 | C | — | — | 56 | 119 |
| KG1278- | NtHAP1a | −103 | Deletion | 3 | GGA | — | 86 | — | — |
| 64 | | −100 | Insertion | 1 | — | T | 87 | — | — |
| | | 1932 | Insertion | 1 | — | A | — | 57 | 120 |
| | | 1932 | Insertion | 1 | — | T | — | 58 | 121 |
| | NtHAP1b | −109 | Deletion | 30 | SEQ ID NO: | — | 88 | — | — |
| | | | | | 92 | | | | |
| | | −100 | Insertion | 1 | — | T | 89 | — | — |
| | NtHAP2 | 21 | Insertion | 1 | — | A | — | 59 | 122 |
| | | 21 | Insertion | 1 | — | T | — | 60 | 123 |
| KG34-02 | NtHAP1a | 2052 | Deletion | 8 | TGGCATGG | — | — | 61 | 124 |
| | | 2063 | Deletion | 17 | SEQ ID NO: | — | — | 62 | 125 |
| | | | | | 93 | | | | |
| KG34-03 | NtHAP1a | 2079 | Insertion | 1 | — | T | — | 63 | 126 |
| KG34-06 | NtHAP1a | 2079 | Insertion | 1 | — | T | — | 64 | 127 |
| | | 2079 | Insertion | 41 | — | SEQ ID | — | 65 | 128 |
| | | | | | | NO: 96 | | | |
| KG34-09 | NtHAP1a | 2023 | Deletion | 1 | C and SEQ | — | — | 66 | 129 |
| | | and | and | and | ID NO: 94 | | | | |
| | | 2025 | Deletion | 53 | | | | | |
| | | 2080 | Insertion | 1 | — | A | — | 67 | 130 |
| KG34-20 | NtHAP1a | 2078 | Deletion | 1 | — | — | — | 68 | 131 |
| | | 2097 | Deletion | 43 | SEQ ID NO: | — | — | 69 | 132 |
| | | | | | 95 | | | | |
| KG34-52 | NtHAP1a | 151 | Deletion | 1765 | SEQ ID NO: | — | — | 70 | 133 |
| | | | | | 536 | | | | |
| | | 2062 | Deletion | 1 | C | — | — | 71 | 134 |

TABLE 14
Izmir NtHAP mutants. Mutation position is measured from the start codon for each
gene (e.g., the first nucleotide of the start codon is position 1). “nt” refers to “nucleotide(s)”.
Reference allele provides the nucleotides that are deleted, when applicable. Mutant allele provides
the nucleotides that are inserted, when applicable. In some instances, a SEQ ID NO is provided
instead of individual nucleotides. The Mutant SEQ ID NO columns provide sequence references
for individual mutant alleles. A “-” symbol indicates “not applicable.”

| | | | | Number | | | | | |
|---|---|---|---|---|---|---|---|---|---|
| Edited | | | | of nt | | | Mutant SEQ ID NO | | |
| Line | Edited | Mutation | Mutation | deleted or | Reference | Mutant | | | Amino |
| Name | Gene | Position | Type | inserted | Allele | Allele | Promoter | Coding | Acid |
| IG12-08 | NtHAP1a | 1932 | Insertion | 1 | — | T | — | 135 | 385 |
| | NtHAP1b | −100 | Insertion | 1 | — | T | 285 | — | — |
| IG12-09 | NtHAP1a | −103 | Deletion | 3 | GGA | — | 286 | — | — |
| | | −100 | Insertion | 1 | — | T | 287 | — | — |
| | | 1909 | Deletion | 22 | SEQ ID | — | — | 136 | 386 |
| | | | | | NO: 352 | | | | |
| | | 1932 | Insertion | 1 | — | A | — | 137 | 387 |
| | NtHAP1b | −101 | Insertion | 1 | — | A | 288 | — | — |
| | | −101 | Deletion | 2 | AT | — | 289 | — | — |
| | | 1887 | Deletion | 76 | SEQ ID | — | — | 138 | 388 |
| | | | | | NO: 353 | | | | |
| | | 1919 | Deletion | 38 | SEQ ID | — | — | 139 | 389 |
| | | | | | NO: 354 | | | | |
| IG12-13 | NtHAP1a | −103 | Deletion | 3 | GGA | — | 290 | — | — |
| | | 1932 | Insertion | 1 | — | A | — | 140 | 390 |
| | | 1932 | Insertion | 1 | — | T | — | 141 | 39 |
| IG12-14 | NtHAP1a | −203 | deletion | 148 | SEQ ID | — | 291 | — | — |
| | | | | | NO: 355 | | | | |
| | | 1932 | Insertion | 1 | | T | — | 142 | 392 |
| | NtHAP1b | −109 | Deletion | 9 | GTGCATG | — | 292 | — | — |
| | | | | | GA | | | | |
| | | −100 | Insertion | 1 | — | T | 293 | — | — |
| | | 1932 | Insertion | 1 | — | T | — | 143 | 393 |
| IG12-15 | NtHAP1a | −104 | Deletion | 10 | SEQ ID | — | 294 | — | — |
| | | | | | NO: 356 | | | | |
| | | −101 | Deletion | 1 | A | — | 295 | — | — |
| | | 1931 | Deletion | 3 | GTT | — | — | 144 | 394 |
| | | 1924 | Deletion | 51 | SEQ ID | — | — | 145 | 395 |
| | | | | | NO: 357 | | | | |
| | NtHAP1b | −101 | Insertion | 1 | — | A | 296 | — | — |
| | | 1932 | Deletion | 1 | T | — | — | 146 | 396 |
| | | 1932 | Deletion | 6 | TTGTTC | — | — | 147 | 397 |
| IG12-17 | NtHAP1a | −107 | Deletion | 7 | GCATGG | — | 297 | — | — |
| | | | | | A | | | | |
| | | 1932 | Insertion | 1 | — | A | — | 148 | 398 |
| | | 1932 | Deletion | 1 | T | — | | 149 | 399 |
| | NtHAP1b | 1899 | Deletion | 78 | SEQ ID | — | — | 150 | 400 |
| | | | | | NO: 358 | | | | |
| | | 1932 | Insertion | 1 | — | T | — | 151 | 401 |
| IG12-30 | NtHAP1a | −181 | Deletion | 160 | SEQ ID | — | 298 | — | — |
| | | | | | NO: 359 | | | | |
| | | 1931 | Deletion | 4 | GTTG | — | — | 152 | 402 |
| | | 1932 | Insertion | 1 | — | A | — | 153 | 403 |
| | NtHAP1b | 1932 | Insertion | 1 | — | T | | 154 | 404 |
| | | 1932 | Deletion | 6 | TTGTTC | | — | 155 | 405 |
| IG12-35 | NtHAP1a | −181 | Deletion | 160 | SEQ ID | — | 299 | — | — |
| | | | | | NO: 360 | | | | |
| | | 1931 | Deletion | 4 | GTTG | — | — | 156 | 406 |
| | | 1932 | Insertion | 1 | — | A | — | 157 | 407 |
| | NtHAP1b | −100 | Insertion | 1 | — | C | 300 | — | — |
| | | 1932 | Insertion | 1 | — | T | — | 158 | 408 |
| | | 1932 | Deletion | 6 | TTGTTC | — | — | 159 | 409 |
| IG12-39 | NtHAP1a | −181 | Deletion | 160 | SEQ ID | — | 301 | — | — |
| | | | | | NO: 361 | | | | |
| | | 1931 | Deletion | 4 | GTTG | — | — | 160 | 410 |
| | | 1932 | Insertion | 1 | — | A | — | 161 | 411 |
| | NtHAP1b | 1932 | Insertion | 1 | — | T | — | 162 | 412 |
| | | 1932 | Deletion | 6 | TTGTTC | — | — | 163 | 413 |
| IG12-41 | NtHAP1a | 1932 | Insertion | 1 | — | A | — | 164 | 414 |
| | NtHAP1b | −101 | Insertion | 1 | — | A | 302 | — | — |
| | | 1932 | Insertion | 1 | — | T | — | 165 | 415 |
| IG12-42 | NtHAP1a | −101 | Insertion | 1 | — | A | 303 | — | — |
| | | −100 | Deletion | 1 | T | — | 304 | — | — |
| | | 1932 | Insertion | 1 | — | A | — | 166 | 416 |
| | | 1932 | Insertion | 1 | — | T | — | 167 | 417 |
| | NtHAP1b | −104 | Deletion | 17 | SEQ ID | — | 305 | — | — |
| | | | | | NO: 362 | | | | |
| | | −101 | Insertion | 1 | — | A | 306 | — | — |
| | | 1932 | Insertion | 1 | — | A | — | 168 | 418 |
| IG12-44 | NtHAP1a | −181 | Deletion | 160 | SEQ ID | — | 307 | — | — |
| | | | | | NO: 363 | | | | |
| | | 1931 | Deletion | 4 | GTTG | — | — | 169 | 419 |
| | | 1932 | Insertion | 1 | — | A | — | 170 | 420 |
| | NtHAP1b | 1932 | Insertion | 1 | — | T | — | 171 | 421 |
| | | 1932 | Deletion | 6 | TTGTTC | | — | 172 | 422 |
| IG12-53 | NtHAP1a | −181 | Deletion | 160 | SEQ ID | — | 308 | — | — |
| | | | | | NO: 364 | | | | |
| | | 1931 | Deletion | 4 | GTTG | — | — | 173 | 423 |
| | | 1932 | Insertion | 1 | — | A | — | 174 | 424 |
| | NtHAP1b | 1932 | Insertion | 1 | — | T | — | 175 | 425 |
| | | 1932 | Deletion | 6 | TTGTTC | — | | 176 | 426 |
| IG12-58 | NtHAP1a | −105 | Deletion | 5 | ATGGA | — | 309 | — | — |
| | | 1932 | Deletion | 1 | T | — | — | 177 | 427 |
| | | 1932 | Deletion | 4 | TTGT | — | — | 178 | 428 |
| | NtHAP1b | 1907 | Deletion | 1 | A | | — | 179 | 429 |
| | | 1909 | Deletion | 23 | SEQ ID | — | — | 180 | 430 |
| | | | | | NO: 365 | | | | |
| IG1278- | NtHAP1a | −101 | Insertion | 1 | — | A | 310 | — | — |
| 105 | NtHAP1b | −101 | Insertion | 1 | — | A | 311 | | — |
| | | −100 | Insertion | 1 | — | C | 312 | — | — |
| | | 1932 | Insertion | 1 | — | T | — | 181 | 431 |
| | NtHAP2 | 21 | Insertion | 1 | — | A | — | 182 | 432 |
| | | 21 | Deletion | 2 | TG | — | — | 183 | 433 |
| IG1278- | NtHAP1a | −101 | Insertion | 1 | — | A | 313 | — | — |
| 106 | | 1932 | Insertion | 1 | — | T | — | 184 | 434 |
| | NtHAP1b | −105 | Deletion | 4 | ATGG | — | 314 | — | — |
| | | −101 | Insertion | 1 | — | A | 315 | — | — |
| | | 1932 | Insertion | 1 | — | T | — | 185 | 435 |
| | | 1932 | Deletion | 4 | TTGT | — | — | 186 | 436 |
| IG1278- | NtHAP2 | 1872 | Insertion | 1 | — | A | | 187 | 437 |
| 107 | | 1872 | Deletion | 1 | C | — | — | 188 | 438 |
| IG1278- | NtHAP1a | −100 | Insertion | 1 | — | T | 316 | — | — |
| 112 | NtHAP2 | 1872 | Insertion | 1 | — | A | — | 189 | 439 |
| | | 1872 | Deletion | 1 | C | — | — | 190 | 440 |
| IG1278- | NtHAP1a | (−159/ | Deletion | 1/108 | C and SEQ | — | 317 | — | — |
| 117 | | −157) | and SNP | | ID NO: 366 | | | | |
| | | −101 | Insertion | 1 | — | A | 318 | — | — |
| | NtHAP1b | (−101/ | Deletion | 1 | G/A | (—/T) | 319 | — | |
| | | −99) | and SNP | | | | | | |
| | | −101 | Insertion | 1 | — | A | 320 | — | — |
| | | 1932 | Insertion | 1 | — | T | — | 191 | 441 |
| | NtHAP2 | 21 | Insertion | 1 | — | T | — | 192 | 442 |
| | | 1872 | Deletion | 1 | C | — | — | 193 | 443 |
| IG1278- | NtHAP1b | 1932 | Insertion | 1 | — | A | — | 194 | 444 |
| 12 | NtHAP2 | −15 | Deletion | 29 | SEQ ID | — | 321 | 534 | — |
| | | | | | NO: 367 | | | | |
| | | 17 | Deletion | 7 | GTTCTGA | | 322 | 535 | — |
| IG1278- | NtHAP1b | −101 | Insertion | 1 | — | A | 323 | — | — |
| 13 | | −100 | Insertion | 1 | — | G | 324 | — | — |
| IG1278- | NtHAP1b | −105 | Deletion | 4 | ATGG | — | 325 | — | — |
| 14 | | 1932 | Insertion | 1 | — | T | — | 195 | 445 |
| | NtHAP2 | 1872 | Deletion | 1 | C | — | — | 196 | 446 |
| | | 1871 | Insertion | 1 | — | T | — | 197 | 447 |
| IG1278- | NtHAP1a | −150 | Deletion | 50 | SEQ ID | — | 326 | — | — |
| | | | | | NO: 368 | | | | |
| 32 | | −101 | Insertion | 1 | — | A | 327 | — | — |
| | | 1932 | Insertion | 1 | — | T | — | 198 | 448 |
| | NtHAP1b | −101 | Deletion | 11 | SEQ ID | — | 328 | — | — |
| | | | | | NO: 369 | | | | |
| | | 1932 | Insertion | 1 | — | A | — | 199 | 449 |
| | NtHAP2 | 1871 | Insertion | 1 | — | T | — | 200 | 450 |
| | | 1872 | Insertion | 1 | — | A | — | 201 | 451 |
| IG1278- | NtHAP1a | −100 | Insertion | 1 | — | T | 329 | — | — |
| 35 | | 1932 | Insertion | 1 | — | T | — | 202 | 452 |
| | NtHAP1b | −100 | Insertion | 1 | — | T | 330 | — | — |
| | | 1932 | Insertion | 1 | — | A | — | 203 | 453 |
| | NtHAP2 | 1871 | Insertion | 1 | — | T | — | 204 | 454 |
| IG1278- | NtHAP1a | −105 | Deletion | 5 | ATGGA | — | 331 | — | — |
| 47 | NtHAP1b | −101 | Insertion | 1 | — | A | 332 | — | |
| | | −100 | Insertion | 1 | — | T | 333 | — | — |
| | | 1930 | Deletion | 8 | AGTTGTT | — | — | 205 | 455 |
| | | | | | C | | | | |
| | NtHAP2 | 1871 | Insertion | 1 | — | T | — | 206 | 456 |
| IG1278- | NtHAP1a | 1884 | Deletion | 48 | SEQ ID | — | — | 207 | 457 |
| | | | | | NO: 370 | | | | |
| 51 | NtHAP1b | −106 | Deletion | 6 | CATGGA | — | 334 | — | — |
| | | −101 | Deletion | 18 | SEQ ID | — | 335 | — | — |
| | | | | | NO: 371 | | | | |
| | NtHAP2 | 1871 | Deletion | 1 | T | — | — | 208 | 458 |
| IG1278- | NtHAP1a | −211 | Deletion | 139 | SEQ ID | — | 336 | — | — |
| | | | | | NO: 372 | | | | |
| 59 | | 1932 | Insertion | 1 | — | A | — | 209 | 459 |
| | NtHAP1b | −142 | Deletion | 45 | SEQ ID | — | 337 | — | |
| | | | | | NO: 373 | | | | |
| | | −101 | Insertion | 1 | — | A | 338 | — | — |
| | | 1932 | Insertion | 1 | — | A | — | 210 | 460 |
| IG1278- | NtHAP1a | −102 | Insertion | 1 | — | A | 339 | — | — |
| 62 | NtHAP1b | −100 | Insertion | 1 | — | C | 340 | — | — |
| | NtHAP2 | 1870 | Deletion | 7 | GTCATAA | — | — | 211 | 461 |
| | | 1872 | Insertion | 1 | | A | — | 212 | 462 |
| IG1278- | NtHAP1a | −100 | Insertion | 1 | — | T | 341 | — | — |
| 69 | NtHAP2 | 1871 | Insertion | 1 | — | T | — | 213 | 463 |
| | | 1872 | Insertion | 1 | — | A | — | 214 | 464 |
| IG1278- | NtHAP1a | 1929 | Deletion | 3 | TAG | — | — | 215 | 465 |
| 73 | NtHAP1b | −100 | Insertion | 1 | — | T | 342 | — | — |
| IG1278- | NtHAP1a | −101 | Insertion | 1 | — | A | 343 | — | — |
| 76 | | 1932 | Insertion | 1 | — | T | — | 216 | 466 |
| | NtHAP1b | −101 | Insertion | 1 | — | A | 344 | — | — |
| | NtHAP2 | 1872 | Insertion | 1 | — | A | — | 217 | 467 |
| IG1278- | NtHAP1a | (−159/ | Deletion | 1/108 | C and SEQ | — | 345 | — | — |
| 90 | | −157) | and SNP | | ID NO: 374 | | | | |
| | | −101 | Insertion | 1 | — | A | 346 | — | — |
| | NtHAP1b | (−101/ | Deletion | 1 | G/A | ( /T) | 347 | — | — |
| | | −99) | and SNP | | | | | | |
| | | −101 | Insertion | 1 | — | A | 348 | — | |
| | | 1932 | Insertion | 1 | — | T | — | 218 | 468 |
| | NtHAP2 | 21 | Insertion | 1 | — | T | — | 219 | 469 |
| | | 1872 | Deletion | 1 | C | — | — | 220 | 470 |
| IG1278- | NtHAP1a | −101 | Insertion | 1 | — | A | 349 | — | — |
| 97 | | −100 | Insertion | 1 | — | T | 350 | — | — |
| | | 1932 | Insertion | 1 | — | T | | 221 | 471 |
| | NtHAP1b | −101 | Insertion | 1 | — | A | 351 | — | — |
| | | 1932 | Insertion | 1 | — | T | — | 222 | 472 |
| IG34-14 | NtHAP1a | 2078 | Deletion | 43 | SEQ ID | — | — | 223 | 473 |
| | | | | | NO: 375 | | | | |
| | | 2080 | Insertion | 1 | — | A | — | 224 | 474 |
| IG34-15 | NtHAP1a | 2073 | Deletion | 7 | AGCAGA | — | — | 225 | 475 |
| | | | | | T | | | | |
| IG34-19 | NtHAP1a | 2047 | Deletion | 2 | GA | — | — | 226 | 476 |
| | | 2051 | Deletion | 3 | ATG | — | — | 227 | 477 |
| | | 2056 | Deletion | 6 | ATGGTT | — | — | 228 | 478 |
| | | 2063 | Deletion | 15 | SEQ ID | — | — | 229 | 479 |
| | | | | | NO: 376 | | | | |
| | | 2080 | Insertion | 1 | — | A | — | 230 | 480 |
| | | 2080 | Deletion | 2 | CG | | — | 231 | 481 |
| | | 2083 | Deletion | 3 | AGG | — | — | 232 | 482 |
| IG34-29 | NtHAP1a | 2073 | Deletion | 7 | AGCAGA | — | — | 233 | 483 |
| | | | | | T | | | | |
| IG34-42 | NtHAP1a | 2002 | Insertion | 1 | — | T | — | 234 | 484 |
| | | 2020 | SNP | 1 | A | C | — | 235 | 485 |
| | | 2032 | SNP | 1 | C | G | — | 236 | 486 |
| | | 2041 | Deletion | 56 | SEQ ID | — | — | 237 | 487 |
| | | | | | NO: 377 | | | | |
| | | 2080 | Insertion | 1 | — | A | — | 238 | 488 |
| IG34-48 | NtHAP1a | 2002 | Insertion | 1 | — | T | — | 239 | 489 |
| | | 2020 | SNP | 1 | A | C | — | 240 | 490 |
| | | 2032 | SNP | 1 | C | G | — | 241 | 491 |
| | | 2041 | Deletion | 56 | SEQ ID | — | — | 242 | 492 |
| | | | | | NO: 378 | | | | |
| | | 2080 | Insertion | 1 | — | A | — | 243 | 493 |
| IG34-65 | NtHAP1a | 2002 | Insertion | 1 | — | T | — | 244 | 494 |
| | | 2020 | SNP | 1 | A | C | — | 245 | 495 |
| | | 2032 | SNP | 1 | C | G | — | 246 | 496 |
| | | 2041 | Deletion | 56 | SEQ ID | — | — | 247 | 497 |
| | | | | | NO: 379 | | | | |
| | | 2080 | Insertion | 1 | — | A | — | 248 | 498 |
| IG34-71 | NtHAP1a | 2002 | Insertion | 1 | — | T | — | 249 | 499 |
| | | 2020 | SNP | 1 | A | C | — | 250 | 500 |
| | | 2032 | SNP | 1 | C | G | — | 251 | 501 |
| | | 2041 | Deletion | 56 | SEQ ID | — | — | 252 | 502 |
| | | | | | NO: 380 | | | | |
| | | 2080 | Insertion | 1 | — | A | — | 253 | 503 |
| IG34-72 | NtHAP1a | 2002 | Insertion | 1 | — | T | — | 254 | 504 |
| | | 2020 | SNP | 1 | A | C | — | 255 | 505 |
| | | 2032 | SNP | 1 | C | G | — | 256 | 506 |
| | | 2041 | Deletion | 56 | SEQ ID | — | — | 257 | 507 |
| | | | | | NO: 381 | | | | |
| IG34-72 | NtHAP1a | 2080 | Insertion | 1 | — | A | — | 258 | 508 |
| IG34-74 | NtHAP1a | 2002 | Insertion | 1 | — | T | — | 259 | 509 |
| | | 2020 | SNP | 1 | A | C | — | 260 | 510 |
| | | 2032 | SNP | 1 | C | G | — | 261 | 511 |
| | | 2041 | Deletion | 56 | SEQ ID | — | — | 262 | 512 |
| | | | | | NO: 382 | | | | |
| | | 2080 | Insertion | 1 | — | A | — | 263 | 513 |
| IG34-79 | NtHAP1a | 2079 | Insertion | 1 | — | T | — | 264 | 514 |
| IG34-94 | NtHAP1a | 2002 | Insertion | 1 | — | T | — | 265 | 515 |
| | | 2020 | SNP | 1 | A | C | — | 266 | 516 |
| | | 2032 | SNP | 1 | C | G | — | 267 | 517 |
| | | 2041 | Deletion | 56 | SEQ ID | — | — | 268 | 518 |
| | | | | | NO: 383 | | | | |
| | | 2080 | Insertion | 1 | — | A | — | 269 | 519 |
| IG56-02 | NtHAP1b | 2080 | Insertion | 1 | — | T | — | 270 | 526 |
| IG56-12 | NtHAP1b | 2070 | Deletion | 52 | SEQ ID | — | — | 271 | 520 |
| | | | | | NO: 384 | | | | |
| | | 2080 | Insertion | 1 | — | A | — | 272 | 521 |
| IG56-20 | NtHAP1b | 2073 | Deletion | 7 | AGCAGA | | — | 273 | 522 |
| | | | | | C | | | | |
| | | 2079 | Deletion | 1 | C | — | — | 274 | 523 |
| IG56-75 | NtHAP1b | 2075 | Deletion | 4 | CAGA | — | — | 275 | 524 |
| | | 2079 | Deletion | 1 | C | — | — | 276 | 525 |
| | | 2080 | Insertion | 1 | — | T | — | 277 | 526 |
| IG78-11 | NtHAP2 | 1872 | Insertion | 1 | — | A | — | 278 | 527 |
| IG78-13 | NtHAP2 | 20 | Deletion | 4 | CTGA | — | — | 279 | 528 |
| IG78-33 | NtHAP2 | 1871 | Insertion | 1 | — | T | — | 280 | 529 |
| | | 1872 | Deletion | 4 | CATA | — | — | 281 | 530 |
| IG78-35 | NtHAP2 | 21 | Insertion | 1 | — | T | — | 282 | 531 |
| | | 1872 | Insertion | 1 | — | A | — | 283 | 532 |
| IG78-47 | NtHAP2 | 1872 | Insertion | 1 | — | A | — | 284 | 533 |

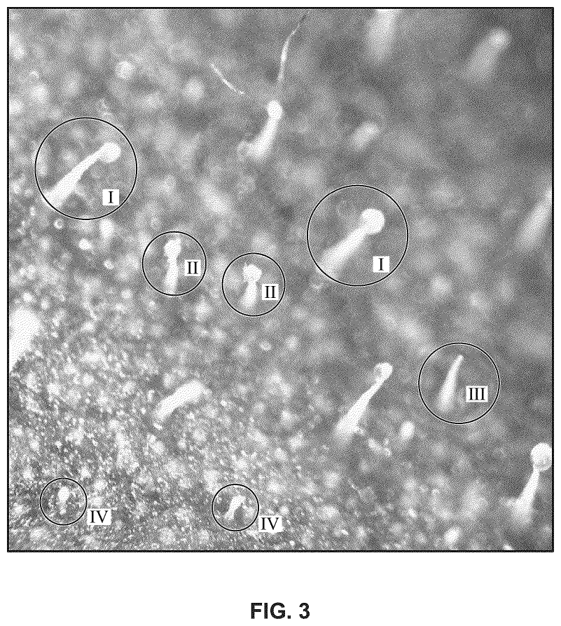

Regenerated tobacco plants are examined for trichome development under a stereomicroscope to identify changes in trichome density and total number in various plant parts compared with control plants. FIG. 3 depicts an image of trichome types observed on an Izmir tobacco leaf.

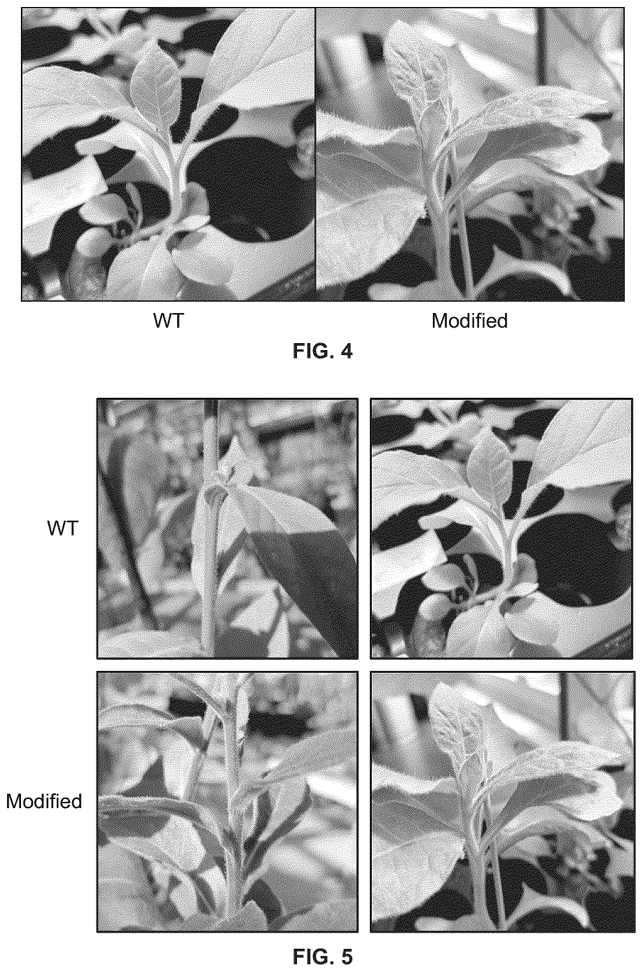

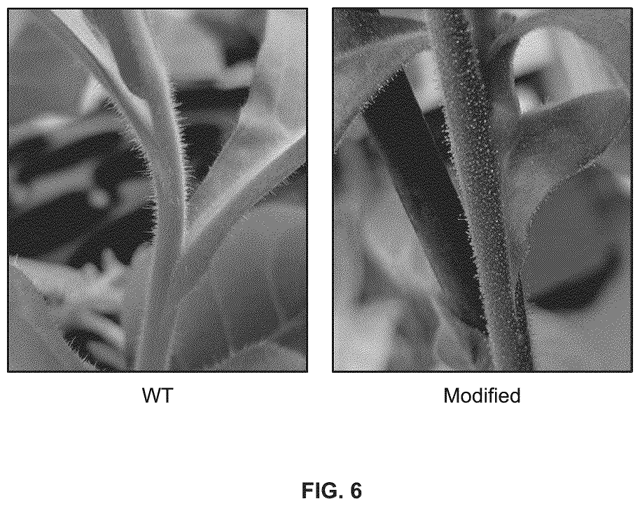

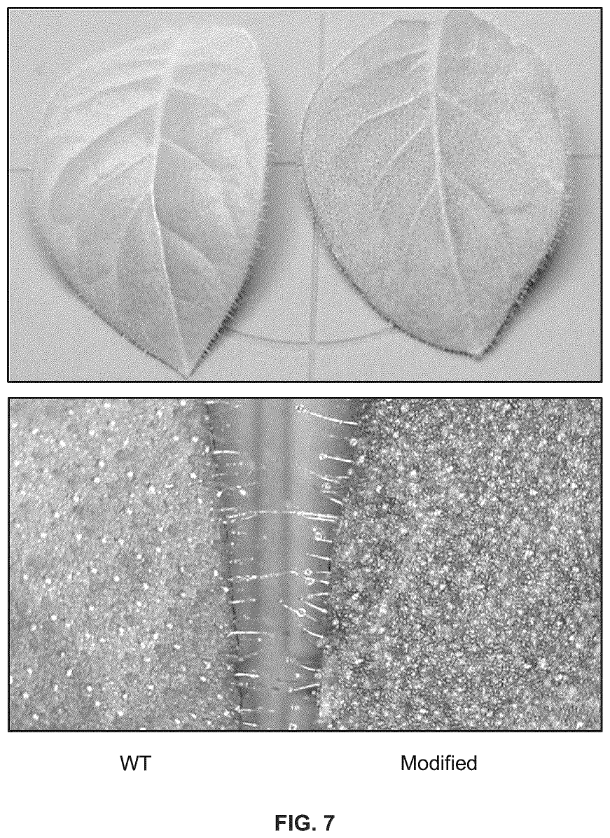

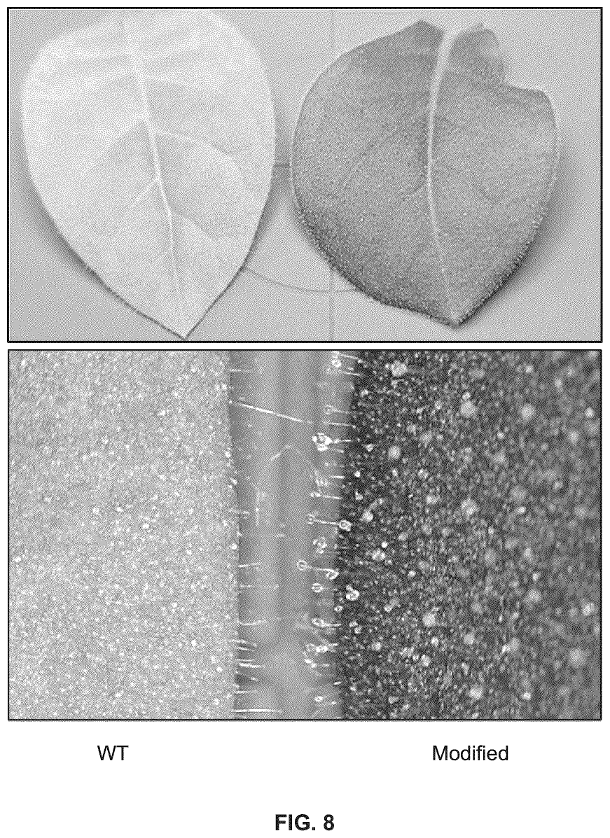

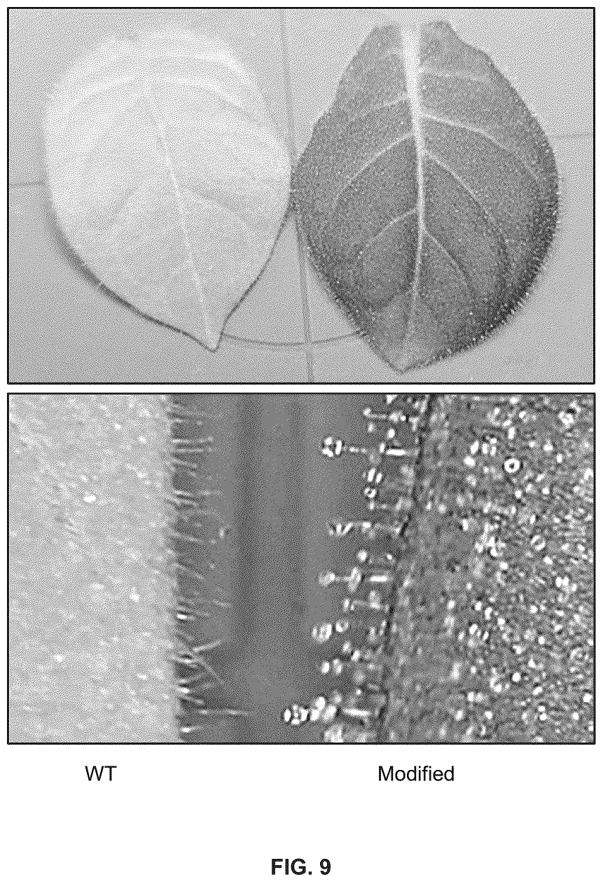

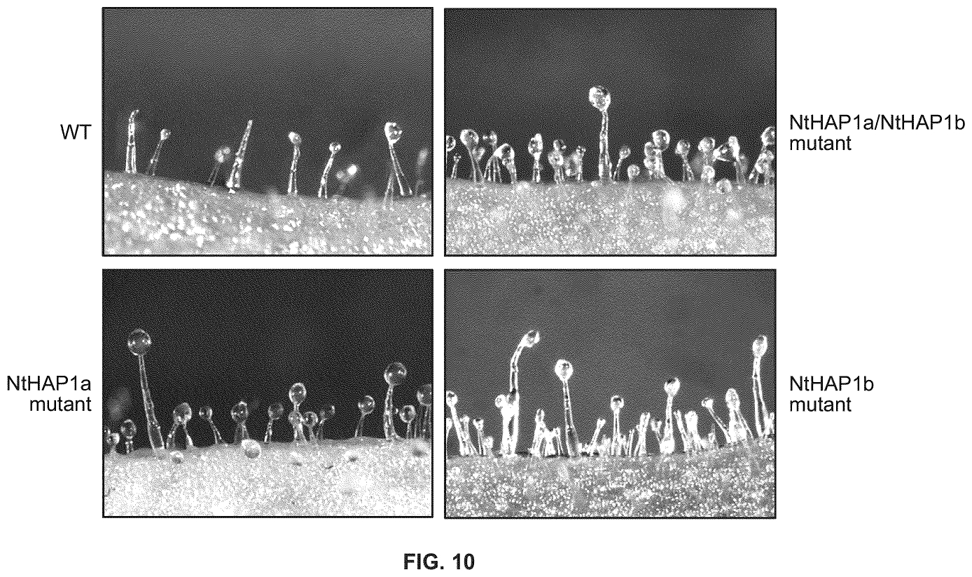

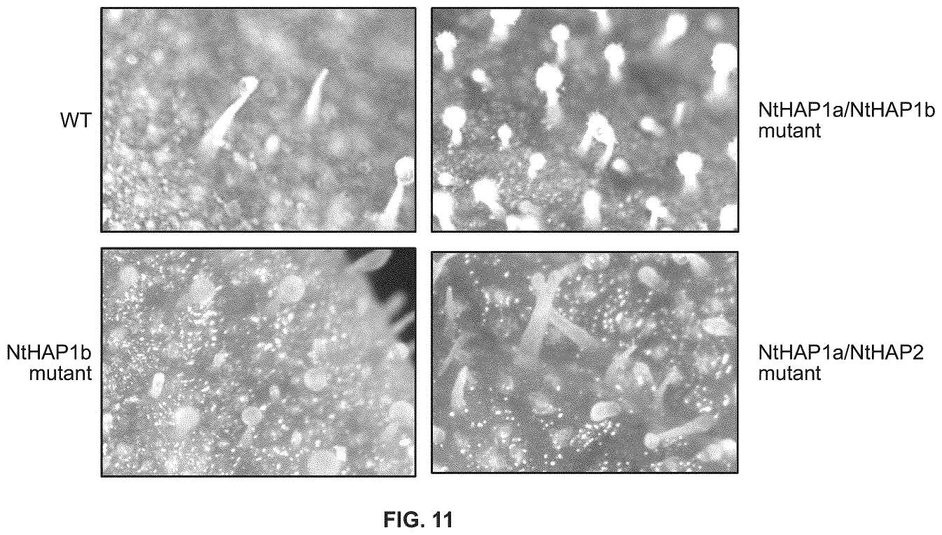

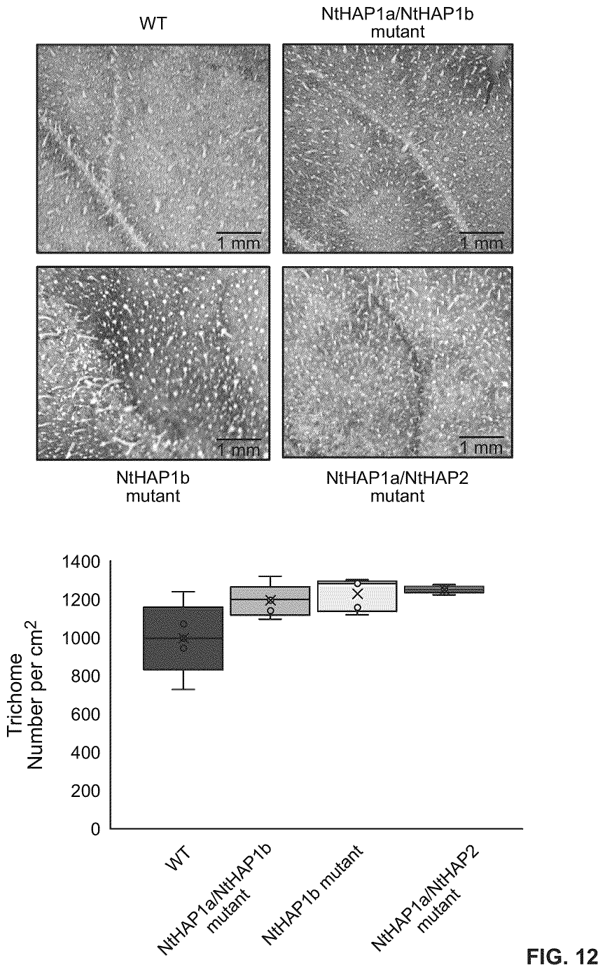

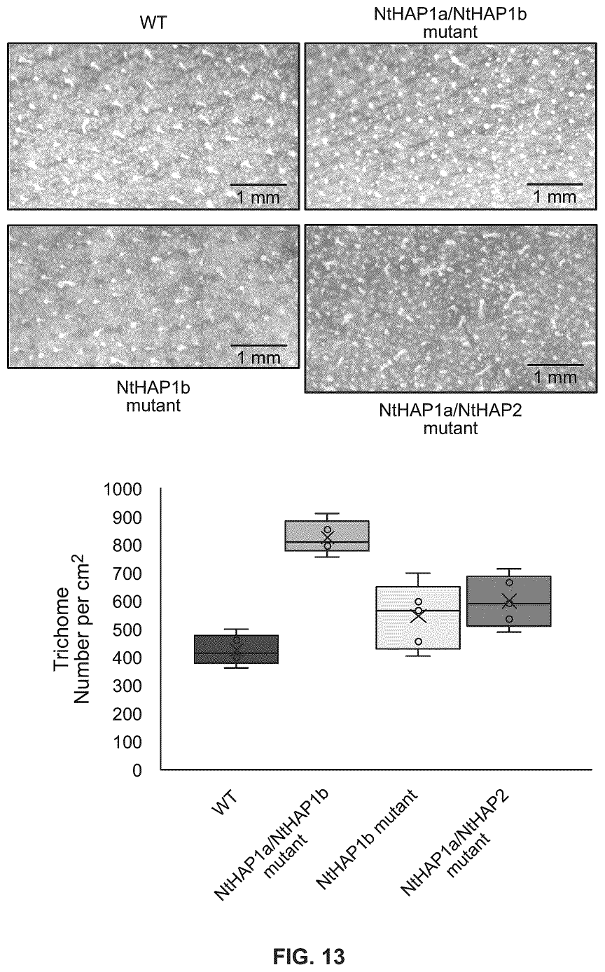

Regenerated tobacco plants comprising induced mutations display increased trichome numbers and densities as compared to wild type (WT) control tobacco plants. See FIG. 4, FIG. 5, FIG. 6, FIG. 7, FIG. 8, FIG. 9, FIG. 10, and FIG. 11. FIG. 12 and FIG. 13 provide quantification of the increased trichome density observed in the modified tobacco plants.

### Example 4. Generation of Hairplus Mutations in Tobacco Using Ethyl Methylsulfonate

Tobacco seeds are treated with ethyl methylsulfonate (EMS) to induce mutagenesis. EMS mutagenesis is known to induce random point mutations in DNA. TILLING (Targeted Induced Local Lesions in Genomes) can be used to rapidly screen large numbers of seeds for EMS-induced mutations. See McCallum et al., Nat. Biotechnol., 18:455-457 (2000).

Eight hundred mutagenized tobacco populations are screened for mutations in any of NtHAP1a, NtHAP1b, and NtHAP2.

Ten individual mutant lines are identified from screening the 800 mutagenized tobacco populations, as shown in Table 15. Seven unique mutations are identified, as some of the identified mutations are identical to each other. Each of the identified mutations results in a truncated NtHAP1a, NtHAP1b, or NtHAP2 protein, respectively.

TABLE 15
EMS-induced NtHAP mutants

| | | | | | | | Mutant | |
|---|---|---|---|---|---|---|---|---|
| | | | | | Amino | | SEQ ID NO | |
| Mutant | NtHAP | Mutation | Control | Mutant | Acid | | Coding | Amino |
| Name | gene | Position | Allele | Allele | Change | Zygosity | Sequence | Acid |
| E511- | NtHAP1a | 1240 | C | T | Glutamine to | Heterozygous | 20 | 27 |
| P3-13 | | | | | Stop Codon at | | | |
| E511- | | | | | position 414 of | | | |
| | | | | | SEQ ID NO: 1 | | | |
| E511- | NtHAP1a | 1953 | G | A | Tryptophan to | Heterozygous | 21 | 28 |
| P5-31 | | | | | Stop Codon at | | | |
| E511- | | | | | position 651 of | | | |
| P6-83 | | | | | SEQ ID NO: 1 | | | |
| E511- | NtHAP1a | 1273 | C | T | Glutamine to | Heterozygous | 22 | 29 |
| P5-58 | | | | | Stop Codon at | | | |
| | | | | | position 425 of | | | |
| | | | | | SEQ ID NO: 1 | | | |
| E511- | NtHAP1a | 1262 | G | A | Tryptophan to | Heterozygous | 23 | 30 |
| P8-73 | | | | | Stop Codon at | | | |
| E511- | | | | | position 421 of | | | |
| P8-78 | | | | | SEQ ID NO: 1 | | | |
| E511- | NtHAP1b | 1952 | G | A | Tryptophan to | Heterozygous | 24 | 31 |
| P9-6 | | | | | Stop Codon at | | | |
| | | | | | position 651 of | | | |
| | | | | | SEQ ID NO: 2 | | | |
| E511- | NtHAP1b | 1953 | G | A | Tryptophan to | Heterozygous | 25 | 32 |
| P9-83 | | | | | Stop Codon at | | | |
| | | | | | position 651 of | | | |
| | | | | | SEQ ID NO: 2 | | | |
| E511- | NtHAP2 | 298 | C | T | Glutamine to | Heterozygous | 26 | 33 |
| P9-67 | | | | | Stop Codon at | | | |
| | | | | | position 100 of | | | |
| | | | | | SEQ ID NO: 3 | | | |

The identified mutated plants from Table 15 are grown and self-pollinated to generate plants that are homozygous for each respective mutation. Homozygous plants are examined to determine trichome phenotype.

Additional crosses are also made to generate double mutant (e.g., NtHAP1a/NtHAP1b; NtHAP1a/NtHAP2; NtHAP1b/NtHAP2) and triple mutant (NtHAP1a/NtHAP1b/NtHAP2) tobacco plants. The double and triple mutant plants are examined to determine trichome phenotype.

### Example 5. RNAi Knockdown of HAP Homologues in Tobacco

Artificial miRNAs or other RNAi constructs are generated to produce a miRNA or another non-coding RNA that is capable of reducing the expression of each of the NtHAP genes. The artificial miRNA or RNAi construct is inserted into a plasmid under the control of a CaMV 35S promoter. The plasmid further comprises a NOS terminator, and a cassette comprising a kanamycin selection marker (NPT II) operably linked to an Actin2 promoter and a NOS terminator. One RNAi construct is designed to simultaneously target NtHAP1a and NtHAP1b (SEQ ID NO: 18). A second RNAi construct is designed to target only NtHAP2 (SEQ ID NO: 19).

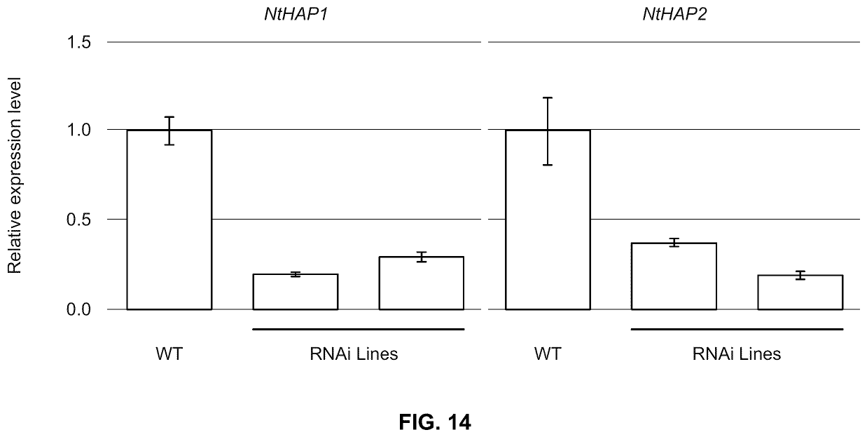

Tobacco leaf discs are transformed, and tobacco plants are regenerated, as described in Example 2. Tobacco leaf discs are transformed with a single RNAi construct (to suppress NtHAP1a and NtHAP1b or to suppress only NtHAP2) or both RNAi constructs (to simultaneously suppress all three genes). RT-PCR is used to confirm suppression of the NtHAP genes in three-week old T1 tobacco plants expressing the RNAi constructs and corresponding wildtype tobacco plants using primers comprising nucleic acid sequences provided as SEQ ID NOs: 606 to 611. Relative expression is normalized to N. tabacum Elongation Factor 1-alpha. See FIG. 14.

Regenerated tobacco plants are examined for trichome development. During the vegetative stage of growth, samples of transgenic lines of NtHAP1a/NtHAP1b, NtHAP2, and NtHAP1a/NtHAP1b/NtHAP2 suppression lines are examined under a stereomicroscope to identify changes in trichome density and total trichrome number in various plant parts compared with control plants.

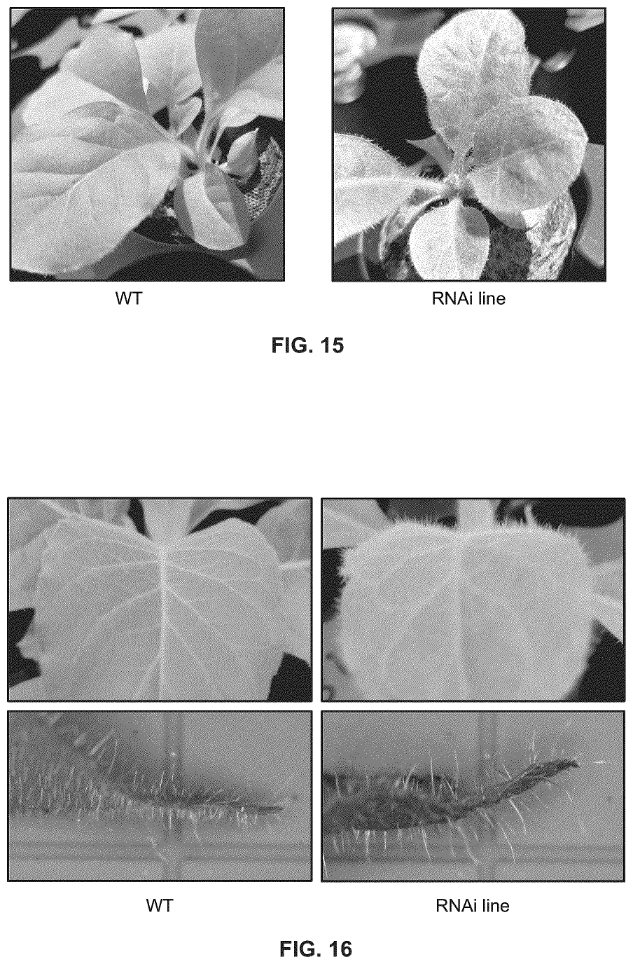

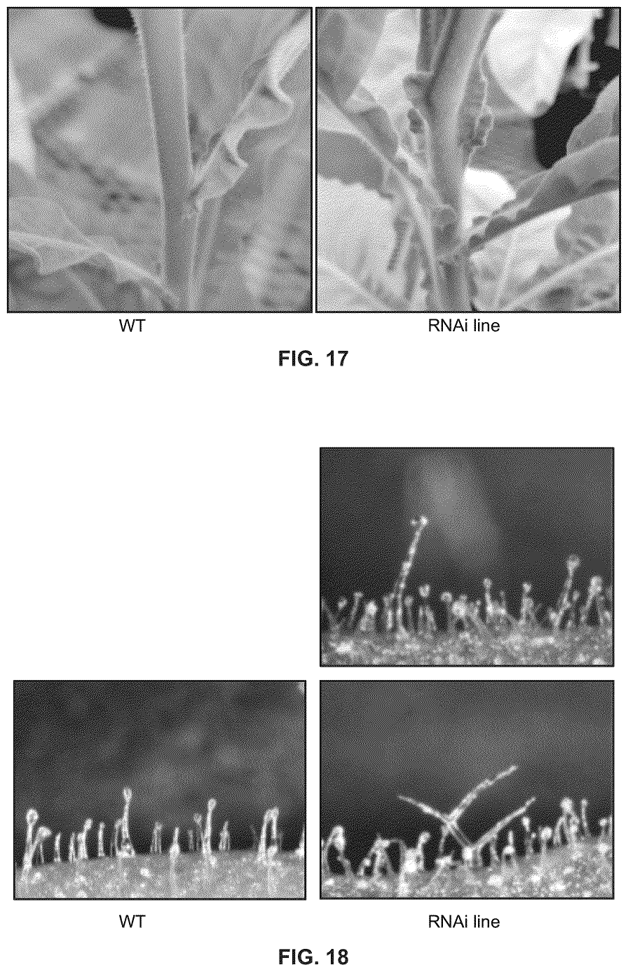

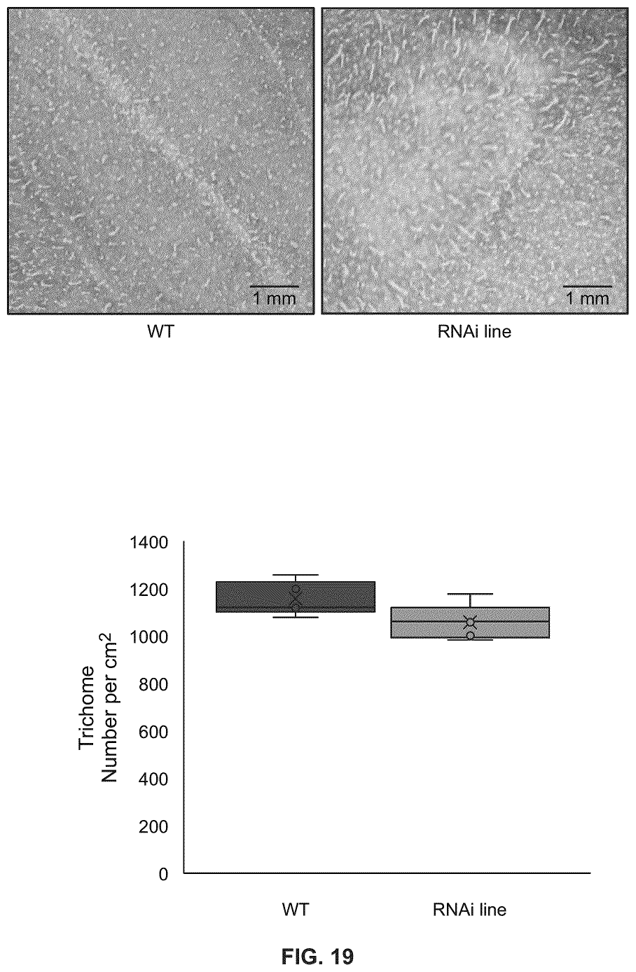

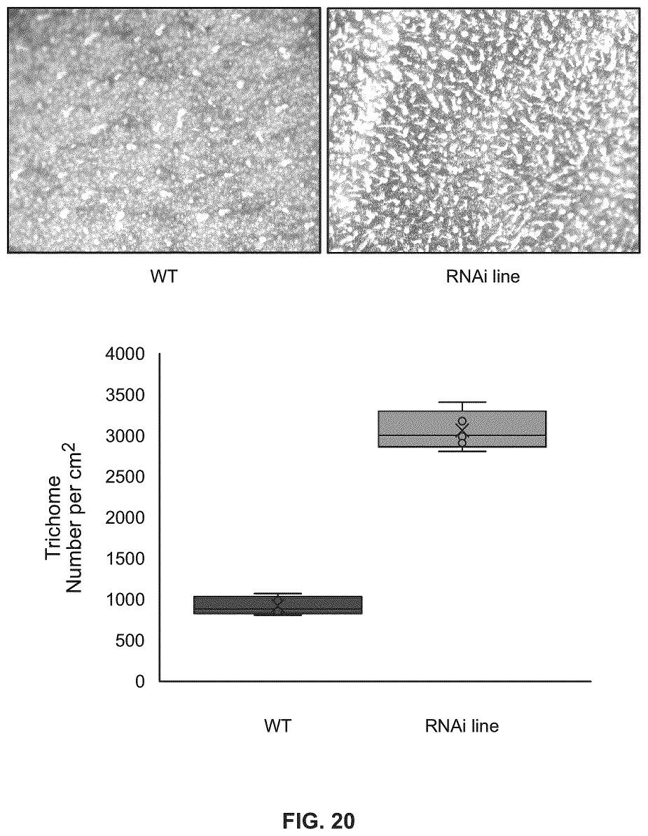

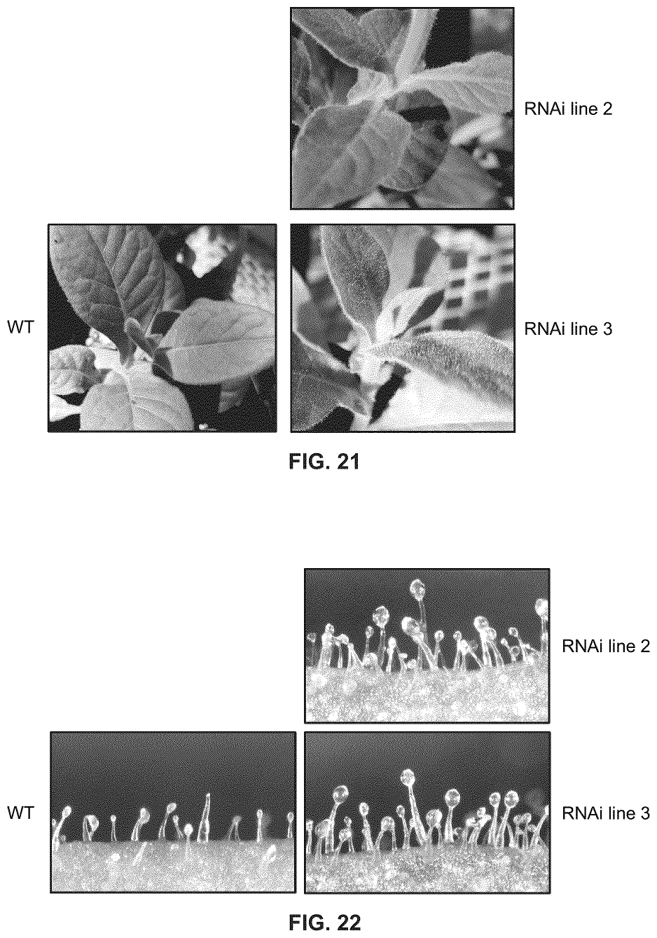

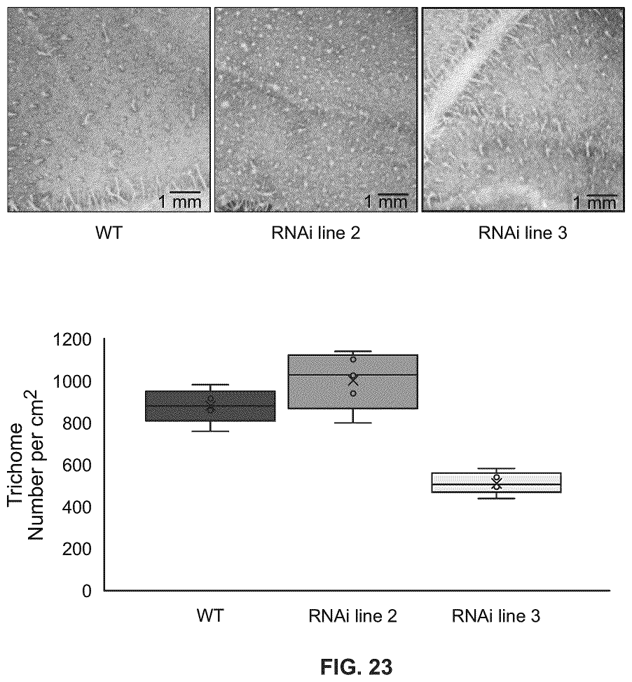

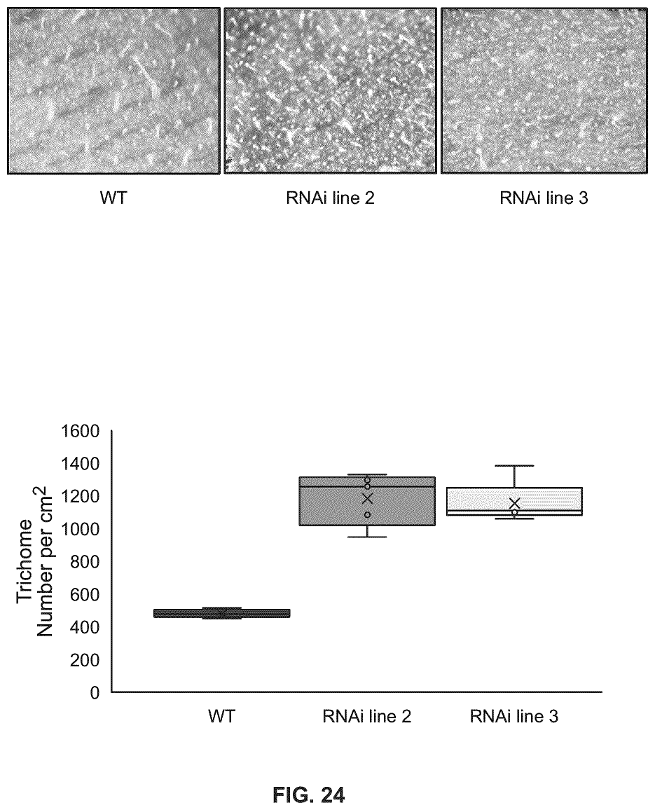

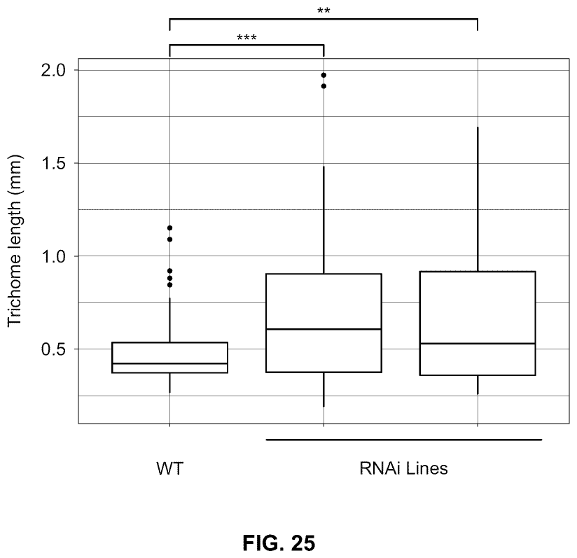

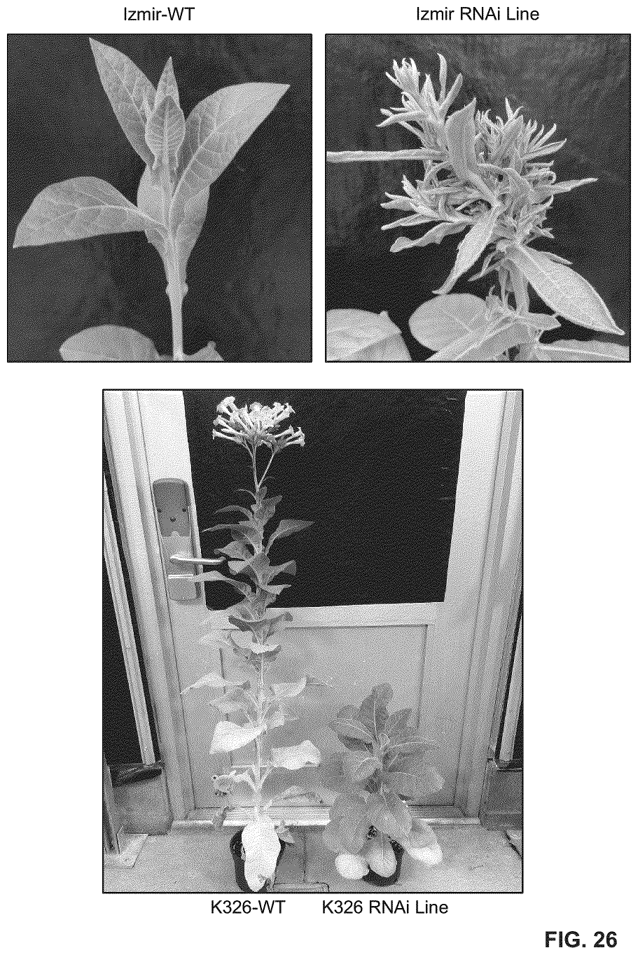

Three independently generated K326 tobacco line comprising both RNAi constructs (SEQ ID NOs: 18 and 19; RNA lines 1, 2, and 3, respectively) exhibit increased trichome numbers and densities as compared to a control. See FIG. 15, FIG. 16, FIG. 17, FIG. 18, FIG. 20, FIG. 21, and FIG. 22. Plants are observed at different stages, and trichome density differs between control (wild type) plants and plants expressing SEQ ID NOs: 18 and 19. See FIG. 19, FIG. 20, FIG. 23, and FIG. 24. Two of the K326 RNAi lines also exhibit a statistically significant increase in average trichome length. See FIG. 25. RNAi lines can also exhibit a shorter plant stature and altered inflorescence development as compared to wildtype plants. See FIG. 26.

### Example 6. Measuring Terpenoid Composition in Modified Tobacco

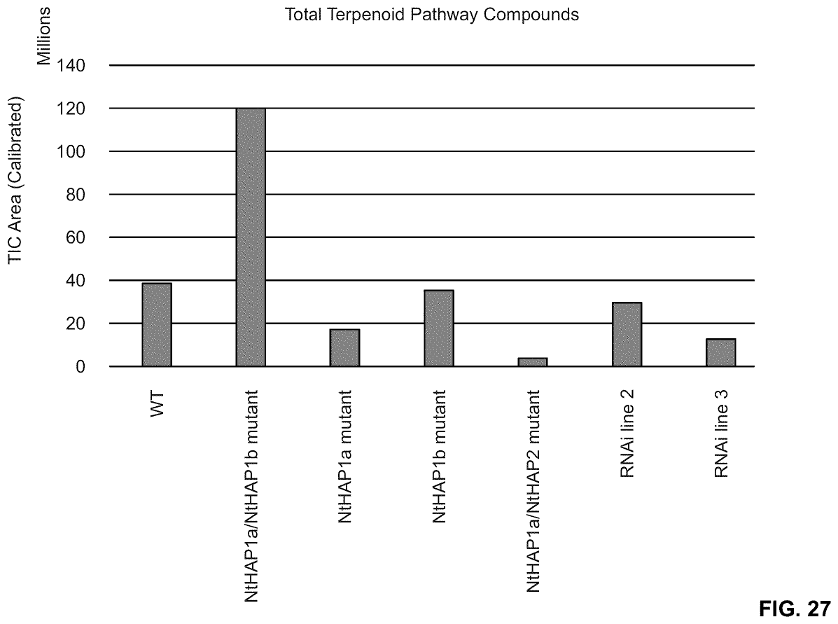

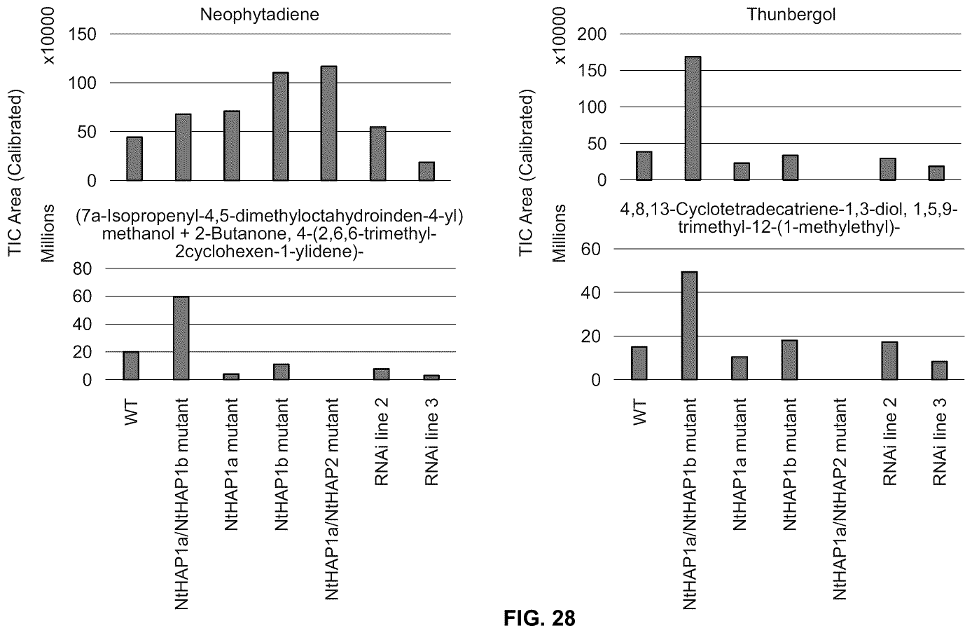

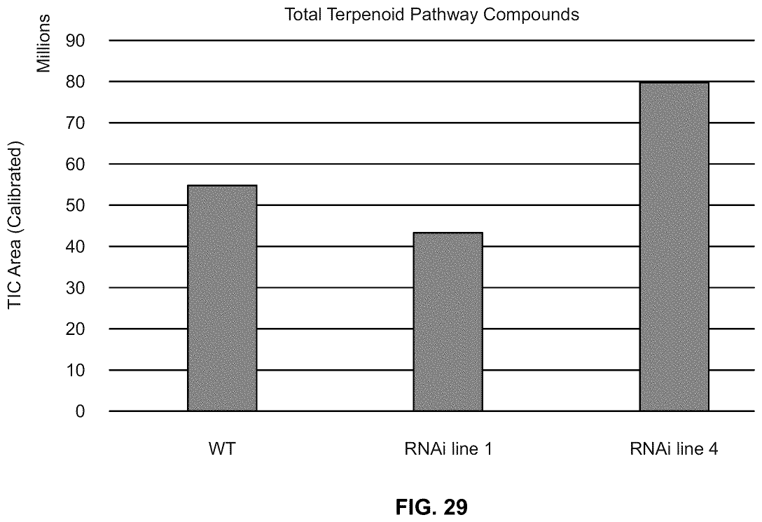

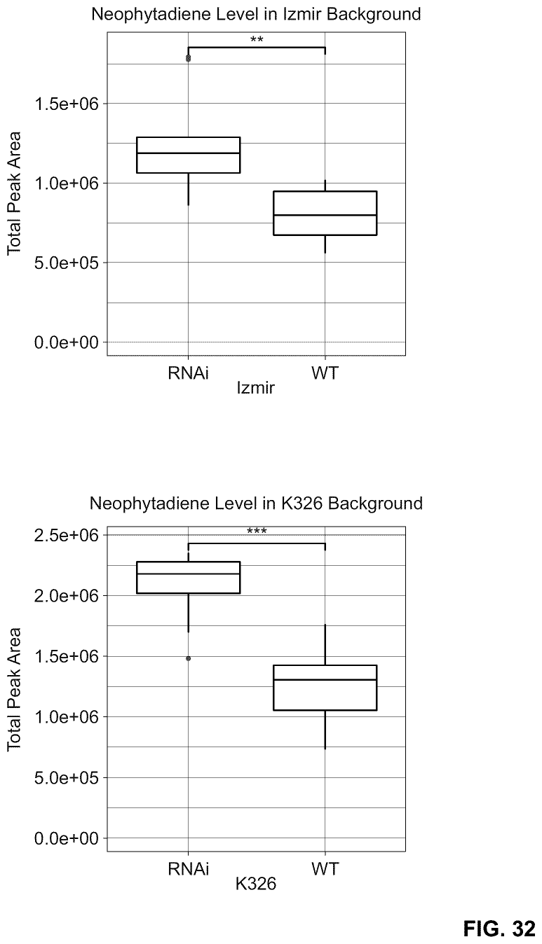

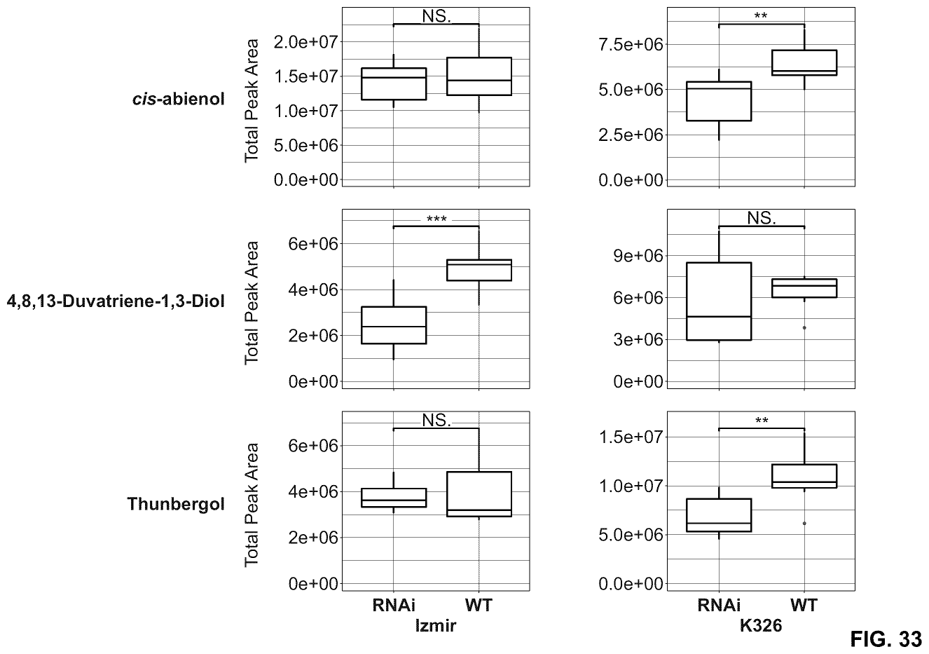

Several edited and RNAi Izmir or K326 tobacco lines provided above are examined for terpenoid composition using gas chromatography-mass spectrometry (GS-MS). Relative quantitative comparisons are made as compared to corresponding wild type plants. See FIG. 27, FIG. 28, FIG. 29, FIG. 32, and FIG. 33. There is a statistically significant decrease in 4,8,13-duvatriene-1,3-diol in both Izmir and K326 RNAi lines as compared to wildtype controls, and a statistically significant increase in neophytadiene in both Izmir and K326 RNAi lines as compared to wildtype controls. There is also a statistically significant decrease in cis-abienol and thunbergol in K326 RNAi lines.

Briefly, to measure terpenoids 0.5 g (+0.01 g) of tissue from the fresh young leaf (fifth or sixth from the top of the plant) is frozen by liquid nitrogen and then ground into fine power with TissueLyser (Qiagen, Hilden, Germany). Terpenoids and other compounds are extracted with 10 mL of hexane:ethyl acetate. As an internal standard, 20 μg of heptadecanol is spiked into each extraction sample. After silica chromatography and filtering, the eluate is dried under a nitrogen stream and resuspend in 400 μL hexane, followed by gas chromatography-mass spectrometry (GC-MS) analysis on a Shimadzu GCMS QP2010 plus system. For each transgenic line, samples are collected from three T1 plant individuals, and three independent extractions are performed for each plant individual. Relative quantification of the compounds is performed by the comparison of the peak areas normalized to the internal standard.

Without being limiting, see Rabara et al., “Identification of Terpene-Related Biosynthetic Gene Clusters in Tobacco through Computational-Based Genomic, Transcriptomic, and Metabolic Analyses,” Agronomy, 13:1632 (2023) for additional information regarding the use of GC-MS to measure terpenoids in tobacco.

1. A modified tobacco plant, or part thereof, comprising a non-natural mutation in an endogenous nucleic acid molecule, wherein the endogenous nucleic acid molecule encodes a HAIRPLUS1a (NtHAP1a) protein comprising the amino acid sequence of SEQ ID NO: 1, and wherein expression or activity of the NtHAP1a protein is reduced in the modified tobacco plant as compared to a control tobacco plant lacking the non-natural mutation when grown under comparable conditions.

2. A modified tobacco plant, or part thereof, comprising a non-natural mutation in an endogenous nucleic acid molecule, wherein the endogenous nucleic acid molecule encodes a HAIRPLUS1b (NtHAP1b) protein comprising the amino acid sequence of SEQ ID NO: 2, and wherein expression or activity of the NtHAP1b protein is reduced in the modified tobacco plant as compared to a control tobacco plant lacking the non-natural mutation when grown under comparable conditions.

3. A modified tobacco plant, or part thereof, comprising a non-natural mutation in an endogenous nucleic acid molecule, wherein the endogenous nucleic acid molecule encodes a HAIRPLUS2 (NtHAP2) protein comprising the amino acid sequence of SEQ ID NO: 3, and wherein expression or activity of the NtHAP2 protein is reduced in the modified tobacco plant as compared to a control tobacco plant lacking the non-natural mutation when grown under comparable conditions.

4. The modified tobacco plant, or part thereof, of claim 1, wherein the endogenous nucleic acid molecule is a coding sequence comprising SEQ ID NO: 4.

5. The modified tobacco plant, or part thereof, of claim 2, wherein the endogenous nucleic acid molecule is a coding sequence comprising SEQ ID NO: 5.

6. The modified tobacco plant, or part thereof, of claim 3, wherein the endogenous nucleic acid molecule is a coding sequence comprising SEQ ID NO: 6.

7. The modified tobacco plant, or part thereof, of claim 1, wherein the endogenous nucleic acid molecule is a genomic sequence comprising SEQ ID NO: 7.

8. The modified tobacco plant, or part thereof, of claim 2, wherein the endogenous nucleic acid molecule is a genomic sequence comprising SEQ ID NO: 8.

9. The modified tobacco plant, or part thereof, of claim 3, wherein the endogenous nucleic acid molecule is a genomic sequence comprising SEQ ID NO: 9.

10. The modified tobacco plant of claim 1, wherein the modified tobacco plant further comprises a second non-natural mutation in an endogenous nucleic acid molecule encoding a HAIRPLUS1b (NtHAP1b) protein, wherein the endogenous NtHAP1b protein comprises the amino acid sequence of SEQ ID NO: 2, and wherein expression or activity of NtHAP1b is reduced in the modified tobacco plant as compared to a control tobacco plant lacking the non-natural mutation when grown under comparable conditions.

11. The modified tobacco plant of claim 10, wherein the modified tobacco plant further comprises a third non-natural mutation in an endogenous nucleic acid molecule encoding a HAIRPLUS2 (NtHAP2) protein, wherein the endogenous NtHAP2 protein comprises the amino acid sequence of SEQ ID NO: 3, and wherein expression or activity of NtHAP2 is reduced in the modified tobacco plant as compared to a control tobacco plant lacking the non-natural mutation when grown under comparable conditions.

12. The modified tobacco plant of claim 1, wherein the modified tobacco plant further comprises a second non-natural mutation in an endogenous nucleic acid molecule encoding a HAIRPLUS2 (NtHAP2) protein, wherein the endogenous NtHAP2 protein comprises the amino acid sequence of SEQ ID NO: 3, and wherein expression or activity of NtHAP2 is reduced in the modified tobacco plant as compared to a control tobacco plant lacking the non-natural mutation when grown under comparable conditions.

13. The modified tobacco plant of claim 2, wherein the modified tobacco plant further comprises a second non-natural mutation in an endogenous nucleic acid molecule encoding a HAIRPLUS2 (NtHAP2) protein, wherein the endogenous NtHAP2 protein comprises the amino acid sequence of SEQ ID NO: 3, and wherein expression or activity of NtHAP2 is reduced in the modified tobacco plant as compared to a control tobacco plant lacking the non-natural mutation when grown under comparable conditions.

14. The modified tobacco plant, or part thereof, of claim 1, wherein the non-natural mutation is a null mutation.

15.-75. (canceled)

76. The modified tobacco plant, or part thereof, of claim 1, wherein the modified tobacco plant is selected from the group consisting of a flue-cured tobacco plant, a bright tobacco plant, a Burley tobacco plant, a Virginia tobacco plant, a Maryland tobacco plant, a dark tobacco plant, a Galpão tobacco plant, an Oriental tobacco plant, and a Turkish tobacco plant.

77. The modified tobacco plant, or part thereof, of claim 1, wherein the modified tobacco plant is selected from the group consisting of the tobacco plants listed in Table 2, Table 3, Table 4, Table 5, Table 6, Table 7, and Table 8.

78.-79. (canceled)

80. The modified tobacco plant, or part thereof, of claim 1, wherein the modified tobacco plant comprises an increased density of trichomes on at least one leaf as compared to the control tobacco plant lacking the non-natural mutation when grown under comparable conditions.

81. The modified tobacco plant, or part thereof, of claim 1, wherein the modified tobacco plant comprises an increased number of trichomes on at least one leaf as compared to the control tobacco plant lacking the non-natural mutation when grown under comparable conditions.

82. The modified tobacco plant, or part thereof, of claim 1, wherein the modified tobacco plant comprises an increased total number of trichomes as compared to the control tobacco plant lacking the non-natural mutation when grown under comparable conditions.

83. A seed obtained from the modified tobacco plant, or part thereof, of any one of claim 1, wherein the seed comprises the non-natural mutation.

84.-152. (canceled)
